SEQUENCE LISTING (70 sequences)

SEQ ID NO: 1 - DNA, 2049 bp, Homo sapiens
atggcgctgtttgtgcgcctgctggcgctggcgctggcgctggcgctgggcccggcggcgaccctggcgggcccggcgaaaagcccgtatcagctggtgctgcagcatagccgcctgcgcggccgccagcatggcccgaacgtgtgcgcggtgcagaaagtgattggcaccaaccgcaaatattttaccaactgcaaacagtggtatcagcgcaaaatttgcggcaaaagcaccgtgattagctatgaatgctgcccgggctatgaaaaagtgccgggcgaaaaaggctgcccggcggcgctgccgctgagcaacctgtatgaaaccctgggcgtggtgggcagcaccaccacccagctgtataccgatcgcaccgaaaaactgcgcccggaaatggaaggcccgggcagctttaccatttttgcgccgagcaacgaagcgtgggcgagcctgccggcggaagtgctggatagcctggtgagcaacgtgaacattgaactgctgaacgcgctgcgctatcatatggtgggccgccgcgtgctgaccgatgaactgaaacatggcatgaccctgaccagcatgtatcagaacagcaacattcagattcatcattatccgaacggcattgtgaccgtgaactgcgcgcgcctgctgaaagcggatcatcatgcgaccaacggcgtggtgcatctgattgataaagtgattagcaccattaccaacaacattcagcagattattgaaattgaagatacctttgaaaccctgcgcgcggcggtggcggcgagcggcctgaacaccatgctggaaggcaacggccagtataccctgctggcgccgaccaacgaagcgtttgaaaaaattccgagcgaaaccctgaaccgcattctgggcgatccggaagcgctgcgcgatctgctgaacaaccatattctgaaaagcgcgatgtgcgcggaagcgattgtggcgggcctgagcgtggaaaccctggaaggcaccaccctggaagtgggctgcagcggcgatatgctgaccattaacggcaaagcgattattagcaacaaagatattctggcgaccaacggcgtgattcattatattgatgaactgctgattccggatagcgcgaaaaccctgtttgaactggcggcggaaagcgatgtgagcaccgcgattgatctgtttcgccaggcgggcctgggcaaccatctgagcggcagcgaacgcctgaccctgctggcgccgctgaacagcgtgtttaaagatggcaccccgccgattgatgcgcatacccgcaacctgctgcgcaaccatattattaaagatcagctggcgagcaaatatctgtatcatggccagaccctggaaaccctgggcggcaaaaaactgcgcgtgtttgtgtatcgcaacagcctgtgcattgaaaacagctgcattgcggcgcatgataaacgcggccgctatggcaccctgtttaccatggatcgcgtgctgaccccgccgatgggcaccgtgatggatgtgctgaaaggcgataaccgctttagcatgctggtggcggcgattcagagcgcgggcctgaccgaaaccctgaaccgcgaaggcgtgtataccgtgtttgcgccgaccaacgaagcgtttcgcgcgctgccgccgcgcgaacgcagccgcctgctgggcgatgcgaaagaactggcgaacattctgaaatatcatattggcgatgaaattctggtgagcggcggcattggcgcgctggtgcgcctgaaaagcctgcagggcgataaactggaagtgagcctgaaaaacaacgtggtgagcgtgaacaaagaaccggtggcggaaccggatattatggcgaccaacggcgtggtgcatgtgattaccaacgtgctgcagccgccggcgaaccgcccgcaggaacgcggcgatgaactggcggatagcgcgctggaaatttttaaacaggcgagcgcgtttagccgcgcgagccagcgcagcgtgcgcctggcgccggtgtatcagaaactgctggaacgcatgaaacat

SEQ ID NO: 2 - protein, 683 aa, Homo sapiens
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 3 - DNA, 954 bp, Homo sapiens
atggcgagcccgggctgcctgctgtgcgtgctgggcctgctgctgtgcggcgcggcgagcctggaactgagccgcccgcatggcgataccgcgaaaaaaccgattattggcattctgatgcagaaatgccgcaacaaagtgatgaaaaactatggccgctattatattgcggcgagctatgtgaaatatctggaaagcgcgggcgcgcgcgtggtgccggtgcgcctggatctgaccgaaaaagattatgaaattctgtttaaaagcattaacggcattctgtttccgggcggcagcgtggatctgcgccgcagcgattatgcgaaagtggcgaaaattttttataacctgagcattcagagctttgatgatggcgattattttccggtgtggggcacctgcctgggctttgaagaactgagcctgctgattagcggcgaatgcctgctgaccgcgaccgataccgtggatgtggcgatgccgctgaactttaccggcggccagctgcatagccgcatgtttcagaactttccgaccgaactgctgctgagcctggcggtggaaccgctgaccgcgaactttcataaatggagcctgagcgtgaaaaactttaccatgaacgaaaaactgaaaaaattttttaacgtgctgaccaccaacaccgatggcaaaattgaatttattagcaccatggaaggctataaatatccggtgtatggcgtgcagtggcatccggaaaaagcgccgtatgaatggaaaaacctggatggcattagccatgcgccgaacgcggtgaaaaccgcgttttatctggcggaattttttgtgaacgaagcgcgcaaaaacaaccatcattttaaaagcgaaagcgaagaagaaaaagcgctgatttatcagtttagcccgatttataccggcaacattagcagctttcagcagtgctatatttttgat

SEQ ID NO: 4 - protein, 318 aa, Homo sapiens
MASPGCLLCVLGLLLCGAASLELSRPHGDTAKKPIIGILMQKCRNKVMKNYGRYYIAASYVKYLESAGARVVPVRLDLTEKDYEILFKSINGILFPGGSVDLRRSDYAKVAKIFYNLSIQSFDDGDYFPVWGTCLGFEELSLLISGECLLTATDTVDVAMPLNFTGGQLHSRMFQNFPTELLLSLAVEPLTANFHKWSLSVKNFTMNEKLKKFFNVLTTNTDGKIEFISTMEGYKYPVYGVQWHPEKAPYEWKNLDGISHAPNAVKTAFYLAEFFVNEARKNNHHFKSESEEEKALIYQFSPIYTGNISSFQQCYIFD

SEQ ID NO: 5 - DNA, 1758 bp, Homo sapiens
atgacccctccgaggctcttctgggtgtggctgctggttgcaggaacccaaggcgtgaacgatggtgacatgcggctggccgatgggggcgccaccaaccagggccgcgtggagatcttctacagaggccagtggggcactgtgtgtgacaacctgtgggacctgactgatgccagcgtcgtctgccgggccctgggcttcgagaacgccacccaggctctgggcagagctgccttcgggcaaggatcaggccccatcatgctggatgaggtccagtgcacgggaaccgaggcctcactggccgactgcaagtccctgggctggctgaagagcaactgcaggcacgagagagacgctggtgtggtctgcaccaatgaaaccaggagcacccacaccctggacctctccagggagctctcggaggcccttggccagatctttgacagccagcggggctgcgacctgtccatcagcgtgaatgtgcagggcgaggacgccctgggcttctgtggccacacggtcatcctgactgccaacctggaggcccaggccctgtggaaggagccgggcagcaatgtcaccatgagtgtggatgctgagtgtgtgcccatggtcagggaccttctcaggtacttctactcccgaaggattgacatcaccctgtcgtcagtcaagtgcttccacaagctggcctctgcctatggggccaggcagctgcagggctactgcgcaagcctctttgccatcctcctcccccaggacccctcgttccagatgcccctggacctgtatgcctatgcagtggccacaggggacgccctgctggagaagctctgcctacagttcctggcctggaacttcgaggccttgacgcaggccgaggcctggcccagtgtccccacagacctgctccaactgctgctgcccaggagcgacctggcggtgcccagcgagctggccctactgaaggccgtggacacctggagctggggggagcgtgcctcccatgaggaggtggagggcttggtggagaagatccgcttccccatgatgctccctgaggagctctttgagctgcagttcaacctgtccctgtactggagccacgaggccctgttccagaagaagactctgcaggccctggaattccacactgtgcccttccagttgctggcccggtacaaaggcctgaacctcaccgaggatacctacaagccccggatttacacctcgcccacctggagtgcctttgtgacagacagttcctggagtgcacggaagtcacaactggtctatcagtccagacgggggcctttggtcaaatattcttctgattacttccaagccccctctgactacagatactacccctaccagtccttccagactccacaacaccccagcttcctcttccaggacaagagggtgtcctggtccctggtctacctccccaccatccagagctgctggaactacggcttctcctgctcctcggacgagctccctgtcctgggcctcaccaagtctggcggctcagatcgcaccattgcctacgaaaacaaagccctgatgctctgcgaagggctcttcgtggcagacgtcaccgatttcgagggctggaaggctgcgattcccagtgccctggacaccaacagctcgaagagcacctcctccttcccctgcccggcagggcacttcaacggcttccgcacggtcatccgccccttctacctgaccaactcctcaggtgtggactag

SEQ ID NO: 6 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 7 - DNA, 597 bp, Homo sapiens
atgagcagcggcaacgcgaaaattggccatccggcgccgaactttaaagcgaccgcggtgatgccggatggccagtttaaagatattagcctgagcgattataaaggcaaatatgtggtgttttttttttatccgctggattttacctttgtgtgcccgaccgaaattattgcgtttagcgatcgcgcggaagaatttaaaaaactgaactgccaggtgattggcgcgagcgtggatagccatttttgccatctggcgtgggtgaacaccccgaaaaaacagggcggcctgggcccgatgaacattccgctggtgagcgatccgaaacgcaccattgcgcaggattatggcgtgctgaaagcggatgaaggcattagctttcgcggcctgtttattattgatgataaaggcattctgcgccagattaccgtgaacgatctgccggtgggccgcagcgtggatgaaaccctgcgcctggtgcaggcgtttcagtttaccgataaacatggcgaagtgtgcccggcgggctggaaaccgggcagcgataccattaaaccggatgtgcagaaaagcaaagaatattttagcaaacagaaa

SEQ ID NO: 8 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 9 - DNA, 3513 bp, Homo sapiens
atggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaa

SEQ ID NO: 10 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 11 - DNA, 2226 bp, Homo sapiens
atggataaattttggtggcatgcggcgtggggcctgtgcctggtgccgctgagcctggcgcagattgatctgaacattacctgccgctttgcgggcgtgtttcatgtggaaaaaaacggccgctatagcattagccgcaccgaagcggcggatctgtgcaaagcgtttaacagcaccctgccgaccatggcgcagatggaaaaagcgctgagcattggctttgaaacctgccgctatggctttattgaaggccatgtggtgattccgcgcattcatccgaacagcatttgcgcggcgaacaacaccggcgtgtatattctgaccagcaacaccagccagtatgatacctattgctttaacgcgagcgcgccgccggaagaagattgcaccagcgtgaccgatctgccgaacgcgtttgatggcccgattaccattaccattgtgaaccgcgatggcacccgctatgtgcagaaaggcgaatatcgcaccaacccggaagatatttatccgagcaacccgaccgatgatgatgtgagcagcggcagcagcagcgaacgcagcagcaccagcggcggctatattttttatacctttagcaccgtgcatccgattccggatgaagatagcccgtggattaccgatagcaccgatcgcattccggcgaccaccctgatgagcaccagcgcgaccgcgaccgaaaccgcgaccaaacgccaggaaacctgggattggtttagctggctgtttctgccgagcgaaagcaaaaaccatctgcataccaccacccagatggcgggcaccagcagcaacaccattagcgcgggctgggaaccgaacgaagaaaacgaagatgaacgcgatcgccatctgagctttagcggcagcggcattgatgatgatgaagattttattagcagcaccattagcaccaccccgcgcgcgtttgatcataccaaacagaaccaggattggacccagtggaacccgagccatagcaacccggaagtgctgctgcagaccaccacccgcatgaccgatgtggatcgcaacggcaccaccgcgtatgaaggcaactggaacccggaagcgcatccgccgctgattcatcatgaacatcatgaagaagaagaaaccccgcatagcaccagcaccattcaggcgaccccgagcagcaccaccgaagaaaccgcgacccagaaagaacagtggtttggcaaccgctggcatgaaggctatcgccagaccccgaaagaagatagccatagcaccaccggcaccgcggcggcgagcgcgcataccagccatccgatgcagggccgcaccaccccgagcccggaagatagcagctggaccgatttttttaacccgattagccatccgatgggccgcggccatcaggcgggccgccgcatggatatggatagcagccatagcattaccctgcagccgaccgcgaacccgaacaccggcctggtggaagatctggatcgcaccggcccgctgagcatgaccacccagcagagcaacagccagagctttagcaccagccatgaaggcctggaagaagataaagatcatccgaccaccagcaccctgaccagcagcaaccgcaacgatgtgaccggcggccgccgcgatccgaaccatagcgaaggcagcaccaccctgctggaaggctataccagccattatccgcataccaaagaaagccgcacctttattccggtgaccagcgcgaaaaccggcagctttggcgtgaccgcggtgaccgtgggcgatagcaacagcaacgtgaaccgcagcctgagcggcgatcaggatacctttcatccgagcggcggcagccataccacccatggcagcgaaagcgatggccatagccatggcagccaggaaggcggcgcgaacaccaccagcggcccgattcgcaccccgcagattccggaatggctgattattctggcgagcctgctggcgctggcgctgattctggcggtgtgcattgcggtgaacagccgccgccgctgcggccagaaaaaaaaactggtgattaacagcggcaacggcgcggtggaagatcgcaaaccgagcggcctgaacggcgaagcgagcaaaagccaggaaatggtgcatctggtgaacaaagaaagcagcgaaaccccggatcagtttatgaccgcggatgaaacccgcaacctgcagaacgtggatatgaaaattggcgtg

SEQ ID NO: 12 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 13 - DNA, 2409 bp, Homo sapiens
atgcgcgcgctgtgggtgctgggcctgtgctgcgtgctgctgacctttggcagcgtgcgcgcggatgatgaagtggatgtggatggcaccgtggaagaagatctgggcaaaagccgcgaaggcagccgcaccgatgatgaagtggtgcagcgcgaagaagaagcgattcagctggatggcctgaacgcgagccagattcgcgaactgcgcgaaaaaagcgaaaaatttgcgtttcaggcggaagtgaaccgcatgatgaaactgattattaacagcctgtataaaaacaaagaaatttttctgcgcgaactgattagcaacgcgagcgatgcgctggataaaattcgcctgattagcctgaccgatgaaaacgcgctgagcggcaacgaagaactgaccgtgaaaattaaatgcgataaagaaaaaaacctgctgcatgtgaccgataccggcgtgggcatgacccgcgaagaactggtgaaaaacctgggcaccattgcgaaaagcggcaccagcgaatttctgaacaaaatgaccgaagcgcaggaagatggccagagcaccagcgaactgattggccagtttggcgtgggcttttatagcgcgtttctggtggcggataaagtgattgtgaccagcaaacataacaacgatacccagcatatttgggaaagcgatagcaacgaatttagcgtgattgcggatccgcgcggcaacaccctgggccgcggcaccaccattaccctggtgctgaaagaagaagcgagcgattatctggaactggataccattaaaaacctggtgaaaaaatatagccagtttattaactttccgatttatgtgtggagcagcaaaaccgaaaccgtggaagaaccgatggaagaagaagaagcggcgaaagaagaaaaagaagaaagcgatgatgaagcggcggtggaagaagaagaagaagaaaaaaaaccgaaaaccaaaaaagtggaaaaaaccgtgtgggattgggaactgatgaacgatattaaaccgatttggcagcgcccgagcaaagaagtggaagaagatgaatataaagcgttttataaaagctttagcaaagaaagcgatgatccgatggcgtatattcattttaccgcggaaggcgaagtgacctttaaaagcattctgtttgtgccgaccagcgcgccgcgcggcctgtttgatgaatatggcagcaaaaaaagcgattatattaaactgtatgtgcgccgcgtgtttattaccgatgattttcatgatatgatgccgaaatatctgaactttgtgaaaggcgtggtggatagcgatgatctgccgctgaacgtgagccgcgaaaccctgcagcagcataaactgctgaaagtgattcgcaaaaaactggtgcgcaaaaccctggatatgattaaaaaaattgcggatgataaatataacgataccttttggaaagaatttggcaccaacattaaactgggcgtgattgaagatcatagcaaccgcacccgcctggcgaaactgctgcgctttcagagcagccatcatccgaccgatattaccagcctggatcagtatgtggaacgcatgaaagaaaaacaggataaaatttattttatggcgggcagcagccgcaaagaagcggaaagcagcccgtttgtggaacgcctgctgaaaaaaggctatgaagtgatttatctgaccgaaccggtggatgaatattgcattcaggcgctgccggaatttgatggcaaacgctttcagaacgtggcgaaagaaggcgtgaaatttgatgaaagcgaaaaaaccaaagaaagccgcgaagcggtggaaaaagaatttgaaccgctgctgaactggatgaaagataaagcgctgaaagataaaattgaaaaagcggtggtgagccagcgcctgaccgaaagcccgtgcgcgctggtggcgagccagtatggctggagcggcaacatggaacgcattatgaaagcgcaggcgtatcagaccggcaaagatattagcaccaactattatgcgagccagaaaaaaacctttgaaattaacccgcgccatccgctgattcgcgatatgctgcgccgcattaaagaagatgaagatgataaaaccgtgctggatctggcggtggtgctgtttgaaaccgcgaccctgcgcagcggctatctgctgccggataccaaagcgtatggcgatcgcattgaacgcatgctgcgcctgagcctgaacattgatccggatgcgaaagtggaagaagaaccggaagaagaaccggaagaaaccgcggaagataccaccgaagataccgaacaggatgaagatgaagaaatggatgtgggcaccgatgaagaagaagaaaccgcgaaagaaagcaccgcggaaaaagatgaactg

SEQ ID NO: 14 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 15 - DNA, 12867 bp, Homo sapiens
atgatgccggcgcagtatgcgctgaccagcagcctggtgctgctggtgctgctgagcaccgcgcgcgcgggcccgtttagcagccgcagcaacgtgaccctgccggcgccgcgcccgccgccgcagccgggcggccataccgtgggcgcgggcgtgggcagcccgagcagccagctgtatgaacataccgtggaaggcggcgaaaaacaggtggtgtttacccatcgcattaacctgccgccgagcaccggctgcggctgcccgccgggcaccgaaccgccggtgctggcgagcgaagtgcaggcgctgcgcgtgcgcctggaaattctggaagaactggtgaaaggcctgaaagaacagtgcaccggcggctgctgcccggcgagcgcgcaggcgggcaccggccagaccgatgtgcgcaccctgtgcagcctgcatggcgtgtttgatctgagccgctgcacctgcagctgcgaaccgggctggggcggcccgacctgcagcgatccgaccgatgcggaaattccgccgagcagcccgccgagcgcgagcggcagctgcccggatgattgcaacgatcagggccgctgcgtgcgcggccgctgcgtgtgctttccgggctataccggcccgagctgcggctggccgagctgcccgggcgattgccagggccgcggccgctgcgtgcagggcgtgtgcgtgtgccgcgcgggctttagcggcccggattgcagccagcgcagctgcccgcgcggctgcagccagcgcggccgctgcgaaggcggccgctgcgtgtgcgatccgggctataccggcgatgattgcggcatgcgcagctgcccgcgcggctgcagccagcgcggccgctgcgaaaacggccgctgcgtgtgcaacccgggctataccggcgaagattgcggcgtgcgcagctgcccgcgcggctgcagccagcgcggccgctgcaaagatggccgctgcgtgtgcgatccgggctataccggcgaagattgcggcacccgcagctgcccgtgggattgcggcgaaggcggccgctgcgtggatggccgctgcgtgtgctggccgggctataccggcgaagattgcagcacccgcacctgcccgcgcgattgccgcggccgcggccgctgcgaagatggcgaatgcatttgcgataccggctatagcggcgatgattgcggcgtgcgcagctgcccgggcgattgcaaccagcgcggccgctgcgaagatggccgctgcgtgtgctggccgggctataccggcaccgattgcggcagccgcgcgtgcccgcgcgattgccgcggccgcggccgctgcgaaaacggcgtgtgcgtgtgcaacgcgggctatagcggcgaagattgcggcgtgcgcagctgcccgggcgattgccgcggccgcggccgctgcgaaagcggccgctgcatgtgctggccgggctataccggccgcgattgcggcacccgcgcgtgcccgggcgattgccgcggccgcggccgctgcgtggatggccgctgcgtgtgcaacccgggctttaccggcgaagattgcggcagccgccgctgcccgggcgattgccgcggccatggcctgtgcgaagatggcgtgtgcgtgtgcgatgcgggctatagcggcgaagattgcagcacccgcagctgcccgggcggctgccgcggccgcggccagtgcctggatggccgctgcgtgtgcgaagatggctatagcggcgaagattgcggcgtgcgccagtgcccgaacgattgcagccagcatggcgtgtgccaggatggcgtgtgcatttgctgggaaggctatgtgagcgaagattgcagcattcgcacctgcccgagcaactgccatggccgcggccgctgcgaagaaggccgctgcctgtgcgatccgggctataccggcccgacctgcgcgacccgcatgtgcccggcggattgccgcggccgcggccgctgcgtgcagggcgtgtgcctgtgccatgtgggctatggcggcgaagattgcggccaggaagaaccgccggcgagcgcgtgcccgggcggctgcggcccgcgcgaactgtgccgcgcgggccagtgcgtgtgcgtggaaggctttcgcggcccggattgcgcgattcagacctgcccgggcgattgccgcggccgcggcgaatgccatgatggcagctgcgtgtgcaaagatggctatgcgggcgaagattgcggcgaagcgcgcgtgccgagcagcgcgagcgcgtatgatcagcgcggcctggcgccgggccaggaatatcaggtgaccgtgcgcgcgctgcgcggcaccagctggggcctgccggcgagcaaaaccattaccaccatgattgatggcccgcaggatctgcgcgtggtggcggtgaccccgaccaccctggaactgggctggctgcgcccgcaggcggaagtggatcgctttgtggtgagctatgtgagcgcgggcaaccagcgcgtgcgcctggaagtgccgccggaagcggatggcaccctgctgaccgatctgatgccgggcgtggaatatgtggtgaccgtgaccgcggaacgcggccgcgcggtgagctatccggcgagcgtgcgcgcgaacaccgaagaacgcgaagaagaaagcccgccgcgcccgagcctgagccagccgccgcgccgcccgtggggcaacctgaccgcggaactgagccgctttcgcggcaccgtgcaggatctggaacgccatctgcgcgcgcatggctatccgctgcgcgcgaaccagacctataccagcgtggcgcgccatattcatgaatatctgcagcgccaggtgctgggcagcagcgcggatggcgcgctgctggtgagcctggatggcctgcgcggccagtttgaacgcgtggtgctgcgctggcgcccgcagccgccggcggaaggcccgggcggcgaactgaccgtgccgggcaccacccgcaccgtgagcctgccggatctgcgcccgggcaccacctatcatgtggaagtgcatggcgtgcgcgcgggccagaccagcaaaagctatgcgtttattaccaccaccggcccgagcaccacccagggcgcgcaggcgccgctgctgcagcagcgcccgcaggaactgggcgaactgcgcgtgctgggccgcgatgaaaccggccgcctgcgcgtggtgtggaccgcgcagccggatacctttgcgtattttcagctgcgcatgcgcgtgccggaaggcccgggcgcgcatgaagaagtgctgccgggcgatgtgcgccaggcgctggtgccgccgccgccgccgggcaccccgtatgaactgagcctgcatggcgtgccgccgggcggcaaaccgagcgatccgattatttatcagggcattatggataaagatgaagaaaaaccgggcaaaagcagcggcccgccgcgcctgggcgaactgaccgtgaccgatcgcaccagcgatagcctgctgctgcgctggaccgtgccggaaggcgaatttgatagctttgtgattcagtataaagatcgcgatggccagccgcaggtggtgccggtggaaggcccgcagcgcagcgcggtgattaccagcctggatccgggccgcaaatataaatttgtgctgtatggctttgtgggcaaaaaacgccatggcccgctggtggcggaagcgaaaattctgccgcagagcgatccgagcccgggcaccccgccgcatctgggcaacctgtgggtgaccgatccgaccccggatagcctgcatctgagctggaccgtgccggaaggccagtttgatacctttatggtgcagtatcgcgatcgcgatggccgcccgcaggtggtgccggtggaaggcccggaacgcagctttgtggtgagcagcctggatccggatcataaatatcgctttaccctgtttggcattgcgaacaaaaaacgctatggcccgctgaccgcggatggcaccaccgcgccggaacgcaaagaagaaccgccgcgcccggaatttctggaacagccgctgctgggcgaactgaccgtgaccggcgtgaccccggatagcctgcgcctgagctggaccgtggcgcagggcccgtttgatagctttatggtgcagtataaagatgcgcagggccagccgcaggcggtgccggtggcgggcgatgaaaacgaagtgaccgtgccgggcctggatccggatcgcaaatataaaatgaacctgtatggcctgcgcggccgccagcgcgtgggcccggaaagcgtggtggcgaaaaccgcgccgcaggaagatgtggatgaaaccccgagcccgaccgaactgggcaccgaagcgccggaaagcccggaagaaccgctgctgggcgaactgaccgtgaccggcagcagcccggatagcctgagcctgttttggaccgtgccgcagggcagctttgatagctttaccgtgcagtataaagatcgcgatggccgcccgcgcgcggtgcgcgtgggcggcaaagaaagcgaagtgaccgtgggcggcctggaaccgggccataaatataaaatgcatctgtatggcctgcatgaaggccagcgcgtgggcccggtgagcgcggtgggcgtgaccgcgccgcagcaggaagaaaccccgccggcgaccgaaagcccgctggaaccgcgcctgggcgaactgaccgtgaccgatgtgaccccgaacagcgtgggcctgagctggaccgtgccggaaggccagtttgatagctttattgtgcagtataaagataaagatggccagccgcaggtggtgccggtggcggcggatcagcgcgaagtgaccgtgtataacctggaaccggaacgcaaatataaaatgaacatgtatggcctgcatgatggccagcgcatgggcccgctgagcgtggtgattgtgaccgcgccggcgaccgaagcgagcaaaccgccgctggaaccgcgcctgggcgaactgaccgtgaccgatattaccccggatagcgtgggcctgagctggaccgtgccggaaggcgaatttgatagctttgtggtgcagtataaagatcgcgatggccagccgcaggtggtgccggtggcggcggatcagcgcgaagtgaccattccggatctggaaccgagccgcaaatataaatttctgctgtttggcattcaggatggcaaacgccgcagcccggtgagcgtggaagcgaaaaccgtggcgcgcggcgatgcgagcccgggcgcgccgccgcgcctgggcgaactgtgggtgaccgatccgaccccggatagcctgcgcctgagctggaccgtgccggaaggccagtttgatagctttgtggtgcagtttaaagataaagatggcccgcaggtggtgccggtggaaggccatgaacgcagcgtgaccgtgaccccgctggatgcgggccgcaaatatcgctttctgctgtatggcctgctgggcaaaaaacgccatggcccgctgaccgcggatggcaccaccgaagcgcgcagcgcgatggatgataccggcaccaaacgcccgccgaaaccgcgcctgggcgaagaactgcaggtgaccaccgtgacccagaacagcgtgggcctgagctggaccgtgccggaaggccagtttgatagctttgtggtgcagtataaagatcgcgatggccagccgcaggtggtgccggtggaaggcagcctgcgcgaagtgagcgtgccgggcctggatccggcgcatcgctataaactgctgctgtatggcctgcatcatggcaaacgcgtgggcccgattagcgcggtggcgattaccgcgggccgcgaagaaaccgaaaccgaaaccaccgcgccgaccccgccggcgccggaaccgcatctgggcgaactgaccgtggaagaagcgaccagccataccctgcatctgagctggatggtgaccgaaggcgaatttgatagctttgaaattcagtataccgatcgcgatggccagctgcagatggtgcgcattggcggcgatcgcaacgatattaccctgagcggcctggaaagcgatcatcgctatctggtgaccctgtatggctttagcgatggcaaacatgtgggcccggtgcatgtggaagcgctgaccgtgccggaagaagaaaaaccgagcgaaccgccgaccgcgaccccggaaccgccgattaaaccgcgcctgggcgaactgaccgtgaccgatgcgaccccggatagcctgagcctgagctggaccgtgccggaaggccagtttgatcattttctggtgcagtatcgcaacggcgatggccagccgaaagcggtgcgcgtgccgggccatgaagaaggcgtgaccattagcggcctggaaccggatcataaatataaaatgaacctgtatggctttcatggcggccagcgcatgggcccggtgagcgtggtgggcgtgaccgaaccgagcatggaagcgccggaaccggcggaagaaccgctgctgggcgaactgaccgtgaccggcagcagcccggatagcctgagcctgagctggaccgtgccgcagggccgctttgatagctttaccgtgcagtataaagatcgcgatggccgcccgcaggtggtgcgcgtgggcggcgaagaaagcgaagtgaccgtgggcggcctggaaccgggccgcaaatataaaatgcatctgtatggcctgcatgaaggccgccgcgtgggcccggtgagcgcggtgggcgtgaccgcgccggaagaagaaagcccggatgcgccgctggcgaaactgcgcctgggccagatgaccgtgcgcgatattaccagcgatagcctgagcctgagctggaccgtgccggaaggccagtttgatcattttctggtgcagtttaaaaacggcgatggccagccgaaagcggtgcgcgtgccgggccatgaagatggcgtgaccattagcggcctggaaccggatcataaatataaaatgaacctgtatggctttcatggcggccagcgcgtgggcccggtgagcgcggtgggcctgaccgcgagcaccgaaccgccgaccccggaaccgccgattaaaccgcgcctggaagaactgaccgtgaccgatgcgaccccggatagcctgagcctgagctggaccgtgccggaaggccagtttgatcattttctggtgcagtataaaaacggcgatggccagccgaaagcgacccgcgtgccgggccatgaagatcgcgtgaccattagcggcctggaaccggataacaaatataaaatgaacctgtatggctttcatggcggccagcgcgtgggcccggtgagcgcgattggcgtgaccgaagaagaaaccccgagcccgaccgaaccgagcatggaagcgccggaaccgccggaagaaccgctgctgggcgaactgaccgtgaccggcagcagcccggatagcctgagcctgagctggaccgtgccgcagggccgctttgatagctttaccgtgcagtataaagatcgcgatggccgcccgcaggtggtgcgcgtgggcggcgaagaaagcgaagtgaccgtgggcggcctggaaccgggccgcaaatataaaatgcatctgtatggcctgcatgaaggccgccgcgtgggcccggtgagcaccgtgggcgtgaccgcgccgcaggaagatgtggatgaaaccccgagcccgaccgaaccgggcaccgaagcgccgggcccgccggaagaaccgctgctgggcgaactgaccgtgaccggcagcagcccggatagcctgagcctgagctggaccgtgccgcagggccgctttgatagctttaccgtgcagtataaagatcgcgatggccgcccgcaggcggtgcgcgtgggcggccaggaaagcaaagtgaccgtgcgcggcctggaaccgggccgcaaatataaaatgcatctgtatggcctgcatgaaggccgccgcctgggcccggtgagcgcggtgggcgtgaccgaagatgaagcggaaaccacccaggcggtgccgaccatgaccccggaaccgccgattaaaccgcgcctgggcgaactgaccatgaccgatgcgaccccggatagcctgagcctgagctggaccgtgccggaaggccagtttgatcattttctggtgcagtatcgcaacggcgatggccagccgaaagcggtgcgcgtgccgggccatgaagatggcgtgaccattagcggcctggaaccggatcataaatataaaatgaacctgtatggctttcatggcggccagcgcgtgggcccgattagcgtgattggcgtgaccgaagaagaaaccccgagcccgaccgaactgagcaccgaagcgccggaaccgccggaagaaccgctgctgggcgaactgaccgtgaccggcagcagcccggatagcctgagcctgagctggaccattccgcagggccattttgatagctttaccgtgcagtataaagatcgcgatggccgcccgcaggtgatgcgcgtgcgcggcgaagaaagcgaagtgaccgtgggcggcctggaaccgggccgcaaatataaaatgcatctgtatggcctgcatgaaggccgccgcgtgggcccggtgagcaccgtgggcgtgaccgtgccgaccaccaccccggaaccgccgaacaaaccgcgcctgggcgaactgaccgtgaccgatgcgaccccggatagcctgagcctgagctggatggtgccggaaggccagtttgatcattttctggtgcagtatcgcaacggcgatggccagccgaaagtggtgcgcgtgccgggccatgaagatggcgtgaccattagcggcctggaaccggatcataaatataaaatgaacctgtatggctttcatggcggccagcgcgtgggcccgattagcgtgattggcgtgaccgaagaagaaaccccggcgccgaccgaaccgagcaccgaagcgccggaaccgccggaagaaccgctgctgggcgaactgaccgtgaccggcagcagcccggatagcctgagcctgagctggaccattccgcagggccgctttgatagctttaccgtgcagtataaagatcgcgatggccgcccgcaggtggtgcgcgtgcgcggcgaagaaagcgaagtgaccgtgggcggcctggaaccgggctgcaaatataaaatgcatctgtatggcctgcatgaaggccagcgcgtgggcccggtgagcgcggtgggcgtgaccgcgccgaaagatgaagcggaaaccacccaggcggtgccgaccatgaccccggaaccgccgattaaaccgcgcctgggcgaactgaccgtgaccgatgcgaccccggatagcctgagcctgagctggatggtgccggaaggccagtttgatcattttctggtgcagtatcgcaacggcgatggccagccgaaagcggtgcgcgtgccgggccatgaagatggcgtgaccattagcggcctggaaccggatcataaatataaaatgaacctgtatggctttcatggcggccagcgcgtgggcccggtgagcgcgattggcgtgaccgaagaagaaaccccgagcccgaccgaaccgagcaccgaagcgccggaagcgccggaagaaccgctgctgggcgaactgaccgtgaccggcagcagcccggatagcctgagcctgagctggaccgtgccgcagggccgctttgatagctttaccgtgcagtataaagatcgcgatggccagccgcaggtggtgcgcgtgcgcggcgaagaaagcgaagtgaccgtgggcggcctggaaccgggccgcaaatataaaatgcatctgtatggcctgcatgaaggccagcgcgtgggcccggtgagcaccgtgggcattaccgcgccgctgccgaccccgctgccggtggaaccgcgcctgggcgaactggcggtggcggcggtgaccagcgatagcgtgggcctgagctggaccgtggcgcagggcccgtttgatagctttctggtgcagtatcgcgatgcgcagggccagccgcaggcggtgccggtgagcggcgatctgcgcgcggtggcggtgagcggcctggatccggcgcgcaaatataaatttctgctgtttggcctgcagaacggcaaacgccatggcccggtgccggtggaagcgcgcaccgcgccggataccaaaccgagcccgcgcctgggcgaactgaccgtgaccgatgcgaccccggatagcgtgggcctgagctggaccgtgccggaaggcgaatttgatagctttgtggtgcagtataaagataaagatggccgcctgcaggtggtgccggtggcggcgaaccagcgcgaagtgaccgtgcagggcctggaaccgagccgcaaatatcgctttctgctgtatggcctgagcggccgcaaacgcctgggcccgattagcgcggatagcaccaccgcgccgctggaaaaagaactgccgccgcatctgggcgaactgaccgtggcggaagaaaccagcagcagcctgcgcctgagctggaccgtggcgcagggcccgtttgatagctttgtggtgcagtatcgcgataccgatggccagccgcgcgcggtgccggtggcggcggatcagcgcaccgtgaccgtggaagatctggaaccgggcaaaaaatataaatttctgctgtatggcctgctgggcggcaaacgcctgggcccggtgagcgcgctgggcatgaccgcgccggaagaagataccccggcgccggaactggcgccggaagcgccggaaccgccggaagaaccgcgcctgggcgtgctgaccgtgaccgataccaccccggatagcatgcgcctgagctggagcgtggcgcagggcccgtttgatagctttgtggtgcagtatgaagataccaacggccagccgcaggcgctgctggtggatggcgatcagagcaaaattctgattagcggcctggaaccgagcaccccgtatcgctttctgctgtatggcctgcatgaaggcaaacgcctgggcccgctgagcgcggaaggcaccaccggcctggcgccggcgggccagaccagcgaagaaagccgcccgcgcctgagccagctgagcgtgaccgatgtgaccaccagcagcctgcgcctgaactgggaagcgccgccgggcgcgtttgatagctttctgctgcgctttggcgtgccgagcccgagcaccctggaaccgcatccgcgcccgctgctgcagcgcgaactgatggtgccgggcacccgccatagcgcggtgctgcgcgatctgcgcagcggcaccctgtatagcctgaccctgtatggcctgcgcggcccgcataaagcggatagcattcagggcaccgcgcgcaccctgagcccggtgctggaaagcccgcgcgatctgcagtttagcgaaattcgcgaaaccagcgcgaaagtgaactggatgccgccgccgagccgcgcggatagctttaaagtgagctatcagctggcggatggcggcgaaccgcagagcgtgcaggtggatggccaggcgcgcacccagaaactgcagggcctgattccgggcgcgcgctatgaagtgaccgtggtgagcgtgcgcggctttgaagaaagcgaaccgctgaccggctttctgaccaccgtgccggatggcccgacccagctgcgcgcgctgaacctgaccgaaggctttgcggtgctgcattggaaaccgccgcagaacccggtggatacctatgatgtgcaggtgaccgcgccgggcgcgccgccgctgcaggcggaaaccccgggcagcgcggtggattatccgctgcatgatctggtgctgcataccaactataccgcgaccgtgcgcggcctgcgcggcccgaacctgaccagcccggcgagcattacctttaccaccggcctggaagcgccgcgcgatctggaagcgaaagaagtgaccccgcgcaccgcgctgctgacctggaccgaaccgccggtgcgcccggcgggctatctgctgagctttcataccccgggcggccagaaccaggaaattctgctgccgggcggcattaccagccatcagctgctgggcctgtttccgagcaccagctataacgcgcgcctgcaggcgatgtggggccagagcctgctgccgccggtgagcaccagctttaccaccggcggcctgcgcattccgtttccgcgcgattgcggcgaagaaatgcagaacggcgcgggcgcgagccgcaccagcaccatttttctgaacggcaaccgcgaacgcccgctgaacgtgttttgcgatatggaaaccgatggcggcggctggctggtgtttcagcgccgcatggatggccagaccgatttttggcgcgattgggaagattatgcgcatggctttggcaacattagcggcgaattttggctgggcaacgaagcgctgcatagcctgacccaggcgggcgattatagcatgcgcgtggatctgcgcgcgggcgatgaagcggtgtttgcgcagtatgatagctttcatgtggatagcgcggcggaatattatcgcctgcatctggaaggctatcatggcaccgcgggcgatagcatgagctatcatagcggcagcgtgtttagcgcgcgcgatcgcgatccgaacagcctgctgattagctgcgcggtgagctatcgcggcgcgtggtggtatcgcaactgccattatgcgaacctgaacggcctgtatggcagcaccgtggatcatcagggcgtgagctggtatcattggaaaggctttgaatttagcgtgccgtttaccgaaatgaaactgcgcccgcgcaactttcgcagcccggcgggcggcggc

SEQ ID NO: 16 - protein, 4289 aa, Homo sapiens
MMPAQYALTSSLVLLVLLSTARAGPFSSRSNVTLPAPRPPPQPGGHTVGAGVGSPSSQLYEHTVEGGEKQVVFTHRINLPPSTGCGCPPGTEPPVLASEVQALRVRLEILEELVKGLKEQCTGGCCPASAQAGTGQTDVRTLCSLHGVFDLSRCTCSCEPGWGGPTCSDPTDAEIPPSSPPSASGSCPDDCNDQGRCVRGRCVCFPGYTGPSCGWPSCPGDCQGRGRCVQGVCVCRAGFSGPDCSQRSCPRGCSQRGRCEGGRCVCDPGYTGDDCGMRSCPRGCSQRGRCENGRCVCNPGYTGEDCGVRSCPRGCSQRGRCKDGRCVCDPGYTGEDCGTRSCPWDCGEGGRCVDGRCVCWPGYTGEDCSTRTCPRDCRGRGRCEDGECICDTGYSGDDCGVRSCPGDCNQRGRCEDGRCVCWPGYTGTDCGSRACPRDCRGRGRCENGVCVCNAGYSGEDCGVRSCPGDCRGRGRCESGRCMCWPGYTGRDCGTRACPGDCRGRGRCVDGRCVCNPGFTGEDCGSRRCPGDCRGHGLCEDGVCVCDAGYSGEDCSTRSCPGGCRGRGQCLDGRCVCEDGYSGEDCGVRQCPNDCSQHGVCQDGVCICWEGYVSEDCSIRTCPSNCHGRGRCEEGRCLCDPGYTGPTCATRMCPADCRGRGRCVQGVCLCHVGYGGEDCGQEEPPASACPGGCGPRELCRAGQCVCVEGFRGPDCAIQTCPGDCRGRGECHDGSCVCKDGYAGEDCGEARVPSSASAYDQRGLAPGQEYQVTVRALRGTSWGLPASKTITTMIDGPQDLRVVAVTPTTLELGWLRPQAEVDRFVVSYVSAGNQRVRLEVPPEADGTLLTDLMPGVEYVVTVTAERGRAVSYPASVRANTEEREEESPPRPSLSQPPRRPWGNLTAELSRFRGTVQDLERHLRAHGYPLRANQTYTSVARHIHEYLQRQVLGSSADGALLVSLDGLRGQFERVVLRWRPQPPAEGPGGELTVPGTTRTVSLPDLRPGTTYHVEVHGVRAGQTSKSYAFITTTGPSTTQGAQAPLLQQRPQELGELRVLGRDETGRLRVVWTAQPDTFAYFQLRMRVPEGPGAHEEVLPGDVRQALVPPPPPGTPYELSLHGVPPGGKPSDPIIYQGIMDKDEEKPGKSSGPPRLGELTVTDRTSDSLLLRWTVPEGEFDSFVIQYKDRDGQPQVVPVEGPQRSAVITSLDPGRKYKFVLYGFVGKKRHGPLVAEAKILPQSDPSPGTPPHLGNLWVTDPTPDSLHLSWTVPEGQFDTFMVQYRDRDGRPQVVPVEGPERSFVVSSLDPDHKYRFTLFGIANKKRYGPLTADGTTAPERKEEPPRPEFLEQPLLGELTVTGVTPDSLRLSWTVAQGPFDSFMVQYKDAQGQPQAVPVAGDENEVTVPGLDPDRKYKMNLYGLRGRQRVGPESVVAKTAPQEDVDETPSPTELGTEAPESPEEPLLGELTVTGSSPDSLSLFWTVPQGSFDSFTVQYKDRDGRPRAVRVGGKESEVTVGGLEPGHKYKMHLYGLHEGQRVGPVSAVGVTAPQQEETPPATESPLEPRLGELTVTDVTPNSVGLSWTVPEGQFDSFIVQYKDKDGQPQVVPVAADQREVTVYNLEPERKYKMNMYGLHDGQRMGPLSVVIVTAPATEASKPPLEPRLGELTVTDITPDSVGLSWTVPEGEFDSFVVQYKDRDGQPQVVPVAADQREVTIPDLEPSRKYKFLLFGIQDGKRRSPVSVEAKTVARGDASPGAPPRLGELWVTDPTPDSLRLSWTVPEGQFDSFVVQFKDKDGPQVVPVEGHERSVTVTPLDAGRKYRFLLYGLLGKKRHGPLTADGTTEARSAMDDTGTKRPPKPRLGEELQVTTVTQNSVGLSWTVPEGQFDSFVVQYKDRDGQPQVVPVEGSLREVSVPGLDPAHRYKLLLYGLHHGKRVGPISAVAITAGREETETETTAPTPPAPEPHLGELTVEEATSHTLHLSWMVTEGEFDSFEIQYTDRDGQLQMVRIGGDRNDITLSGLESDHRYLVTLYGFSDGKHVGPVHVEALTVPEEEKPSEPPTATPEPPIKPRLGELTVTDATPDSLSLSWTVPEGQFDHFLVQYRNGDGQPKAVRVPGHEEGVTISGLEPDHKYKMNLYGFHGGQRMGPVSVVGVTEPSMEAPEPAEEPLLGELTVTGSSPDSLSLSWTVPQGRFDSFTVQYKDRDGRPQVVRVGGEESEVTVGGLEPGRKYKMHLYGLHEGRRVGPVSAVGVTAPEEESPDAPLAKLRLGQMTVRDITSDSLSLSWTVPEGQFDHFLVQFKNGDGQPKAVRVPGHEDGVTISGLEPDHKYKMNLYGFHGGQRVGPVSAVGLTASTEPPTPEPPIKPRLEELTVTDATPDSLSLSWTVPEGQFDHFLVQYKNGDGQPKATRVPGHEDRVTISGLEPDNKYKMNLYGFHGGQRVGPVSAIGVTEEETPSPTEPSMEAPEPPEEPLLGELTVTGSSPDSLSLSWTVPQGRFDSFTVQYKDRDGRPQVVRVGGEESEVTVGGLEPGRKYKMHLYGLHEGRRVGPVSTVGVTAPQEDVDETPSPTEPGTEAPGPPEEPLLGELTVTGSSPDSLSLSWTVPQGRFDSFTVQYKDRDGRPQAVRVGGQESKVTVRGLEPGRKYKMHLYGLHEGRRLGPVSAVGVTEDEAETTQAVPTMTPEPPIKPRLGELTMTDATPDSLSLSWTVPEGQFDHFLVQYRNGDGQPKAVRVPGHEDGVTISGLEPDHKYKMNLYGFHGGQRVGPISVIGVTEEETPSPTELSTEAPEPPEEPLLGELTVTGSSPDSLSLSWTIPQGHFDSFTVQYKDRDGRPQVMRVRGEESEVTVGGLEPGRKYKMHLYGLHEGRRVGPVSTVGVTVPTTTPEPPNKPRLGELTVTDATPDSLSLSWMVPEGQFDHFLVQYRNGDGQPKVVRVPGHEDGVTISGLEPDHKYKMNLYGFHGGQRVGPISVIGVTEEETPAPTEPSTEAPEPPEEPLLGELTVTGSSPDSLSLSWTIPQGRFDSFTVQYKDRDGRPQVVRVRGEESEVTVGGLEPGCKYKMHLYGLHEGQRVGPVSAVGVTAPKDEAETTQAVPTMTPEPPIKPRLGELTVTDATPDSLSLSWMVPEGQFDHFLVQYRNGDGQPKAVRVPGHEDGVTISGLEPDHKYKMNLYGFHGGQRVGPVSAIGVTEEETPSPTEPSTEAPEAPEEPLLGELTVTGSSPDSLSLSWTVPQGRFDSFTVQYKDRDGQPQVVRVRGEESEVTVGGLEPGRKYKMHLYGLHEGQRVGPVSTVGITAPLPTPLPVEPRLGELAVAAVTSDSVGLSWTVAQGPFDSFLVQYRDAQGQPQAVPVSGDLRAVAVSGLDPARKYKFLLFGLQNGKRHGPVPVEARTAPDTKPSPRLGELTVTDATPDSVGLSWTVPEGEFDSFVVQYKDKDGRLQVVPVAANQREVTVQGLEPSRKYRFLLYGLSGRKRLGPISADSTTAPLEKELPPHLGELTVAEETSSSLRLSWTVAQGPFDSFVVQYRDTDGQPRAVPVAADQRTVTVEDLEPGKKYKFLLYGLLGGKRLGPVSALGMTAPEEDTPAPELAPEAPEPPEEPRLGVLTVTDTTPDSMRLSWSVAQGPFDSFVVQYEDTNGQPQALLVDGDQSKILISGLEPSTPYRFLLYGLHEGKRLGPLSAEGTTGLAPAGQTSEESRPRLSQLSVTDVTTSSLRLNWEAPPGAFDSFLLRFGVPSPSTLEPHPRPLLQRELMVPGTRHSAVLRDLRSGTLYSLTLYGLRGPHKADSIQGTARTLSPVLESPRDLQFSEIRETSAKVNWMPPPSRADSFKVSYQLADGGEPQSVQVDGQARTQKLQGLIPGARYEVTVVSVRGFEESEPLTGFLTTVPDGPTQLRALNLTEGFAVLHWKPPQNPVDTYDVQVTAPGAPPLQAETPGSAVDYPLHDLVLHTNYTATVRGLRGPNLTSPASITFTTGLEAPRDLEAKEVTPRTALLTWTEPPVRPAGYLLSFHTPGGQNQEILLPGGITSHQLLGLFPSTSYNARLQAMWGQSLLPPVSTSFTTGGLRIPFPRDCGEEMQNGAGASRTSTIFLNGNRERPLNVFCDMETDGGGWLVFQRRMDGQTDFWRDWEDYAHGFGNISGEFWLGNEALHSLTQAGDYSMRVDLRAGDEAVFAQYDSFHVDSAAEYYRLHLEGYHGTAGDSMSYHSGSVFSARDRDPNSLLISCAVSYRGAWWYRNCHYANLNGLYGSTVDHQGVSWYHWKGFEFSVPFTEMKLRPRNFRSPAGGG

SEQ ID NO: 17 - DNA, 1347 bp, Homo sapiens
atgatgaaaaccctgctgctgtttgtgggcctgctgctgacctgggaaagcggccaggtgctgggcgatcagaccgtgagcgataacgaactgcaggaaatgagcaaccagggcagcaaatatgtgaacaaagaaattcagaacgcggtgaacggcgtgaaacagattaaaaccctgattgaaaaaaccaacgaagaacgcaaaaccctgctgagcaacctggaagaagcgaaaaaaaaaaaagaagatgcgctgaacgaaacccgcgaaagcgaaaccaaactgaaagaactgccgggcgtgtgcaacgaaaccatgatggcgctgtgggaagaatgcaaaccgtgcctgaaacagacctgcatgaaattttatgcgcgcgtgtgccgcagcggcagcggcctggtgggccgccagctggaagaatttctgaaccagagcagcccgttttatttttggatgaacggcgatcgcattgatagcctgctggaaaacgatcgccagcagacccatatgctggatgtgatgcaggatcattttagccgcgcgagcagcattattgatgaactgtttcaggatcgcttttttacccgcgaaccgcaggatacctatcattatctgccgtttagcctgccgcatcgccgcccgcattttttttttccgaaaagccgcattgtgcgcagcctgatgccgtttagcccgtatgaaccgctgaactttcatgcgatgtttcagccgtttctggaaatgattcatgaagcgcagcaggcgatggatattcattttcatagcccggcgtttcagcatccgccgaccgaatttattcgcgaaggcgatgatgatcgcaccgtgtgccgcgaaattcgccataacagcaccggctgcctgcgcatgaaagatcagtgcgataaatgccgcgaaattctgagcgtggattgcagcaccaacaacccgagccaggcgaaactgcgccgcgaactggatgaaagcctgcaggtggcggaacgcctgacccgcaaatataacgaactgctgaaaagctatcagtggaaaatgctgaacaccagcagcctgctggaacagctgaacgaacagtttaactgggtgagccgcctggcgaacctgacccagggcgaagatcagtattatctgcgcgtgaccaccgtggcgagccataccagcgatagcgatgtgccgagcggcgtgaccgaagtggtggtgaaactgtttgatagcgatccgattaccgtgaccgtgccggtggaagtgagccgcaaaaacccgaaatttatggaaaccgtggcggaaaaagcgctgcaggaatatcgcaaaaaacatcgcgaagaa

SEQ ID NO: 18 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 19 - DNA, 873 bp, Homo sapiens
atgcagcgcgcgcgcccgaccctgtgggcggcggcgctgaccctgctggtgctgctgcgcggcccgccggtggcgcgcgcgggcgcgagcagcgcgggcctgggcccggtggtgcgctgcgaaccgtgcgatgcgcgcgcgctggcgcagtgcgcgccgccgccggcggtgtgcgcggaactggtgcgcgaaccgggctgcggctgctgcctgacctgcgcgctgagcgaaggccagccgtgcggcatttataccgaacgctgcggcagcggcctgcgctgccagccgagcccggatgaagcgcgcccgctgcaggcgctgctggatggccgcggcctgtgcgtgaacgcgagcgcggtgagccgcctgcgcgcgtatctgctgccggcgccgccggcgccgggcaacgcgagcgaaagcgaagaagatcgcagcgcgggcagcgtggaaagcccgagcgtgagcagcacccatcgcgtgagcgatccgaaatttcatccgctgcatagcaaaattattattattaaaaaaggccatgcgaaagatagccagcgctataaagtggattatgaaagccagagcaccgatacccagaactttagcagcgaaagcaaacgcgaaaccgaatatggcccgtgccgccgcgaaatggaagataccctgaaccatctgaaatttctgaacgtgctgagcccgcgcggcgtgcatattccgaactgcgataaaaaaggcttttataaaaaaaaacagtgccgcccgagcaaaggccgcaaacgcggcttttgctggtgcgtggataaatatggccagccgctgccgggctataccaccaaaggcaaagaagatgtgcattgctatagcatgcagagcaaa

SEQ ID NO: 20 - protein, 291 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 21 - DNA, 4710 bp, Homo sapiens
atggcgccgcatcgcccggcgccggcgctgctgtgcgcgctgagcctggcgctgtgcgcgctgagcctgccggtgcgcgcggcgaccgcgagccgcggcgcgagccaggcgggcgcgccgcagggccgcgtgccggaagcgcgcccgaacagcatggtggtggaacatccggaatttctgaaagcgggcaaagaaccgggcctgcagatttggcgcgtggaaaaatttgatctggtgccggtgccgaccaacctgtatggcgatttttttaccggcgatgcgtatgtgattctgaaaaccgtgcagctgcgcaacggcaacctgcagtatgatctgcattattggctgggcaacgaatgcagccaggatgaaagcggcgcggcggcgatttttaccgtgcagctggatgattatctgaacggccgcgcggtgcagcatcgcgaagtgcagggctttgaaagcgcgacctttctgggctattttaaaagcggcctgaaatataaaaaaggcggcgtggcgagcggctttaaacatgtggtgccgaacgaagtggtggtgcagcgcctgtttcaggtgaaaggccgccgcgtggtgcgcgcgaccgaagtgccggtgagctgggaaagctttaacaacggcgattgctttattctggatctgggcaacaacattcatcagtggtgcggcagcaacagcaaccgctatgaacgcctgaaagcgacccaggtgagcaaaggcattcgcgataacgaacgcagcggccgcgcgcgcgtgcatgtgagcgaagaaggcaccgaaccggaagcgatgctgcaggtgctgggcccgaaaccggcgctgccggcgggcaccgaagataccgcgaaagaagatgcggcgaaccgcaaactggcgaaactgtataaagtgagcaacggcgcgggcaccatgagcgtgagcctggtggcggatgaaaacccgtttgcgcagggcgcgctgaaaagcgaagattgctttattctggatcatggcaaagatggcaaaatttttgtgtggaaaggcaaacaggcgaacaccgaagaacgcaaagcggcgctgaaaaccgcgagcgattttattaccaaaatggattatccgaaacagacccaggtgagcgtgctgccggaaggcggcgaaaccccgctgtttaaacagttttttaaaaactggcgcgatccggatcagaccgatggcctgggcctgagctatctgagcagccatattgcgaacgtggaacgcgtgccgtttgatgcggcgaccctgcataccagcaccgcgatggcggcgcagcatggcatggatgatgatggcaccggccagaaacagatttggcgcattgaaggcagcaacaaagtgccggtggatccggcgacctatggccagttttatggcggcgatagctatattattctgtataactatcgccatggcggccgccagggccagattatttataactggcagggcgcgcagagcacccaggatgaagtggcggcgagcgcgattctgaccgcgcagctggatgaagaactgggcggcaccccggtgcagagccgcgtggtgcagggcaaagaaccggcgcatctgatgagcctgtttggcggcaaaccgatgattatttataaaggcggcaccagccgcgaaggcggccagaccgcgccggcgagcacccgcctgtttcaggtgcgcgcgaacagcgcgggcgcgacccgcgcggtggaagtgctgccgaaagcgggcgcgctgaacagcaacgatgcgtttgtgctgaaaaccccgagcgcggcgtatctgtgggtgggcaccggcgcgagcgaagcggaaaaaaccggcgcgcaggaactgctgcgcgtgctgcgcgcgcagccggtgcaggtggcggaaggcagcgaaccggatggcttttgggaagcgctgggcggcaaagcggcgtatcgcaccagcccgcgcctgaaagataaaaaaatggatgcgcatccgccgcgcctgtttgcgtgcagcaacaaaattggccgctttgtgattgaagaagtgccgggcgaactgatgcaggaagatctggcgaccgatgatgtgatgctgctggatacctgggatcaggtgtttgtgtgggtgggcaaagatagccaggaagaagaaaaaaccgaagcgctgaccagcgcgaaacgctatattgaaaccgatccggcgaaccgcgatcgccgcaccccgattaccgtggtgaaacagggctttgaaccgccgagctttgtgggctggtttctgggctgggatgatgattattggagcgtggatccgctggatcgcgcgatggcggaactggcggcgggctgcggctgcggctgctgctgcggctgcaccggctgcgcgggcggctgcggctgcaccggctgcaccggcggcgcgaccggcggctgctgcggctgcggcggctgctgcaccggcaccggctgcggcaccggcgcggcgtgcggctgcggcgcgggctgcggctgcggcggcaccggcgcgggctgctgcggctgctgcaccggctgcggctgcggctgcggcaccgcgacctgcaccggctgcaccggctgctgcggcggctgcggctgctgcggctgctgcggcggctgcggctgctgcggcggcggctgcggcgcggcgtgctgcggctgctgcggcggctgcggctgctgcggcggcggctgcgcggcgtgcggctgcggcgcgggctgcggcgcggcggcgggctgcggcgcggcgggcgcggcgggcgcgacctgcggctgcgcgggctgcggctgcggcggcggctgcgcgggctgcggcaccggcggcgcggcggcgggctgctgctgcggcgcgggctgcggcaccggcgcgggctgcgcgggctgcgcgtgctgctgcgcgacctgcggctgcggcaccggcgcgggctgcggcgcgacctgctgcggcgcggcggcgaccaccacctgcgcgacctgctgcggctgcaccggctgcgcgaccgcgggctgcgcggcggcggcgaccaccgcgaccaccgcgaccaccgcgaccaccgcggcggcggcggcggcgggcggctgctgcgcgaccggctgcggcgcggcggcgggcgcgaccgcgggctgctgcgcgggctgcggctgcaccgcgaccgcggcggcgggcaccggcggcgcgaccaccgcgaccggcgcggcggcgggctgctgcgcgggcgcgggctgcgcgtgctgcggcgcgaccgcgtgctgctgcgcgggcgcggcgtgcaccaccaccgcgggctgcgcgggctgcggcgcggcggcgggctgcgcggcggcgtgcggctgcggcgcggcggcgtgctgcggcgcggcgaccgcgaccggcggctgctgctgcggcaccggctgctgcggctgctgcggctgcggcgcggcggcgaccggcggcgcggcgggcgcgaccgcgtgctgctgcaccggcgcggcgtgctgcgcgacctgcaccggcgcggcggcgaccaccacctgcaccggcgcggcgtgcggcaccggctgcaccggcgcgggctgctgctgcggctgcggctgcggcggctgcggcaccggctgcgcgaccgcgaccacctgctgcggcgcggcgtgcaccggctgcggcgcgaccgcggcggcggcggcggcgggcggctgcaccaccaccaccgcgaccgcggcggcggcggcggcggcggcggcgtgcgcgggcaccggctgctgcggctgctgctgcggcgcgggctgcgcggcggcgggcggctgctgcggctgcgcggcggcgtgcggctgcggcggctgcaccaccaccaccggctgcaccggcggcaccggctgcggcaccggcggcgcgaccgcggcggcgaccgcgaccggcggctgctgcgcgggctgctgcggctgcaccggctgctgcggcggcggctgcaccgcgaccgcgtgctgcgcgtgctgcgcggcggcgggcggctgcgcggcggcgggcgcggcgggcgcgaccggcaccggctgcgcgaccaccggctgcaccgcgaccgcgggctgcgcgaccggctgcgcgggcgcgggctgcgcggcggcggcgcgcccgctgcaggcgctgctggatggccgcggcctgtgcgtgaacgcgagcgcggtgagccgcctgcgcgcgtatctgctgccggcgccgccggcgccgggcgaaccgccggcgccgggcaacgcgagcgaaagcgaagaagatcgcagcgcgggcagcgtggaaagcccgagcgtgagcagcacccatcgcgtgagcgatccgaaatttcatccgctgcatagcaaaattattattattaaaaaaggccatgcgaaagatagccagcgctataaagtggattatgaaagccagagcaccgatacccagaactttagcagcgaaagcaaacgcgaaaccgaatatggcccgtgccgccgcgaaatggaagataccctgaaccatctgaaatttctgaacgtgctgagcccgcgcggcgtgcatattccgaactgcgataaaaaaggcttttataaaaaaaaacagtgccgcccgagcaaaggccgcaaacgcggcttttgctggtgcgtggataaatatggccagccgctgccgggctataccaccaaaggcaaagaagatgtgcattgctatagcatgcagagcaaa

SEQ ID NO: 22 - protein, 1373 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAAGCGCGCCCGCTGCAGGCGCTGCTGGATGGCCGCGGCCTGTGCGTGAACGCGAGCGCGGTGAGCCGCCTGCGCGCGTATCTGCTGCCGGCGCCGCCGGCGCCGGGCGAACCGCCGGCGCCGGGCAACGCGAGCGAAAGCGAAGAAGATCGCAGCGCGGGCAGCGTGGAAAGCCCGAGCGTGAGCAGCACCCATCGCGTGAGCGATCCGAAATTTCATCCGCTGCATAGCAAAATTATTATTATTAAAAAAGGCCATGCGAAAGATAGCCAGCGCTATAAAGTGGATTATGAAAGCCAGAGCACCGATACCCAGAACTTTAGCAGCGAAAGCAAACGCGAAACCGAATATGGCCCGTGCCGCCGCGAAATGGAAGATACCCTGAACCATCTGAAATTTCTGAACGTGCTGAGCCCGCGCGGCGTGCATATTCCGAACTGCGATAAAAAAGGCTTTTATAAAAAAAAACAGTGCCGCCCGAGCAAAGGCCGCAAACGCGGCTTTTGCTGGTGCGTGGATAAATATGGCCAGCCGCTGCCGGGCTATACCACCAAAGGCAAAGAAGATGTGCATTGCTATAGCATGCAGAGCAAA

SEQ ID NO: 23 - protein, 2097 aa, Homo sapiens
ATGCGCTGGCTGCTGCTGTATTATGCGCTGTGCTTTAGCCTGAGCAAAGCGAGCGCGCATACCGTGGAACTGAACAACATGTTTGGCCAGATTCAGAGCCCGGGCTATCCGGATAGCTATCCGAGCGATAGCGAAGTGACCTGGAACATTACCGTGCCGGATGGCTTTCGCATTAAACTGTATTTTATGCATTTTAACCTGGAAAGCAGCTATCTGTGCGAATATGATTATGTGAAAGTGGAAACCGAAGATCAGGTGCTGGCGACCTTTTGCGGCCGCGAAACCACCGATACCGAACAGACCCCGGGCCAGGAAGTGGTGCTGAGCCCGGGCAGCTTTATGAGCATTACCTTTCGCAGCGATTTTAGCAACGAAGAACGCTTTACCGGCTTTGATGCGCATTATATGGCGGTGGATGTGGATGAATGCAAAGAACGCGAAGATGAAGAACTGAGCTGCGATCATTATTGCCATAACTATATTGGCGGCTATTATTGCAGCTGCCGCTTTGGCTATATTCTGCATACCGATAACCGCACCTGCCGCGTGGAATGCAGCGATAACCTGTTTACCCAGCGCACCGGCGTGATTACCAGCCCGGATTTTCCGAACCCGTATCCGAAAAGCAGCGAATGCCTGTATACCATTGAACTGGAAGAAGGCTTTATGGTGAACCTGCAGTTTGAAGATATTTTTGATATTGAAGATCATCCGGAAGTGCCGTGCCCGTATGATTATATTAAAATTAAAGTGGGCCCGAAAGTGCTGGGCCCGTTTTGCGGCGAAAAAGCGCCGGAACCGATTAGCACCCAGAGCCATAGCGTGCTGATTCTGTTTCATAGCGATAACAGCGGCGAAAACCGCGGCTGGCGCCTGAGCTATCGCGCGGCGGGCAACGAATGCCCGGAACTGCAGCCGCCGGTGCATGGCAAAATTGAACCGAGCCAGGCGAAATATTTTTTTAAAGATCAGGTGCTGGTGAGCTGCGATACCGGCTATAAAGTGCTGAAAGATAACGTGGAAATGGATACCTTTCAGATTGAATGCCTGAAAGATGGCACCTGGAGCAACAAAATTCCGACCTGCAAAATTGTGGATTGCCGCGCGCCGGGCGAACTGGAACATGGCCTGATTACCTTTAGCACCCGCAACAACCTGACCACCTATAAAAGCGAAATTAAATATAGCTGCCAGGAACCGTATTATAAAATGCTGAACAACAACACCGGCATTTATACCTGCAGCGCGCAGGGCGTGTGGATGAACAAAGTGCTGGGCCGCAGCCTGCCGACCTGCCTGCCGGTGTGCGGCCTGCCGAAATTTAGCCGCAAACTGATGGCGCGCATTTTTAACGGCCGCCCGGCGCAGAAAGGCACCACCCCGTGGATTGCGATGCTGAGCCATCTGAACGGCCAGCCGTTTTGCGGCGGCAGCCTGCTGGGCAGCAGCTGGATTGTGACCGCGGCGCATTGCCTGCATCAGAGCCTGGATCCGGAAGATCCGACCCTGCGCGATAGCGATCTGCTGAGCCCGAGCGATTTTAAAATTATTCTGGGCAAACATTGGCGCCTGCGCAGCGATGAAAACGAACAGCATCTGGGCGTGAAACATACCACCCTGCATCCGCAGTATGATCCGAACACCTTTGAAAACGATGTGGCGCTGGTGGAACTGCTGGAAAGCCCGGTGCTGAACGCGTTTGTGATGCCGATTTGCCTGCCGGAAGGCCCGCAGCAGGAAGGCGCGATGGTGATTGTGAGCGGCTGGGGCAAACAGTTTCTGCAGCGCTTTCCGGAAACCCTGATGGAAATTGAAATTCCGATTGTGGATCATAGCACCTGCCAGAAAGCGTATGCGCCGCTGAAAAAAAAAGTGACCCGCGATATGATTTGCGCGGGCGAAAAAGAAGGCGGCAAAGATGCGTGCGCGGGCGATAGCGGCGGCCCGATGGTGACCCTGAACCGCGAACGCGGCCAGTGGTATCTGGTGGGCACCGTGAGCTGGGGCGATGATTGCGGCAAAAAAGATCGCTATGGCGTGTATAGCTATATTCATCATAACAAAGATTGGATTCAGCGCGTGACCGGCGTGCGCAAC

SEQ ID NO: 24 - protein, 699 aa, Homo sapiens
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIEDHPEVPCPYDYIKIKVGPKVLGPFCGEKAPEPISTQSHSVLILFHSDNSGENRGWRLSYRAAGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPVCGLPKFSRKLMARIFNGRPAQKGTTPWIAMLSHLNGQPFCGGSLLGSSWIVTAAHCLHQSLDPEDPTLRDSDLLSPSDFKIILGKHWRLRSDENEQHLGVKHTTLHPQYDPNTFENDVALVELLESPVLNAFVMPICLPEGPQQEGAMVIVSGWGKQFLQRFPETLMEIEIPIVDHSTCQKAYAPLKKKVTRDMICAGEKEGGKDACAGDSGGPMVTLNRERGQWYLVGTVSWGDDCGKKDRYGVYSYIHHNKDWIQRVTGVRN

SEQ ID NO: 25 - DNA, 5262 bp, Homo sapiens
atggcgccgtatccgtgcggctgccatattctgctgctgctgttttgctgcctggcggcggcgcgcgcgaacctgctgaacctgaactggctgtggtttaacaacgaagataccagccatgcggcgaccaccattccggaaccgcagggcccgctgccggtgcagccgaccgcggataccaccacccatgtgaccccgcgcaacggcagcaccgaaccggcgaccgcgccgggcagcccggaaccgccgagcgaactgctggaagatggccaggataccccgaccagcgcggaaagcccggatgcgccggaagaaaacattgcgggcgtgggcgcggaaattctgaacgtggcgaaaggcattcgcagctttgtgcagctgtggaacgataccgtgccgaccgaaagcctggcgcgcgcggaaaccctggtgctggaaaccccggtgggcccgctggcgctggcgggcccgagcagcaccccgcaggaaaacggcaccaccctgtggccgagccgcggcattccgagcagcccgggcgcgcataccaccgaagcgggcaccctgccggcgccgaccccgagcccgccgagcctgggccgcccgtgggcgccgctgaccggcccgagcgtgccgccgccgagcagcggccgcgcgagcctgagcagcctgctgggcggcgcgccgccgtggggcagcctgcaggatccggatagccagggcctgagcccggcggcggcggcgccgagccagcagctgcagcgcccggatgtgcgcctgcgcaccccgctgctgcatccgctggtgatgggcagcctgggcaaacatgcggcgccgagcgcgtttagcagcggcctgccgggcgcgctgagccaggtggcggtgaccaccctgacccgcgatagcggcgcgtgggtgagccatgtggcgaacagcgtgggcccgggcctggcgaacaacagcgcgctgctgggcgcggatccggaagcgccggcgggccgctgcctgccgctgccgccgagcctgccggtgtgcggccatctgggcattagccgcttttggctgccgaaccatctgcatcatgaaagcggcgaacaggtgcgcgcgggcgcgcgcgcgtggggcggcctgctgcagacccattgccatccgtttctggcgtggtttttttgcctgctgctggtgccgccgtgcggcagcgtgccgccgccggcgccgccgccgtgctgccagttttgcgaagcgctgcaggatgcgtgctggagccgcctgggcggcggccgcctgccggtggcgtgcgcgagcctgccgacccaggaagatggctattgcgtgctgattggcccggcggcggaacgcattagcgaagaagtgggcctgctgcagctgctgggcgatccgccgccgcagcaggtgacccagaccgatgatccggatgtgggcctggcgtatgtgtttggcccggatgcgaacagcggccaggtggcgcgctatcattttccgagcctgttttttcgcgattttagcctgctgtttcatattcgcccggcgaccgaaggcccgggcgtgctgtttgcgattaccgatagcgcgcaggcgatggtgctgctgggcgtgaaactgagcggcgtgcaggatggccatcaggatattagcctgctgtataccgaaccgggcgcgggccagacccataccgcggcgagctttcgcctgccggcgtttgtgggccagtggacccatctggcgctgagcgtggcgggcggctttgtggcgctgtatgtggattgcgaagaatttcagcgcatgccgctggcgcgcagcagccgcggcctggaactggaaccgggcgcgggcctgtttgtggcgcaggcgggcggcgcggatccggataaatttcagggcgtgattgcggaactgaaagtgcgccgcgatccgcaggtgagcccgatgcattgcctggatgaagaaggcgatgatagcgatggcgcgagcggcgatagcggcagcggcctgggcgatgcgcgcgaactgctgcgcgaagaaaccggcgcggcgctgaaaccgcgcctgccggcgccgccgccggtgaccaccccgccgctggcgggcggcagcagcaccgaagatagccgcagcgaagaagtggaagaacagaccaccgtggcgagcctgggcgcgcagaccctgccgggcagcgatagcgtgagcacctgggatggcagcgtgcgcaccccgggcggccgcgtgaaagaaggcggcctgaaaggccagaaaggcgaaccgggcgtgccgggcccgccgggccgcgcgggcccgccgggcagcccgtgcctgccgggcccgccgggcctgccgtgcccggtgagcccgctgggcccggcgggcccggcgctgcagaccgtgccgggcccgcagggcccgccgggcccgccgggccgcgatggcaccccgggccgcgatggcgaaccgggcgatccgggcgaagatggcaaaccgggcgataccggcccgcagggctttccgggcaccccgggcgatgtgggcccgaaaggcgataaaggcgatccgggcgtgggcgaacgcggcccgccgggcccgcagggcccgccgggcccgccgggcccgagctttcgccatgataaactgacctttattgatatggaaggcagcggctttggcggcgatctggaagcgctgcgcggcccgcgcggctttccgggcccgccgggcccgccgggcgtgccgggcctgccgggcgaaccgggccgctttggcgtgaacagcagcgatgtgccgggcccggcgggcctgccgggcgtgccgggccgcgaaggcccgccgggctttccgggcctgccgggcccgccgggcccgccgggccgcgaaggcccgccgggccgcaccggccagaaaggcagcctgggcgaagcgggcgcgccgggccataaaggcagcaaaggcgcgccgggcccggcgggcgcgcgcggcgaaagcggcctggcgggcgcgccgggcccggcgggcccgccgggcccgccgggcccgccgggcccgccgggcccgggcctgccggcgggctttgatgatatggaaggcagcggcggcccgttttggagcaccgcgcgcagcgcggatggcccgcagggcccgccgggcctgccgggcctgaaaggcgatccgggcgtgccgggcctgccgggcgcgaaaggcgaagtgggcgcggatggcgtgccgggctttccgggcctgccgggccgcgaaggcattgcgggcccgcagggcccgaaaggcgatcgcggcagccgcggcgaaaaaggcgatccgggcaaagatggcgtgggccagccgggcctgccgggcccgccgggcccgccgggcccggtggtgtatgtgagcgaacaggatggcagcgtgctgagcgtgccgggcccggaaggccgcccgggctttgcgggctttccgggcccggcgggcccgaaaggcaacctgggcagcaaaggcgaacgcggcagcccgggcccgaaaggcgaaaaaggcgaaccgggcagcatttttagcccggatggcggcgcgctgggcccggcgcagaaaggcgcgaaaggcgaaccgggctttcgcggcccgccgggcccgtatggccgcccgggctataaaggcgaaattggctttccgggccgcccgggccgcccgggcatgaacggcctgaaaggcgaaaaaggcgaaccgggcgatgcgagcctgggctttggcatgcgcggcatgccgggcccgccgggcccgccgggcccgccgggcccgccgggcaccccggtgtatgatagcaacgtgtttgcggaaagcagccgcccgggcccgccgggcctgccgggcaaccagggcccgccgggcccgaaaggcgcgaaaggcgaagtgggcccgccgggcccgccgggccagtttccgtttgattttctgcagctggaagcggaaatgaaaggcgaaaaaggcgatcgcggcgatgcgggccagaaaggcgaacgcggcgaaccgggcggcggcggcttttttggcagcagcctgccgggcccgccgggcccgccgggcccgccgggcccgcgcggctatccgggcattccgggcccgaaaggcgaaagcattcgcggccagccgggcccgccgggcccgcagggcccgccgggcattggctatgaaggccgccagggcccgccgggcccgccgggcccgccgggcccgccgagctttccgggcccgcatcgccagaccattagcgtgccgggcccgccgggcccgccgggcccgccgggcccgccgggcaccatgggcgcgagcagcggcgtgcgcctgtgggcgacccgccaggcgatgctgggccaggtgcatgaagtgccggaaggctggctgatttttgtggcggaacaggaagaactgtatgtgcgcgtgcagaacggctttcgcaaagtgcagctggaagcgcgcaccccgctgccgcgcggcaccgataacgaagtggcggcgctgcagccgccggtggtgcagctgcatgatagcaacccgtatccgcgccgcgaacatccgcatccgaccgcgcgcccgtggcgcgcggatgatattctggcgagcccgccgcgcctgccggaaccgcagccgtatccgggcgcgccgcatcatagcagctatgtgcatctgcgcccggcgcgcccgaccagcccgccggcgcatagccatcgcgattttcagccggtgctgcatctggtggcgctgaacagcccgctgagcggcggcatgcgcggcattcgcggcgcggattttcagtgctttcagcaggcgcgcgcggtgggcctggcgggcacctttcgcgcgtttctgagcagccgcctgcaggatctgtatagcattgtgcgccgcgcggatcgcgcggcggtgccgattgtgaacctgaaagatgaactgctgtttccgagctgggaagcgctgtttagcggcagcgaaggcccgctgaaaccgggcgcgcgcatttttagctttgatggcaaagatgtgctgcgccatccgacctggccgcagaaaagcgtgtggcatggcagcgatccgaacggccgccgcctgaccgaaagctattgcgaaacctggcgcaccgaagcgccgagcgcgaccggccaggcgagcagcctgctgggcggccgcctgctgggccagagcgcggcgagctgccatcatgcgtatattgtgctgtgcattgaaaacagctttatgaccgcgagcaaa

SEQ ID NO: 26 - protein, 1754 aa, Homo sapiens
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSGRASLSSLLGGAPPWGSLQDPDSQGLSPAAAAPSQQLQRPDVRLRTPLLHPLVMGSLGKHAAPSAFSSGLPGALSQVAVTTLTRDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAAERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 27 - DNA, 1962 bp, Homo sapiens
atgaaactgagcctggtggcggcgatgctgctgctgctgagcgcggcgcgcgcggaagaagaagataaaaaagaagatgtgggcaccgtggtgggcattgatctgggcaccacctatagctgcgtgggcgtgtttaaaaacggccgcgtggaaattattgcgaacgatcagggcaaccgcattaccccgagctatgtggcgtttaccccggaaggcgaacgcctgattggcgatgcggcgaaaaaccagctgaccagcaacccggaaaacaccgtgtttgatgcgaaacgcctgattggccgcacctggaacgatccgagcgtgcagcaggatattaaatttctgccgtttaaagtggtggaaaaaaaaaccaaaccgtatattcaggtggatattggcggcggccagaccaaaacctttgcgccggaagaaattagcgcgatggtgctgaccaaaatgaaagaaaccgcggaagcgtatctgggcaaaaaagtgacccatgcggtggtgaccgtgccggcgtattttaacgatgcgcagcgccaggcgaccaaagatgcgggcaccattgcgggcctgaacgtgatgcgcattattaacgaaccgaccgcggcggcgattgcgtatggcctggataaacgcgaaggcgaaaaaaacattctggtgtttgatctgggcggcggcacctttgatgtgagcctgctgaccattgataacggcgtgtttgaagtggtggcgaccaacggcgatacccatctgggcggcgaagattttgatcagcgcgtgatggaacattttattaaactgtataaaaaaaaaaccggcaaagatgtgcgcaaagataaccgcgcggtgcagaaactgcgccgcgaagtggaaaaagcgaaacgcgcgctgagcagccagcatcaggcgcgcattgaaattgaaagcttttatgaaggcgaagattttagcgaaaccctgacccgcgcgaaatttgaagaactgaacatggatctgtttcgcagcaccatgaaaccggtgcagaaagtgctggaagatagcgatctgaaaaaaagcgatattgatgaaattgtgctggtgggcggcagcacccgcattccgaaaattcagcagctggtgaaagaattttttaacggcaaagaaccgagccgcggcattaacccggatgaagcggtggcgtatggcgcggcggtgcaggcgggcgtgctgagcggcgatcaggataccggcgatctggtgctgctggatgtgtgcccgctgaccctgggcattgaaaccgtgggcggcgtgatgaccaaactgattccgcgcaacaccgtggtgccgaccaaaaaaagccagatttttagcaccgcgagcgataaccagccgaccgtgaccattaaagtgtatgaaggcgaacgcccgctgaccaaagataaccatctgctgggcacctttgatctgaccggcattccgccggcgccgcgcggcgtgccgcagattgaagtgacctttgaaattgatgtgaacggcattctgcgcgtgaccgcggaagataaaggcaccggcaacaaaaacaaaattaccattaccaacgatcagaaccgcctgaccccggaagaaattgaacgcatggtgaacgatgcggaaaaatttgcggaagaagataaaaaactgaaagaacgcattgatacccgcaacgaactggaaagctatgcgtatagcctgaaaaaccagattggcgataaagaaaaactgggcggcaaactgagcagcgaagataaagaaaccatggaaaaagcggtggaagaaaaaattgaatggctggaaagccatcaggatgcggatattgaagattttaaagcgaaaaaaaaagaactggaagaaattgtgcagccgattattagcaaactgtatggcagcgcgggcccgccgccgaccggcgaagaagataccgcggaaaaagatgaactg

SEQ ID NO: 28 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 29 - DNA, 2928 bp, Homo sapiens
atgcgcggcgcgcgcggcgcgtgggattttctgtgcgtgctgctgctgctgctgcgcgtgcagaccggcagcagccagccgagcgtgagcccgggcgaaccgagcccgccgagcattcatccgggcaaaagcgatctgattgtgcgcgtgggcgatgaaattcgcctgctgtgcaccgatccgggctttgtgaaatggacctttgaaattctggatgaaaccaacgaaaacaaacagaacgaatggattaccgaaaaagcggaagcgaccaacaccggcaaatatacctgcaccaacaaacatggcctgagcaacagcatttatgtgtttgtgcgcgatccggcgaaactgtttctggtggatcgcagcctgtatggcaaagaagataacgataccctggtgcgctgcccgctgaccgatccggaagtgaccaactatagcctgaaaggctgccagggcaaaccgctgccgaaagatctgcgctttattccggatccgaaagcgggcattatgattaaaagcgtgaaacgcgcgtatcatcgcctgtgcctgcattgcagcgtggatcaggaaggcaaaagcgtgctgagcgaaaaatttattctgaaagtgcgcccggcgtttaaagcggtgccggtggtgagcgtgagcaaagcgagctatctgctgcgcgaaggcgaagaatttaccgtgacctgcaccattaaagatgtgagcagcagcgtgtatagcacctggaaacgcgaaaacagccagaccaaactgcaggaaaaatataacagctggcatcatggcgattttaactatgaacgccaggcgaccctgaccattagcagcgcgcgcgtgaacgatagcggcgtgtttatgtgctatgcgaacaacacctttggcagcgcgaacgtgaccaccaccctggaagtggtggataaaggctttattaacatttttccgatgattaacaccaccgtgtttgtgaacgatggcgaaaacgtggatctgattgtggaatatgaagcgtttccgaaaccggaacatcagcagtggatttatatgaaccgcacctttaccgataaatgggaagattatccgaaaagcgaaaacgaaagcaacattcgctatgtgagcgaactgcatctgacccgcctgaaaggcaccgaaggcggcacctatacctttctggtgagcaacagcgatgtgaacgcggcgattgcgtttaacgtgtatgtgaacaccaaaccggaaattctgacctatgatcgcctggtgaacggcatgctgcagtgcgtggcggcgggctttccggaaccgaccattgattggtatttttgcccgggcaccgaacagcgctgcagcgcgagcgtgctgccggtggatgtgcagaccctgaacagcagcggcccgccgtttggcaaactggtggtgcagagcagcattgatagcagcgcgtttaaacataacggcaccgtggaatgcaaagcgtataacgatgtgggcaaaaccagcgcgtattttaactttgcgtttaaaggcaacaacaaagaacagattcatccgcataccctgtttaccccgctgctgattggctttgtgattgtggcgggcatgatgtgcattattgtgatgattctgacctataaatatctgcagaaaccgatgtatgaagtgcagtggaaagtggtggaagaaattaacggcaacaactatgtgtatattgatccgacccagctgccgtatgatcataaatgggaatttccgcgcaaccgcctgagctttggcaaaaccctgggcgcgggcgcgtttggcaaagtggtggaagcgaccgcgtatggcctgattaaaagcgatgcggcgatgaccgtggcggtgaaaatgctgaaaccgagcgcgcatctgaccgaacgcgaagcgctgatgagcgaactgaaagtgctgagctatctgggcaaccatatgaacattgtgaacctgctgggcgcgtgcaccattggcggcccgaccctggtgattaccgaatattgctgctatggcgatctgctgaactttctgcgccgcaaacgcgatagctttatttgcagcaaacaggaagatcatgcggaagcggcgctgtataaaaacctgctgcatagcaaagaaagcagctgcagcgatagcaccaacgaatatatggatatgaaaccgggcgtgagctatgtggtgccgaccaaagcggataaacgccgcagcgtgcgcattggcagctatattgaacgcgatgtgaccccggcgattatggaagatgatgaactggcgctggatctggaagatctgctgagctttagctatcaggtggcgaaaggcatggcgtttctggcgagcaaaaactgcattcatcgcgatctggcggcgcgcaacattctgctgacccatggccgcattaccaaaatttgcgattttggcctggcgcgcgatattaaaaacgatagcaactatgtggtgaaaggcaacgcgcgcctgccggtgaaatggatggcgccggaaagcatttttaactgcgtgtatacctttgaaagcgatgtgtggagctatggcatttttctgtgggaactgtttagcctgggcagcagcccgtatccgggcatgccggtggatagcaaattttataaaatgattaaagaaggctttcgcatgctgagcccggaacatgcgccggcggaaatgtatgatattatgaaaacctgctgggatgcggatccgctgaaacgcccgacctttaaacagattgtgcagctgattgaaaaacagattagcgaaagcaccaaccatatttatagcaacctggcgaactgcagcccgaaccgccagaaaccggtggtggatcatagcgtgcgcattaacagcgtgggcagcaccgcgagcagcagccagccgctgctggtgcatgatgatgtg

SEQ ID NO: 30 - protein, 976 aa, Homo sapiens [sequence omitted]

SEQ ID NO: 31 - DNA, 420 bp, Homo sapiens
atggcgggctggaacgcgtatattgataacctgatggcggatggcacctgccaggatgcggcgattgtgggctataaagatagcccgagcgtgtgggcggcggtgccgggcaaaacctttgtgaacattaccccggcggaagtgggcgtgctggtgggcaaagatcgcagcagcttttatgtgaacggcctgaccctgggcggccagaaatgcagcgtgattcgcgatagcctgctgcaggatggcgaatttagcatggatctgcgcaccaaaagcaccggcggcgcgccgacctttaacgtgaccgtgaccaaaaccgataaaaccctggtgctgctgatgggcaaagaaggcgtgcatggcggcctgattaacaaaaaatgctatgaaatggcgagccatctgcgccgcagccagtat

SEQ ID NO: 32 - protein, 140 aa, Homo sapiens
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 33 - DNA, 1254 bp, Homo sapiens
atgcaggcgctggtgctgctgctgtgcattggcgcgctgctgggccatagcagctgccagaacccggcgagcccgccggaagaaggcagcccggatccggatagcaccggcgcgctggtggaagaagaagatccgttttttaaagtgccggtgaacaaactggcggcggcggtgagcaactttggctatgatctgtatcgcgtgcgcagcagcaccagcccgaccaccaacgtgctgctgagcccgctgagcgtggcgaccgcgctgagcgcgctgagcctgggcgcggaacagcgcaccgaaagcattattcatcgcgcgctgtattatgatctgattagcagcccggatattcatggcacctataaagaactgctggataccgtgaccgcgccgcagaaaaacctgaaaagcgcgagccgcattgtgtttgaaaaaaaactgcgcattaaaagcagctttgtggcgccgctggaaaaaagctatggcacccgcccgcgcgtgctgaccggcaacccgcgcctggatctgcaggaaattaacaactgggtgcaggcgcagatgaaaggcaaactggcgcgcagcaccaaagaaattccggatgaaattagcattctgctgctgggcgtggcgcattttaaaggccagtgggtgaccaaatttgatagccgcaaaaccagcctggaagatttttatctggatgaagaacgcaccgtgcgcgtgccgatgatgagcgatccgaaagcggtgctgcgctatggcctggatagcgatctgagctgcaaaattgcgcagctgccgctgaccggcagcatgagcattattttttttctgccgctgaaagtgacccagaacctgaccctgattgaagaaagcctgaccagcgaatttattcatgatattgatcgcgaactgaaaaccgtgcaggcggtgctgaccgtgccgaaactgaaactgagctatgaaggcgaagtgaccaaaagcctgcaggaaatgaaactgcagagcctgtttgatagcccggattttagcaaaattaccggcaaaccgattaaactgacccaggtggaacatcgcgcgggctttgaatggaacgaagatggcgcgggcaccaccccgagcccgggcctgcagccggcgcatctgacctttccgctggattatcatctgaaccagccgtttatttttgtgctgcgcgataccgataccggcgcgctgctgtttattggcaaaattctggatccgcgcggcccg

SEQ ID NO: 34 - protein, 418 aa, Homo sapiens
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 35 - DNA, 1014 bp, Homo sapiens
atgagcctgagcgcgtttaccctgtttctggcgctgattggcggcaccagcggccagtattatgattatgattttccgctgagcatttatggccagagcagcccgaactgcgcgccggaatgcaactgcccggaaagctatccgagcgcgatgtattgcgatgaactgaaactgaaaagcgtgccgatggtgccgccgggcattaaatatctgtatctgcgcaacaaccagattgatcatattgatgaaaaagcgtttgaaaacgtgaccgatctgcagtggctgattctggatcataacctgctggaaaacagcaaaattaaaggccgcgtgtttagcaaactgaaacagctgaaaaaactgcatattaaccataacaacctgaccgaaagcgtgggcccgctgccgaaaagcctggaagatctgcagctgacccataacaaaattaccaaactgggcagctttgaaggcctggtgaacctgacctttattcatctgcagcataaccgcctgaaagaagatgcggtgagcgcggcgtttaaaggcctgaaaagcctggaatatctggatctgagctttaaccagattgcgcgcctgccgagcggcctgccggtgagcctgctgaccctgtatctggataacaacaaaattagcaacattccggatgaatattttaaacgctttaacgcgctgcagtatctgcgcctgagccataacgaactggcggatagcggcattccgggcaacagctttaacgtgagcagcctggtggaactggatctgagctataacaaactgaaaaacattccgaccgtgaacgaaaacctggaaaactattatctggaagtgaaccagctggaaaaatttgatattaaaagcttttgcaaaattctgggcccgctgagctatagcaaaattaaacatctgcgcctggatggcaaccgcattagcgaaaccagcctgccgccggatatgtatgaatgcctgcgcgtggcgaacgaagtgaccctgaac

SEQ ID NO: 36 - protein, 338 aa, Homo sapiens
MSLSAFTLFLALIGGTSGQYYDYDFPLSIYGQSSPNCAPECNCPESYPSAMYCDELKLKSVPMVPPGIKYLYLRNNQIDHIDEKAFENVTDLQWLILDHNLLENSKIKGRVFSKLKQLKKLHINHNNLTESVGPLPKSLEDLQLTHNKITKLGSFEGLVNLTFIHLQHNRLKEDAVSAAFKGLKSLEYLDLSFNQIARLPSGLPVSLLTLYLDNNKISNIPDEYFKRFNALQYLRLSHNELADSGIPGNSFNVSSLVELDLSYNKLKNIPTVNENLENYYLEVNQLEKFDIKSFCKILGPLSYSKIKHLRLDGNRISETSLPPDMYECLRVANEVTLN

SEQ ID NO: 37 - protein, 3468 aa, Homo sapiens
ATGAGCAAACTGCGCATGGTGCTGCTGGAAGATAGCGGCAGCGCGGATTTTCGCCGCCATTTTGTGAACCTGAGCCCGTTTACCATTACCGTGGTGCTGCTGCTGAGCGCGTGCTTTGTGACCAGCAGCCTGGGCGGCACCGATAAAGAACTGCGCCTGGTGGATGGCGAAAACAAATGCAGCGGCCGCGTGGAAGTGAAAGTGCAGGAAGAATGGGGCACCGTGTGCAACAACGGCTGGAGCATGGAAGCGGTGAGCGTGATTTGCAACCAGCTGGGCTGCCCGACCGCGATTAAAGCGCCGGGCTGGGCGAACAGCAGCGCGGGCAGCGGCCGCATTTGGATGGATCATGTGAGCTGCCGCGGCAACGAAAGCGCGCTGTGGGATTGCAAACATGATGGCTGGGGCAAACATAGCAACTGCACCCATCAGCAGGATGCGGGCGTGACCTGCAGCGATGGCAGCAACCTGGAAATGCGCCTGACCCGCGGCGGCAACATGTGCAGCGGCCGCATTGAAATTAAATTTCAGGGCCGCTGGGGCACCGTGTGCGATGATAACTTTAACATTGATCATGCGAGCGTGATTTGCCGCCAGCTGGAATGCGGCAGCGCGGTGAGCTTTAGCGGCAGCAGCAACTTTGGCGAAGGCAGCGGCCCGATTTGGTTTGATGATCTGATTTGCAACGGCAACGAAAGCGCGCTGTGGAACTGCAAACATCAGGGCTGGGGCAAACATAACTGCGATCATGCGGAAGATGCGGGCGTGATTTGCAGCAAAGGCGCGGATCTGAGCCTGCGCCTGGTGGATGGCGTGACCGAATGCAGCGGCCGCCTGGAAGTGCGCTTTCAGGGCGAATGGGGCACCATTTGCGATGATGGCTGGGATAGCTATGATGCGGCGGTGGCGTGCAAACAGCTGGGCTGCCCGACCGCGGTGACCGCGATTGGCCGCGTGAACGCGAGCAAAGGCTTTGGCCATATTTGGCTGGATAGCGTGAGCTGCCAGGGCCATGAACCGGCGATTTGGCAGTGCAAACATCATGAATGGGGCAAACATTATTGCAACCATAACGAAGATGCGGGCGTGACCTGCAGCGATGGCAGCGATCTGGAACTGCGCCTGCGCGGCGGCGGCAGCCGCTGCGCGGGCACCGTGGAAGTGGAAATTCAGCGCCTGCTGGGCAAAGTGTGCGATCGCGGCTGGGGCCTGAAAGAAGCGGATGTGGTGTGCCGCCAGCTGGGCTGCGGCAGCGCGCTGAAAACCAGCTATCAGGTGTATAGCAAAATTCAGGCGACCAACACCTGGCTGTTTCTGAGCAGCTGCAACGGCAACGAAACCAGCCTGTGGGATTGCAAAAACTGGCAGTGGGGCGGCCTGACCTGCGATCATTATGAAGAAGCGAAAATTACCTGCAGCGCGCATCGCGAACCGCGCCTGGTGGGCGGCGATATTCCGTGCAGCGGCCGCGTGGAAGTGAAACATGGCGATACCTGGGGCAGCATTTGCGATAGCGATTTTAGCCTGGAAGCGGCGAGCGTGCTGTGCCGCGAACTGCAGTGCGGCACCGTGGTGAGCATTCTGGGCGGCGCGCATTTTGGCGAAGGCAACGGCCAGATTTGGGCGGAAGAATTTCAGTGCGAAGGCCATGAAAGCCATCTGAGCCTGTGCCCGGTGGCGCCGCGCCCGGAAGGCACCTGCAGCCATAGCCGCGATGTGGGCGTGGTGTGCAGCCGCTATACCGAAATTCGCCTGGTGAACGGCAAAACCCCGTGCGAAGGCCGCGTGGAACTGAAAACCCTGGGCGCGTGGGGCAGCCTGTGCAACAGCCATTGGGATATTGAAGATGCGCATGTGCTGTGCCAGCAGCTGAAATGCGGCGTGGCGCTGAGCACCCCGGGCGGCGCGCGCTTTGGCAAAGGCAACGGCCAGATTTGGCGCCATATGTTTCATTGCACCGGCACCGAACAGCATATGGGCGATTGCCCGGTGACCGCGCTGGGCGCGAGCCTGTGCCCGAGCGAACAGGTGGCGAGCGTGATTTGCAGCGGCAACCAGAGCCAGACCCTGAGCAGCTGCAACAGCAGCAGCCTGGGCCCGACCCGCCCGACCATTCCGGAAGAAAGCGCGGTGGCGTGCATTGAAAGCGGCCAGCTGCGCCTGGTGAACGGCGGCGGCCGCTGCGCGGGCCGCGTGGAAATTTATCATGAAGGCAGCTGGGGCACCATTTGCGATGATAGCTGGGATCTGAGCGATGCGCATGTGGTGTGCCGCCAGCTGGGCTGCGGCGAAGCGATTAACGCGACCGGCAGCGCGCATTTTGGCGAAGGCACCGGCCCGATTTGGCTGGATGAAATGAAATGCAACGGCAAAGAAAGCCGCATTTGGCAGTGCCATAGCCATGGCTGGGGCCAGCAGAACTGCCGCCATAAAGAAGATGCGGGCGTGATTTGCAGCGAATTTATGAGCCTGCGCCTGACCAGCGAAGCGAGCCGCGAAGCGTGCGCGGGCCGCCTGGAAGTGTTTTATAACGGCGCGTGGGGCACCGTGGGCAAAAGCAGCATGAGCGAAACCACCGTGGGCGTGGTGTGCCGCCAGCTGGGCTGCGCGGATAAAGGCAAAATTAACCCGGCGAGCCTGGATAAAGCGATGAGCATTCCGATGTGGGTGGATAACGTGCAGTGCCCGAAAGGCCCGGATACCCTGTGGCAGTGCCCGAGCAGCCCGTGGGAAAAACGCCTGGCGAGCCCGAGCGAAGAAACCTGGATTACCTGCGATAACAAAATTCGCCTGCAGGAAGGCCCGACCAGCTGCAGCGGCCGCGTGGAAATTTGGCATGGCGGCAGCTGGGGCACCGTGTGCGATGATAGCTGGGATCTGGATGATGCGCAGGTGGTGTGCCAGCAGCTGGGCTGCGGCCCGGCGCTGAAAGCGTTTAAAGAAGCGGAATTTGGCCAGGGCACCGGCCCGATTTGGCTGAACGAAGTGAAATGCAAAGGCAACGAAAGCAGCCTGTGGGATTGCCCGGCGCGCCGCTGGGGCCATAGCGAATGCGGCCATAAAGAAGATGCGGCGGTGAACTGCACCGATATTAGCGTGCAGAAAACCCCGCAGAAAGCGACCACCGGCCGCAGCAGCCGCCAGAGCAGCTTTATTGCGGTGGGCATTCTGGGCGTGGTGCTGCTGGCGATTTTTGTGGCGCTGTTTTTTCTGACCAAAAAACGCCGCCAGCGCCAGCGCCTGGCGGTGAGCAGCCGCGGCGAAAACCTGGTGCATCAGATTCAGTATCGCGAAATGAACAGCTGCCTGAACGCGGATGATCTGGATCTGATGAACAGCAGCGAAAACAGCCATGAAAGCGCGGATTTTAGCGCGGCGGAACTGATTAGCGTGAGCAAATTTCTGCCGATTAGCGGCATGGAAAAAGAAGCGATTCTGAGCCATACCGAAAAAGAAAACGGCAACCTG

SEQ ID NO: 38 - protein, 1156 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 39 - DNA, 4011 bp, Homo sapiens
atgaaaccggcggcgcgcgaagcgcgcctgccgccgcgcagcccgggcctgcgctgggcgctgccgctgctgctgctgctgctgcgcctgggccagattctgtgcgcgggcggcaccccgagcccgattccggatccgagcgtggcgaccgtggcgaccggcgaaaacggcattacccagattagcagcaccgcggaaagctttcataaacagaacggcaccggcaccccgcaggtggaaaccaacaccagcgaagatggcgaaagcagcggcgcgaacgatagcctgcgcaccccggaacagggcagcaacggcaccgatggcgcgagccagaaaaccccgagcagcaccggcccgagcccggtgtttgatattaaagcggtgagcattagcccgaccaacgtgattctgacctggaaaagcaacgataccgcggcgagcgaatataaatatgtggtgaaacataaaatggaaaacgaaaaaaccattaccgtggtgcatcagccgtggtgcaacattaccggcctgcgcccggcgaccagctatgtgtttagcattaccccgggcattggcaacgaaacctggggcgatccgcgcgtgattaaagtgattaccgaaccgattccggtgagcgatctgcgcgtggcgctgaccggcgtgcgcaaagcggcgctgagctggagcaacggcaacggcaccgcgagctgccgcgtgctgctggaaagcattggcagccatgaagaactgacccaggatagccgcctgcaggtgaacattagcggcctgaaaccgggcgtgcagtataacattaacccgtatctgctgcagagcaacaaaaccaaaggcgatccgctgggcaccgaaggcggcctggatgcgagcaacaccgaacgcagccgcgcgggcagcccgaccgcgccggtgcatgatgaaagcctggtgggcccggtggatccgagcagcggccagcagagccgcgataccgaagtgctgctggtgggcctggaaccgggcacccgctataacgcgaccgtgtatagccaggcggcgaacggcaccgaaggccagccgcaggcgattgaatttcgcaccaacgcgattcaggtgtttgatgtgaccgcggtgaacattagcgcgaccagcctgaccctgatttggaaagtgagcgataacgaaagcagcagcaactatacctataaaattcatgtggcgggcgaaaccgatagcagcaacctgaacgtgagcgaaccgcgcgcggtgattccgggcctgcgcagcagcaccttttataacattaccgtgtgcccggtgctgggcgatattgaaggcaccccgggctttctgcaggtgcataccccgccggtgccggtgagcgattttcgcgtgaccgtggtgagcaccaccgaaattggcctggcgtggagcagccatgatgcggaaagctttcagatgcatattacccaggaaggcgcgggcaacagccgcgtggaaattaccaccaaccagagcattattattggcggcctgtttccgggcaccaaatattgctttgaaattgtgccgaaaggcccgaacggcaccgaaggcgcgagccgcaccgtgtgcaaccgcaccgtgccgagcgcggtgtttgatattcatgtggtgtatgtgaccaccaccgaaatgtggctggattggaaaagcccggatggcgcgagcgaatatgtgtatcatctggtgattgaaagcaaacatggcagcaaccataccagcacctatgataaagcgattaccctgcagggcctgattccgggcaccctgtataacattaccattagcccggaagtggatcatgtgtggggcgatccgaacagcaccgcgcagtatacccgcccgagcaacgtgagcaacattgatgtgagcaccaacaccaccgcggcgaccctgagctggcagaactttgatgatgcgagcccgacctatagctattgcctgctgattgaaaaagcgggcaacagcagcaacgcgacccaggtggtgaccgatattggcattaccgatgcgaccgtgaccgaactgattccgggcagcagctataccgtggaaatttttgcgcaggtgggcgatggcattaaaagcctggaaccgggccgcaaaagcttttgcaccgatccggcgagcatggcgagctttgattgcgaagtggtgccgaaagaaccggcgctggtgctgaaatggacctgcccgccgggcgcgaacgcgggctttgaactggaagtgagcagcggcgcgtggaacaacgcgacccatctggaaagctgcagcagcgaaaacggcaccgaatatcgcaccgaagtgacctatctgaactttagcaccagctataacattagcattaccaccgtgagctgcggcaaaatggcggcgccgacccgcaacacctgcaccaccggcattaccgatccgccgccgccggatggcagcccgaacattaccagcgtgagccataacagcgtgaaagtgaaatttagcggctttgaagcgagccatggcccgattaaagcgtatgcggtgattctgaccaccggcgaagcgggccatccgagcgcggatgtgctgaaatatacctatgaagattttaaaaaaggcgcgagcgatacctatgtgacctatctgattcgcaccgaagaaaaaggccgcagccagagcctgagcgaagtgctgaaatatgaaattgatgtgggcaacgaaagcaccaccctgggctattataacggcaaactggaaccgctgggcagctatcgcgcgtgcgtggcgggctttaccaacattacctttcatccgcagaacaaaggcctgattgatggcgcggaaagctatgtgagctttagccgctatagcgatgcggtgagcctgccgcaggatccgggcgtgatttgcggcgcggtgtttggctgcatttttggcgcgctggtgattgtgaccgtgggcggctttattttttggcgcaaaaaacgcaaagatgcgaaaaacaacgaagtgagctttagccagattaaaccgaaaaaaagcaaactgattcgcgtggaaaactttgaagcgtattttaaaaaacagcaggcggatagcaactgcggctttgcggaagaatatgaagatctgaaactggtgggcattagccagccgaaatatgcggcggaactggcggaaaaccgcggcaaaaaccgctataacaacgtgctgccgtatgatattagccgcgtgaaactgagcgtgcagacccatagcaccgatgattatattaacgcgaactatatgccgggctatcatagcaaaaaagattttattgcgacccagggcccgctgccgaacaccctgaaagatttttggcgcatggtgtgggaaaaaaacgtgtatgcgattattatgctgaccaaatgcgtggaacagggccgcaccaaatgcgaagaatattggccgagcaaacaggcgcaggattatggcgatattaccgtggcgatgaccagcgaaattgtgctgccggaatggaccattcgcgattttaccgtgaaaaacattcagaccagcgaaagccatccgctgcgccagtttcattttaccagctggccggatcatggcgtgccggataccaccgatctgctgattaactttcgctatctggtgcgcgattatatgaaacagagcccgccggaaagcccgattctggtgcattgcagcgcgggcgtgggccgcaccggcacctttattgcgattgatcgcctgatttatcagattgaaaacgaaaacaccgtggatgtgtatggcattgtgtatgatctgcgcatgcatcgcccgctgatggtgcagaccgaagatcagtatgtgtttctgaaccagtgcgtgctggatattgtgcgcagccagaaagatagcaaagtggatctgatttatcagaacaccaccgcgatgaccatttatgaaaacctggcgccggtgaccacctttggcaaaaccaacggctatattgcg

SEQ ID NO: 40 - protein, 1337 aa, Homo sapiens
MKPAAREARLPPRSPGLRWALPLLLLLLRLGQILCAGGTPSPIPDPSVATVATGENGITQISSTAESFHKQNGTGTPQVETNTSEDGESSGANDSLRTPEQGSNGTDGASQKTPSSTGPSPVFDIKAVSISPTNVILTWKSNDTAASEYKYVVKHKMENEKTITVVHQPWCNITGLRPATSYVFSITPGIGNETWGDPRVIKVITEPIPVSDLRVALTGVRKAALSWSNGNGTASCRVLLESIGSHEELTQDSRLQVNISGLKPGVQYNINPYLLQSNKTKGDPLGTEGGLDASNTERSRAGSPTAPVHDESLVGPVDPSSGQQSRDTEVLLVGLEPGTRYNATVYSQAANGTEGQPQAIEFRTNAIQVFDVTAVNISATSLTLIWKVSDNESSSNYTYKIHVAGETDSSNLNVSEPRAVIPGLRSSTFYNITVCPVLGDIEGTPGFLQVHTPPVPVSDFRVTVVSTTEIGLAWSSHDAESFQMHITQEGAGNSRVEITTNQSIIIGGLFPGTKYCFEIVPKGPNGTEGASRTVCNRTVPSAVFDIHVVYVTTTEMWLDWKSPDGASEYVYHLVIESKHGSNHTSTYDKAITLQGLIPGTLYNITISPEVDHVWGDPNSTAQYTRPSNVSNIDVSTNTTAATLSWQNFDDASPTYSYCLLIEKAGNSSNATQVVTDIGITDATVTELIPGSSYTVEIFAQVGDGIKSLEPGRKSFCTDPASMASFDCEVVPKEPALVLKWTCPPGANAGFELEVSSGAWNNATHLESCSSENGTEYRTEVTYLNFSTSYNISITTVSCGKMAAPTRNTCTTGITDPPPPDGSPNITSVSHNSVKVKFSGFEASHGPIKAYAVILTTGEAGHPSADVLKYTYEDFKKGASDTYVTYLIRTEEKGRSQSLSEVLKYEIDVGNESTTLGYYNGKLEPLGSYRACVAGFTNITFHPQNKGLIDGAESYVSFSRYSDAVSLPQDPGVICGAVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 41 - DNA, 1092 bp, Homo sapiens
atgccgtatcagtatccggcgctgaccccggaacagaaaaaagaactgagcgatattgcgcatcgcattgtggcgccgggcaaaggcattctggcggcggatgaaagcaccggcagcattgcgaaacgcctgcagagcattggcaccgaaaacaccgaagaaaaccgccgcttttatcgccagctgctgctgaccgcggatgatcgcgtgaacccgtgcattggcggcgtgattctgtttcatgaaaccctgtatcagaaagcggatgatggccgcccgtttccgcaggtgattaaaagcaaaggcggcgtggtgggcattaaagtggataaaggcgtggtgccgctggcgggcaccaacggcgaaaccaccacccagggcctggatggcctgagcgaacgctgcgcgcagtataaaaaagatggcgcggattttgcgaaatggcgctgcgtgctgaaaattggcgaacataccccgagcgcgctggcgattatggaaaacgcgaacgtgctggcgcgctatgcgagcatttgccagcagaacggcattgtgccgattgtggaaccggaaattctgccggatggcgatcatgatctgaaacgctgccagtatgtgaccgaaaaagtgctggcggcggtgtataaagcgctgagcgatcatcatatttatctggaaggcaccctgctgaaaccgaacatggtgaccccgggccatgcgtgcacccagaaatttagccatgaagaaattgcgatggcgaccgtgaccgcgctgcgccgcaccgtgccgccggcggtgaccggcattacctttctgagcggcggccagagcgaagaagaagcgagcattaacctgaacgcgattaacaaatgcccgctgctgaaaccgtgggcgctgacctttagctatggccgcgcgctgcaggcgagcgcgctgaaagcgtggggcggcaaaaaagaaaacctgaaagcggcgcaggaagaatatgtgaaacgcgcgctggcgaacagcctggcgtgccagggcaaatataccccgagcggccaggcgggcgcggcggcgagcgaaagcctgtttgtgagcaaccatgcgtat

SEQ ID NO: 42 - protein, 364 aa, Homo sapiens
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 43 - DNA, 522 bp, Homo sapiens
agcagccagattcgccagaactatagcaccgatgtggaagcggcggtgaacagcctggtgaacctgtatctgcaggcgagctatacctatctgagcctgggcttttattttgatcgcgatgatgtggcgctggaaggcgtgagccatttttttcgcgaactggcggaagaaaaacgcgaaggctatgaacgcctgctgaaaatgcagaaccagcgcggcggccgcgcgctgtttcaggatattaaaaaaccggcggaagatgaatggggcaaaaccccggatgcgatgaaagcggcgatggcgctggaaaaaaaactgaaccaggcgctgctggatctgcatgcgctgggcagcgcgcgcaccgatccgcatctgtgcgattttctggaaacccattttctggatgaagaagtgaaactgattaaaaaaatgggcgatcatctgaccaacctgcatcgcctgggcggcccggaagcgggcctgggcgaatatctgtttgaacgcctgaccctgaaacatgat

SEQ ID NO: 44 - protein, 175 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 45 - protein, 8 aa, Artificial Sequence
  synthetic polypeptide
ALQASALK

SEQ ID NO: 46 - protein, 12 aa, Artificial Sequence
  synthetic polypeptide
LTLLAPLNSVFK

SEQ ID NO: 47 - protein, 9 aa, Artificial Sequence
  synthetic polypeptide
ATVNPSAPR

SEQ ID NO: 48 - protein, 9 aa, Artificial Sequence
  synthetic polypeptide
AVGLAGTFR

SEQ ID NO: 49 - protein, 9 aa, Artificial Sequence
  synthetic polypeptide
SGYLLPDTK

SEQ ID NO: 50 - protein, 15 aa, Artificial Sequence
  synthetic polypeptide
LGGPEAGLGEYLFER

SEQ ID NO: 51 - protein, 9 aa, Artificial Sequence
  synthetic polypeptide
YYIAASYVK

SEQ ID NO: 52 - protein, 12 aa, Artificial Sequence
  synthetic polypeptide
TWNDPSVQQDIK

SEQ ID NO: 53 - protein, 8 aa, Artificial Sequence
  synthetic polypeptide
FLNVLSPR

SEQ ID NO: 54 - protein, 13 aa, Artificial Sequence
  synthetic polypeptide
ALPGTPVASSQPR

SEQ ID NO: 55 - protein, 9 aa, Artificial Sequence
  Synthesized Polypeptide
YVSELHLTR

SEQ ID NO: 56 - protein, 6 aa, Artificial Sequence
  synthetic polypeptide
VEIFYR

SEQ ID NO: 57 - protein, 16 aa, Artificial Sequence
  synthetic polypeptide
TVLWPNGLSLDIPAGR

SEQ ID NO: 58 - protein, 11 aa, Artificial Sequence
  synthetic polypeptide
QITVNDLPVGR

SEQ ID NO: 59 - protein, 14 aa, Artificial Sequence
  synthetic polypeptide
STGGAPTFNVTVTK

SEQ ID NO: 60 - protein, 9 aa, Artificial Sequence
  synthetic polypeptide
YEVTVVSVR

SEQ ID NO: 61 - protein, 9 aa, Artificial Sequence
  synthetic polypeptide
GFLLLASLR

SEQ ID NO: 62 - protein, 12 aa, Artificial Sequence
  synthetic polypeptide
YGFIEGHVVIPR

SEQ ID NO: 63 - protein, 12 aa, Artificial Sequence
  synthetic polypeptide
ASSIIDELFQDR

SEQ ID NO: 64 - protein, 8 aa, Artificial Sequence
  synthetic polypeptide
TASDFITK

SEQ ID NO: 65 - protein, 15 aa, Artificial Sequence
  Synthetic Polypeptide
TGVITSPDFPNPYPK

SEQ ID NO: 66 - protein, 12 aa, Artificial Sequence
  Synthetic Polypeptide
LDTLAQEVALLK

SEQ ID NO: 67 - protein, 8 aa, Artificial Sequence
  Synthetic Polypeptide
INPASLDK

SEQ ID NO: 68 - protein, 12 aa, Artificial Sequence
  Synthetic Polypeptide
LQSLFDSPDFSK

SEQ ID NO: 69 - protein, 11 aa, Artificial Sequence
  Synthetic Polypeptide
SLEDLQLTHNK

SEQ ID NO: 70 - protein, 12 aa, Artificial Sequence
  Synthetic Polypeptide
VITEPIPVSDLR

(19) United States
(12) Patent Application Publication
(10) Pub. No.: US 2017/0168058 A1
(43) Pub. Date: Jun. 15, 2017

(54) COMPOSITIONS, METHODS AND KITS FOR DIAGNOSIS OF LUNG CANCER

(71) Applicant: Integrated Diagnostics, Inc., Seattle, WA (US)

(72) Inventors: Paul EDWARD Kearney, Seattle, WA (US); Clive Hayward, Seattle, WA (US); Xiao-Jun Li, Bellevue, WA (US)

(21) Appl. No.: 15/427,883

(22) Filed: Feb. 8, 2017

Related U.S. Application Data
(63) Continuation of application No. 14/491,446, filed on Sep. 19, 2014, now abandoned.
(60) Provisional application No. 61/880,507, filed on Sep. 20, 2013.

(51) Int. Cl. G01N 33/574; G06F 19/00; G01N 33/49

(52) U.S. Cl. CPC G01N 33/57423; G01N 33/492; G06F 19/3431

(57) ABSTRACT

Methods are provided for identifying biomarker proteins that exhibit differential expression in subjects with a first lung condition versus healthy subjects or subjects with a second lung condition. Also provided are compositions comprising these biomarker proteins and methods of using these biomarker proteins or panels thereof to diagnose, classify, and monitor various lung conditions. The methods and compositions provided herein may be used to diagnose or classify a subject as having lung cancer or a non-cancerous condition, and to distinguish between different types of cancer (e.g., malignant versus benign, SCLC versus NSCLC).

### RELATED APPLICATIONS

This application is a continuation of U.S. application Ser. No. 14/491,446, filed Sep. 19, 2014, which claims the benefit of, and priority to, U.S. Provisional Application No. 61/880,507 filed Sep. 20, 2013, the content of which is incorporated herein by reference in its entirety.

### INCORPORATION-BY-REFERENCE OF SEQUENCE LISTING

The contents of the text file named “IDIA-010_001US_SEQ.txt”, which was created on Jun. 7, 2016 and is 281 KB in size, are hereby incorporated by reference in their entireties.

### BACKGROUND

Lung conditions and particularly lung cancer present significant diagnostic challenges. In many asymptomatic patients, radiological screens such as computed tomography (CT) scanning are a first step in the diagnostic paradigm. Pulmonary nodules (PNs) or indeterminate nodules are located in the lung and are often discovered during screening of both high risk patients or incidentally. The number of PNs identified is expected to rise due to increased numbers of patients with access to health care, the rapid adoption of screening techniques and an aging population. It is estimated that over 3 million PNs are identified annually in the US. Although the majority of PNs are benign, some are malignant leading to additional interventions. For patients considered low risk for malignant nodules, current medical practice dictates scans every three to six months for at least two years to monitor for lung cancer. The time period between identification of a PN and diagnosis is a time of medical surveillance or “watchful waiting” and may induce stress on the patient and lead to significant risk and expense due to repeated imaging studies. If a biopsy is performed on a patient who is found to have a benign nodule, the costs and potential for harm to the patient increase unnecessarily. Major surgery is indicated in order to excise a specimen for tissue biopsy and diagnosis. All of these procedures are associated with risk to the patient including: illness, injury and death as well as high economic costs.

Frequently, PNs cannot be biopsied to determine if they are benign or malignant due to their size and/or location in the lung. However, PNs are connected to the circulatory system, and so if malignant, protein markers of cancer can enter the blood and provide a signal for determining if a PN is malignant or not.

Diagnostic methods that can replace or complement current diagnostic methods for patients presenting with PNs are needed to improve diagnostics, reduce costs and minimize invasive procedures and complications to patients.

### SUMMARY

The present invention provides novel compositions, methods and kits for identifying protein markers to identify, diagnose, classify and monitor lung conditions, and particularly lung cancer. The present invention uses a multiplexed assay to distinguish benign pulmonary nodules from malignant pulmonary nodules to classify patients with or without lung cancer. The present invention may be used in patients who present with symptoms of lung cancer, but do not have pulmonary nodules.

The present invention provides a method of determining the likelihood that a lung condition in a subject is cancer by assessing the expression of proteins in a sample obtained from the subject; calculating a score based on the protein abundance; and comparing the score from the biological sample to a plurality of scores obtained from a reference population, wherein the comparison provides a determination that the lung condition is cancer. When cancer is ruled in, the subject receives a treatment protocol. Treatment protocol includes for example pulmonary function test (PFT), pulmonary imaging, a biopsy, a surgery, a chemotherapy, a radiotherapy, or any combination thereof. In some embodiments, the imaging is an x-ray, a chest computed tomography (CT) scan, or a positron emission tomography (PET) scan.

The present invention provides a method of determining that a lung condition in a subject is cancer by assessing the expression of a plurality of proteins comprising determining the protein expression level of at least each of BGH3_HUMAN, GGH_HUMAN, LG3BP_HUMAN, PRDX1_HUMAN and TSP1_HUMAN from a biological sample obtained from the subject; calculating a score from the protein expression of at least each of BGH3_HUMAN, GGH_HUMAN, LG3BP_HUMAN, PRDX1_HUMAN and TSP1_HUMAN from the biological sample from the previous step; and comparing the score from the biological sample to a plurality of scores obtained from a reference population, wherein the comparison provides a determination that the lung condition is cancer.

In one embodiment the subject has a pulmonary nodule, wherein the pulmonary nodule has a diameter of 30 mm or less. Preferably, the pulmonary nodule has a diameter of about 8 and 30 mm. In one embodiment, the lung condition of the subject is cancer or a non-cancerous lung condition. In another embodiment, the lung cancer is non-small cell lung cancer. The non-cancerous lung conditions include chronic obstructive pulmonary disease, hamartoma, fibroma, neurofibroma, granuloma, sarcoidosis, bacterial infection or fungal infection.

The subject can be a mammal. Preferably, the subject is a human.

The biological sample can be any sample obtained from the subject, e.g., tissue, cell, fluid. Preferably, the biological sample is tissue, blood plasma, serum, whole blood, urine, saliva, genital secretions, cerebrospinal fluid, sweat, excreta or bronchoalveolar lavage.

The method of the present invention includes assessing the expression level of at least each of BGH3_HUMAN, GGH_HUMAN, LG3BP_HUMAN, PRDX1_HUMAN and TSP1_HUMAN and fragmenting each protein to generate at least one peptide. The method of fragmentation can include trypsin digestion. The methods of the current invention can include various manners to assess the expression of a plurality of proteins, including mass spectrometry (MS), liquid chromatography-selected reaction monitoring/mass spectrometry (LC-SRM-MS), reverse transcriptase-polymerase chain reaction (RT-PCR), microarray, serial analysis of gene expression (SAGE), gene expression analysis by massively parallel signature sequencing (MPSS), immunoassays, immunohistochemistry (IHC), transcriptomics, or proteomics. A preferred embodiment of the current invention is assessing the expression of a plurality of proteins by liquid chromatography-selected reaction monitoring/mass spectrometry (LC-SRM-MS). In another aspect of the invention, at least one transition for each peptide is determined by liquid chromatography-selected reaction monitoring/mass spectrometry (LC-SRM-MS). In one embodiment, the peptide transitions comprise at least LTLLAPLNSVFK £SEQ ID No.: 46) (658.4, 804.5), YYIAASYVK (SEQ ID No.: 51) (539.28, 638.4), VEIFYR (SEQ ID No.: 56) (413.73, 598.3), QITVNDLPVGR (SEQ ID No.: 58) (606.3, 970.5), and GFLLLASLR (SEQ ID No.: 61) (495.31, 559.4).

The methods of the current invention provide a means to determine a score, wherein said score is determined as score=1/[1+exp(−α−Σᵢ₌₁⁵βᵢ*\check{P}ᵢ)], wherein

$${\tilde{P}}_{i} = \frac{{P}_{i}^{{λ}_{i}} - 1.0}{{λ}_{i}} ,$$

and \check{P}ᵢ is the Box-Cox transformed and normalized intensity of peptide transition i in said sample, βᵢ is the corresponding logistic regression coefficient, λᵢ is the corresponding Box-Cox transformation, α is a panel-specific constant, and N is the total number of transitions of the assessed proteins. In one embodiment, the reference population comprises at least 100 subjects with a lung condition and wherein each subject in the reference population has been assigned a score based on the protein expression of at least each of BGH3_HUMAN, GGH_HUMAN, LG3BP_HUMAN, PRDX1_HUMAN and TSP1_HUMAN obtained from a biological sample.

The methods of the current invention can further include normalizing the protein measurements. The methods of the current invention can further include normalizing the protein expression level of at least each of BGH3_HUMAN, GGH_HUMAN, LG3BP_HUMAN, PRDX1_HUMAN and TSP1_HUMAN against the protein expression level of at least one of PEDF_HUMAN, MASP1_HUMAN, GELS_HUMAN, LUM_HUMAN, C163A_HUMAN, PTPRJ_HUMAN, CD44_HUMAN, TENX_HUMAN, CLUS_HUMAN, and IBP3_HUMAN in the sample.

In another aspect of the current invention, the score from the biological sample from the subject is calculated from a logistic regression model applied to the determined protein expression levels. In another embodiment, the plurality of scores obtained from a reference population provides a single pre-determined score, and wherein if the score from the biological sample from the subject is equal or greater than the pre-determined score, the lung condition is cancer. In another embodiment, the score is within a range of possible values and the predetermined score is approximately 65% of the magnitude of the range. In another aspect, the score from the biological sample provides a positive predictive value (PPV) of at least 30%. In another aspect, the score from the biological sample provides a positive predictive value (PPV) of at least 50%.

Another aspect of the current invention comprises treating the subject if the lung condition is cancer. The methods of the invention provide for treatment of the subject if the lung condition is cancer, wherein said treatment is a pulmonary function test (PFT), pulmonary imaging, a biopsy, a surgery, a chemotherapy, a radiotherapy, or any combination thereof. In one embodiment of the current invention, the imaging includes an x-ray, a chest computed tomography (CT) scan, or a positron emission tomography (PET) scan. Another aspect of the current invention can include at least one step performed on a computer system.

Although methods and materials similar or equivalent to those described herein can be used in the practice or testing of the present invention, suitable methods and materials are described below. All publications, patent applications, patents, and other references mentioned herein are incorporated by reference in their entirety. The references cited herein are not admitted to be prior art to the claimed invention. In the case of conflict, the present specification, including definitions, will control. In addition, the materials, methods, and examples are illustrative only and are not intended to be limiting. Other features and advantages of the invention will be apparent from the following detailed description and claim.

### BRIEF DESCRIPTION OF THE DRAWINGS

FIG. 1 is a panel of graphs explaining calculation of partial AUC (pAUC) factor. Panel A shows ROC curve of the performance of a classifier. Panel B shows the expected random partial AUC at 20% false positive rate (FPR). Panel C shows the actual partial AUC at 20% FPR.

FIG. 2 is a graph showing pAUC of overall 1 million panels' performance.

FIG. 3A is a graph showing panels with pAUC factor >=1.5.

FIG. 3B is a graph showing panels with pAUC factor >=1.75.

FIG. 4 is a graph showing performance of all 7-protein panels.

FIG. 5A is a graph showing performance of panel 1.

FIG. 5B is a graph showing performance of panel 2.

FIG. 5C is a graph showing performance of panel 3.

FIG. 5D is a graph showing performance of panel 4.

FIG. 5E is a graph showing performance of panel 5.

FIG. 5F is a graph showing performance of panel 6.

FIG. 6 is a graph showing performance of panel 4.

### DETAILED DESCRIPTION

The disclosed invention derives from the surprising discovery that in patients presenting with pulmonary nodule(s), a small panel of protein markers in the blood is able to specifically identify and distinguish malignant and benign lung nodules with high positive predictive value (PPV) and sensitivity. The classifiers described herein demonstrate remarkable independence and accuracy. Particularly, these classifiers (a.k.a., rule-in classifiers) are useful to identify cancer patients among those who cannot be ruled out by the rule-out classifiers.

Accordingly the invention provides unique advantages to the patient associated with early detection of lung cancer in a patient, including increased life span, decreased morbidity and mortality, decreased exposure to radiation during screening and repeat screenings and a minimally invasive diagnostic model. Importantly, the methods of the invention allow for a patient to avoid invasive procedures.

The routine clinical use of chest computed tomography (CT) scans identifies millions of pulmonary nodules annually, of which only a small minority are malignant but contribute to the dismal 15% five-year survival rate for patients diagnosed with non-small cell lung cancer (NSCLC). The early diagnosis of lung cancer in patients with pulmonary nodules is a top priority, as decision-making based on clinical presentation, in conjunction with current non-invasive diagnostic options such as chest CT and positron emission tomography (PET) scans, and other invasive alternatives, has not altered the clinical outcomes of patients with Stage I NSCLC. The subgroup of pulmonary nodules between 8 mm and 20 mm in size is increasingly recognized as being “intermediate” relative to the lower rate of malignancies below 8 mm and the higher rate of malignancies above 20 mm. Invasive sampling of the lung nodule by biopsy using transthoracic needle aspiration or bronchoscopy may provide a cytopathologic diagnosis of NSCLC, but are also associated with both false-negative and non-diagnostic results. In summary, a key unmet clinical need for the management of pulmonary nodules is a non-invasive diagnostic test that discriminates between malignant and benign processes in patients with indeterminate pulmonary nodules (IPNs), especially between 8 mm and 20 mm in size.

The clinical decision to be more or less aggressive in treatment is based on risk factors, primarily nodule size, smoking history and age in addition to imaging. As these are not conclusive, there is a great need for a molecular-based blood test that would be both non-invasive and provide complementary information to risk factors and imaging.

Accordingly, these and related embodiments will find uses in screening methods for lung conditions, and particularly lung cancer diagnostics. More importantly, the invention finds use in determining the clinical management of a patient. That is, the method of invention is particularly useful in ruling in a particular treatment protocol for an individual subject.

Cancer biology requires a molecular strategy to address the unmet medical need for an assessment of lung cancer risk. The field of diagnostic medicine has evolved with technology and assays that provide sensitive mechanisms for detection of changes in proteins. The methods described herein use a LC-SRM-MS technology for measuring the concentration of blood plasma proteins that are collectively changed in patients with a malignant PN. This protein signature is indicative of lung cancer. LC-SRM-MS is one method that provides for both quantification and identification of circulating proteins in plasma. Changes in protein expression levels, such as but not limited to signaling factors, growth factors, cleaved surface proteins and secreted proteins, can be detected using such a sensitive technology to assay cancer. Presented herein is a blood-based classification test to determine the likelihood that a patient presenting with a pulmonary nodule has a nodule that is benign or malignant. The present invention presents a classification algorithm that predicts the relative likelihood of the PN being benign or malignant.

More broadly, it is demonstrated that there are many variations on this invention that are also diagnostic tests for the likelihood that a PN or a pulmonary mass is benign or malignant. These are variations on the panel of proteins, protein standards, measurement methodology and/or classification algorithm.

As disclosed herein, archival plasma samples from subjects presenting with PNs were analyzed for differential protein expression by mass spectrometry and the results were used to identify biomarker proteins and panels of biomarker proteins that are differentially expressed in conjunction with various lung conditions (cancer vs. non-cancer).

In one aspect of the invention, the panel comprises at least 2, 3, 4, 5, or more protein markers with at least one protein-protein interaction. In some embodiments, the panel comprises 5 protein markers. For example, the panel comprises BGH3_HUMAN, GGH_HUMAN, LG3BP_HUMAN, PRDX1_HUMAN, and TSP1_HUMAN. Alternatively, the panel comprises COIA1_HUMAN, ENPL_HUMAN, GGH_HUMAN, PRDX1_HUMAN, and TSP1_HUMAN. In some embodiments, the panel comprises 6 biomarkers. For example, the panel comprises BGH3_HUMAN, COIA1_HUMAN, ENPL_HUMAN, GGH_HUMAN, PRDX1_HUMAN, and TSP1_HUMAN.

Additional biomarkers that can be used herein are described in WO13/096845, the contents of which are incorporated herein by reference in their entireties.

The term “pulmonary nodules” (PNs) refers to lung lesions that can be visualized by radiographic techniques. A pulmonary nodule is any nodules less than or equal to three centimeters in diameter. In one example a pulmonary nodule has a diameter of about 0.8 cm to 2 cm.

The term “masses” or “pulmonary masses” refers to lung nodules that are greater than three centimeters maximal diameter.

The term “blood biopsy” refers to a diagnostic study of the blood to determine whether a patient presenting with a nodule has a condition that may be classified as either benign or malignant.

The term “acceptance criteria” refers to the set of criteria to which an assay, test, diagnostic or product should conform to be considered acceptable for its intended use. As used herein, acceptance criteria are a list of tests, references to analytical procedures, and appropriate measures, which are defined for an assay or product that will be used in a diagnostic. For example, the acceptance criteria for the classifier refer to a set of predetermined ranges of coefficients.

The term “partial AUC factor or pAUC factor” is greater than expected by random prediction. At specificity=0.80 the pAUC factor is the trapezoidal area under the ROC curve from 0.0 to 0.2 False Positive Rate/(0.2*0.2/2).

The term “incremental information” refers to information that may be used with other diagnostic information to enhance diagnostic accuracy. Incremental information is independent of clinical factors such as including nodule size, age, or gender.

The term “score” or “scoring” refers to calculating a probability likelihood for a sample. For the present invention, values closer to 1.0 are used to represent the likelihood that a sample is cancer, values closer to 0.0 represent the likelihood that a sample is benign.

The term “robust” refers to a test or procedure that is not seriously disturbed by violations of the assumptions on which it is based. For the present invention, a robust test is a test wherein the proteins or transitions of the mass spectrometry chromatograms have been manually reviewed and are “generally” free of interfering signals.

The term “coefficients” refers to the weight assigned to each protein used to in the logistic regression model to score a sample.

In certain embodiments of the invention, it is contemplated that in terms of the logistic regression model of MC CV, the model coefficient and the coefficient of variation (CV) of each protein's model coefficient may increase or decrease, dependent upon the method (or model) of measurement of the protein classifier. For each of the listed proteins in the panels, there is about, at least, at least about, or at most about a 2-, 3-, 4-, 5-, 6-, 7-, 8-, 9-, or 10-, -fold or any range derivable therein for each of the coefficient and CV. Alternatively, it is contemplated that quantitative embodiments of the invention may be discussed in terms of as about, at least, at least about, or at most about 10, 20, 30, 40, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein.

The term “best team players” refers to the proteins that rank the best in the random panel selection algorithm, i.e., perform well on panels. When combined into a classifier these proteins can segregate cancer from benign samples. “Best team player proteins” are synonymous with “cooperative proteins”. The term “cooperative proteins” refers to proteins that appear more frequently on high performing panels of proteins than expected by chance. This gives rise to a protein's cooperative score which measures how (in) frequently it appears on high performing panels. For example, a protein with a cooperative score of 1.5 appears on high performing panels 1.5× more than would be expected by chance alone.

The term “classifying” as used herein with regard to a lung condition refers to the act of compiling and analyzing expression data for using statistical techniques to provide a classification to aid in diagnosis of a lung condition, particularly lung cancer.

The term “classifier” as used herein refers to an algorithm that discriminates between disease states with a predetermined level of statistical significance. A two-class classifier is an algorithm that uses data points from measurements from a sample and classifies the data into one of two groups. In certain embodiments, the data used in the classifier is the relative expression of proteins in a biological sample. Protein expression levels in a subject can be compared to levels in patients previously diagnosed as disease free or with a specified condition. Table 5 lists representative rule-in classifiers (e.g., panels 1, 4, and 5).

The “classifier” maximizes the probability of distinguishing a randomly selected cancer sample from a randomly selected benign sample, i.e., the AUC of ROC curve.

In addition to the classifier's constituent proteins with differential expression, it may also include proteins with minimal or no biologic variation to enable assessment of variability, or the lack thereof, within or between clinical specimens; these proteins may be termed endogenous proteins and serve as internal controls for the other classifier proteins.

The term “normalization” or “normalizer” as used herein refers to the expression of a differential value in terms of a standard value to adjust for effects which arise from technical variation due to sample handling, sample preparation and mass spectrometry measurement rather than biological variation of protein concentration in a sample. For example, when measuring the expression of a differentially expressed protein, the absolute value for the expression of the protein can be expressed in terms of an absolute value for the expression of a standard protein that is substantially constant in expression. This prevents the technical variation of sample preparation and mass spectrometry measurement from impeding the measurement of protein concentration levels in the sample. A skilled artisan could readily recognize that any normalization methods and/or normalizers suitable for the present invention can be utilized.

The term “condition” as used herein refers generally to a disease, event, or change in health status.

The term “treatment protocol” as used herein includes further diagnostic testing typically performed to determine whether a pulmonary nodule is benign or malignant. Treatment protocols include diagnostic tests typically used to diagnose pulmonary nodules or masses such as for example, CT scan, positron emission tomography (PET) scan, bronchoscopy or tissue biopsy. Treatment protocol as used herein is also meant to include therapeutic treatments typically used to treat malignant pulmonary nodules and/or lung cancer such as for example, chemotherapy, radiation or surgery.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore the term diagnosis includes: a. prediction (determining if a patient will likely develop a hyperproliferative disease); b. prognosis (predicting whether a patient will likely have a better or worse outcome at a pre-selected time in the future); c. therapy selection; d. therapeutic drug monitoring; and e. relapse monitoring.

In some embodiments, for example, classification of a biological sample as being derived from a subject with a lung condition may refer to the results and related reports generated by a laboratory, while diagnosis may refer to the act of a medical professional in using the classification to identify or verify the lung condition.

The term “providing” as used herein with regard to a biological sample refers to directly or indirectly obtaining the biological sample from a subject. For example, “providing” may refer to the act of directly obtaining the biological sample from a subject (e.g., by a blood draw, tissue biopsy, lavage and the like). Likewise, “providing” may refer to the act of indirectly obtaining the biological sample. For example, providing may refer to the act of a laboratory receiving the sample from the party that directly obtained the sample, or to the act of obtaining the sample from an archive.

As used herein, “lung cancer” preferably refers to cancers of the lung, but may include any disease or other disorder of the respiratory system of a human or other mammal. Respiratory neoplastic disorders include, for example small cell carcinoma or small cell lung cancer (SCLC), non-small cell carcinoma or non-small cell lung cancer (NSCLC), squamous cell carcinoma, adenocarcinoma, broncho-alveolar carcinoma, mixed pulmonary carcinoma, malignant pleural mesothelioma, undifferentiated large cell carcinoma, giant cell carcinoma, synchronous tumors, large cell neuroendocrine carcinoma, adenosquamous carcinoma, undifferentiated carcinoma; and small cell carcinoma, including oat cell cancer, mixed small cell/large cell carcinoma, and combined small cell carcinoma; as well as adenoid cystic carcinoma, hamartomas, mucoepidermoid tumors, typical carcinoid lung tumors, atypical carcinoid lung tumors, peripheral carcinoid lung tumors, central carcinoid lung tumors, pleural mesotheliomas, and undifferentiated pulmonary carcinoma and cancers that originate outside the lungs such as secondary cancers that have metastasized to the lungs from other parts of the body. Lung cancers may be of any stage or grade. Preferably the term may be used to refer collectively to any dysplasia, hyperplasia, neoplasia, or metastasis in which the protein biomarkers expressed above normal levels as may be determined, for example, by comparison to adjacent healthy tissue.

Examples of non-cancerous lung condition include chronic obstructive pulmonary disease (COPD), benign tumors or masses of cells (e.g., hamartoma, fibroma, neurofibroma), granuloma, sarcoidosis, and infections caused by bacterial (e.g., tuberculosis) or fungal (e.g., histoplasmosis) pathogens. In certain embodiments, a lung condition may be associated with the appearance of radiographic PNs.

As used herein, “lung tissue” and “lung cancer” refer to tissue or cancer, respectively, of the lungs themselves, as well as the tissue adjacent to and/or within the strata underlying the lungs and supporting structures such as the pleura, intercostal muscles, ribs, and other elements of the respiratory system. The respiratory system itself is taken in this context as representing nasal cavity, sinuses, pharynx, larynx, trachea, bronchi, lungs, lung lobes, aveoli, aveolar ducts, aveolar sacs, aveolar capillaries, bronchioles, respiratory bronchioles, visceral pleura, parietal pleura, pleural cavity, diaphragm, epiglottis, adenoids, tonsils, mouth and tongue, and the like. The tissue or cancer may be from a mammal and is preferably from a human, although monkeys, apes, cats, dogs, cows, horses and rabbits are within the scope of the present invention. The term “lung condition” as used herein refers to a disease, event, or change in health status relating to the lung, including for example lung cancer and various non-cancerous conditions.

“Accuracy” refers to the degree of conformity of a measured or calculated quantity (a test reported value) to its actual (or true) value. Clinical accuracy relates to the proportion of true outcomes (true positives (TP) or true negatives (TN)) versus misclassified outcomes (false positives (FP) or false negatives (FN)), and may be stated as a sensitivity, specificity, positive predictive values (PPV) or negative predictive values (NPV), or as a likelihood, odds ratio, among other measures. The term “biological sample” as used herein refers to any sample of biological origin potentially containing one or more biomarker proteins. Examples of biological samples include tissue, organs, or bodily fluids such as whole blood, plasma, serum, tissue, lavage or any other specimen used for detection of disease.

The term “subject” as used herein refers to a mammal, preferably a human.

The term “biomarker protein” as used herein refers to a polypeptide in a biological sample from a subject with a lung condition versus a biological sample from a control subject. A biomarker protein includes not only the polypeptide itself, but also minor variations thereof, including for example one or more amino acid substitutions or modifications such as glycosylation or phosphorylation.

The term “biomarker protein panel” as used herein refers to a plurality of biomarker proteins. In certain embodiments, the expression levels of the proteins in the panels can be correlated with the existence of a lung condition in a subject. In certain embodiments, biomarker protein panels comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 proteins. In certain embodiments, the biomarker proteins panels comprise 2-5 proteins, 5-10 proteins, 10-20 proteins or more.

“Treating” or “treatment” as used herein with regard to a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

Biomarker levels may change due to treatment of the disease. The changes in biomarker levels may be measured by the present invention. Changes in biomarker levels may be used to monitor the progression of disease or therapy.

“Altered”, “changed” or “significantly different” refer to a detectable change or difference from a reasonably comparable state, profile, measurement, or the like. One skilled in the art should be able to determine a reasonable measurable change. Such changes may be all or none. They may be incremental and need not be linear. They may be by orders of magnitude. A change may be an increase or decrease by 1%, 5%, 10%, 20%, 30%, 40%, 50%, 60%, 70%, 80%, 90%, 95%, 99%, 100%, or more, or any value in between 0% and 100%. Alternatively the change may be 1-fold, 1.5-fold 2-fold, 3-fold, 4-fold, 5-fold or more, or any values in between 1-fold and five-fold. The change may be statistically significant with a p value of 0.1, 0.05, 0.001, or 0.0001.

Using the methods of the current invention, a clinical assessment of a patient is first performed. If there exists is a higher likelihood for cancer, the clinician may rule in the disease which will require the pursuit of diagnostic testing options yielding data which increase and/or substantiate the likelihood of the diagnosis. “Rule in” of a disease requires a test with a high specificity.

“FN” is false negative, which for a disease state test means classifying a disease subject incorrectly as non-disease or normal.

“FP” is false positive, which for a disease state test means classifying a normal subject incorrectly as having disease.

The term “rule in” refers to a diagnostic test with high specificity that optionally coupled with a clinical assessment indicates a higher likelihood for cancer. If the clinical assessment is a lower likelihood for cancer, the clinician may adopt a stance to rule out the disease, which will require diagnostic tests which yield data that decrease the likelihood of the diagnosis. “Rule out” requires a test with a high sensitivity. Accordingly, the term “ruling in” as used herein is meant that the subject is selected to receive a treatment protocol.

The term “rule out” refers to a diagnostic test with high sensitivity that optionally coupled with a clinical assessment indicates a lower likelihood for cancer. Accordingly, the term “ruling out” as used herein is meant that the subject is selected not to receive a treatment protocol.

The term “sensitivity of a test” refers to the probability that a patient with the disease will have a positive test result. This is derived from the number of patients with the disease who have a positive test result (true positive) divided by the total number of patients with the disease, including those with true positive results and those patients with the disease who have a negative result, i.e., false negative.

The term “specificity of a test” refers to the probability that a patient without the disease will have a negative test result. This is derived from the number of patients without the disease who have a negative test result (true negative) divided by all patients without the disease, including those with a true negative result and those patients without the disease who have a positive test result, e.g., false positive. While the sensitivity, specificity, true or false positive rate, and true or false negative rate of a test provide an indication of a test's performance, e.g., relative to other tests, to make a clinical decision for an individual patient based on the test's result, the clinician requires performance parameters of the test with respect to a given population.

The term “positive predictive value” (PPV) refers to the probability that a positive result correctly identifies a patient who has the disease, which is the number of true positives divided by the sum of true positives and false positives.

The term “negative predictive value” or “NPV” is calculated by TN/(TN+FN) or the true negative fraction of all negative test results. It also is inherently impacted by the prevalence of the disease and pre-test probability of the population intended to be tested. The term NPV refers to the probability that a negative test correctly identifies a patient without the disease, which is the number of true negatives divided by the sum of true negatives and false negatives. A positive result from a test with a sufficient PPV can be used to rule in the disease for a patient, while a negative result from a test with a sufficient NPV can be used to rule out the disease, if the disease prevalence for the given population, of which the patient can be considered a part, is known.

The term “disease prevalence” refers to the number of all new and old cases of a disease or occurrences of an event during a particular period. Prevalence is expressed as a ratio in which the number of events is the numerator and the population at risk is the denominator.

The term disease incidence refers to a measure of the risk of developing some new condition within a specified period of time; the number of new cases during some time period, it is better expressed as a proportion or a rate with a denominator.

Lung cancer risk according to the “National Lung Screening Trial” is classified by age and smoking history. High risk—age ≧55 and ≧30 pack-years smoking history; Moderate risk—age ≧50 and ≧20 pack-years smoking history; Low risk—<age 50 or <20 pack-years smoking history.

The clinician must decide on using a diagnostic test based on its intrinsic performance parameters, including sensitivity and specificity, and on its extrinsic performance parameters, such as positive predictive value and negative predictive value, which depend upon the disease's prevalence in a given population.

Additional parameters which may influence clinical assessment of disease likelihood include the prior frequency and closeness of a patient to a known agent, e.g., exposure risk, that directly or indirectly is associated with disease causation, e.g., second hand smoke, radiation, etc., and also the radiographic appearance or characterization of the pulmonary nodule exclusive of size. A nodule's description may include solid, semi-solid or ground glass which characterizes it based on the spectrum of relative gray scale density employed by the CT scan technology.

“Mass spectrometry” refers to a method comprising employing an ionization source to generate gas phase ions from an analyte presented on a sample presenting surface of a probe and detecting the gas phase ions with a mass spectrometer.

In some embodiments of the invention, two panels of 5 proteins (BGH3_HUMAN, GGH_HUMAN, LG3BP_HUMAN, PRDX1_HUMAN, and TSP1_HUMAN; or COIA1_HUMAN, ENPL_HUMAN, GGH_HUMAN, PRDX1_HUMAN, and TSP1_HUMAN) or a panel of 6 proteins (BGH3_HUMAN, COIA1_HUMAN, ENPL_HUMAN, GGH_HUMAN, PRDX1_HUMAN, and TSP1_HUMAN) effectively distinguishes between samples derived from patients with benign and malignant nodules less than 2 cm diameter, particularly identifying cancer patients among those who cannot be ruled out by the rule-out classifiers.

Bioinformatic and biostatistical analyses were used first to identify individual proteins with statistically significant differential expression, and then using these proteins to derive one or more combinations of proteins or panels of proteins, which collectively demonstrated superior discriminatory performance compared to any individual protein. Bioinformatic and biostatistical methods are used to derive coefficients (C) for each individual protein in the panel that reflects its relative expression level, i.e., increased or decreased, and its weight or importance with respect to the panel's net discriminatory ability, relative to the other proteins. The quantitative discriminatory ability of the panel can be expressed as a mathematical algorithm with a term for each of its constituent proteins being the product of its coefficient and the protein's plasma expression level (P) (as measured by LC-SRM-MS), e.g., C×P, with an algorithm consisting of n proteins described as: C1×P1+C2×P2+C3×P3++Cn×Pn. An algorithm that discriminates between disease states with a predetermined level of statistical significance may be refers to a “disease classifier”. In addition to the classifier's constituent proteins with differential expression, it may also include proteins with minimal or no biologic variation to enable assessment of variability, or the lack thereof, within or between clinical specimens; these proteins may be termed typical native proteins and serve as internal controls for the other classifier proteins.

In certain embodiments, expression levels are measured by MS. MS analyzes the mass spectrum produced by an ion after its production by the vaporization of its parent protein and its separation from other ions based on its mass-to-charge ratio. The most common modes of acquiring MS data are 1) full scan acquisition resulting in the typical total ion current plot (TIC), 2) selected ion monitoring (SIM), and 3) selected reaction monitoring (SRM).

In certain embodiments of the methods provided herein, biomarker protein expression levels are measured by LC-SRM-MS. LC-SRM-MS is a highly selective method of tandem mass spectrometry which has the potential to effectively filter out all molecules and contaminants except the desired analyte(s). This is particularly beneficial if the analysis sample is a complex mixture which may comprise several isobaric species within a defined analytical window. LC-SRM-MS methods may utilize a triple quadrupole mass spectrometer which, as is known in the art, includes three quadrupole rod sets. A first stage of mass selection is performed in the first quadrupole rod set, and the selectively transmitted ions are fragmented in the second quadrupole rod set. The resultant transition (product) ions are conveyed to the third quadrupole rod set, which performs a second stage of mass selection. The product ions transmitted through the third quadrupole rod set are measured by a detector, which generates a signal representative of the numbers of selectively transmitted product ions. The RF and DC potentials applied to the first and third quadrupoles are tuned to select (respectively) precursor and product ions that have m/z values lying within narrow specified ranges. By specifying the appropriate transitions (m/z values of precursor and product ions), a peptide corresponding to a targeted protein may be measured with high degrees of sensitivity and selectivity. Signal-to-noise ratio is superior to conventional tandem mass spectrometry (MS/MS) experiments, which select one mass window in the first quadrupole and then measure all generated transitions in the ion detector. LC-SRM-MS.

In certain embodiments, an SRM-MS assay for use in diagnosing or monitoring lung cancer as disclosed herein may utilize one or more peptides and/or peptide transitions derived from the proteins BGH3_HUMAN, GGH_HUMAN, LG3BP_HUMAN, PRDX1_HUMAN, and TSP1_HUMAN (see, for example, Tables 1-5). In certain embodiments, the assay may utilize one or more peptides and/or peptide transitions derived from the proteins COIA1_HUMAN, ENPL_HUMAN, GGH_HUMAN, PRDX1_HUMAN, and TSP1_HUMAN. In certain embodiments, it may utilize one or more peptides and/or peptide transitions derived from the proteins BGH3_HUMAN, COIA1_HUMAN, ENPL_HUMAN, GGH_HUMAN, PRDX1_HUMAN, and TSP1_HUMAN. Exemplary peptide transitions derived from these proteins are shown in Tables 10A-10C and 11A-11M.

The expression level of a biomarker protein can be measured using any suitable method known in the art, including but not limited to mass spectrometry (MS), reverse transcriptase-polymerase chain reaction (RT-PCR), microarray, serial analysis of gene expression (SAGE), gene expression analysis by massively parallel signature sequencing (MPSS), immunoassays (e.g., ELISA), immunohistochemistry (IHC), transcriptomics, and proteomics.

To evaluate the diagnostic performance of a particular set of peptide transitions, a ROC curve is generated for each significant transition.

An “ROC curve” as used herein refers to a plot of the true positive rate (sensitivity) against the false positive rate (specificity) for a binary classifier system as its discrimination threshold is varied. A ROC curve can be represented equivalently by plotting the fraction of true positives out of the positives (TPR=true positive rate) versus the fraction of false positives out of the negatives (FPR=false positive rate). Each point on the ROC curve represents a sensitivity/specificity pair corresponding to a particular decision threshold.

AUC represents the area under the ROC curve. The AUC is an overall indication of the diagnostic accuracy of 1) a biomarker or a panel of biomarkers and 2) a ROC curve. AUC is determined by the “trapezoidal rule.” For a given curve, the data points are connected by straight line segments, perpendiculars are erected from the abscissa to each data point, and the sum of the areas of the triangles and trapezoids so constructed is computed. In certain embodiments of the methods provided herein, a biomarker protein has an AUC in the range of about 0.75 to 1.0. In certain of these embodiments, the AUC is in the range of about 0.8 to 0.85, 0.85 to 0.9, 0.9 to 0.95, or 0.95 to 1.0.

The methods provided herein are minimally invasive and pose little or no risk of adverse effects. As such, they may be used to diagnose, monitor and provide clinical management of subjects who do not exhibit any symptoms of a lung condition and subjects classified as low risk for developing a lung condition. For example, the methods disclosed herein may be used to diagnose lung cancer in a subject who does not present with a PN and/or has not presented with a PN in the past, but who nonetheless deemed at risk of developing a PN and/or a lung condition. Similarly, the methods disclosed herein may be used as a strictly precautionary measure to diagnose healthy subjects who are classified as low risk for developing a lung condition.

The present invention provides a method of determining the likelihood that a lung condition in a subject is cancer by measuring the abundance of a panel of proteins in a sample obtained from the subject; calculating a probability of cancer score based on the protein measurements and ruling in cancer for the subject if the score is equal or higher than a predetermined score, when cancer is ruled in the subject receives a treatment protocol. Treatment protocols include for example pulmonary function test (PFT), pulmonary imaging, a biopsy, a surgery, a chemotherapy, a radiotherapy, or any combination thereof. In some embodiments, the imaging is an x-ray, a chest computed tomography (CT) scan, or a positron emission tomography (PET) scan.

In another aspect the invention further provides a method of determining the likelihood of the presence of a lung condition in a subject by measuring the abundance of panel of proteins in a sample obtained from the subject, calculating a probability of cancer score based on the protein measurements and concluding the presence of this lung condition if the score is equal or greater than a pre-determined score. The lung condition is lung cancer such as for example, non-small cell lung cancer (NSCLC). The subject may be at risk of developing lung cancer.

For example, the panel may include proteins BGH3_HUMAN, GGH_HUMAN, LG3BP_HUMAN, PRDX1_HUMAN, and TSP1_HUMAN. The panel may include proteins COIA1_HUMAN, ENPL_HUMAN, GGH_HUMAN, PRDX1_HUMAN, and TSP1_HUMAN. Alternatively, the panel may comprise BGH3_HUMAN, COIA1_HUMAN, ENPL_HUMAN, GGH_HUMAN, PRDX1_HUMAN, and TSP1_HUMAN.

In merely illustrative embodiments, the methods described herein include steps of (a) measuring the abundance (intensity) of one representative peptide transition derived from each of the proteins comprising BGH3_HUMAN, GGH_HUMAN, LG3BP_HUMAN, PRDX1_HUMAN, and TSP1_HUMAN in a sample obtained from a subject; (b) determining the coefficient for each representative peptide transition; (c) calculating a sum of the products of Box-Cox transformed (and optionally normalized) intensity of each transition and its corresponding coefficient; and (d) calculating a probability of cancer score based on the sum calculated in step (c).

In some embodiments, the representative peptide transitions for proteins BGH3_HUMAN, GGH_HUMAN, LG3BP_HUMAN, PRDX1_HUMAN, and TSP1_HUMAN are LTLLAPLNSVFK (SEQ ID No.: 46) (658.4, 804.5), YYIAASYVK (SEQ ID No.: 51) (539.28, 638.4), VEIFYR (SEQ ID No.: 56) (413.73, 598.3), QITVNDLPVGR (SEQ ID No.: 58) (606.3, 970.5), and GFLLLASLR (SEQ ID No.: 61) (495.31, 559.4), respectively. Their corresponding coefficient and Box-Cox transformation are listed in Table 7. Representative peptides and their transitions derived from other panel proteins described herein are listed in Table 1.

In some embodiments, the measuring step of any method described herein is performed by detecting transitions comprising LTLLAPLNSVFK (SEQ ID No.: 46) (658.4, 804.5), YYIAASYVK (SEQ ID No.: 51) (539.28, 638.4), VEIFYR (SEQ ID No.: 56) (413.73, 598.3), QITVNDLPVGR (SEQ ID No.: 58) (606.3, 970.5), and GFLLLASLR (SEQ ID No.: 61) (495.31, 559.4).

The subject has or is suspected of having a pulmonary nodule or a pulmonary mass. The pulmonary nodule has a diameter of less than or equal to 3.0 cm. The pulmonary mass has a diameter of greater than 3.0 cm. In some embodiments, the pulmonary nodule has a diameter of about 0.8 cm to 2.0 cm. The subject may have stage IA lung cancer (i.e., the tumor is smaller than 3 cm).

The probability score is calculated from a logistic regression model applied to the protein measurements. For example, the score is determined by EQN 1:

score=1/[1+exp(−α−Σᵢ₌₁^{N}βᵢ\check{P}ᵢ)], (EQN 1) wherein

$${\tilde{P}}_{i} = \frac{{P}_{i}^{{λ}_{i}} - 1.0}{{λ}_{i}} ,$$

and \check{P}ᵢ is Box-Cox transformed and normalized intensity of peptide transition i in said sample, βᵢ is the corresponding logistic regression coefficient, λᵢ is the corresponding Box-Cox transformation, a is a panel-specific constant, and N is the total number of transitions in the panel. The score determined has a positive predictive value (PPV) of at least about 30%, at least 40% or higher (50%, 60%, 70%, 80%, 90% or higher). A score equal to approximately 0.65 provides a PPV of 30%. A score equal to approximately 0.72 provides a PPV of 40%. A score equal to approximately 0.75 provides a classifier PPV of approximately 50%. Any suitable normalization methods known in the art can be used in calculating the probability score.

In various embodiments, the method of the present invention further comprises normalizing the protein measurements. For example, the protein measurements are normalized by one or more proteins selected from PEDF_HUMAN, MASP1_HUMAN, GELS_HUMAN, LUM_HUMAN, C163A_HUMAN and PTPRJ_HUMAN, CD44 HUMAN, TENX_HUMAN, CLUS_HUMAN, and 113P3_HUMAN. A skilled artisan could readily determine any other suitable proteins as normalizers according to the standard methods available in the art.

The biological sample includes such as for example tissue, blood, plasma, serum, whole blood, urine, saliva, genital secretion, cerebrospinal fluid, sweat and excreta.

In some embodiments, the determining the likelihood of cancer is determined by the sensitivity, specificity, negative predictive value or positive predictive value associated with the score.

The measuring step is performed by selected reaction monitoring mass spectrometry, using a compound that specifically binds the protein being detected or a peptide transition. In one embodiment, the compound that specifically binds to the protein being measured is an antibody or an aptamer.

In specific embodiments, the diagnostic methods disclosed herein are used to rule in a treatment protocol for a subject, measuring the abundance of a panel of proteins in a sample obtained from the subject, calculating a probability of cancer score based on the protein measurements and ruling in the treatment protocol for the subject if the score determined in the sample is equal or higher than a pre-determined score. In some embodiments the panel contains BGH3_HUMAN, GGH_HUMAN, LG3BP_HUMAN, PRDX1_HUMAN, and TSP1_HUMAN.

In certain embodiments, the diagnostic methods disclosed herein can be used in combination with other clinical assessment methods, including for example various radiographic and/or invasive methods. Similarly, in certain embodiments, the diagnostic methods disclosed herein can be used to identify candidates for other clinical assessment methods, or to assess the likelihood that a subject will benefit from other clinical assessment methods.

The high abundance of certain proteins in a biological sample such as plasma or serum can hinder the ability to assay a protein of interest, particularly where the protein of interest is expressed at relatively low concentrations. Several methods are available to circumvent this issue, including enrichment, separation, and depletion. Enrichment uses an affinity agent to extract proteins from the sample by class, e.g., removal of glycosylated proteins by glycocapture. Separation uses methods such as gel electrophoresis or isoelectric focusing to divide the sample into multiple fractions that largely do not overlap in protein content. Depletion typically uses affinity columns to remove the most abundant proteins in blood, such as albumin, by utilizing advanced technologies such as IgY14/Supermix (SigmaSt. Louis, Mo.) that enable the removal of the majority of the most abundant proteins.

In certain embodiments of the methods provided herein, a biological sample may be subjected to enrichment, separation, and/or depletion prior to assaying biomarker or putative biomarker protein expression levels. In certain of these embodiments, blood proteins may be initially processed by a glycocapture method, which enriches for glycosylated proteins, allowing quantification assays to detect proteins in the high pg/ml to low ng/ml concentration range. Exemplary methods of glycocapture are well known in the art (see, e.g., U.S. Pat. No. 7,183,188; U.S. Patent Appl. Publ. No. 2007/0099251; U.S. Patent Appl. Publ. No. 2007/0202539; U.S. Patent Appl. Publ. No. 2007/0269895; and U.S. Patent Appl. Publ. No. 2010/0279382). In other embodiments, blood proteins may be initially processed by a protein depletion method, which allows for detection of commonly obscured biomarkers in samples by removing abundant proteins. In one such embodiment, the protein depletion method is a Supermix (Sigma) depletion method.

In certain embodiments, a biomarker protein panel comprises two to 100 biomarker proteins. In certain of these embodiments, the panel comprises 2 to 5, 6 to 10, 11 to 15, 16 to 20, 21-25, 5 to 25, 26 to 30, 31 to 40, 41 to 50, 25 to 50, 51 to 75, 76 to 100, biomarker proteins. In certain embodiments, a biomarker protein panel comprises one or more subpanels of biomarker proteins that each comprises at least two biomarker proteins. For example, biomarker protein panel may comprise a first subpanel made up of biomarker proteins that are overexpressed in a particular lung condition and a second subpanel made up of biomarker proteins that are under-expressed in a particular lung condition.

In certain embodiments of the methods, compositions, and kits provided herein, a biomarker protein may be a protein that exhibits differential expression in conjunction with lung cancer.

In other embodiments, the diagnosis methods disclosed herein may be used to distinguish between two different lung conditions. For example, the methods may be used to classify a lung condition as malignant lung cancer versus benign lung cancer, NSCLC versus SCLC, or lung cancer versus non-cancer condition (e.g., inflammatory condition).

In certain embodiments, kits are provided for diagnosing a lung condition in a subject. These kits are used to detect expression levels of one or more biomarker proteins. Optionally, a kit may comprise instructions for use in the form of a label or a separate insert. The kits can contain reagents that specifically bind to proteins in the panels described, herein. These reagents can include antibodies. The kits can also contain reagents that specifically bind to mRNA expressing proteins in the panels described, herein. These reagents can include nucleotide probes. The kits can also include reagents for the detection of reagents that specifically bind to the proteins in the panels described herein. These reagents can include fluorophores.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention

### EXAMPLES

### Example 1: Identification of a Robust Rule-in Classifier that Distinguishes Malignant and Benign Lung Nodule

1. Determine which Proteins to Use

There are 24 proteins in the dataset that have heavy peptides. Six proteins are normalizers so 18 proteins are available for the panel development analysis. The following Table 1 lists the candidate proteins and corresponding transitions.

TABLE 1
Candidate Proteins

| Protein | Peptide | Q1 | Q3 |
|---|---|---|---|
| ALDOA_HUMAN | ALQASALK | 401.25 | 617.4 |
| | (SEQ ID No.: 45) | | |
| BGH3_HUMAN | LTLLAPLNSVFK | 658.4 | 804.5 |
| | (SEQ ID No.: 46) | | |
| CD14_HUMAN | ATVNPSAPR | 456.8 | 527.3 |
| | (SEQ ID No.: 47) | | |
| COIA1_HUMAN | AVGLAGTFR | 446.26 | 721.4 |
| | (SEQ ID No.: 48) | | |
| ENPL_HUMAN | SGYLLPDTK | 497.27 | 308.1 |
| | (SEQ ID No.: 49) | | |
| FRIL_HUMAN | LGGPEAGLGEYLFER | 804.4 | 1083.6 |
| | (SEQ ID No.: 50) | | |
| GGH_HUMAN | YYIAASYVK | 539.28 | 638.4 |
| | (SEQ ID No.: 51) | | |
| GRP78_HUMAN | TWNDPSVQQDIK | 715.85 | 288.1 |
| | (SEQ ID No.: 52) | | |
| IBP3_HUMAN | FLNVLSPR | 473.28 | 685.4 |
| | (SEQ ID No.: 53) | | |
| ISLR_HUMAN | ALPGTPVASSQPR | 640.85 | 841.5 |
| | (SEQ ID No.: 54) | | |
| KIT_HUMAN | YVSELHLTR | 373.21 | 428.3 |
| | (SEQ ID No.: 55) | | |
| LG3BP_HUMAN | VEIFYR | 413.73 | 598.3 |
| | (SEQ ID No.: 56) | | |
| LRP1_HUMAN | TVLWPNGLSLDIPAGR | 855 | 1209.7 |
| | (SEQ ID No.: 57) | | |
| PRDX1_HUMAN | QITVNDLPVGR | 606.3 | 970.5 |
| | (SEQ ID No.: 58) | | |
| PROF1_HUMAN | STGGAPTFNVTVTK | 690.4 | 1006.6 |
| | (SEQ ID No.: 59) | | |
| TENX_HUMAN | YEVTVVSVR | 526.29 | 293.1 |
| | (SEQ ID No.: 60) | | |
| TETN_HUMAN | LDTLAQEVALLK | 657.39 | 871.5 |
| | (SEQ ID No.: 66) | | |
| TSP1_HUMAN | GFLLLASLR | 495.31 | 559.4 |
| | (SEQ ID No.: 61) | | |

2. Subset Data to Relevant Proteins (Normalization)

The normalization procedure is described in PCT/US2012/071387 (WO13/096845), the contents of which are incorporated herein by reference in their entireties. It includes 115 Samples, 91 Clinical Samples usable for training and 3 clinical samples not usable in training and 20 HGS samples, 4 per batch. The samples come from three sites Laval, NYU and UPenn. The samples all have a nodule size in the range 8 mm to 20 mm.

Six normalizing proteins were identified that had a transition detected in all samples of the study and with low coefficient of variation. For each protein the transition with highest median intensity across samples was selected as the representative transition for the protein. These proteins and transitions are found in Table 2.

TABLE 2
Normalizing Factors

| Protein (Uniprot ID) | Peptide (Amino Acid Sequence) | Transition (m/z) |
|---|---|---|
| CD44_HUMAN | YGFIEGHVVIPR (SEQ ID No.: 62) | 272.2 |
| TENX_HUMAN | YEVTVVSVR (SEQ ID No.: 60) | 759.5 |
| CLUS_HUMAN | ASSIIDELFQDR (SEQ ID No.: 63) | 565.3 |
| IBP3_HUMAN | FLNVLSPR (SEQ ID No.: 53) | 685.4 |
| GELS_HUMAN | TASDFITK (SEQ ID No.: 64) | 710.4 |
| MASP1_HUMAN | TGVITSPDFPNPYPK (SEQ ID No.: 65) | 258.10 |

We refer to the transitions in Table 2 as normalizing factors (NFs). Each of the 1550 transitions were normalized by each of the six normalizing factors where the new intensity of a transition t in a sample s by NF f, denoted New(s,t,f), is calculated as follows:

New(s,t,f)=Raw(s,t)*Median(f)/Raw(s,f)

where Raw(s,t) is the original intensity of transition tin sample s; Median(f) is the median intensity of the NF f across all samples; and Raw(s,f) is the original intensity of the NF f in sample s.

For each protein and normalized transition, the AUC of each batch was calculated. The NF that minimized the coefficient of variation across the batches was selected as the NF for that protein and for all transitions of that protein. Consequently, every protein (and all of its transitions) are now normalized by a single NF.

3. Generate 1 Million Panels with 18 Proteins.

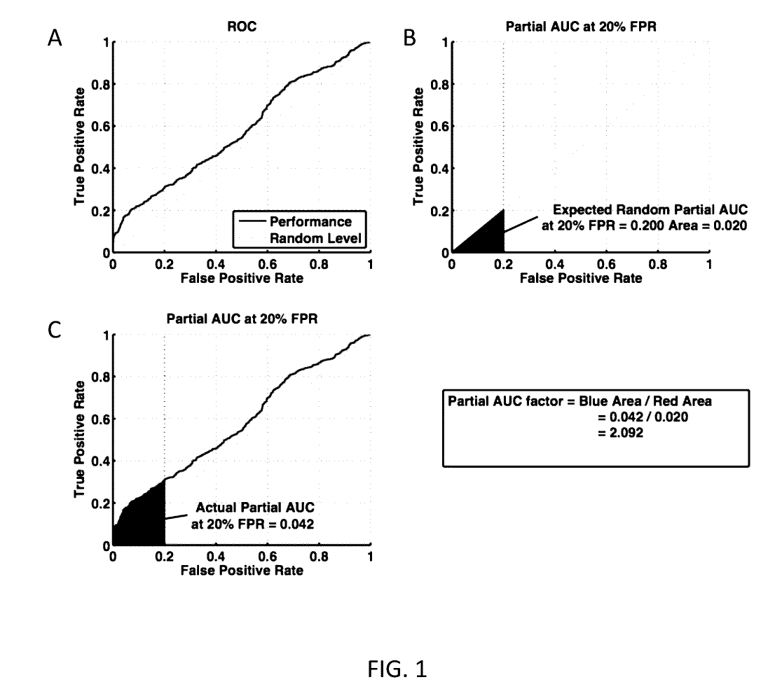

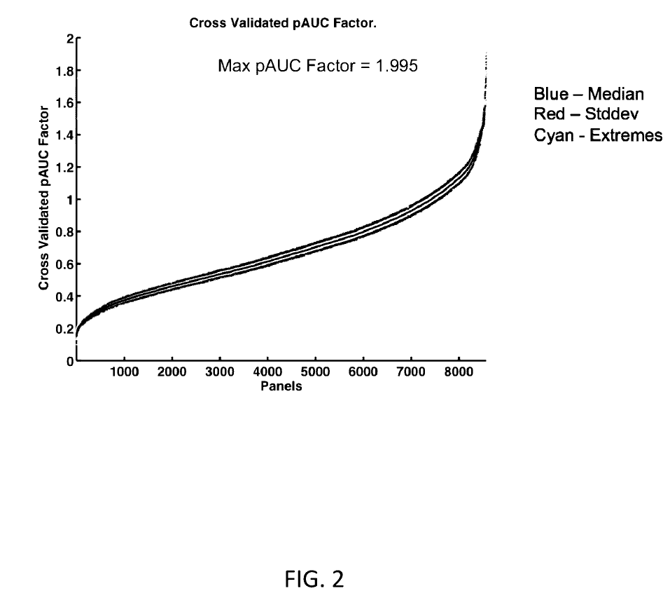

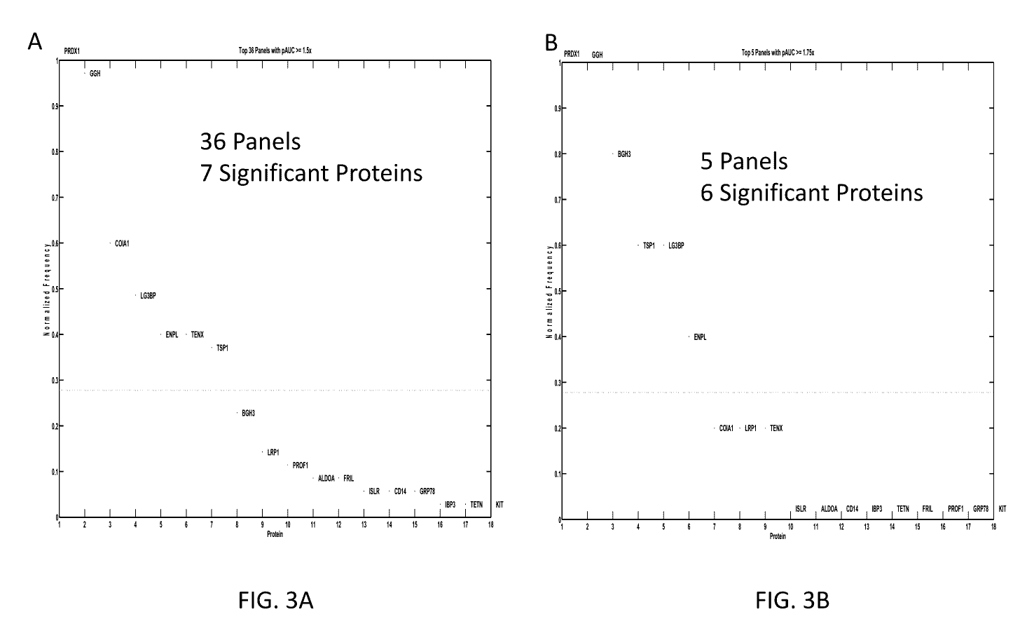

A million random panels of 5 proteins each are generated and the partial AUC tracked using a specificity of 0.8 using a hold out rate of 20%. There are (18/5)=8568 panels and each panel has multiple measurements. The panels are ranked by Partial AUC factor at a False Positive Rate (FPR) of 20%. FIGS. 1A-1C describe how partial AUC factor is calculated.

Accordingly, panels with >=1.5 pAUC Factor comprise proteins listed in Table 3 below.

TABLE 3
Panels with >= 1.5 pAUC Factor

| | | Performance_ | Performance_ | Beats_ |
|---|---|---|---|---|
| Protein | Transition | Number | Normalized | Expectations |
| PRDX1_HUMAN | QITVNDLPVGR_606.30_970.50 | 35 | 1.0000 | 1 |
| | (SEQ ID No.: 58) | | | |
| GGH_HUMAN | YYIAASYVK_539.28_638.40 | 34 | 0.9714 | 1 |
| | (SEQ ID No.: 51) | | | |
| COIA1_HUMAN | AVGLAGTFR_446.26_721.40 | 21 | 0.6000 | 1 |
| | (SEQ ID No.: 48) | | | |
| LG3BP_HUMAN | VEIFYR_413.73_598.30 | 17 | 0.4857 | 1 |
| | (SEQ ID No.: 56) | | | |
| ENPL_HUMAN | SGYLLPDTK_497.27_308.10 | 14 | 0.4000 | 1 |
| | (SEQ ID No.: 49) | | | |
| TENX_HUMAN | YEVTVVSVR_526.29_293.10 | 14 | 0.4000 | 1 |
| | (SEQ ID No.: 60) | | | |
| TSP1_HUMAN | GFLLLASLR_495.31_559.40 | 13 | 0.3714 | 1 |
| | (SEQ ID No.: 61) | | | |
| BGH3_HUMAN | LTLLAPLNSVFK_658.40_804.50 | 8 | 0.2286 | 0 |
| | (SEQ ID No.: 46) | | | |
| LRP1_HUMAN | TVLWPNGLSLDIPAGR_855.00_1209.70 | 5 | 0.1429 | 0 |
| | (SEQ ID No.: 57) | | | |
| PROF1_HUMAN | STGGAPTFNVTVTK_690.40_1006.60 | 4 | 0.1143 | 0 |
| | (SEQ ID No.: 59) | | | |
| ALDOA_HUMAN | ALQASALK_401.25_617.40 | 3 | 0.0857 | 0 |
| | (SEQ ID No.: 45) | | | |
| FRIL_HUMAN | LGGPEAGLGEYLFER_804.40_1083.60 | 3 | 0.0857 | 0 |
| | (SEQ ID No.: 50) | | | |
| ISLR_HUMAN | ALPGTPVASSQPR_640.85_841.50 | 2 | 0.0571 | 0 |
| | (SEQ ID No.: 54) | | | |
| CD14_HUMAN | ATVNPSAPR_456.80_527.30 | 2 | 0.0571 | 0 |
| | (SEQ ID No.: 47) | | | |
| GRP78_HUMAN | TWNDPSVQQDIK_715.85_288.10 | 2 | 0.0571 | 0 |
| | (SEQ ID No.: 52) | | | |
| IBP3_HUMAN | FLNVLSPR_473.28_685.40 | 1 | 0.0286 | 0 |
| | (SEQ ID No.: 53) | | | |
| TETN_HUMAN | LDTLAQEVALLK_657.39_871.50 | 1 | 0.0286 | 0 |
| | (SEQ ID No.: 66) | | | |
| KIT_HUMAN | YVSELHLTR_373.21_428.30 | 1 | 0.0286 | 0 |
| | (SEQ ID No.: 55) | | | |

Panels with >=1.75 pAUC Factor comprise proteins listed in Table 4 below.

TABLE 4
Panels with >= 1.75 pAUC Factor

| | | Performance_ | Performance_ | Beats_ |
|---|---|---|---|---|
| Protein | Transition | Number | Normalized | Expectations |
| PRDX1_HUMAN | QITVNDLPVGR_606.30_970.50 | 5 | 1.0000 | 1 |
| | (SEQ ID No.: 58) | | | |
| GGH_HUMAN | YYIAASYVK_539.28_638.40 | 5 | 1.000 | 1 |
| | (SEQ ID No.: 51) | | | |
| BGH3_HUMAN | LTLLAPLNSVFK_658.40_804.50 | 4 | 0.8000 | 1 |
| | (SEQ ID No.: 46) | | | |
| TSP1_HUMAN | GFLLLASLR_495.31_559.40 | 3 | 0.6000 | 1 |
| | (SEQ ID No.: 61) | | | |
| LG3BP_HUMAN | VEIFYR_413.73_598.30 | 3 | 0.6000 | 1 |
| | (SEQ ID No.: 56) | | | |
| ENPL_HUMAN | SGYLLPDTK_497.27_308.10 | 2 | 0.4000 | 1 |
| | (SEQ ID No.: 49) | | | |
| COIA1_HUMAN | AVGLAGTFR_446.26_721.40 | 1 | 0.2000 | 0 |
| | (SEQ ID No.: 48) | | | |
| LRP1_HUMAN | TVLWPNGLSLDIPAGR_855.00_1209.70 | 1 | 0.2000 | 0 |
| | (SEQ ID No.: 57) | | | |
| TENX_HUMAN | YEVTVVSVR_526.29_293.10 | 1 | 0.2000 | 0 |
| | (SEQ ID No.: 60) | | | |
| ISL4_HUMAN | ALPGTPVASSQPR_640.85_841.50 | 0 | 0.0000 | 0 |
| | (SEQ ID No.: 54) | | | |
| ALDOA_HUMAN | ALQASALK_401.25_617.40 | 0 | 0.0000 | 0 |
| | (SEQ ID No.: 45) | | | |
| CD14_HUMAN | ATVNPSAPR_456.80_527.30 | 0 | 0.0000 | 0 |
| | (SEQ ID No.: 47) | | | |
| IBP3_HUMAN | FLNVLSPR_473.28_685.40 | 0 | 0.0000 | 0 |
| | (SEQ ID No.: 53) | | | |
| TETN_HUMAN | LDTLAQEVALLK_657.39_871.50 | 0 | 0.0000 | 0 |
| | (SEQ ID No.: 66) | | | |
| FRIL_HUMAN | LGGPEAGLGEYLFER_804.40_1083.60 | 0 | 0.0000 | 0 |
| | (SEQ ID No.: 50) | | | |
| PROF1_HUMAN | STGGAPTFNVTVTK_690.40_1006.60 | 0 | 0.0000 | 0 |
| | (SEQ ID No.: 59) | | | |
| GRP78_HUMAN | TWNDPSVQQDIK_715.85_288.10 | 0 | 0.0000 | 0 |
| | (SEQ ID No.: 52) | | | |
| KIT_HUMAN | YVSELHLTR_373.21_428.30 | 0 | 0.0000 | 0 |
| | (SEQ ID No.: 55) | | | |

4. Proteins Keep

The proteins kept are the union of 1.5× and 1.75× panels that are significant, i.e., COIA1_HUMAN, ENPL_HUMAN, GGH_HUMAN, LG3BP_HUMAN, PRDX1_HUMAN, TENX_HUMAN, and TSP1_HUMAN.

5. Analytical Validation of Proteins

A separate experiment was carried out to determine how well the proteins varied as columns changed and depletion position changed.

6. Take the 7 Remaining Proteins and Exhaustively Search all Panels

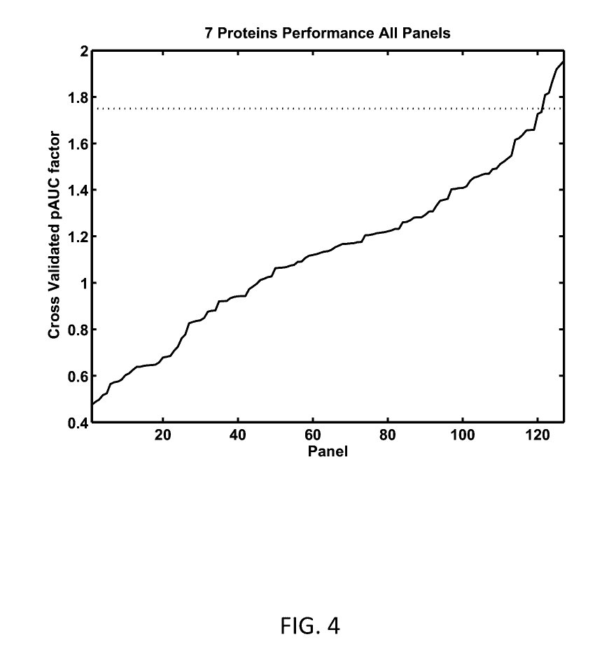

Form every possible 127 panel combinations of the remaining 7 proteins. The performance of all panels of these 7 proteins is shown in FIG. 4. Each panel is tested tracking the partial AUC, distribution of coefficients, etc. Measuring the partial AUC factor of the panels with better that 1.75× resulted in 6 panels (Table 5).

TABLE 5
Best 6 panels

| | | | | | Crossvalidated |
|---|---|---|---|---|---|
| | | Maximum CV | Maximum | | pAUC |
| Name | Proteins | Protein | Model CV | ALPHA CV | factor |
| RuleIn_1 | BGH3_HUMAN, | COIA1_HUMAN | 0.6571 | 46.2498320216908 | 1.96523447802469 |
| | COIA1_HUMAN, | | | | |
| | ENPL_HUMAN, | | | | |
| | GGH_HUMAN, | | | | |
| | PRDX1_HUMAN, | | | | |
| | TSP1_HUMAN | | | | |
| RuleIn_2 | BGH3_HUMAN, | COIA1_HUMAN | 0.6397 | 0.979908242041881 | 1.93097955555555 |
| | COIA1_HUMAN, | | | | |
| | ENPL_HUMAN, | | | | |
| | GGH_HUMAN, | | | | |
| | LG3BP_HUMAN, | | | | |
| | PRDX1_HUMAN, | | | | |
| | TSP1_HUMAN | | | | |
| RuleIn_3 | BGH3_HUMAN, | TSP1_HUMAN | 0.4861 | 1.53959755683128 | 1.90957520987654 |
| | ENPL_HUMAN, | | | | |
| | GGH_HUMAN, | | | | |
| | LG3BP_HUMAN, | | | | |
| | PRDX1_HUMAN, | | | | |
| | TSP1_HUMAN | | | | |
| RuleIn_4 | BGH3_HUMAN, | TSP1_HUMAN | 0.5461 | 0.341327685172249 | 1.87271083555556 |
| | GGH_HUMAN, | | | | |
| | LG3BP_HUMAN, | | | | |
| | PRDX1_HUMAN, | | | | |
| | TSP1_HUMAN | | | | |
| RuleIn_5 | COIA1_HUMAN, | COIA1_HUMAN | 0.5854 | 1.40331399560408 | 1.8062064908642 |
| | ENPL_HUMAN, | | | | |
| | GGH_HUMAN, | | | | |
| | PRDX1_HUMAN, | | | | |
| | TSP1_HUMAN | | | | |
| RuleIn_6 | BGH3_HUMAN, | TSP1_HUMAN | 0.4152 | 2.07823201290617 | 1.81452772641975 |
| | ENPL_HUMAN, | | | | |
| | GGH_HUMAN, | | | | |
| | PRDX1_HUMAN, | | | | |
| | TSP1_HUMAN | | | | |

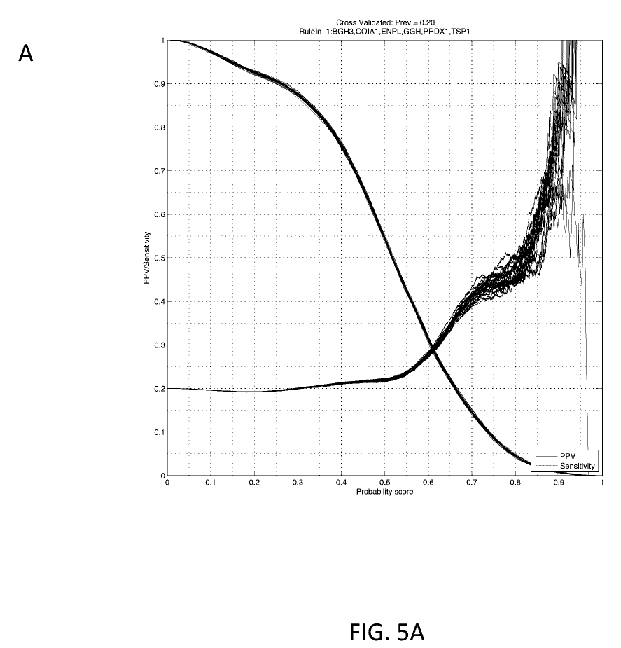

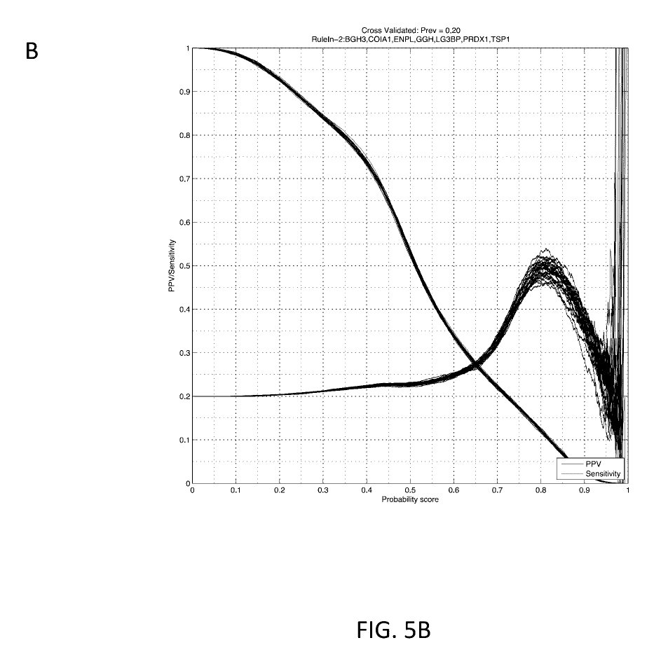

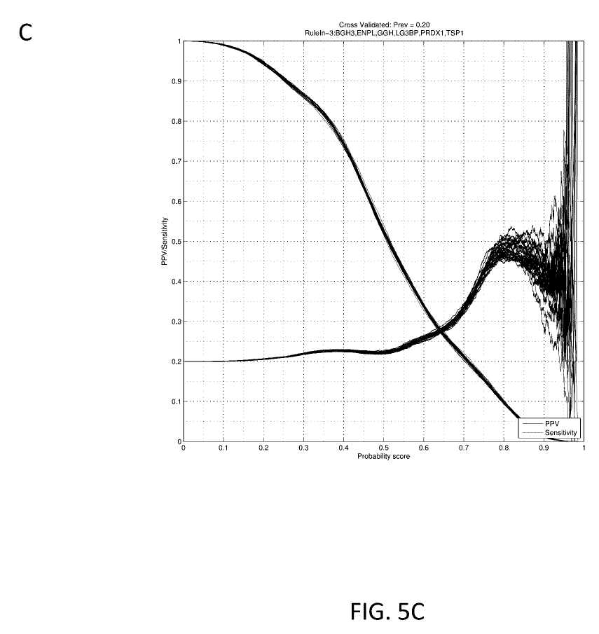

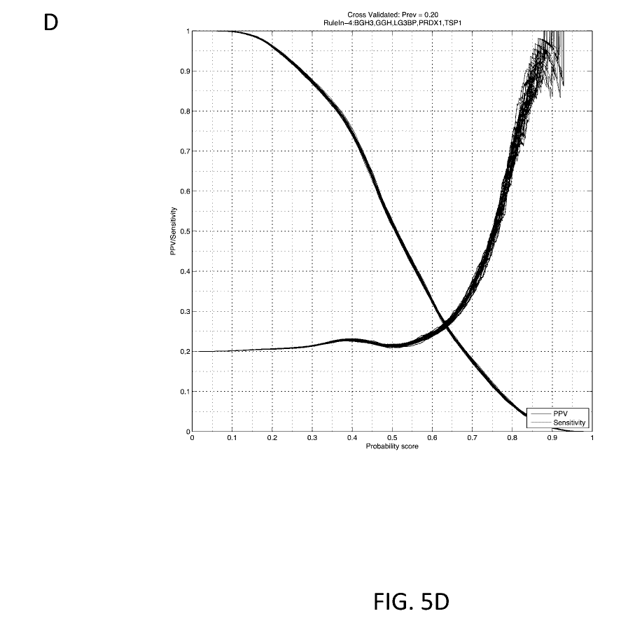

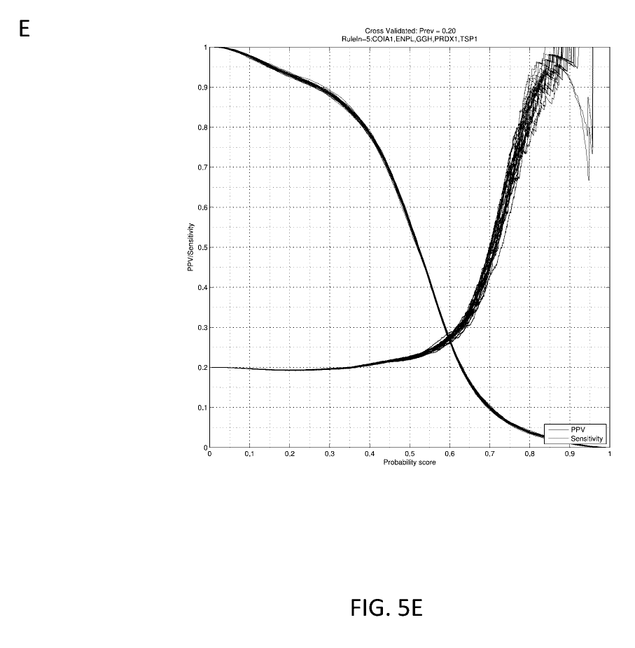

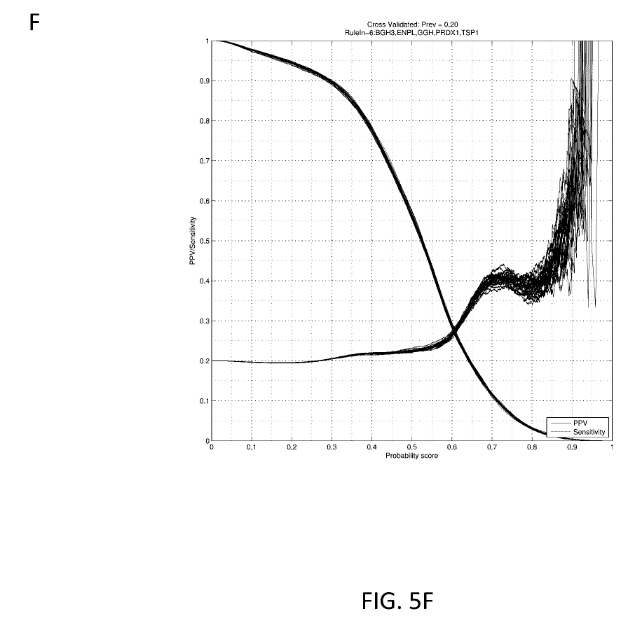

The cross validated performance (Positive Predictive Value (PPV) and Sensitivity) was measured for each of the six panels. By training the models and recording the performance based off of stacking 25,000 models worth of held out test data. Their cross validated performances are shown in FIGS. 5A-5F. Three panels were excluded (Panels 2, 3, and 6) because their cross validated performance has dips, indicating that the panel didn't work well in a subset of the samples.

7. Model Tested on Analytical Data

The remaining three models were applied to the analytical dataset and the column to column and position to position variability of the model was measured. Panel 4 had the best correlation in both categories.

8. Summary of 3 Panels (Table 6)

TABLE 6
Summary of panels 1, 4, and 5

| Panel | PPV 30% | PPV 40% | PPV 50% | Analytical Results |
|---|---|---|---|---|
| 1 | 27% | 16% | 3% | Unfavorable |
| 4 | 22% | 14% | 10% | Favorable |
| 5 | 26% | 12% | 8% | Unfavorable |

Therefore panel 4 is selected as the best rule-in classifier. It contains 5 proteins (BGH3_HUMAN, GGH_HUMAN, LG3BP_HUMAN, PRDX1_HUMAN, and TSP1_HUMAN).

10. Model Definition

A rule-in classifer consisting for lung cancer including five proteins was generated using a logistic regression model according to EQN 2:

$$Classifier \text{:} 5 Proteins \\ Logistic regression model \\ score = \frac{1}{1 + exp ( - W )} \\ W = α + {\sum }_{i = 1}^{5} {β}_{i} * {\tilde{P}}_{i} {\tilde{P}}_{i} = \frac{{P}_{i}^{{λ}_{i}} - 1.0}{{λ}_{i}} \\ Normalized , Box \text{-} Cox transformed protein abundance {\tilde{P}}_{i} can be negative . \qquad ( EQN 2 )$$

wherein \check{P}ᵢ is the Box-Cox transformed, and normalized intensity of peptide transition i in said sample, βᵢ is the corresponding logistic regression coefficient, and λᵢ is the corresponding Box-Cox transformation.

The panel-specifical constant (α), logistic regression coefficient (βᵢ) and Box-Cox transformation (λ) for panel 4 was calculated according to the logistic regression model of EQN 2. The variables for the rule-in classific based on panel 4 are listed in Table 7.

TABLE 7
Rule-in classifier based on Panel 4

| | | | | Coefficient | Box Cox |
|---|---|---|---|---|---|
| Protein | Peptide | Q1 | Q3 | (β) | (λ) |
| BGH3_HUMAN | LTLLAPLNSVFK | 658.4 | 804.5 | 1.012353821 | 0.37 |
| | (SEQ ID No.: 46) | | | | |
| GGH_HUMAN | YYIAASYVK | 539.28 | 638.4 | 2.673287672 | 0.31 |
| | (SEQ ID No.: 51) | | | | |
| LG3BP_HUMAN | VEIFYR | 413.73 | 598.3 | −1.331698432 | −0.63 |
| | (SEQ ID No.: 56) | | | | |
| PRDX1_HUMAN | QITVNDLPVGR | 606.3 | 970.5 | −0.641405539 | −0.14 |
| | (SEQ ID No.: 58) | | | | |
| TSP1_HUMAN | GFLLLASLR | 495.31 | 559.4 | 0.284343479 | 0.02 |
| | (SEQ ID No.: 61) | | | | |
| ALPHA | | | | α = 2.500395391 | |

A sample was classified as benign if the probability of cancer score was less than a pre-determined score or decision threshold. The decision threshold can be increased or decreased depending on the desired PPV. To define the classifier, the panel of transitions (i.e. proteins), their coefficients, the normalization transitions, classifier coefficient α and the decision threshold may be learned (i.e. trained) from a discovery study and then confirmed using a validation study.

11. Performance of Panel 4 (Rule-in Classifier)

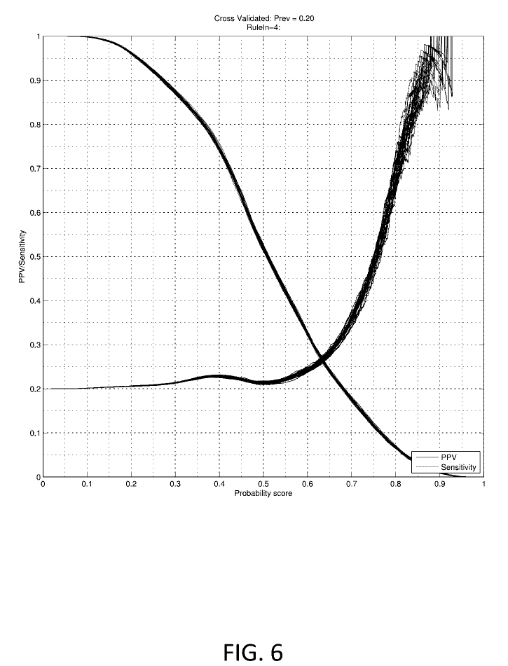

The performance of panel 4 is shown in FIG. 6.

As shown in FIG. 6, a probability of cancer score=0.65 decision threshold provides a classifier PPV of approximately 30%. A probability of cancer score=0.72 decision threshold provides a classifier PPV of approximately 40%. A probability of cancer score=0.75 decision threshold provides a classifier PPV of approximately 50%.

Table 8 shows the sensitivity of panel 4 at different level of PPV and the percentage of population that cannot be ruled out by the rule-out classifier, but that can be identified as cancer patients by this rule-in classifier.

TABLE 8
Performance of Panel 4

| PPV | Sensitivity | Population |
|---|---|---|
| 30% | 22% | 15% |
| 40% | 14% | 7% |
| 50% | 10% | 4% |

Table 9 depicts the performance of the rule-out classifier and the rule-in classifer. The rule-out classifer includes a method of determining the likelihood that a lung condition in a subject is cancer by assessing the expression of a plurality of proteins comprising determining the protein expression level of at least each of ALDOA_HUMAN, FRIL_HUMAN, LG3BP_HUMAN, TSP1_HUMAN and COIA1_HUMAN from a biological sample obtained from a subject; calculating a score from the protein expression of at least each of ALDOA_HUMAN, FRIL_HUMAN, LG3BP_HUMAN, TSP1_HUMAN and COIA1_HUMAN from the biological sample determined in the preceding step; and comparing the score from the biological sample to a plurality of scores obtained from a reference population, wherein the comparison provides a determination that the lung condition is not concer.

TABLE 9
Performance of the rule-out classifier and the rule-in classifier

| | Rule-out | Indeterminate | Rule-in |
|---|---|---|---|
| Population | 40% | ~45-55% | ~15, 7, 4% |
| Performance | NPV: 87% | | PPV: 30, 40, 50% |

TABLE 10A
All data for the 18 candidate proteins (Box Cox transformed and normalized)

| | | ALPGTPVASS- | | AT- |
|---|---|---|---|---|
| msfile- | | QPR_640.85_841.50 | ALQASALK_401.25_617.40 | VNPSAPR_456.80_527.30 |
| name | Group | (SEQ ID No.: 54) | (SEQ ID No.: 45) | (SEQ ID No.: 47) |
| PC_01 | | −2.784263895 | −0.513204312 | −0.704971561 |
| ZCO491_03 | Cancer | −2.75727098 | 0.784933743 | −0.614376856 |
| ZCO415_03 | Benign | −2.680545115 | 1.181691249 | −0.200714857 |
| ZCO377_03 | Cancer | −3.089810045 | −0.398353331 | −0.568038788 |
| ZCO482_03 | Benign | −2.504744002 | 0.787441476 | −0.675544537 |
| ZCO371_03 | Benign | −2.899836726 | 0.362448117 | −0.197452873 |
| ZCO460_03 | Cancer | −2.910586434 | 0.227151983 | −0.145522413 |
| PC_02 | | −2.690384259 | −0.643733763 | −0.616319695 |
| ZCO531_01 | Cancer | −3.010037962 | −0.536429117 | −0.791760403 |
| ZCO422_03 | Benign | −2.947508157 | −0.885615583 | −0.979068939 |
| ZCO474_03 | Benign | −3.002579978 | 0.603913437 | −1.473883307 |
| ZCO539_03 | Cancer | −3.144491206 | 0.25393171 | −1.266702624 |
| ZCO464_03 | Benign | −2.831346776 | −0.573333479 | −0.928230586 |
| ZCO455_03 | Cancer | −2.852113183 | −0.587540023 | −0.780298433 |
| ZCO542_03 | Cancer | −3.164489489 | 0.533735226 | −0.840531166 |
| ZCO369_03 | Benign | −2.877284738 | −0.273990975 | −0.935052482 |
| PC_03 | | −2.807782819 | −0.664551407 | −0.776547284 |
| ZCO498_03 | Benign | −2.884132267 | −0.119878696 | −0.685613811 |
| ZCO430_03 | Cancer | −2.410086363 | 0.596052018 | −0.400081837 |
| ZCO434_03 | Cancer | −2.707727142 | 0.482978922 | −0.815665074 |
| ZCO405_03 | Benign | −1.898017731 | 0.596444247 | 0.2674756 |
| ZCO518_03 | Benign | −2.452842401 | 0.421384621 | −0.439118905 |
| ZCO388_03 | Cancer | −2.947809702 | −1.137350025 | −0.1040406 |
| PC_04 | | −2.926819692 | −0.383759077 | −0.675828051 |
| PC_01 | | −2.856174592 | −0.701301918 | −0.747538278 |
| ZCO529_02 | Cancer | −2.608415869 | −0.131152282 | −1.3391951 |
| ZCO472_02 | Benign | −2.838879945 | 0.645540071 | −0.713484997 |
| ZCO421_02 | Benign | −2.703957077 | −0.314820047 | −0.600669916 |
| ZCO517_02 | Cancer | −2.482786226 | 0.823060539 | −0.489659037 |
| ZCO414_02 | Cancer | −2.572707711 | 0.218310959 | −0.332704095 |
| ZCO467_02 | Benign | −2.120568668 | −0.131506795 | −1.178970522 |
| PC_02 | | −2.995944005 | −0.677948163 | −0.784676364 |
| ZCO538_02 | Benign | −2.461211468 | −0.74329599 | −0.494137705 |
| ZCO490_02 | Cancer | −2.749244243 | −0.626595231 | −0.899995183 |
| ZCO513_02 | Benign | −2.960810542 | 0.416212671 | −1.15671717 |
| ZCO368_02 | Cancer | −2.882760767 | −0.726491688 | −0.670577295 |
| ZCO478_02 | Benign | −3.462231929 | −0.775260583 | −1.54136049 |
| ZCO509_02 | Cancer | −3.425397519 | 0.589997632 | −1.000355571 |
| ZCO457_02 | Benign | −2.993673472 | 0.274256767 | −0.8506676 |
| ZCO384_02 | Cancer | −2.481295103 | −0.480824029 | −0.559267713 |
| PC_03 | | −2.915900307 | −0.636087686 | −0.710351323 |
| ZCO364_02 | Benign | −2.804799817 | −0.716221197 | −0.556992563 |
| ZCO392_02 | Cancer | −3.084300524 | −0.841568558 | −0.717882956 |
| ZCO401_02 | Cancer | −2.712351788 | −0.746712453 | −0.600323949 |
| ZCO544_02 | Benign | −3.112609502 | −0.031890482 | −0.427524429 |
| ZCO526_01 | Benign | −3.643501599 | −0.318902302 | −0.743509213 |
| ZCO445_02 | Cancer | −2.331441104 | 0.332420966 | −0.622523309 |
| PC_04 | | −2.507435668 | −0.028465151 | −0.580436007 |
| PC_01 | | −2.975924334 | −0.974164536 | −0.925021721 |
| CAP00721-09 | Benign | −3.320348365 | −1.191297249 | −1.24733595 |
| CAP00749-09 | Cancer | −2.532997922 | −0.362810416 | −0.647660241 |
| CAP00132-07 | Cancer | −2.560199759 | −0.72444247 | −0.515319045 |
| CAP02123-09 | Benign | −2.664488201 | −1.05273991 | −0.916975616 |
| CAP03009-08 | Benign | −2.8140739 | −0.578526633 | −1.004995502 |
| CAP01154-06 | Cancer | −2.795541436 | −0.76152897 | −1.191300457 |
| PC_02 | | −2.831484668 | −0.658389628 | −0.868371708 |
| CAP02208-05 | Benign | −2.515521098 | −1.163958883 | −0.816494043 |
| CAP00157-07 | Cancer | −3.195590468 | −1.682656452 | −0.980963914 |
| CAP00369-10 | Benign | −2.599714888 | −1.178861297 | −0.864831174 |
| CAP03006-08 | Cancer | −2.51741894 | −0.366332102 | −0.682527569 |
| CAP01799-08 | Benign | −2.483202761 | −0.957783104 | −0.574591873 |
| CAP02126-09 | Benign | −2.420357959 | −1.065815505 | −0.831422448 |
| PC_03 | | −2.92253495 | −0.723841011 | −0.805703785 |
| CAP01129-06 | Cancer | −2.418317307 | −1.033109959 | −1.238749304 |
| CAP01791-08 | Cancer | −1.975785528 | −0.192835023 | −0.865873926 |
| PC_04 | | −2.657657131 | −0.83639568 | −0.476964249 |
| PC_01 | | −2.64178703 | −0.203268296 | −0.60835134 |
| NYU_16 | Cancer | −2.765927482 | −1.379671565 | −1.032592583 |
| NYU_24 | Benign | −2.691628754 | 0.665189877 | −0.159436729 |
| NYU_514 | Benign | −2.502736019 | 0.554570418 | −0.226503612 |
| NYU_349 | Cancer | −2.922719299 | −0.405535171 | −0.80890645 |
| NYU_379 | Cancer | −2.715372965 | 0.072717025 | −0.616380062 |
| NYU_1145 | Benign | −2.396309675 | 0.267871762 | −0.313873633 |
| PC_02 | | −2.855372673 | −0.548857095 | −0.711361472 |
| NYU_696 | Cancer | −2.798888572 | −0.306145932 | −0.634564204 |
| NYU_84 | Benign | −2.526405093 | −0.452362276 | −0.211486953 |
| NYU_907 | Cancer | −2.068154205 | −0.262418236 | −0.411920341 |
| NYU_332 | Benign | −2.491414639 | 0.505717241 | −0.477051323 |
| NYU_173 | Benign | −2.024008719 | −1.830470251 | −0.898965857 |
| NYU_427 | Cancer | −3.037814652 | −0.062617856 | −0.43098363 |
| NYU_184 | Cancer | −2.752840585 | −0.049130794 | −0.59050779 |
| NYU_1001 | Benign | −2.209344901 | −0.416753024 | −0.901519025 |
| PC_03 | | −2.78147023 | −0.786435787 | −0.705150487 |
| NYU_453 | Benign | −2.694841411 | 0.66610542 | −0.547970741 |
| NYU_1141 | Cancer | −3.093608079 | −0.1027147 | −0.290625872 |
| NYU_1096 | Cancer | −2.6566636 | −0.399544864 | −0.995074996 |
| NYU_500 | Benign | −2.816104908 | 0.609371863 | −0.167363046 |
| NYU_1317 | Cancer | −2.885418437 | 0.218459687 | −0.793700606 |
| NYU_841 | Benign | −3.047488561 | −0.068078386 | −0.627329599 |
| PC_04 | | −2.94827646 | 0.47610704 | −0.755175074 |
| PC_01 | | −2.701682878 | −0.554717305 | −0.672162757 |
| NYU_28 | Benign | −2.807002674 | −0.498479033 | −0.893516236 |
| NYU_1559S | Cancer | −2.435455565 | −0.855099592 | −0.470130406 |
| NYU_440 | Benign | −2.689065693 | 0.013016259 | −0.812958589 |
| NYU_1176 | Cancer | −2.18567791 | −1.103770287 | −0.258856517 |
| NYU_831 | Cancer | −2.382166564 | 0.034330521 | −0.354053284 |
| NYU_71 | Benign | −2.339701655 | −0.542993731 | −0.51455545 |
| PC_02 | | −2.796375205 | −0.834237524 | −0.79059082 |
| NYU_111 | Cancer | −2.879596594 | −0.703232422 | −0.782682644 |
| NYU_423 | Benign | −2.894795626 | −0.160685009 | −0.295223446 |
| NYU_834 | Benign | −3.060257281 | −1.102989681 | −1.017704792 |
| NYU_830 | Cancer | −2.538245897 | 0.059933094 | −0.361560127 |
| NYU560 | Cancer | −2.435279885 | −0.415972091 | −0.924578302 |
| NYU_281 | Benign | −3.084507437 | 1.000569367 | −1.065193179 |
| NYU_613S | Cancer | −2.7703315 | 0.252825766 | −0.251086279 |
| NYU_513 | Benign | −2.41937926 | −0.013350489 | −0.652862825 |
| PC_03 | | −2.888524004 | −0.519986717 | −0.649520684 |
| NYU_661 | Cancer | −2.186698404 | 0.344191537 | −0.455408844 |
| NYU_1168 | Benign | −2.775589696 | −0.160638434 | −0.764998685 |
| NYU_968 | Benign | −2.373171563 | −0.022948899 | −0.696358068 |
| NYU_410 | Cancer | −2.52362406 | 0.179203243 | −0.738739815 |
| NYU_1098 | Benign | −3.531881869 | −0.450282695 | −0.724295727 |
| NYU_636 | Cancer | −2.643251321 | −0.153100106 | −0.620523759 |
| PC_04 | | −2.265503821 | 0.316884546 | −0.465645933 |
| | | AVGLAG- | FLNVL- | |
| msfile- | | TFR_446.26_721.40 | SPR_473.28_685.40 | GFLLLASLR_495.31_559.40 |
| name | Group | (SEQ ID No.: 48) | (SEQ ID No.: 53) | (SEQ ID No.: 61) |
| PC_01 | | −0.595890021 | −0.265729819 | −1.227938611 |
| ZCO491_03 | Cancer | −0.493826203 | −0.233737651 | 0.439492333 |
| ZCO415_03 | Benign | −0.823000238 | 0.091894715 | 1.340113429 |
| ZCO377_03 | Cancer | −0.461474084 | −0.132175156 | −0.681534193 |
| ZCO482_03 | Benign | −0.737284294 | −0.58444912 | 0.923867912 |
| ZCO371_03 | Benign | −0.797397915 | 0.317300363 | −0.481856091 |
| ZCO460_03 | Cancer | −1.430807772 | −0.032029072 | 0.500660403 |
| PC_02 | | −0.993447772 | −0.195869013 | −0.938750954 |
| ZCO531_01 | Cancer | −1.774211298 | −0.625129185 | −1.995990867 |
| ZCO422_03 | Benign | −1.433510857 | −0.486337724 | 0.585086518 |
| ZCO474_03 | Benign | −1.659664379 | −0.221449913 | 0.746310197 |
| ZCO539_03 | Cancer | −1.416249439 | −0.219375837 | −0.066860698 |
| ZCO464_03 | Benign | −1.453154863 | −0.283049865 | −1.341826923 |
| ZCO455_03 | Cancer | −1.417849438 | −0.329158386 | −0.844994252 |
| ZCO542_03 | Cancer | −1.004198948 | 0.274861427 | 0.84877582 |
| ZCO369_03 | Benign | −1.18343402 | −0.467548253 | −1.203726773 |
| PC_03 | | −1.402272843 | −0.314765199 | −1.146715028 |
| ZCO498_03 | Benign | −1.30773121 | −0.492803879 | −0.964660865 |
| ZCO430_03 | Cancer | −0.869971006 | −0.463504287 | 0.322733413 |
| ZCO434_03 | Cancer | −1.212392338 | −0.371335974 | 0.238258078 |
| ZCO405_03 | Benign | −0.064479432 | −0.185739668 | 0.545179554 |
| ZCO518_03 | Benign | −1.035789291 | 0.167231603 | 0.017710448 |
| ZCO388_03 | Cancer | −0.771674787 | −0.650352962 | −0.928048507 |
| PC_04 | | −1.28883251 | −0.256942282 | −0.947073186 |
| PC_01 | | −1.276607504 | −0.322049701 | −1.299878125 |
| ZCO529_02 | Cancer | −0.62776486 | −0.905207191 | −0.526568846 |
| ZCO472_02 | Benign | −0.605614802 | 0.126773047 | 0.433003945 |
| ZCO421_02 | Benign | −1.138589459 | 0.155481463 | −0.695976049 |
| ZCO517_02 | Cancer | −0.894491725 | −0.223724725 | 1.270103256 |
| ZCO414_02 | Cancer | −0.993697086 | −0.14111493 | 0.081328415 |
| ZCO467_02 | Benign | −0.819366943 | −0.490629365 | −0.928608152 |
| PC_02 | | −1.436376666 | −0.280759895 | −1.183046899 |
| ZCO538_02 | Benign | −1.207268932 | −0.386945256 | −0.765638772 |
| ZCO490_02 | Cancer | −1.030815431 | −0.200863024 | −0.045772283 |
| ZCO513_02 | Benign | −1.446577584 | 0.101495876 | 0.263179228 |
| ZCO368_02 | Cancer | −1.011497064 | −0.077313902 | −0.817280471 |
| ZCO478_02 | Benign | −0.929110875 | −0.313439436 | −1.152980215 |
| ZCO509_02 | Cancer | −1.221437963 | −0.144234708 | 1.446374387 |
| ZCO457_02 | Benign | −0.675001825 | −0.168245386 | −0.123898077 |
| ZCO384_02 | Cancer | −0.587121499 | 0.068090374 | −0.918140631 |
| PC_03 | | −1.129611582 | −0.253833885 | −1.048234464 |
| ZCO364_02 | Benign | −0.899323396 | −0.109305344 | −0.876575171 |
| ZCO392_02 | Cancer | −1.562758707 | −0.386231201 | −1.129221844 |
| ZCO401_02 | Cancer | −0.935061409 | 0.03449271 | −0.946289131 |
| ZCO544_02 | Benign | −1.236519156 | 0.004737955 | 0.547125485 |
| ZCO526_01 | Benign | −1.121391929 | −0.089897078 | −0.354297368 |
| ZCO445_02 | Cancer | −0.853079604 | −0.441785009 | −0.283911223 |
| PC_04 | | −1.005768423 | −0.276367058 | −0.545990681 |
| PC_01 | | −1.194120072 | −0.314610004 | −1.268580087 |
| CAP00721-09 | Benign | −0.824206097 | −0.47179435 | −1.101995516 |
| CAP00749-09 | Cancer | −0.768932709 | 0.108943371 | −2.128318991 |
| CAP00132-07 | Cancer | −0.678356278 | −0.082058675 | 1.103324917 |
| CAP02123-09 | Benign | −1.197971179 | 0.040954009 | 0.408728205 |
| CAP03009-08 | Benign | −0.885766805 | −0.353007615 | −1.165057287 |
| CAP01154-06 | Cancer | −1.428146543 | 0.017893842 | −0.455169138 |
| PC_02 | | −1.044387873 | −0.341323718 | −1.406951978 |
| CAP02208-05 | Benign | −1.207518317 | 0.451938799 | 0.493262196 |
| CAP00157-07 | Cancer | −1.311667116 | −0.124985079 | 1.135970035 |
| CAP00369-10 | Benign | −1.424174984 | 0.391201664 | 0.534919725 |
| CAP03006-08 | Cancer | −1.390241853 | 0.209163016 | 0.229804786 |
| CAP01799-08 | Benign | −0.990656682 | −0.489945704 | −0.494679252 |
| CAP02126-09 | Benign | −0.981067505 | 0.166388215 | −0.963792991 |
| PC_03 | | −1.162911567 | −0.245007085 | −1.303405184 |
| CAP01129-06 | Cancer | −1.268049258 | 0.25760536 | 0.134030297 |
| CAP01791-08 | Cancer | −0.594428216 | −0.203457711 | −2.008333133 |
| PC_04 | | −0.937807496 | −0.079449244 | −0.846820515 |
| PC_01 | | −1.219374441 | −0.091919823 | −0.467348275 |
| NYU_16 | Cancer | −1.36137085 | 0.207247052 | 0.724456565 |
| NYU_24 | Benign | −1.152680046 | 0.716802974 | 0.276967129 |
| NYU_514 | Benign | −0.809327936 | −0.267999594 | 0.79001039 |
| NYU_349 | Cancer | −0.949845868 | −0.197363148 | 0.748057357 |
| NYU_379 | Cancer | −0.961355236 | −0.146887632 | 0.9653112 |
| NYU_1145 | Benign | −0.923639264 | −0.258406777 | 0.240206185 |
| PC_02 | | −1.342214257 | 0.035521329 | −1.081834406 |
| NYU_696 | Cancer | −0.897617421 | −0.006344278 | 1.649572769 |
| NYU_84 | Benign | −0.677879294 | 0.056526843 | 1.268123508 |
| NYU_907 | Cancer | −0.246833145 | −0.038704509 | 2.099011291 |
| NYU_332 | Benign | −0.926869344 | −0.319735087 | 1.663214016 |
| NYU_173 | Benign | −1.030068495 | −0.807532008 | −0.178594739 |
| NYU_427 | Cancer | −1.393845675 | −0.633845789 | 0.316608124 |
| NYU_184 | Cancer | −0.83550514 | −0.190615839 | 0.286138544 |
| NYU_1001 | Benign | −0.506419063 | −0.229858435 | −0.316528934 |
| PC_03 | | −1.19408064 | 0.015317538 | −1.015068301 |
| NYU_453 | Benign | −0.91187095 | −0.170780258 | 1.489578321 |
| NYU_1141 | Cancer | −0.711310697 | 0.528907512 | 1.25748375 |
| NYU_1096 | Cancer | −0.607458144 | 0.287065436 | 0.392346406 |
| NYU_500 | Benign | −1.178820948 | 0.280265177 | 0.689462768 |
| NYU_1317 | Cancer | −1.151712261 | −0.152397769 | 1.50321441 |
| NYU_841 | Benign | −2.179336556 | −0.956730113 | 0.448863259 |
| PC_04 | | −1.198922197 | −0.14924787 | 0.721947796 |
| PC_01 | | −0.881639537 | 0.079079308 | −0.526578831 |
| NYU_28 | Benign | −1.050978886 | −0.294892351 | 0.72984141 |
| NYU_1559S | Cancer | −0.979266794 | 0.364329627 | 1.076154804 |
| NYU_440 | Benign | −0.348677875 | −0.458820954 | 0.290461965 |
| NYU_1176 | Cancer | −0.293039083 | 0.300632063 | −1.0105483 |
| NYU_831 | Cancer | −0.511136376 | 0.116878637 | 1.238081773 |
| NYU_71 | Benign | −0.243455164 | 0.018694084 | −0.043670603 |
| PC_02 | | −1.301607447 | −0.057143347 | −1.075310922 |
| NYU_111 | Cancer | −0.917017163 | −0.230720462 | 1.274187125 |
| NYU_423 | Benign | −0.906923167 | 0.088502384 | 0.451915417 |
| NYU_834 | Benign | −1.117107311 | −0.194921982 | 0.05579903 |
| NYU_830 | Cancer | −0.68391899 | −0.00446209 | 0.803045616 |
| NYU560 | Cancer | −0.896225773 | −0.118188113 | 0.070278604 |
| NYU_281 | Benign | −1.327094178 | 0.334784157 | 0.768467564 |
| NYU_613S | Cancer | −1.070806068 | −0.495089863 | 1.143325267 |
| NYU_513 | Benign | −0.851456769 | −0.194865065 | −0.803577665 |
| PC_03 | | −1.029706497 | −0.008146198 | −1.054012744 |
| NYU_661 | Cancer | −0.614340916 | −0.114660609 | 0.653634439 |
| NYU_1168 | Benign | −1.244404731 | −0.419660819 | 0.136578755 |
| NYU_968 | Benign | −0.634740466 | 0.237646596 | 1.716207592 |
| NYU_410 | Cancer | −0.559048148 | −0.468820154 | 0.523467245 |
| NYU_1098 | Benign | −1.238653158 | 0.282757837 | 1.512945197 |
| NYU_636 | Cancer | −1.365503969 | −0.121142723 | 0.29600241 |
| PC_04 | | −0.678759122 | −0.128255466 | 0.474154241 |

TABLE 10B
All data for the 18 candidate proteins (Box Cox transformed and normalized)

| | | LDTLAQE- | LGG- | |
|---|---|---|---|---|
| msfile- | | VALLK_657.39_871.50 | PEAGLGEYLFER_804.40_1083.60 | LTLLAPLNSVFK_658.40_804.50 |
| name | Group | (SEQ ID No.: 66) | (SEQ ID No.: 50) | (SEQ ID No.: 46) |
| PC_01 | | 0.619233775 | −3.688218544 | 0.320149361 |
| ZCO491_03 | Cancer | 0.307041039 | −2.495871594 | 0.634187197 |
| ZCO415_03 | Benign | 0.149791503 | −1.839735407 | 0.087355699 |
| ZCO377_03 | Cancer | −0.319268537 | −2.353210558 | −0.238039285 |
| ZCO482_03 | Benign | −0.109132038 | −3.89810845 | 0.491491092 |
| ZCO371_03 | Benign | 0.535371292 | −3.396987038 | 0.501177683 |
| ZCO460_03 | Cancer | 0.375108688 | −2.591187408 | 0.163636871 |
| PC_02 | | 0.259423835 | −3.467473208 | 0.388379979 |
| ZCO531_01 | Cancer | 0.353435158 | −0.863765461 | 0.134451448 |
| ZCO422_03 | Benign | 0.267899548 | −4.128960152 | −0.036398134 |
| ZCO474_03 | Benign | 0.11239326 | −2.008626279 | 0.049305919 |
| ZCO539_03 | Cancer | −0.144515562 | −2.409318593 | 0.178753247 |
| ZCO464_03 | Benign | 0.322619955 | −2.803572494 | 0.141936263 |
| ZCO455_03 | Cancer | 0.164885913 | −1.645442718 | −0.194675578 |
| ZCO542_03 | Cancer | 0.126503625 | −1.345123378 | −0.010132403 |
| ZCO369_03 | Benign | 0.323985529 | −1.147298656 | 0.394215825 |
| PC_03 | | 0.243236055 | −3.464681928 | 0.252725085 |
| ZCO498_03 | Benign | 0.009387339 | −2.20373592 | −0.028545713 |
| ZCO430_03 | Cancer | 0.155120044 | −2.564247278 | 0.117113156 |
| ZCO434_03 | Cancer | 0.203836126 | −2.127566504 | 0.326654093 |
| ZCO405_03 | Benign | 0.229845196 | −0.852835223 | 0.879718032 |
| ZCO518_03 | Benign | 0.599055389 | −2.870829067 | 0.127530727 |
| ZCO388_03 | Cancer | 0.471424676 | −2.412924032 | 0.008756886 |
| PC_04 | | 0.129995335 | −2.752431012 | 0.186571819 |
| PC_01 | | 0.422932853 | −3.695102369 | 0.206164614 |
| ZCO529_02 | Cancer | 0.235706327 | −1.648601545 | 0.081950191 |
| ZCO472_02 | Benign | 0.351197234 | −0.988396993 | 0.44684055 |
| ZCO421_02 | Benign | 0.243069031 | −3.149469001 | −0.12736403 |
| ZCO517_02 | Cancer | 0.379359109 | −2.685656021 | 0.320454182 |
| ZCO414_02 | Cancer | 0.084138401 | −2.552751017 | 0.553682137 |
| ZCO467_02 | Benign | 0.352364221 | −4.466156537 | 0.065072261 |
| PC_02 | | 0.357615874 | −3.796356148 | 0.223966665 |
| ZCO538_02 | Benign | 0.388669004 | −3.028978417 | −0.005175742 |
| ZCO490_02 | Cancer | 0.198993161 | −2.458856922 | 0.37064057 |
| ZCO513_02 | Benign | 0.376467361 | −3.872414593 | −0.220383484 |
| ZCO368_02 | Cancer | −0.030242782 | −3.707959588 | −0.030270885 |
| ZCO478_02 | Benign | 0.234687564 | −1.735399165 | 0.216377484 |
| ZCO509_02 | Cancer | 0.16439562 | −1.813156102 | 0.456046049 |
| ZCO457_02 | Benign | −0.084654579 | −2.873426534 | 0.121193021 |
| ZCO384_02 | Cancer | −0.046133487 | −2.190926774 | 0.319872593 |
| PC_03 | | 0.206759546 | −3.340738983 | 0.173434124 |
| ZCO364_02 | Benign | 0.054668973 | −2.557147438 | −0.035159443 |
| ZCO392_02 | Cancer | 0.524123185 | −1.563637637 | −0.280254089 |
| ZCO401_02 | Cancer | 0.410914218 | −2.210733391 | −0.292704095 |
| ZCO544_02 | Benign | 0.164354649 | −1.889319319 | 0.297890338 |
| ZCO526_01 | Benign | 0.293123237 | −0.882390871 | 0.383353727 |
| ZCO445_02 | Cancer | 0.244665703 | −2.350289612 | 0.024075876 |
| PC_04 | | 0.313710958 | −2.346884066 | 0.016758546 |
| PC_01 | | 0.262212362 | −3.691638396 | 0.244499792 |
| CAP00721-09 | Benign | −0.154679077 | −1.784515505 | 0.137664468 |
| CAP00749-09 | Cancer | 0.372492851 | −2.784820594 | 0.28247611 |
| CAP00132-07 | Cancer | 0.28491549 | −1.757602443 | 0.793614607 |
| CAP02123-09 | Benign | 0.330319388 | −2.110871926 | 0.242968905 |
| CAP03009-08 | Benign | 0.591620089 | −1.103935587 | 0.79962435 |
| CAP01154-06 | Cancer | 0.183180678 | −1.881252857 | 0.473490727 |
| PC_02 | | 0.169136305 | −3.449506953 | 0.270539903 |
| CAP02208-05 | Benign | 0.236085021 | −4.709549056 | 0.386213217 |
| CAP00157-07 | Cancer | 0.235820707 | −2.617548641 | 0.342553135 |
| CAP00369-10 | Benign | 0.318863669 | −4.714011647 | 0.376834146 |
| CAP03006-08 | Cancer | 0.572399135 | −2.385458597 | 0.517799646 |
| CAP01799-08 | Benign | −0.419881689 | −2.814919092 | 0.184932647 |
| CAP02126-09 | Benign | 0.146597672 | −2.897762178 | 0.195005917 |
| PC_03 | | 0.231415489 | −3.543298868 | 0.323335189 |
| CAP01129-06 | Cancer | 0.376771378 | −2.105630759 | 0.166595661 |
| CAP01791-08 | Cancer | 0.085133472 | −1.85760384 | 0.218233976 |
| PC_04 | | 0.201396288 | −3.062576057 | 0.258350651 |
| PC_01 | | 0.176770024 | −3.396924804 | 0.191863897 |
| NYU_16 | Cancer | −0.123352366 | −1.750514304 | −0.513844018 |
| NYU_24 | Benign | −0.023134978 | −1.569304668 | 0.338163528 |
| NYU_514 | Benign | −0.243131868 | −2.200905151 | −0.155816279 |
| NYU_349 | Cancer | −0.534556315 | −3.270221957 | −0.202861839 |
| NYU_379 | Cancer | 0.696129534 | −2.774806808 | −0.044444522 |
| NYU_1145 | Benign | 0.83082744 | −3.571871911 | 0.106521723 |
| PC_02 | | 0.138103809 | −3.534763675 | 0.205869061 |
| NYU_696 | Can cer | −0.035605577 | −4.107452495 | −0.127288324 |
| NYU_84 | Benign | −0.233151821 | −3.902153927 | 0.384839283 |
| NYU_907 | Cancer | −0.496383559 | −4.026681756 | −0.159095297 |
| NYU_332 | Benign | −0.141236556 | −3.25467451 | 0.075657348 |
| NYU_173 | Benign | −0.058655255 | −3.515427331 | 0.402438598 |
| NYU_427 | Cancer | 0.148908128 | −2.815392807 | 0.309347149 |
| NYU_184 | Cancer | −0.14532559 | −2.135696527 | 0.314590618 |
| NYU_1001 | Benign | 0.171635645 | −1.536862239 | −0.145970589 |
| PC_03 | | 0.04799084 | −3.462930927 | 0.238054547 |
| NYU_453 | Benign | 0.611264436 | −2.949077132 | 0.382972022 |
| NYU_1141 | Cancer | 0.124894126 | −1.02035875 | 0.598092919 |
| NYU_1096 | Cancer | 0.966928872 | −2.978084235 | 0.157857946 |
| NYU_500 | Benign | 0.65801761 | −1.847727564 | 0.348766683 |
| NYU_1317 | Cancer | 0.222332442 | −2.365186434 | 0.230568054 |
| NYU_841 | Benign | 0.726482601 | −2.134033408 | 0.189484038 |
| PC_04 | | −0.26227648 | −3.108583393 | 0.182130085 |
| PC_01 | | 0.203599121 | −3.093371492 | 0.403602931 |
| NYU_28 | Benign | −0.062320069 | −2.237263003 | 0.246989699 |
| NYU_1559S | Cancer | −0.001186789 | −1.248911767 | 0.601965515 |
| NYU_440 | Benign | −0.302850212 | −2.251273516 | 0.30677522 |
| NYU_1176 | Cancer | −0.435270851 | −3.779661486 | 0.146132312 |
| NYU_831 | Cancer | 0.047253239 | −2.644442757 | 0.42264776 |
| NYU_71 | Benign | −0.114865443 | −3.351976972 | −0.007703574 |
| PC_02 | | 0.020529227 | −3.630372194 | 0.169697886 |
| NYU_111 | Cancer | 0.697156707 | −1.900586292 | 0.37342108 |
| NYU_423 | Benign | 0.7282604 | −3.90111154 | −0.060128323 |
| NYU_834 | Benign | −0.511576596 | −1.294826096 | −0.056567679 |
| NYU_830 | Cancer | 0.164584549 | −2.771863627 | 0.275831467 |
| NYU560 | Cancer | −0.195713033 | −2.940360322 | 0.252223315 |
| NYU_281 | Benign | −0.195309228 | −2.067542099 | 0.083312654 |
| NYU_613S | Cancer | −0.15309093 | −2.714972675 | 0.098970272 |
| NYU_513 | Benign | −0.463079716 | −3.745439731 | −0.10376122 |
| PC_03 | | 0.021256222 | −3.432587168 | 0.332445129 |
| NYU_661 | Cancer | −0.085425612 | −2.394966353 | 0.319005642 |
| NYU_1168 | Benign | −0.320494963 | −2.594487321 | 0.041207713 |
| NYU_968 | Benign | 0.083348208 | −3.137744896 | 0.360562139 |
| NYU_410 | Cancer | −0.26731122 | −2.334222045 | 0.053360464 |
| NYU_1098 | Benign | −0.074197702 | −3.228962629 | 0.11680201 |
| NYU_636 | Cancer | 0.051966268 | −4.088190766 | 0.128561131 |
| PC_04 | | 0.080290769 | −2.246697937 | 0.227614323 |
| | | | SGYLL- | |
| msfile- | | QITVNDLPVG12_606.30_970.50 | PDTK_497.27_308.10 | STGGAPTFNVTVTK_690.40_1006.60 |
| name | Group | (SEQ ID No.: 58) | (SEQ ID No.: 49) | (SEQ ID No.: 59) |
| PC_01 | | −2.891612367 | −1.080959644 | −1.563214627 |
| ZCO491_03 | Cancer | −1.390227225 | −0.664673284 | 1.883575359 |
| ZCO415_03 | Benign | −0.756482415 | −0.404031778 | 2.605320253 |
| ZCO377_03 | Cancer | −1.804984584 | −1.820635725 | −0.295190198 |
| ZCO482_03 | Benign | −0.823352463 | −0.826182586 | 1.826299936 |
| ZCO371_03 | Benign | 1.421923229 | −1.290725633 | 0.251635695 |
| ZCO460_03 | Cancer | −0.769020246 | −1.433746671 | 0.764149828 |
| PC_02 | | −2.716860962 | −1.325149529 | −1.78210178 |
| ZCO531_01 | Cancer | −0.799181192 | −1.570588988 | −0.689527945 |
| ZCO422_03 | Benign | −0.276194137 | −1.786474285 | −1.640722668 |
| ZCO474_03 | Benign | 0.008102262 | −0.909990561 | 1.35122707 |
| ZCO539_03 | Cancer | −0.659432607 | −1.510617135 | 0.826262044 |
| ZCO464_03 | Benign | −1.068769153 | −1.800141318 | −0.309099375 |
| ZCO455_03 | Cancer | −0.866387159 | −1.713182691 | −0.582501025 |
| ZCO542_03 | Cancer | −1.137442396 | −1.064580314 | 1.515635323 |
| ZCO369_03 | Benign | −1.03008142 | −1.787664318 | −0.467494732 |
| PC_03 | | −3.002697246 | −1.347957626 | −1.965485574 |
| ZCO498_03 | Benign | −1.266038826 | −1.401799831 | 0.52968454 |
| ZCO430_03 | Cancer | −1.526637891 | −1.061050922 | 1.338378154 |
| ZCO434_03 | Cancer | −1.838641592 | −1.471497069 | 1.126873172 |
| ZCO405_03 | Benign | −0.525607784 | −0.679142563 | −0.459693172 |
| ZCO518_03 | Benign | −1.58042355 | −1.304539697 | 1.042552217 |
| ZCO388_03 | Cancer | −3.064354935 | −1.625729712 | −0.860063029 |
| PC_04 | | −1.966223678 | −1.28762834 | −0.364224566 |
| PC_01 | | −2.902280553 | −1.469478783 | −1.814501543 |
| ZCO529_02 | Cancer | −0.844243555 | −1.602762256 | 0.177099462 |
| ZCO472_02 | Benign | 0.803140338 | −1.281194903 | 1.328464271 |
| ZCO421_02 | Benign | −1.947459763 | −1.958257722 | 0.142671565 |
| ZCO517_02 | Cancer | 1.758999873 | −1.085977989 | 1.358696265 |
| ZCO414_02 | Cancer | −1.499932157 | −1.169549543 | 0.838450287 |
| ZCO467_02 | Benign | −2.167510431 | −1.189206525 | 0.613140688 |
| PC_02 | | −2.940483716 | −1.397843336 | −1.94687562 |
| ZCO538_02 | Benign | −1.525332131 | −1.59904916 | −0.338298177 |
| ZCO490_02 | Cancer | −0.198670437 | −2.096558675 | −0.255046928 |
| ZCO513_02 | Benign | −1.139247249 | −1.458818554 | 0.964364891 |
| ZCO368_02 | Cancer | −2.272808964 | −1.46764769 | −0.83985844 |
| ZCO478_02 | Benign | −0.191763267 | −1.679313206 | −1.169041219 |
| ZCO509_02 | Cancer | −0.316397016 | −1.272972633 | 1.455572928 |
| ZCO457_02 | Benign | 0.543742944 | −1.530599909 | 0.026349653 |
| ZCO384_02 | Cancer | −2.035163296 | −1.854325703 | 0.00081698 |
| PC_03 | | −2.735971874 | −1.434037091 | −1.55088974 |
| ZCO364_02 | Benign | −1.931528987 | −1.440982972 | −0.485952795 |
| ZCO392_02 | Cancer | −2.824001264 | −1.900747845 | −1.504953093 |
| ZCO401_02 | Cancer | −2.327798886 | −1.662750263 | −0.667249982 |
| ZCO544_02 | Benign | −0.75288953 | −1.427253932 | 0.588778937 |
| ZCO526_01 | Benign | −1.789785814 | −1.28937802 | 0.204801157 |
| ZCO445_02 | Cancer | −1.515797719 | −1.361795562 | 0.916434865 |
| PC_04 | | −1.73270517 | −1.424939058 | 0.580950059 |
| PC_01 | | −2.765279484 | −1.423835901 | −1.758581707 |
| CAP00721-09 | Benign | 0.025773455 | −1.763848125 | −2.211000583 |
| CAP00749-09 | Cancer | −0.351523725 | −1.078982456 | −1.9583196 |
| CAP00132-07 | Cancer | −0.200739783 | −1.291033643 | −1.687401442 |
| CAP02123-09 | Benign | −0.602336309 | −1.473024257 | −2.344440189 |
| CAP03009-08 | Benign | 0.045193986 | −0.727892075 | −1.417467134 |
| CAP01154-06 | Cancer | 0.105202154 | −1.059761743 | −2.437542559 |
| PC_02 | | −2.904480906 | −1.255362611 | −1.729402887 |
| CAP02208-05 | Benign | −0.692241817 | NaN | −1.389228182 |
| CAP00157-07 | Cancer | −1.224626639 | −1.265807451 | −2.715970496 |
| CAP00369-10 | Benign | −0.688014126 | −1.488720118 | −2.563264892 |
| CAP03006-08 | Cancer | −0.198551987 | −1.446714381 | −2.305369727 |
| CAP01799-08 | Benign | −1.204051747 | −1.439494226 | −1.291294706 |
| CAP02126-09 | Benign | −0.775704249 | NaN | −1.599954765 |
| PC_03 | | −2.854624327 | −1.318896418 | −2.082855904 |
| CAP01129-06 | Cancer | −0.716992148 | NaN | −1.508815804 |
| CAP01791-08 | Cancer | 0.122849694 | NaN | −1.46205964 |
| PC_04 | | −2.150100934 | −1.292026556 | −1.940560701 |
| PC_01 | | −1.816734459 | −1.261527785 | −0.26531624 |
| NYU_16 | Cancer | −2.251382348 | −2.500171462 | −1.043382774 |
| NYU_24 | Benign | −0.335164877 | −0.784881708 | 1.044512297 |
| NYU_514 | Benign | −1.172282762 | −1.370423174 | 1.179119361 |
| NYU_349 | Cancer | −1.157483658 | −1.554467518 | 0.06068016 |
| NYU_379 | Cancer | −0.694040661 | −1.433986027 | 0.778694649 |
| NYU_1145 | Benign | −0.71308125 | −1.093348407 | 0.789734251 |
| PC_02 | | −2.95583845 | −1.437095505 | −1.425350921 |
| NYU_696 | Can cer | −0.877306921 | −1.558736364 | 0.317467612 |
| NYU_84 | Benign | −1.115260124 | −0.9957724 | −0.36426484 |
| NYU_907 | Cancer | −0.20355606 | −1.679642601 | −0.005678103 |
| NYU_332 | Benign | −0.5025212 | −0.990935203 | 1.189897923 |
| NYU_173 | Benign | −2.535910655 | −1.562605379 | −1.809679759 |
| NYU_427 | Cancer | −0.246174546 | −1.55778677 | 1.263278086 |
| NYU_184 | Cancer | −0.604766494 | −1.064945228 | 0.287602207 |
| NYU_1001 | Benign | −1.562785061 | −1.478752531 | −0.896051519 |
| PC_03 | | −2.854992558 | −1.52388584 | −1.530762249 |
| NYU_453 | Benign | 0.583365159 | −1.278012286 | 1.675519887 |
| NYU_1141 | Cancer | −0.782690488 | −1.1385726 | −0.143136066 |
| NYU_1096 | Cancer | −1.901179155 | −1.578904855 | 0.056418244 |
| NYU_500 | Benign | 0.847016964 | −1.667371491 | 1.054635955 |
| NYU_1317 | Cancer | 1.239532381 | −1.332441731 | −1.12831205 |
| NYU_841 | Benign | −0.717251365 | −1.411929774 | 0.063549113 |
| PC_04 | | 0.230854049 | −1.43150263 | 1.204555236 |
| PC_01 | | −2.279540872 | −1.274941266 | −1.674599694 |
| NYU_28 | Benign | −0.98952403 | −1.451732567 | −0.164978062 |
| NYU_1559S | Cancer | −0.778814767 | −1.07906308 | −1.7446435 |
| NYU_440 | Benign | −0.450044112 | −1.110524505 | 1.216363397 |
| NYU_1176 | Cancer | −1.723078562 | −1.704385196 | −1.191450487 |
| NYU_831 | Cancer | −0.092952375 | −1.115545496 | 0.645325629 |
| NYU_71 | Benign | −0.334485707 | −1.221599855 | −0.842015315 |
| PC_02 | | −2.860062402 | −1.505589369 | −2.143608494 |
| NYU_111 | Cancer | −1.512394115 | −1.167821392 | −1.245799127 |
| NYU_423 | Benign | 0.78473187 | −1.775954255 | −0.22661634 |
| NYU_834 | Benign | −2.293573315 | −1.315638673 | −0.948358856 |
| NYU_830 | Cancer | −0.035604276 | −1.329456481 | 0.436512527 |
| NYU560 | Cancer | −1.075336391 | −1.525596457 | 0.036794864 |
| NYU_281 | Benign | −0.46084342 | −1.573182855 | 2.380374367 |
| NYU_613S | Cancer | −0.266865396 | −1.268093092 | 0.825792761 |
| NYU_513 | Benign | −0.841390086 | −1.480688037 | 0.101324615 |
| PC_03 | | −2.803095384 | −1.330731523 | −1.924656883 |
| NYU_661 | Cancer | −0.242682514 | −1.253645775 | 1.009591296 |
| NYU_1168 | Benign | −0.180996049 | −1.278353979 | 0.582964648 |
| NYU_968 | Benign | 0.569857281 | −1.702836751 | −0.466910999 |
| NYU_410 | Cancer | −0.532022467 | −1.796316817 | 1.287501522 |
| NYU_1098 | Benign | −1.231081633 | −1.674118957 | −0.125061054 |
| NYU_636 | Cancer | −1.390354201 | −1.223856327 | −0.135261231 |
| PC_04 | | −0.549538189 | −0.954431811 | 1.104866601 |

TABLE 10C
All data for the 18 candidate proteins (Box Cox transformed and normalized)

| msfile- | | TVLWPNGLSLDIPAGR_855.00_1209.70 | TWNDPSVQQDIK_715.85_288.10 | VEIFYR_413.73_598.30 |
|---|---|---|---|---|
| name | Group | (SEQ ID No.: 57) | (SEQ ID No.: 52) | (SEQ ID No.: 56) |
| PC_01 | | −2.840242783 | −2.176578096 | 0.235769891 |
| ZCO491_03 | Cancer | −3.482057591 | −1.956092764 | −0.439872384 |
| ZCO415_03 | Benign | −3.384554903 | −0.926370183 | −0.061587364 |
| ZCO377_03 | Cancer | −4.676912038 | −2.865805989 | 0.541114982 |
| ZCO482_03 | Benign | −3.470264584 | −1.660530957 | 0.697209475 |
| ZCO371_03 | Benign | −4.02116434 | −2.871246146 | 0.586191904 |
| ZCO460_03 | Cancer | −3.27744164 | −2.425791961 | 0.088834939 |
| PC_02 | | −2.703138285 | −2.288243168 | 0.346599314 |
| ZCO531_01 | Cancer | −2.505350313 | −2.355195184 | 0.435333138 |
| ZCO422_03 | Benign | −3.206993546 | −2.246840872 | −0.266603189 |
| ZCO474_03 | Benign | −2.392278512 | −2.097016205 | 0.880435954 |
| ZCO539_03 | Cancer | −2.302714823 | −2.212563 | 0.147060039 |
| ZCO464_03 | Benign | −3.18257124 | −2.770680835 | −0.112410971 |
| ZCO455_03 | Cancer | −3.385642375 | −2.39453886 | 0.182584408 |
| ZCO542_03 | Cancer | −2.832452611 | −2.010258875 | −0.389953486 |
| ZCO369_03 | Benign | −2.902571098 | −2.962547593 | 0.966322127 |
| PC_03 | | −2.720871742 | −2.249287591 | 0.196449067 |
| ZCO498_03 | Benign | −3.265537767 | −2.41227993 | 0.090606402 |
| ZCO430_03 | Cancer | −3.707731095 | −1.816943622 | 0.252058542 |
| ZCO434_03 | Cancer | −3.069371069 | −2.377595312 | 0.078324606 |
| ZCO405_03 | Benign | −3.059458744 | −2.955033898 | 0.142767191 |
| ZCO518_03 | Benign | −2.590793736 | −2.097971626 | −0.336340707 |
| ZCO388_03 | Cancer | −3.161507078 | −2.970309442 | 0.276789044 |
| PC_04 | | −2.477112012 | −2.360615772 | 0.199190053 |
| PC_01 | | −2.965810076 | −2.482123128 | 0.151344036 |
| ZCO529_02 | Cancer | −2.234309986 | −2.724299187 | 0.202929465 |
| ZCO472_02 | Benign | −3.382551936 | −2.156224909 | 0.73670206 |
| ZCO421_02 | Benign | −3.673286559 | −2.675217691 | 0.824945036 |
| ZCO517_02 | Cancer | −2.850764593 | −2.311995036 | −0.343912022 |
| ZCO414_02 | Cancer | −2.804088977 | −2.334575865 | 0.154752291 |
| ZCO467_02 | Benign | −2.72602792 | −2.958864094 | 0.332422704 |
| PC_02 | | −2.805444388 | −2.288974802 | 0.140712724 |
| ZCO538_02 | Benign | −2.473300084 | −2.593641507 | −0.023878244 |
| ZCO490_02 | Cancer | −3.559067756 | −2.358523324 | 0.499171809 |
| ZCO513_02 | Benign | −2.796155264 | −1.801656273 | −0.414019564 |
| ZCO368_02 | Cancer | −3.321506554 | −2.997123731 | 0.49305375 |
| ZCO478_02 | Benign | −3.274139788 | −2.939579006 | 0.276359484 |
| ZCO509_02 | Cancer | −3.557757608 | −1.817206163 | −0.752415077 |
| ZCO457_02 | Benign | −3.819289816 | −2.087937624 | 0.164722479 |
| ZCO384_02 | Cancer | −3.894370789 | −2.750272321 | −0.182884258 |
| PC_03 | | −3.075698429 | −2.215431221 | 0.058439151 |
| ZCO364_02 | Benign | −3.347518192 | −2.713380391 | 0.36829733 |
| ZCO392_02 | Cancer | −3.698051173 | −2.862068204 | −0.144884886 |
| ZCO401_02 | Cancer | −4.208091339 | −2.855015859 | −0.310269045 |
| ZCO544_02 | Benign | −3.286401353 | −2.233987781 | −0.092815592 |
| ZCO526_01 | Benign | −2.946478376 | −2.226484226 | −0.26941901 |
| ZCO445_02 | Cancer | −3.392583406 | −2.047150606 | −0.122855246 |
| PC_04 | | −4.137501224 | −1.964010142 | 0.014682455 |
| PC_01 | | −2.444230208 | −2.312341692 | 0.194442703 |
| CAP00721-09 | Benign | −3.373279653 | −3.279318571 | −0.014104321 |
| CAP00749-09 | Cancer | −2.080239374 | −2.547431417 | −0.404521849 |
| CAP00132-07 | Cancer | −2.557406753 | −2.599913502 | 0.086243743 |
| CAP02123-09 | Benign | −2.22619151 | −2.887411963 | −0.110700863 |
| CAP03009-08 | Benign | −2.097549879 | −2.638008248 | 1.038552428 |
| CAP01154-06 | Cancer | −0.599913154 | −2.491348462 | −0.064112311 |
| PC_02 | | −2.333747655 | −2.094278877 | 0.186303863 |
| CAP02208-05 | Benign | −2.826110671 | −2.451742183 | 0.625897784 |
| CAP00157-07 | Cancer | −1.997178841 | −2.25472442 | 0.065225407 |
| CAP00369-10 | Benign | −3.160084337 | −2.789155086 | 0.623888644 |
| CAP03006-08 | Cancer | −2.235657894 | −2.180367368 | −0.236616097 |
| CAP01799-08 | Benign | −2.586851264 | −2.514836093 | 0.102158093 |
| CAP02126-09 | Benign | −2.152543713 | −2.825647732 | 0.134178863 |
| PC_03 | | −2.201921094 | −2.108691181 | 0.244854194 |
| CAP01129-06 | Cancer | −2.133293575 | −2.459117389 | −0.146614889 |
| CAP01791-08 | Cancer | −1.985201146 | −2.451935406 | 0.02936058 |
| PC_04 | | −2.123858431 | −1.961824761 | 0.307697524 |
| PC_01 | | −2.868585357 | −2.451793786 | 0.139567381 |
| NYU_16 | Cancer | −5.217314008 | −3.647120634 | −0.250758122 |
| NYU_24 | Benign | −4.151449744 | −1.886572173 | 0.525038922 |
| NYU_514 | Benign | −4.44817412 | −2.090526634 | 0.362030623 |
| NYU_349 | Cancer | −4.522788735 | −2.825922282 | 0.214022036 |
| NYU_379 | Cancer | −3.656553516 | −2.639836281 | 0.299954118 |
| NYU_1145 | Benign | −3.016893529 | −2.389606375 | 0.061744966 |
| PC_02 | | −2.523598572 | −2.285039262 | 0.216875846 |
| NYU_696 | Cancer | −2.997701491 | −2.408130714 | 0.569379895 |
| NYU_84 | Benign | −3.453769009 | −2.243435341 | 0.487779235 |
| NYU_907 | Cancer | −3.65802143 | −2.14857613 | 0.552819037 |
| NYU_332 | Benign | −4.1942367 | −2.097513372 | 0.43102388 |
| NYU_173 | Benign | −3.674973494 | −2.751931751 | 0.989466593 |
| NYU_427 | Cancer | −4.0278829 | −2.714916823 | 0.035938333 |
| NYU_184 | Cancer | −2.904851738 | −1.604414615 | 0.282859107 |
| NYU_1001 | Benign | −2.150077192 | −2.901137469 | −0.468744436 |
| PC_03 | | −3.053283217 | −2.040653191 | 0.217092411 |
| NYU_453 | Benign | −3.577645661 | −2.107714914 | 0.737241032 |
| NYU_1141 | Cancer | −2.948893334 | −2.125786815 | −0.226706292 |
| NYU_1096 | Cancer | −3.105624526 | −2.08815406 | 0.101708958 |
| NYU_500 | Benign | −2.926910767 | −2.02451037 | −0.349285544 |
| NYU_1317 | Cancer | −3.233020084 | −1.813682983 | −0.305035753 |
| NYU_841 | Benign | −1.986128205 | −2.034585896 | 0.325299893 |
| PC_04 | | −3.672172295 | −2.258669838 | 0.57977164 |
| PC_01 | | −2.702403872 | −2.183962224 | 0.237568119 |
| NYU_28 | Benign | −2.814893326 | −2.615293625 | −0.369557833 |
| NYU_1559S | Cancer | −2.96988681 | −3.195396714 | 0.569701508 |
| NYU_440 | Benign | −3.788331302 | −2.212834014 | 0.279358219 |
| NYU_1176 | Cancer | −2.772918723 | −2.835713174 | −0.03258978 |
| NYU_831 | Cancer | −3.601945958 | −2.414315763 | 0.363715053 |
| NYU_71 | Benign | −3.073918447 | −2.447684579 | 0.103567059 |
| PC_02 | | −2.942645472 | −2.30296314 | 0.138257047 |
| NYU_111 | Cancer | −1.491277854 | −2.310219565 | 0.030710147 |
| NYU_423 | Benign | −3.772250967 | −2.311517368 | −0.331236285 |
| NYU_834 | Benign | −1.758231185 | −2.880053781 | 0.346428361 |
| NYU_830 | Cancer | −3.436085517 | −2.347758514 | 0.138201066 |
| NYU560 | Cancer | −2.92380194 | −2.139973479 | 0.584319661 |
| NYU_281 | Benign | −3.215243914 | −2.607654246 | 0.293153827 |
| NYU_613S | Cancer | −3.315364874 | −2.449523441 | 0.077708676 |
| NYU_513 | Benign | −2.4821582 | −2.177312923 | 0.697210548 |
| PC_03 | | −2.608003487 | −2.160869025 | 0.21004925 |
| NYU_661 | Cancer | −3.092538726 | −2.327335546 | 0.059735909 |
| NYU_1168 | Benign | −2.604658409 | −2.326906594 | 0.170066144 |
| NYU_968 | Benign | −2.680436297 | −2.514319365 | −0.862746155 |
| NYU_410 | Cancer | −3.593342893 | −2.417399622 | 0.314502654 |
| NYU_1098 | Benign | −2.390332481 | −2.303175406 | −0.1836735 |
| NYU_636 | Cancer | −2.804958414 | −2.123545 | 0.334555033 |
| PC_04 | | −3.521584136 | −2.300116276 | −0.087460504 |
| | | YEV- | | |
| msfile- | | TVVSVR_526.29_293.10 | YVSELHLTR_373.21_428.30 | YYIAASYVK_539.28_638.40 |
| name | Group | (SEQ ID No.: 60) | (SEQ ID No.: 55) | (SEQ ID No.: 51) |
| PC_01 | | −0.16059136 | −0.588866587 | −0.985213754 |
| ZCO491_03 | Cancer | −0.20930411 | −0.857616199 | −1.018864244 |
| ZCO415_03 | Benign | −0.470264726 | −0.664246104 | −1.326357245 |
| ZCO377_03 | Cancer | −0.587776602 | −0.906852 | −0.978465968 |
| ZCO482_03 | Benign | −0.448347375 | −0.742102195 | −1.076891981 |
| ZCO371_03 | Benign | −0.202780497 | −0.692331274 | −1.088937238 |
| ZCO460_03 | Cancer | −0.398866766 | −0.72722677 | −1.028594397 |
| PC_02 | | −0.08393231 | −0.497637353 | −0.960213483 |
| ZCO531_01 | Cancer | −0.23020465 | −0.824688496 | −0.972100295 |
| ZCO422_03 | Benign | −0.596628695 | −0.775862754 | −1.174394609 |
| ZCO474_03 | Benign | −0.40835494 | −0.811781472 | −0.786590152 |
| ZCO539_03 | Cancer | −0.362460799 | −0.944796038 | −0.996375152 |
| ZCO464_03 | Benign | −0.263639531 | −0.625304957 | −1.446551741 |
| ZCO455_03 | Cancer | −0.440729056 | −0.902388499 | −1.050279108 |
| ZCO542_03 | Cancer | −0.57251411 | −0.755315917 | −1.277918828 |
| ZCO369_03 | Benign | −0.360074119 | −0.590701986 | −1.198020558 |
| PC_03 | | −0.221100546 | −0.568085385 | −0.942651197 |
| ZCO498_03 | Benign | −0.519286726 | −0.892295374 | −1.063763542 |
| ZCO430_03 | Cancer | −0.32185586 | −0.523940038 | −1.265036458 |
| ZCO434_03 | Cancer | −0.447210025 | −0.755196866 | −1.557660343 |
| ZCO405_03 | Benign | −0.492895359 | −0.710767382 | −1.316026726 |
| ZCO518_03 | Benign | −0.251139353 | −0.517274836 | −1.163936651 |
| ZCO388_03 | Cancer | −0.247175262 | −0.51758 | −1.25879944 |
| PC_04 | | −0.374949208 | −0.656873299 | −0.993927903 |
| PC_01 | | −0.27007669 | −0.56564187 | −0.98842698 |
| ZCO529_02 | Cancer | −0.416373928 | −0.791509912 | −1.442462225 |
| ZCO472_02 | Benign | −0.23297013 | −0.645726884 | −0.8260147 |
| ZCO421_02 | Benign | −0.423381339 | −0.505145394 | −1.164069333 |
| ZCO517_02 | Cancer | −0.372575345 | −0.556340708 | −1.20698192 |
| ZCO414_02 | Cancer | −0.388031724 | −0.65121192 | −1.013120145 |
| ZCO467_02 | Benign | −0.461632913 | −0.99726608 | −1.095273954 |
| PC_02 | | −0.145161128 | −0.574516244 | −0.944738595 |
| ZCO538_02 | Benign | −0.347503119 | −0.748151348 | −1.042632905 |
| ZCO490_02 | Cancer | −0.598883758 | −0.691175528 | −0.87920997 |
| ZCO513_02 | Benign | −0.142482236 | −0.410052979 | −1.241249356 |
| ZCO368_02 | Cancer | −0.309992577 | −0.422943911 | −1.037469869 |
| ZCO478_02 | Benign | −0.488769538 | −0.818621056 | −1.567811677 |
| ZCO509_02 | Cancer | −0.188171628 | −0.894847978 | −1.271173383 |
| ZCO457_02 | Benign | −0.521314531 | −0.894271778 | −1.239273761 |
| ZCO384_02 | Cancer | −0.296390287 | −0.682509086 | −1.079857133 |
| PC_03 | | −0.251630738 | −0.500125292 | −1.032718954 |
| ZCO364_02 | Benign | −0.347866416 | −0.47086587 | −1.032660552 |
| ZCO392_02 | Cancer | −0.252063704 | −0.574025566 | −0.806100634 |
| ZCO401_02 | Cancer | −0.132504022 | −0.647029213 | −1.301671863 |
| ZCO544_02 | Benign | −0.368664283 | −0.672364832 | −1.472766757 |
| ZCO526_01 | Benign | −0.524571926 | −0.666631963 | −1.383128046 |
| ZCO445_02 | Cancer | −0.229911542 | −0.506073597 | −1.290583154 |
| PC_04 | | −0.286102664 | −0.553237018 | −1.217972655 |
| PC_01 | | −0.31356777 | −0.539978288 | −1.082575152 |
| CAP00721-09 | Benign | −0.501084005 | −0.728723301 | −1.149277133 |
| CAP00749-09 | Cancer | −0.496792682 | −0.577869823 | −1.312484076 |
| CAP00132-07 | Cancer | −0.460252478 | −0.76357788 | −1.028059777 |
| CAP02123-09 | Benign | −0.54453159 | −0.777615954 | −1.007644529 |
| CAP03009-08 | Benign | −0.394971324 | −0.726387101 | −1.142302706 |
| CAP01154-06 | Cancer | −0.357449975 | −0.775375543 | −1.320366397 |
| PC_02 | | −0.248905574 | −0.51572773 | −1.208732576 |
| CAP02208-05 | Benign | −0.343695562 | −0.655781964 | −1.320528809 |
| CAP00157-07 | Cancer | −0.337483681 | −0.571898143 | −1.193780243 |
| CAP00369-10 | Benign | −0.442560845 | −0.686172987 | −1.100160796 |
| CAP03006-08 | Cancer | −0.352543382 | −0.540429487 | −1.232673051 |
| CAP01799-08 | Benign | −0.830419504 | −0.933560247 | −0.945791064 |
| CAP02126-09 | Benign | −0.668159912 | −0.800461386 | −0.67100192 |
| PC_03 | | −0.28630386 | −0.54234207 | −0.946457441 |
| CAP01129-06 | Cancer | −0.43828658 | −0.378314541 | −1.216679031 |
| CAP01791-08 | Cancer | −0.562235576 | −0.815486382 | −1.035268464 |
| PC_04 | | −0.334878353 | −0.569035778 | −1.060444583 |
| PC_01 | | −0.195143298 | −0.520211725 | −1.002839316 |
| NYU_16 | Cancer | −0.078526144 | −0.70336114 | −1.114970529 |
| NYU_24 | Benign | 0.006323696 | −0.375710898 | −1.230795754 |
| NYU_514 | Benign | −0.268389301 | −0.794532396 | −1.235073104 |
| NYU_349 | Cancer | −0.504234989 | −0.578983947 | −1.182736305 |
| NYU_379 | Cancer | −0.431704637 | −0.624567199 | −1.049707731 |
| NYU_1145 | Benign | −0.319544508 | −0.451316228 | −1.002441178 |
| PC_02 | | −0.196540816 | −0.550392492 | −1.007360547 |
| NYU_696 | Cancer | −0.358046893 | −0.492867011 | −1.345996607 |
| NYU_84 | Benign | −0.550203448 | −0.747189348 | −1.275085151 |
| NYU_907 | Cancer | −0.487176409 | −0.951976197 | −0.546222505 |
| NYU_332 | Benign | −0.431720139 | −0.668177756 | −0.984184808 |
| NYU_173 | Benign | −0.449846576 | −0.764085786 | −1.30593322 |
| NYU_427 | Cancer | −0.415169759 | −0.596224061 | −1.415831228 |
| NYU_184 | Cancer | −0.508175378 | −0.707038294 | −1.150010415 |
| NYU_1001 | Benign | −0.447162732 | −0.69813124 | −1.36190081 |
| PC_03 | | −0.147116854 | −0.52595103 | −1.002590543 |
| NYU_453 | Benign | −0.367234009 | −0.811961442 | −1.11629685 |
| NYU_1141 | Cancer | −0.339347891 | −0.630536716 | −1.101450339 |
| NYU_1096 | Cancer | −0.424856366 | −0.69223078 | −1.472915096 |
| NYU_500 | Benign | −0.401749374 | −0.65337254 | −1.014509252 |
| NYU_1317 | Cancer | −0.343105781 | −0.628854086 | −1.047541736 |
| NYU_841 | Benign | −0.368808387 | −0.896801378 | −1.016557624 |
| PC_04 | | −0.423880292 | −0.78648124 | −1.118217377 |
| PC_01 | | −0.211241946 | −0.524959807 | −1.0386507 |
| NYU_28 | Benign | −0.389227141 | −0.827037564 | −1.472629617 |
| NYU_1559S | Cancer | −0.43190517 | −0.68333436 | −1.402708194 |
| NYU_440 | Benign | −0.569408215 | −0.860428248 | −1.376923309 |
| NYU_1176 | Cancer | −0.578120225 | −0.881051969 | −0.913199971 |
| NYU_831 | Cancer | −0.442555491 | −0.771810553 | −1.136855913 |
| NYU_71 | Benign | −0.558980665 | −0.771047022 | −1.194045648 |
| PC_02 | | −0.32092235 | −0.571674597 | −1.052726215 |
| NYU_111 | Cancer | −0.35566628 | −0.485882973 | −1.252266571 |
| NYU_423 | Benign | −0.335884086 | −0.477686905 | −1.180804412 |
| NYU_834 | Benign | −0.524007503 | −0.926252041 | −1.181941715 |
| NYU_830 | Cancer | −0.403945569 | −0.716303543 | −1.1490005 |
| NYU560 | Cancer | −0.516957916 | −0.741373104 | −1.137736748 |
| NYU_281 | Benign | −0.546607576 | −0.73542324 | −1.032943398 |
| NYU_613S | Cancer | −0.457346638 | −0.672998228 | −1.080379369 |
| NYU_513 | Benign | −0.347077198 | −0.676011695 | −1.171521544 |
| PC_03 | | −0.231309763 | −0.45309845 | −1.02238549 |
| NYU_661 | Cancer | −0.540086698 | −0.803170123 | −1.017870154 |
| NYU_1168 | Benign | −0.377643861 | −0.784735481 | −1.177297293 |
| NYU_968 | Benign | −0.430532434 | −0.691207605 | −1.323385768 |
| NYU_410 | Cancer | −0.436124313 | −0.936293593 | −1.126584437 |
| NYU_1098 | Benign | −0.387059897 | −0.627952718 | −1.491294635 |
| NYU_636 | Cancer | −0.365115387 | −0.399577964 | −1.019992268 |
| PC_04 | | −0.394888144 | −0.798145476 | −1.063609486 |

TABLE 11A
PV2 fidelity small nodule batch all transitions (normalized)

| | | ALPGTPVASS- | ALPGTPVASS- | ALPGTPVASS- | ALQASALK_401.25_185.10 |
|---|---|---|---|---|---|
| msfile- | | QPR_640.85_185.10 | QPR_640.85_440.30 | QPR_640.85_841.50 | (SEQ ID No.: |
| name | status | (SEQ ID No.: 54) | (SEQ ID No.: 54) | (SEQ ID No.: 54) | 45) |
| PC_01 | | 0.072481908 | 0.113723027 | 0.114185527 | 1.104056731 |
| ZCO489_02 | Benign | 0.096687357 | 0.12833692 | 0.123520886 | 2.505383025 |
| ZCO436_02 | Cancer | 0.175900905 | 0.153036185 | 0.141876401 | 1.022008353 |
| ZCO512_02 | Cancer | 0.165422766 | 0.115499177 | 0.112783456 | 1.809774524 |
| ZCO475_02 | Benign | 0.020929229 | 0.117760584 | 0.115724014 | 1.45178974 |
| ZCO485_02 | Benign | 0.172154733 | 0.141065752 | 0.127981073 | 1.126646851 |
| ZCO536_02 | Cancer | 0.079545801 | 0.12688509 | 0.099691651 | 1.372594438 |
| PC_02 | | 0.144464483 | 0.104540439 | 0.099909759 | 0.570158949 |
| ZCO496_02 | Benign | 0.186731479 | 0.138624849 | 0.138123536 | 1.0877756 |
| ZCO502_02 | Cancer | 0.166799714 | 0.207401234 | 0.208648996 | 4.289444175 |
| ZCO382_02 | Benign | 0.052741617 | 0.126173724 | 0.106884057 | 0.742880387 |
| ZCO431_02 | Cancer | 0.11746052 | 0.086230586 | 0.095294864 | 2.759952104 |
| ZCO449_02 | Cancer | 0.021338221 | 0.093127082 | 0.096621539 | 2.119548876 |
| ZCO537_02 | Benign | 0.15168794 | 0.085758182 | 0.09513695 | 1.778541716 |
| ZCO362_02 | Benign | 0.166434619 | 0.130847541 | 0.103731549 | 0.500682848 |
| ZCO488_02 | Benign | 0.03773585 | 0.130035911 | 0.115317637 | 1.248930596 |
| PC_03 | | 0.043905454 | 0.103505534 | 0.128472249 | 0.583700424 |
| ZCO535_02 | Benign | 0.064443293 | 0.094776693 | 0.090581319 | 1.240370401 |
| ZCO443_02 | Cancer | 0.081472483 | 0.109663279 | 0.098436694 | 4.327131943 |
| ZCO393_02 | Benign | 0.037641224 | 0.110792301 | 0.096732074 | 0.748655274 |
| ZCO503_02 | Cancer | 0.031717637 | 0.153131384 | 0.141291671 | 2.0365338 |
| ZCO438_02 | Cancer | 0.257589409 | 0.139366076 | 0.117717494 | 2.490783377 |
| ZCO406_02 | Benign | 0.313760117 | 0.246885952 | 0.198346056 | 1.778565031 |
| PC_04 | | 0.139192591 | 0.125345674 | 0.12146445 | 0.6206359 |
| PC_01 | | 0.032854207 | 0.111385997 | 0.117494828 | 0.699259064 |
| 00082_07 | Cancer | 0.019841042 | 0.137128337 | 0.124959902 | 0.36884965 |
| 02286_07 | Benign | 0.108146504 | 0.138304617 | 0.136311272 | 0.378315451 |
| 02280_06 | Cancer | 0.030207178 | 0.114696236 | 0.106509355 | 0.344164424 |
| 01123_06 | Benign | 0.097340937 | 0.130575774 | 0.12590349 | 0.422455943 |
| 00156_07 | Cancer | 0.099055099 | 0.10758475 | 0.098752735 | 0.394029589 |
| 00781_09 | Benign | 0.113120132 | 0.124652335 | 0.121664894 | 0.477100471 |
| 00539_08 | Cancer | 0.191671411 | 0.123020001 | 0.130842261 | 0.550427075 |
| 02241_07 | Cancer | 0.22705995 | 0.146427909 | 0.142606122 | 0.397118813 |
| 02226_05 | Benign | 0.091982898 | 0.184879682 | 0.097659474 | 0.357293528 |
| PC_03 | | 0.155433794 | 0.104908646 | 0.107830802 | 0.620704861 |
| 00542_08 | NA | 0.023768339 | 0.083108762 | 0.081409514 | 0.348957783 |
| 02497_10 | NA | 0.12461502 | 0.091882185 | 0.094349037 | 0.310013188 |
| 02224_05 | Benign | 0.166455134 | 0.117225234 | 0.095221667 | 0.346682411 |
| 00748_09 | Cancer | 0.173113995 | 0.092426494 | 0.099657833 | 0.377867563 |
| 03630_09 | Benign | 0.163027974 | 0.138165406 | 0.136837465 | 0.500873729 |
| 02279_07 | Cancer | 0.154381017 | 0.141251604 | 0.134240545 | 0.560889545 |
| PC_04 | | 0.15216329 | 0.110843419 | 0.100417917 | 0.520482442 |
| PC_01 | | 0.090621435 | 0.109606492 | 0.106342907 | 0.603469727 |
| NYU806 | Benign | 0.083361378 | 0.120466716 | 0.10479075 | 1.193023537 |
| NYU777 | Cancer | 0.102578671 | 0.132414016 | 0.108105448 | 0.990005531 |
| NYU176 | Benign | 0.118623857 | 0.112882719 | 0.086169336 | 0.64992424 |
| NYU888 | Cancer | 1.051043345 | 0.179198758 | 0.149871425 | 0.624811178 |
| NYU1117 | Benign | 0.124315822 | 0.114306848 | 0.118946556 | 0.382648491 |
| NYU1201 | Cancer | 0.188865868 | 0.097604131 | 0.127325538 | 0.489872435 |
| PC_02 | | 0.064639837 | 0.085501438 | 0.097459191 | 0.572502535 |
| NYU887 | Cancer | 0.065580518 | 0.110794347 | 0.104610841 | 0.545640243 |
| NYU815 | Benign | 0.137562675 | 0.073686776 | 0.081694792 | 0.656169467 |
| NYU927 | Cancer | 0.440720193 | 0.294725239 | 0.250755809 | 0.873587542 |
| NYU1030 | Benign | 0.131926586 | 0.184096253 | 0.153705653 | 0.426077965 |
| NYU1151 | Cancer | 0.101287972 | 0.118852417 | 0.117167631 | 0.595478882 |
| NYU1005 | Benign | 0.071434457 | 0.11023886 | 0.08990643 | 1.32690047 |
| NYU522 | Benign | 0.0462317 | 0.111544673 | 0.082789283 | 1.563426942 |
| NYU389 | Cancer | 0.070096926 | 0.138667591 | 0.101185001 | 1.309339617 |
| PC_03 | | 0.124156164 | 0.116180769 | 0.101723471 | 0.578049717 |
| NYU729 | Cancer | 0.319014556 | 0.206906013 | 0.136786261 | 1.171981607 |
| NYU430 | Benign | 0.099772187 | 0.10523163 | 0.099401633 | 0.62923911 |
| NYU144 | Benign | 0.251269192 | 0.142890674 | 0.129469934 | 1.012127218 |
| NYU256 | Cancer | 0.11320516 | 0.11062707 | 0.110373612 | 0.426960724 |
| NYU1000 | Benign | 0.174645479 | 0.155090317 | 0.142656303 | 0.791369662 |
| NYU575 | Cancer | 0.083776109 | 0.146926408 | 0.117293186 | 3.539453856 |
| PC_04 | | 0.154661511 | 0.12635077 | 0.121087937 | 0.669431205 |

| | | | | AT- |
|---|---|---|---|---|
| | | ALQASALK_401.25_489.30 | ALQASALK_401.25_617.40 | VNPSAPR_456.80_386.20 |
| msfile- | | (SEQ ID No.: | (SEQ ID No.: | (SEQ ID No.: |
| name | status | 45) | 45) | 47) |
| PC_01 | | 1.013714768 | 0.997003501 | 0.513190922 |
| ZCO489_02 | Benign | 2.48957508 | 2.475361887 | 0.484191391 |
| ZCO436_02 | Cancer | 0.884283215 | 0.941295682 | 0.510892497 |
| ZCO512_02 | Cancer | 1.835667867 | 1.762379443 | 0.486408258 |
| ZCO475_02 | Benign | 1.261706074 | 1.432702764 | 0.604057454 |
| ZCO485_02 | Benign | 1.183038102 | 1.110417336 | 0.642058773 |
| ZCO536_02 | Cancer | 1.195337479 | 1.350378186 | 0.76209092 |
| PC_02 | | 0.524625346 | 0.566255019 | 0.483881017 |
| ZCO496_02 | Benign | 1.054769834 | 1.123342506 | 0.48130832 |
| ZCO502_02 | Cancer | 4.131978903 | 4.808895277 | 0.766300173 |
| ZCO382_02 | Benign | 0.620959101 | 0.686212655 | 0.536594739 |
| ZCO431_02 | Cancer | 2.999228632 | 2.670892954 | 0.52272151 |
| ZCO449_02 | Cancer | 1.822591849 | 2.29946133 | 0.409845148 |
| ZCO537_02 | Benign | 1.641773423 | 1.825637212 | 0.46477433 |
| ZCO362_02 | Benign | 0.460425029 | 0.495840777 | 0.488311608 |
| ZCO488_02 | Benign | 1.268964485 | 1.267486846 | 0.634140411 |
| PC_03 | | 0.576457637 | 0.641518967 | 0.539489248 |
| ZCO535_02 | Benign | 1.112334351 | 1.264916516 | 0.597070961 |
| ZCO443_02 | Cancer | 4.146180928 | 4.845153552 | 0.604529755 |
| ZCO393_02 | Benign | 0.675383716 | 0.746970867 | 0.580525256 |
| ZCO503_02 | Cancer | 1.874909124 | 2.004130039 | 0.564575514 |
| ZCO438_02 | Cancer | 2.431852281 | 2.349048088 | 0.857019612 |
| ZCO406_02 | Benign | 1.72007119 | 1.934236248 | 1.303030376 |
| PC_04 | | 0.542198431 | 0.573190384 | 0.5364696 |
| PC_01 | | 0.589246404 | 0.61082259 | 0.522477935 |
| 00082_07 | Cancer | 0.325172092 | 0.293861994 | 0.508267589 |
| 02286_07 | Benign | 0.318440954 | 0.386308647 | 0.62822393 |
| 02280_06 | Cancer | 0.309306972 | 0.314934681 | 0.570945741 |
| 01123_06 | Benign | 0.454116112 | 0.45399105 | 0.749329059 |
| 00156_07 | Cancer | 0.323103636 | 0.387953902 | 0.884455539 |
| 00781_09 | Benign | 0.388429093 | 0.455908149 | 0.563459111 |
| 00539_08 | Cancer | 0.487164394 | 0.52838435 | 0.459851826 |
| 02241_07 | Cancer | 0.318777488 | 0.386103989 | 0.472661051 |
| 02226_05 | Benign | 0.316772323 | 0.344240011 | 0.840015283 |
| PC_03 | | 0.603580671 | 0.625066231 | 0.534207137 |
| 00542_08 | NA | 0.345598358 | 0.33541418 | 0.667521756 |
| 02497_10 | NA | 0.278995049 | 0.290460208 | 0.48646257 |
| 02224_05 | Benign | 0.312426569 | 0.304574879 | 0.523490901 |
| 00748_09 | Cancer | 0.39689637 | 0.391418879 | 0.609023679 |
| 03630_09 | Benign | 0.442983902 | 0.526994597 | 0.563638991 |
| 02279_07 | Cancer | 0.489175005 | 0.532363923 | 0.655010149 |
| PC_04 | | 0.560558283 | 0.609682293 | 0.507126105 |
| PC_01 | | 0.528483638 | 0.663838665 | 0.495675135 |
| NYU806 | Benign | 1.261666557 | 1.240430039 | 0.579992581 |
| NYU777 | Cancer | 1.003134176 | 1.009614175 | 0.583341352 |
| NYU176 | Benign | 0.595816173 | 0.698598041 | 0.747040121 |
| NYU888 | Cancer | 0.509965043 | 0.663718883 | 0.494604682 |
| NYU1117 | Benign | 0.376210799 | 0.429162668 | 0.731869104 |
| NYU1201 | Cancer | 0.35859916 | 0.42326631 | 0.427956567 |
| PC_02 | | 0.487693412 | 0.547612202 | 0.47819389 |
| NYU887 | Cancer | 0.537866657 | 0.655884621 | 0.717019677 |
| NYU815 | Benign | 0.629902077 | 0.776867877 | 0.400780665 |
| NYU927 | Cancer | 0.776705204 | 0.863727015 | 0.666649816 |
| NYU1030 | Benign | 0.382964729 | 0.448280951 | 0.54458903 |
| NYU1151 | Cancer | 0.57111884 | 0.635248583 | 0.633861746 |
| NYU1005 | Benign | 1.307802373 | 1.398163465 | 0.687652295 |
| NYU522 | Benign | 1.407437596 | 1.642302899 | 0.521104986 |
| NYU389 | Cancer | 1.389960041 | 1.426092349 | 0.500413229 |
| PC_03 | | 0.465809931 | 0.551736272 | 0.500168216 |
| NYU729 | Cancer | 1.13928185 | 1.36629717 | 1.210689889 |
| NYU430 | Benign | 0.591077344 | 0.628934814 | 0.640061645 |
| NYU144 | Benign | 0.825998602 | 0.992671611 | 0.507360064 |
| NYU256 | Cancer | 0.434267093 | 0.439500398 | 0.577409722 |
| NYU1000 | Benign | 0.687445175 | 0.869719639 | 0.711623196 |
| NYU575 | Cancer | 3.644707754 | 4.467733427 | 0.537925663 |
| PC_04 | | 0.583460482 | 0.580551675 | 0.532829927 |

TABLE 11B
PV2 fidelity small nodule batch all transitions (normalized)

| | | AT- | AT- | | | | | |
|---|---|---|---|---|---|---|---|---|
| | | VNPSAPR_456.80_527.30 | VNPSAPR_456.80_641.30 | AVGLAG- | AVGLAG- | AVGLAG- | FLNVL- | FLNVL- |
| msfile- | | (SEQ ID No.: | (SEQ ID No.: | TFR_446.26_171.10 | TFR_446.26_551.30 | TFR_446.26_721.40 | SPR_473.28_261.20 | SPR_473.28_359.20 |
| name | status | 47) | 47) | (SEQ ID No.: 48) | (SEQ ID No.: 48) | (SEQ ID No.: 48) | (SEQ ID No.: 53) | (SEQ ID No.: 53) |
| PC_01 | | 0.534705132 | 0.556029313 | 0.521368243 | 0.407451172 | 0.472061615 | 0.659851606 | 0.693508934 |
| ZCO489_02 | Benign | 0.482318094 | 0.475201398 | 0.522018684 | 0.452615161 | 0.499287286 | 0.578287015 | 0.689088709 |
| ZCO436_02 | Cancer | 0.514449693 | 0.545843817 | 0.632989338 | 0.524636454 | 0.641716719 | 0.2803719 | 0.251519267 |
| ZCO512_02 | Cancer | 0.527165261 | 0.535412625 | 0.522545648 | 0.448051016 | 0.521255341 | 0.426434093 | 0.490820038 |
| ZCO475_02 | Benign | 0.639866769 | 0.621499097 | 0.546707079 | 0.626010052 | 0.559634393 | 0.610607983 | 0.734750979 |
| ZCO485_02 | Benign | 0.653147283 | 0.676510235 | 0.468132743 | 0.590018133 | 0.459453576 | 0.834981224 | 0.976278166 |
| ZCO536_02 | Cancer | 0.802586342 | 0.810655596 | 0.379167868 | 0.411930635 | 0.410554004 | 0.931915761 | 0.971028818 |
| PC_02 | | 0.519399286 | 0.543890152 | 0.402610916 | 0.439806134 | 0.411006249 | 0.686777309 | 0.780299233 |
| ZCO496_02 | Benign | 0.496948161 | 0.515356904 | 0.389430587 | 0.516516939 | 0.374180692 | 0.403038335 | 0.439364688 |
| ZCO502_02 | Cancer | 0.822044279 | 0.79893068 | 1.239508496 | 0.850583699 | 1.223932288 | 0.195336991 | 0.216408904 |
| ZCO382_02 | Benign | 0.554581921 | 0.572190917 | 0.568877336 | 0.516434804 | 0.457232927 | 1.10238215 | 1.059221941 |
| ZCO431_02 | Cancer | 0.549898921 | 0.539544372 | 0.45403555 | 0.513856201 | 0.45247875 | 0.437009904 | 0.438916828 |
| ZCO449_02 | Cancer | 0.432266772 | 0.440126926 | 0.378515001 | 0.444003858 | 0.333184598 | 0.916884231 | 0.863834158 |
| ZCO537_02 | Benign | 0.476290726 | 0.491289611 | 0.260220859 | 0.233797112 | 0.298742102 | 0.886985593 | 0.785839458 |
| ZCO362_02 | Benign | 0.498542645 | 0.525116363 | 0.245920046 | 0.281374625 | 0.310211704 | 0.789566819 | 0.806105263 |
| ZCO488_02 | Benign | 0.682210993 | 0.692695541 | 0.453308605 | 0.406349653 | 0.488950184 | 0.946649022 | 1.003056249 |
| PC_03 | | 0.568294726 | 0.567493126 | 0.318915614 | 0.358057825 | 0.361830621 | 0.822368397 | 0.840722458 |
| ZCO535_02 | Benign | 0.647971471 | 0.662547365 | 0.798383184 | 0.890191643 | 0.847833146 | 1.304258661 | 1.188867443 |
| ZCO443_02 | Cancer | 0.643699865 | 0.649812874 | 0.452731952 | 0.417789856 | 0.481004303 | 0.648941719 | 0.673496319 |
| ZCO393_02 | Benign | 0.61904843 | 0.627457531 | 0.668107364 | 0.54322302 | 0.593920699 | 0.681111044 | 0.80765317 |
| ZCO503_02 | Cancer | 0.590229529 | 0.602542555 | 0.535530898 | 0.490241963 | 0.634218853 | 1.2058718 | 1.252303266 |
| ZCO438_02 | Cancer | 0.912188376 | 0.95315307 | 0.475409001 | 0.510026239 | 0.656194907 | 0.606970886 | 0.672953235 |
| ZCO406_02 | Benign | 1.298365814 | 1.330381291 | 1.044205596 | 0.877045873 | 1.194473175 | 0.680656188 | 0.768931451 |
| PC_04 | | 0.552761658 | 0.581562023 | 0.303366109 | 0.364335973 | 0.365520875 | 0.71088783 | 0.711923687 |
| PC_01 | | 0.538541262 | 0.57260015 | 0.426945346 | 0.478315214 | 0.428569635 | 0.760647908 | 0.71651464 |
| 00082_07 | Cancer | 0.543302499 | 0.562089243 | 0.946767063 | 0.583568191 | 0.91718407 | 0.612409076 | 0.624535669 |
| 02286_07 | Benign | 0.671717323 | 0.685529249 | 0.698505849 | 0.553612361 | 0.696297466 | 1.278630924 | 1.230331798 |
| 02280_06 | Cancer | 0.586914146 | 0.597233235 | 0.360943511 | 0.26113329 | 0.38354143 | 1.012206752 | 1.044029917 |
| 01123_06 | Benign | 0.757012671 | 0.802068208 | 0.342087204 | 0.319614916 | 0.447898911 | 0.815870399 | 0.788618185 |
| 00156_07 | Cancer | 0.865757892 | 0.894388314 | 0.374941061 | 0.366266317 | 0.424463824 | 0.79844669 | 0.728295532 |
| 00781_09 | Benign | 0.588383312 | 0.597446673 | 0.545946881 | 0.457306352 | 0.488288192 | 1.101171259 | 1.011372243 |
| 00539_08 | Cancer | 0.465060835 | 0.476773557 | 0.306456604 | 0.255326981 | 0.30219437 | 0.444152803 | 0.458880188 |
| 02241_07 | Cancer | 0.47412833 | 0.485547515 | 0.589090796 | 0.527425678 | 0.571003806 | 0.616442009 | 0.630452537 |
| 02226_05 | Benign | 0.866731342 | 0.888171466 | 0.749658415 | 0.560987099 | 0.742955897 | 0.58593488 | 0.631433663 |
| PC_03 | | 0.566021828 | 0.566064793 | 0.410888953 | 0.359402773 | 0.40720557 | 0.845748701 | 0.739904352 |
| 00542_08 | NA | 0.676384847 | 0.687800246 | 0.44994986 | 0.352204998 | 0.54174426 | 1.049568254 | 1.181891215 |
| 02497_10 | NA | 0.490686754 | 0.505297177 | 0.265728783 | 0.237966704 | 0.328020998 | 0.976950827 | 0.944481582 |
| 02224_05 | Benign | 0.534286642 | 0.555368423 | 0.33870544 | 0.282135824 | 0.347677514 | 0.805874155 | 0.908383331 |
| 00748_09 | Cancer | 0.622472749 | 0.633487331 | 0.506977549 | 0.330183096 | 0.465868684 | 0.662049001 | 0.62822455 |
| 03630_09 | Benign | 0.595768233 | 0.6132442 | 0.413348998 | 0.295009953 | 0.395394062 | 0.847902287 | 0.750865475 |
| 02279_07 | Cancer | 0.667792071 | 0.669611895 | 0.417906413 | 0.308832173 | 0.489637626 | 0.606182353 | 0.628477668 |
| PC_04 | | 0.527231853 | 0.529821173 | 0.321302634 | 0.360017545 | 0.334083497 | 0.740584546 | 0.781709443 |
| PC_01 | | 0.51185576 | 0.520682898 | 0.427285773 | 0.42398113 | 0.437887104 | 0.789251932 | 0.841900999 |
| NYU806 | Benign | 0.621566799 | 0.628629929 | 0.370751646 | 0.256455366 | 0.411305617 | 0.784744003 | 0.88341846 |
| NYU777 | Cancer | 0.640403675 | 0.63946396 | 0.40039404 | 0.307538019 | 0.417859414 | 0.779512208 | 0.803076214 |
| NYU176 | Benign | 0.811134003 | 0.846501907 | 0.47783873 | 0.474865466 | 0.522995888 | 0.870665071 | 0.907609154 |
| NYU888 | Cancer | 0.524949845 | 0.52233603 | 0.409648134 | 0.287618542 | 0.536147824 | 0.809656582 | 0.858531807 |
| NYU1117 | Benign | 0.770626518 | 0.799053901 | 0.647044209 | 0.550035882 | 0.650731937 | 1.017201564 | 1.082866921 |
| NYU1201 | Cancer | 0.455662402 | 0.455067228 | 0.383442328 | 0.295022773 | 0.374266169 | 1.153594716 | 1.142319157 |
| PC_02 | | 0.508003119 | 0.51543261 | 0.291674169 | 0.286295453 | 0.318550966 | 0.700985385 | 0.747352074 |
| NYU887 | Cancer | 0.72446972 | 0.757576957 | 0.291845896 | 0.329657487 | 0.326034113 | 0.936022461 | 0.962609294 |
| NYU815 | Benign | 0.421478948 | 0.433741701 | 0.351639129 | 0.345566606 | 0.416303817 | 1.194743186 | 1.252121118 |
| NYU927 | Cancer | 0.716616472 | 0.706170721 | 0.773547512 | 0.862203004 | 0.763196557 | 0.455641838 | 0.475382877 |
| NYU1030 | Benign | 0.577724009 | 0.562417202 | 0.571048537 | 0.53259461 | 0.611157458 | 0.529638286 | 0.5845219 |
| NYU1151 | Cancer | 0.656998477 | 0.707576402 | 0.550926896 | 0.389812034 | 0.548490319 | 0.538515974 | 0.530037022 |
| NYU1005 | Benign | 0.710673557 | 0.755953396 | 0.356180044 | 0.278382778 | 0.375437353 | 1.061725085 | 1.089004601 |
| NYU522 | Benign | 0.537855571 | 0.538883533 | 0.302643305 | 0.201354994 | 0.314789049 | 1.085919754 | 1.055072892 |
| NYU389 | Cancer | 0.543516944 | 0.566261626 | 0.556142958 | 0.485807729 | 0.636248948 | 0.837939224 | 0.906153882 |
| PC_03 | | 0.549860606 | 0.544846659 | 0.307346441 | 0.319876614 | 0.339163972 | 0.658220539 | 0.733488807 |
| NYU729 | Cancer | 1.289813605 | 1.319182379 | 0.471636782 | 0.415466283 | 0.550002098 | 0.545856132 | 0.593263842 |
| NYU430 | Benign | 0.6766729 | 0.692138591 | 0.334200396 | 0.304617929 | 0.396001906 | 0.570416109 | 0.511151972 |
| NYU144 | Benign | 0.525849025 | 0.566159596 | 0.696505641 | 0.482405382 | 0.730920139 | 1.145307161 | 1.357796744 |
| NYU256 | Cancer | 0.59767304 | 0.603714812 | 0.243495164 | 0.248415657 | 0.266061157 | 0.52183018 | 0.648488973 |
| NYU1000 | Benign | 0.724665149 | 0.744379705 | 0.40253419 | 0.383996187 | 0.478071928 | 0.485964459 | 0.475382266 |
| NYU575 | Cancer | 0.57072014 | 0.612794772 | 0.469750397 | 0.410979992 | 0.614193715 | 0.790171504 | 0.806540998 |
| PC_04 | | 0.55734964 | 0.586255643 | 0.345976693 | 0.361853153 | 0.310204199 | 0.811758135 | 0.755532329 |

TABLE 11C
PV2 fidelity small nodule batch all transitions (normalized)

| | | FLNVL- | FLNVL- | GFLLLASLR_495.31_318.20 | GFLLLASLR_495.31_446.30 |
|---|---|---|---|---|---|
| msfile- | | SPR_473.28_472.30 | SPR_473.28_685.40 | (SEQ ID | (SEQ ID |
| name | status | (SEQ ID No.: 53) | (SEQ ID No.: 53) | No.: 61) | No.: 61) |
| PC_01 | | 0.691981582 | 0.720732962 | 0.342167365 | 0.314422112 |
| ZCO489_02 | Benign | 0.605287789 | 0.65078866 | 0.859783085 | 0.821168835 |
| ZCO436_02 | Cancer | 0.248428527 | 0.273491247 | 0.223525612 | 0.234001826 |
| ZCO512_02 | Cancer | 0.434528592 | 0.414608533 | 1.696599511 | 1.742552568 |
| ZCO475_02 | Benign | 0.646258857 | 0.627829619 | 1.147836544 | 1.082338999 |
| ZCO485_02 | Benign | 0.879277454 | 0.862590838 | 0.493331238 | 0.523185029 |
| ZCO536_02 | Cancer | 1.061547744 | 1.023078885 | 1.300843206 | 1.152133544 |
| PC_02 | | 0.701343473 | 0.793152647 | 0.29057686 | 0.280086529 |
| ZCO496_02 | Benign | 0.387291455 | 0.407516867 | 0.836504722 | 0.795963922 |
| ZCO502_02 | Cancer | 0.180052439 | 0.200398054 | 2.700856929 | 2.594915099 |
| ZCO382_02 | Benign | 1.04006184 | 1.032352624 | 0.338185874 | 0.2837697 |
| ZCO431_02 | Cancer | 0.40882763 | 0.443256396 | 1.388161576 | 1.540533044 |
| ZCO449_02 | Cancer | 0.819848841 | 0.839724894 | 0.93711654 | 0.86013574 |
| ZCO537_02 | Benign | 0.750983489 | 0.823874374 | 1.425510223 | 1.399688316 |
| ZCO362_02 | Benign | 0.809646895 | 0.842014404 | 0.28868153 | 0.279271806 |
| ZCO488_02 | Benign | 1.003370131 | 1.021486996 | 0.639495367 | 0.682112744 |
| PC_03 | | 0.76233059 | 0.854208853 | 0.317881757 | 0.291284882 |
| ZCO535_02 | Benign | 1.161896025 | 1.194064604 | 0.648841312 | 0.655865069 |
| ZCO443_02 | Cancer | 0.614529243 | 0.652022796 | 2.728330195 | 2.461806843 |
| ZCO393_02 | Benign | 0.739593896 | 0.807623353 | 0.670000429 | 0.664602591 |
| ZCO503_02 | Cancer | 1.190519599 | 1.187750675 | 2.664925758 | 2.624223153 |
| ZCO438_02 | Cancer | 0.59728587 | 0.665227738 | 1.802976602 | 1.732439351 |
| ZCO406_02 | Benign | 0.655956 | 0.849782405 | 1.229147311 | 1.149613176 |
| PC_04 | | 0.721041262 | 0.744556741 | 0.353587214 | 0.339581216 |
| PC_01 | | 0.712078659 | 0.725057033 | 0.316141016 | 0.301482209 |
| 00082_07 | Cancer | 0.570305967 | 0.620069042 | 1.201392543 | 1.286592675 |
| 02286_07 | Benign | 1.213507246 | 1.319378592 | 1.894049273 | 1.98468928 |
| 02280_06 | Cancer | 0.899298833 | 0.983820418 | 1.276247055 | 1.440737251 |
| 01123_06 | Benign | 0.711614502 | 0.772422192 | 1.34239276 | 1.331966067 |
| 00156_07 | Cancer | 0.779075514 | 0.784053617 | 0.328273854 | 0.328521415 |
| 00781_09 | Benign | 0.994751468 | 1.051467616 | 0.533182864 | 0.56232441 |
| 00539_08 | Cancer | 0.452869256 | 0.479326651 | 1.372633176 | 1.443965208 |
| 02241_07 | Cancer | 0.570374561 | 0.633648884 | 0.484740669 | 0.492724316 |
| 02226_05 | Benign | 0.597871564 | 0.610065523 | 1.612026099 | 1.592469515 |
| PC_03 | | 0.828672158 | 0.808060907 | 0.365914791 | 0.369628535 |
| 00542_08 | NA | 1.168713681 | 1.146708251 | 0.311383616 | 0.290225844 |
| 02497_10 | NA | 0.917391832 | 0.91569795 | 0.571776807 | 0.569150593 |
| 02224_05 | Benign | 0.833252073 | 0.885169529 | 0.690318247 | 0.672504291 |
| 00748_09 | Cancer | 0.585228392 | 0.645389405 | 0.643584598 | 0.610412621 |
| 03630_09 | Benign | 0.755397991 | 0.803677987 | 0.647856006 | 0.590942425 |
| 02279_07 | Cancer | 0.677392643 | 0.669161404 | 0.651598555 | 0.590778799 |
| PC_04 | | 0.75988882 | 0.785502241 | 0.338403296 | 0.329147176 |
| PC_01 | | 0.745344878 | 0.809784221 | 0.342972712 | 0.38366931 |
| NYU806 | Benign | 0.820469011 | 0.884822086 | 6.664158715 | 4.630699561 |
| NYU777 | Cancer | 0.663614708 | 0.813427528 | 4.105501739 | 4.052418417 |
| NYU176 | Benign | 0.918352647 | 0.911620438 | 1.681155207 | 1.669534825 |
| NYU888 | Cancer | 0.737762116 | 0.81095489 | 4.951991286 | 4.739682362 |
| NYU1117 | Benign | 1.085918695 | 0.955350038 | 2.04230216 | 1.931305652 |
| NYU1201 | Cancer | 1.051534544 | 1.230115601 | 0.784171746 | 0.668141656 |
| PC_02 | | 0.738475273 | 0.792056489 | 0.354546336 | 0.31012861 |
| NYU887 | Cancer | 0.964355435 | 0.990907259 | 4.092478957 | 3.914256725 |
| NYU815 | Benign | 1.144783274 | 1.304636407 | 0.47515795 | 0.525400342 |
| NYU927 | Cancer | 0.426994013 | 0.490195635 | 0.922026899 | 0.935018393 |
| NYU1030 | Benign | 0.572526274 | 0.621599721 | 0.312142527 | 0.334559507 |
| NYU1151 | Cancer | 0.500237238 | 0.562995164 | 3.385593779 | 3.420730919 |
| NYU1005 | Benign | 1.060271913 | 1.175165129 | 7.689991257 | 7.476638332 |
| NYU522 | Benign | 1.033063365 | 1.127453845 | 2.626451718 | 2.385238589 |
| NYU389 | Cancer | 0.810023432 | 0.881237 | 4.969507998 | 4.879833728 |
| PC_03 | | 0.697463389 | 0.734952718 | 0.365487948 | 0.403732526 |
| NYU729 | Cancer | 0.490526587 | 0.534210846 | 9.817611923 | 9.659929885 |
| NYU430 | Benign | 0.503227078 | 0.575604606 | 1.323573206 | 1.255175389 |
| NYU144 | Benign | 1.179607464 | 1.18587984 | 2.409172734 | 2.292341372 |
| NYU256 | Cancer | 0.650288293 | 0.586537175 | 0.682773589 | 0.709002898 |
| NYU1000 | Benign | 0.421582002 | 0.532016419 | 1.167693053 | 1.266238809 |
| NYU575 | Cancer | 0.792571593 | 0.761693263 | 2.313843701 | 2.247030621 |
| PC_04 | | 0.839901554 | 0.851345846 | 0.350458346 | 0.42039804 |

| msfile- | | GFLLLASLR_495.31_559.40 | INPASLDK_429.24_228.10 | INPASLDK_429.24_462.30 |
|---|---|---|---|---|
| name | status | (SEQ ID No.: 61) | (SEQ ID No.: 67) | (SEQ ID No.: 67) |
| PC_01 | | 0.340263802 | 0.37810668 | 0.458465671 |
| ZCO489_02 | Benign | 0.888489155 | 0.398199696 | 0.320039699 |
| ZCO436_02 | Cancer | 0.230499872 | 0.455033635 | 0.456280913 |
| ZCO512_02 | Cancer | 1.711010398 | 0.473543721 | 0.458740024 |
| ZCO475_02 | Benign | 1.0614724 | 0.438608111 | 0.397818698 |
| ZCO485_02 | Benign | 0.565283055 | 0.472828123 | 0.47632891 |
| ZCO536_02 | Cancer | 1.330206484 | 0.282594548 | 0.220945725 |
| PC_02 | | 0.286424331 | 0.390133878 | 0.367380405 |
| ZCO496_02 | Benign | 0.821965253 | 0.591262978 | 0.574871317 |
| ZCO502_02 | Cancer | 2.820589292 | 0.56525324 | 0.424258773 |
| ZCO382_02 | Benign | 0.340794925 | 0.432895305 | 0.341679129 |
| ZCO431_02 | Cancer | 1.610766695 | 0.433954714 | 0.344861755 |
| ZCO449_02 | Cancer | 0.913229868 | 0.345681021 | 0.344177213 |
| ZCO537_02 | Benign | 1.290874731 | 0.44315624 | 0.393036455 |
| ZCO362_02 | Benign | 0.295683453 | 0.568791128 | 0.508212761 |
| ZCO488_02 | Benign | 0.688601012 | 0.307664047 | 0.229467979 |
| PC_03 | | 0.314852946 | 0.374073721 | 0.389236187 |
| ZCO535_02 | Benign | 0.655555727 | 0.473660676 | 0.53901155 |
| ZCO443_02 | Cancer | 2.716329467 | 0.729555139 | 0.66750816 |
| ZCO393_02 | Benign | 0.647738274 | 0.491946833 | 0.466602329 |
| ZCO503_02 | Cancer | 2.810381227 | 0.452919305 | 0.350472374 |
| ZCO438_02 | Cancer | 1.872001648 | 1.118807359 | 0.925793835 |
| ZCO406_02 | Benign | 1.10418014 | 0.403367923 | 0.538183076 |
| PC_04 | | 0.33108052 | 0.404307977 | 0.416598959 |
| PC_01 | | 0.313122033 | 0.421204527 | 0.397107212 |
| 00082_07 | Cancer | 1.396458385 | 0.610531593 | 0.472285801 |
| 02286_07 | Benign | 1.955162614 | 0.336607992 | 0.296903259 |
| 02280_06 | Cancer | 1.335856568 | 0.500893538 | 0.396566024 |
| 01123_06 | Benign | 1.30303188 | 0.283264675 | 0.239651555 |
| 00156_07 | Cancer | 0.317571569 | 0.569361783 | 0.497428196 |
| 00781_09 | Benign | 0.521007818 | 0.448634196 | 0.41903525 |
| 00539_08 | Cancer | 1.468603986 | 0.642132174 | 0.567502712 |
| 02241_07 | Cancer | 0.524372392 | 0.43424081 | 0.260567028 |
| 02226_05 | Benign | 1.6902868 | 0.471948866 | 0.559620128 |
| PC_03 | | 0.346302974 | 0.42232798 | 0.41037486 |
| 00542_08 | NA | 0.307130705 | 0.491994912 | 0.594067468 |
| 02497_10 | NA | 0.67191397 | 0.348786965 | 0.35891839 |
| 02224_05 | Benign | 0.694573879 | 0.386615091 | 0.329363336 |
| 00748_09 | Cancer | 0.661566205 | 0.510768098 | 0.395267241 |
| 03630_09 | Benign | 0.626098786 | 0.388687007 | 0.381351725 |
| 02279_07 | Cancer | 0.595214365 | 0.400885329 | 0.396289138 |
| PC_04 | | 0.326166352 | 0.381452485 | 0.429176204 |
| PC_01 | | 0.366427903 | 0.38184938 | 0.339192846 |
| NYU806 | Benign | 5.061642045 | 0.520814311 | 0.442142913 |
| NYU777 | Cancer | 4.189556977 | 0.462157946 | 0.495113266 |
| NYU176 | Benign | 1.686515801 | 0.628388709 | 0.622855521 |
| NYU888 | Cancer | 4.835654266 | 0.577638172 | 0.468849359 |
| NYU1117 | Benign | 2.17141165 | 0.369285189 | 0.322737033 |
| NYU1201 | Cancer | 0.69727139 | 0.494924505 | 0.440950082 |
| PC_02 | | 0.31205844 | 0.358292797 | 0.353934567 |
| NYU887 | Cancer | 4.363006538 | 0.458013654 | 0.366363189 |
| NYU815 | Benign | 0.483134144 | 0.324670709 | 0.312260442 |
| NYU927 | Cancer | 0.963827038 | 0.41790394 | 0.392013003 |
| NYU1030 | Benign | 0.334192054 | 0.768447019 | 0.657559403 |
| NYU1151 | Cancer | 3.641732461 | 0.501225367 | 0.557755283 |
| NYU1005 | Benign | 7.290468401 | 0.36606111 | 0.343489515 |
| NYU522 | Benign | 2.651138755 | 0.380855259 | 0.331702566 |
| NYU389 | Cancer | 4.781103782 | 0.746129428 | 0.745929888 |
| PC_03 | | 0.410220916 | 0.398219674 | 0.360205717 |
| NYU729 | Cancer | 10.16806557 | 0.650190373 | 0.676875771 |
| NYU430 | Benign | 1.331232129 | 0.530193787 | 0.414020569 |
| NYU144 | Benign | 2.435929958 | 0.6547869 | 0.674026092 |
| NYU256 | Cancer | 0.715759485 | 0.697362278 | 0.705920708 |
| NYU1000 | Benign | 1.241755547 | 0.463665408 | 0.395720265 |
| NYU575 | Cancer | 2.056567034 | 0.452553353 | 0.439474833 |
| PC_04 | | 0.395652864 | 0.428097462 | 0.287222773 |

TABLE 11D
PV2 fidelity small nodule batch all transitions (normalized)

| | | | | LDTLAQE- | LDTLAQE- |
|---|---|---|---|---|---|
| msfile- | | INPASLDK_429.24_630.30 | INPASLDK_429.24_744.40 | VALLK_657.39_229.10 | VALLK_657.39_330.20 |
| name | status | (SEQ ID No.: 67) | (SEQ ID No.: 67) | (SEQ ID No.: 66) | (SEQ ID No.: 66) |
| PC_01 | | 0.363735797 | 0.428688366 | 0.852842762 | 0.864372452 |
| ZCO489_02 | Benign | 0.343504887 | 0.322042591 | 0.688898088 | 0.683271522 |
| ZCO436_02 | Cancer | 0.394523842 | 0.505190828 | 0.503107835 | 0.540139387 |
| ZCO512_02 | Cancer | 0.410484072 | 0.547592288 | 0.472049093 | 0.456026487 |
| ZCO475_02 | Benign | 0.373983172 | 0.384733283 | 0.656230813 | 0.655829761 |
| ZCO485_02 | Benign | 0.403353031 | 0.494610614 | 0.753010819 | 0.825964619 |
| ZCO536_02 | Cancer | 0.266980134 | 0.286580444 | 0.93016632 | 0.890720543 |
| PC_02 | | 0.343689781 | 0.368552668 | 0.741743535 | 0.737061342 |
| ZCO496_02 | Benign | 0.562612295 | 0.620279709 | 0.548457453 | 0.596136956 |
| ZCO502_02 | Cancer | 0.41478149 | 0.452785667 | 0.437177039 | 0.416922838 |
| ZCO382_02 | Benign | 0.366526715 | 0.378798806 | 0.673068272 | 0.657476873 |
| ZCO431_02 | Cancer | 0.381970005 | 0.396582628 | 0.993836317 | 1.149811021 |
| ZCO449_02 | Cancer | 0.312941244 | 0.349823643 | 0.658940922 | 0.661913662 |
| ZCO537_02 | Benign | 0.3594776 | 0.416595564 | 0.678733461 | 0.587012469 |
| ZCO362_02 | Benign | 0.486810602 | 0.529863821 | 0.680112422 | 0.701322149 |
| ZCO488_02 | Benign | 0.273829963 | 0.319282348 | 0.708560978 | 0.760405448 |
| PC_03 | | 0.332753598 | 0.404900508 | 0.846177887 | 0.773159181 |
| ZCO535_02 | Benign | 0.406352625 | 0.447093453 | 0.62231948 | 0.591895539 |
| ZCO443_02 | Cancer | 0.644864665 | 0.69995906 | 0.585433046 | 0.600321797 |
| ZCO393_02 | Benign | 0.412438594 | 0.449876317 | 0.727733419 | 0.718800403 |
| ZCO503_02 | Cancer | 0.384648002 | 0.465001148 | 0.590094777 | 0.592033667 |
| ZCO438_02 | Cancer | 0.993508564 | 1.206714171 | 0.456538877 | 0.460899802 |
| ZCO406_02 | Benign | 0.359856429 | 0.378045334 | 0.471484206 | 0.447564405 |
| PC_04 | | 0.364682747 | 0.395717106 | 0.796150595 | 0.704025463 |
| PC_01 | | 0.353303739 | 0.388682498 | 0.889503601 | 0.871245127 |
| 00082_07 | Cancer | 0.528381439 | 0.537937253 | 0.420534929 | 0.457636372 |
| 02286_07 | Benign | 0.30880205 | 0.374089935 | 0.557489452 | 0.544980319 |
| 02280_06 | Cancer | 0.398488287 | 0.44991999 | 0.68934591 | 0.665235792 |
| 01123_06 | Benign | 0.237138595 | 0.298588226 | 0.9041684 | 0.96038976 |
| 00156_07 | Cancer | 0.490352058 | 0.61972889 | 0.433147562 | 0.449100459 |
| 00781_09 | Benign | 0.367161488 | 0.343929845 | 0.697950521 | 0.691559596 |
| 00539_08 | Cancer | 0.573748716 | 0.559185986 | 0.707643837 | 0.707811609 |
| 02241_07 | Cancer | 0.377731536 | 0.487992107 | 0.820252098 | 0.766892092 |
| 02226_05 | Benign | 0.38092763 | 0.498275906 | 0.472955469 | 0.463330275 |
| PC_03 | | 0.368004213 | 0.385192085 | 0.967363627 | 0.890275077 |
| 00542_08 | NA | 0.421547034 | 0.455192601 | 0.653642447 | 0.697794063 |
| 02497_10 | NA | 0.292919106 | 0.355624546 | 0.765647237 | 0.756196014 |
| 02224_05 | Benign | 0.323247418 | 0.37932085 | 0.78816019 | 0.769221817 |
| 00748_09 | Cancer | 0.392264009 | 0.455267153 | 0.589262766 | 0.630145683 |
| 03630_09 | Benign | 0.340098151 | 0.392828634 | 0.733679224 | 0.758161938 |
| 02279_07 | Cancer | 0.364172908 | 0.397191766 | 0.501156817 | 0.530411443 |
| PC_04 | | 0.325266925 | 0.353077566 | 0.823177428 | 0.762357207 |
| PC_01 | | 0.349755825 | 0.366449226 | 0.949833946 | 0.984318669 |
| NYU806 | Benign | 0.481091003 | 0.519753096 | 0.485580312 | 0.511892188 |
| NYU777 | Cancer | 0.407028773 | 0.492822475 | 0.666536856 | 0.674248936 |
| NYU176 | Benign | 0.486992045 | 0.614138716 | 0.680362518 | 0.655072704 |
| NYU888 | Cancer | 0.477552132 | 0.638814219 | 0.548957225 | 0.603720153 |
| NYU1117 | Benign | 0.360139883 | 0.368261098 | 0.592191821 | 0.653468736 |
| NYU1201 | Cancer | 0.42107192 | 0.471797917 | 0.689150671 | 0.709821955 |
| PC_02 | | 0.335736773 | 0.30690412 | 0.832761797 | 0.888404889 |
| NYU887 | Cancer | 0.474047732 | 0.535284992 | 0.797859166 | 0.803093081 |
| NYU815 | Benign | 0.274161099 | 0.364368097 | 0.713604238 | 0.637545343 |
| NYU927 | Cancer | 0.36239794 | 0.440310431 | 0.592818164 | 0.581656898 |
| NYU1030 | Benign | 0.669200731 | 0.579338094 | 0.752638223 | 0.759192937 |
| NYU1151 | Cancer | 0.471140022 | 0.527524938 | 0.449757714 | 0.465773553 |
| NYU1005 | Benign | 0.347833855 | 0.374620273 | 1.071485111 | 1.178779337 |
| NYU522 | Benign | 0.340458208 | 0.412637937 | 0.885750821 | 0.918034199 |
| NYU389 | Cancer | 0.641152466 | 0.680741525 | 0.45235022 | 0.506598818 |
| PC_03 | | 0.359080514 | 0.379344063 | 0.807217591 | 0.815280326 |
| NYU729 | Cancer | 0.706055083 | 0.836520244 | 0.501131433 | 0.506455475 |
| NYU430 | Benign | 0.426973875 | 0.517276242 | 0.970523424 | 0.870521485 |
| NYU144 | Benign | 0.604709232 | 0.610266777 | 0.766590581 | 0.795909496 |
| NYU256 | Cancer | 0.599927593 | 0.692324539 | 0.730300014 | 0.774238336 |
| NYU1000 | Benign | 0.367591711 | 0.472316076 | 0.88548905 | 0.843154492 |
| NYU575 | Cancer | 0.389054834 | 0.448580659 | 0.840423345 | 0.696859969 |
| PC_04 | | 0.357303411 | 0.357374777 | 0.879853377 | 0.956282697 |

| | | LDTLAQE- | LDTLAQE- | LGG- |
|---|---|---|---|---|
| msfile- | | VALLK_657.39_800.50 | VALLK_657.39_871.50 | PEAGLGEYLFER_804.40_1083.60 |
| name | status | (SEQ ID No.: 66) | (SEQ ID No.: 66) | (SEQ ID No.: 50) |
| PC_01 | | 0.800249812 | 0.870566218 | 0.030665666 |
| ZCO489_02 | Benign | 0.64836569 | 0.70122662 | 0.053075563 |
| ZCO436_02 | Cancer | 0.51224076 | 0.517813349 | 0.07550509 |
| ZCO512_02 | Cancer | 0.480698222 | 0.476402358 | 0.191646835 |
| ZCO475_02 | Benign | 0.624588491 | 0.697240825 | 0.134482993 |
| ZCO485_02 | Benign | 0.811252913 | 0.799106554 | 0.090174478 |
| ZCO536_02 | Cancer | 0.91837766 | 1.082088276 | 0.183240953 |
| PC_02 | | 0.699423116 | 0.745035088 | 0.022925279 |
| ZCO496_02 | Benign | 0.600896169 | 0.657352334 | 0.021442904 |
| ZCO502_02 | Cancer | 0.404813293 | 0.405778053 | 0.148612156 |
| ZCO382_02 | Benign | 0.557214039 | 0.690776063 | 0.081047236 |
| ZCO431_02 | Cancer | 0.953207847 | 1.140177817 | 0.061379876 |
| ZCO449_02 | Cancer | 0.689169741 | 0.781056025 | 0.603542675 |
| ZCO537_02 | Benign | 0.573674137 | 0.62049249 | 0.105417554 |
| ZCO362_02 | Benign | 0.708166429 | 0.732398997 | 0.013723205 |
| ZCO488_02 | Benign | 0.701838025 | 0.737813133 | 0.008516135 |
| PC_03 | | 0.821135084 | 0.878483826 | 0.025780526 |
| ZCO535_02 | Benign | 0.573655568 | 0.613856381 | 0.221268745 |
| ZCO443_02 | Cancer | 0.588534929 | 0.664811475 | 0.149205132 |
| ZCO393_02 | Benign | 0.693087711 | 0.793332826 | 0.14010071 |
| ZCO503_02 | Cancer | 0.564901531 | 0.603672888 | 0.083669807 |
| ZCO438_02 | Cancer | 0.428532546 | 0.451887945 | 0.258177146 |
| ZCO406_02 | Benign | 0.432278392 | 0.486251452 | 0.740287916 |
| PC_04 | | 0.710300175 | 0.726184807 | 0.025156047 |
| PC_01 | | 0.778059465 | 0.833109187 | 0.035948333 |
| 00082_07 | Cancer | 0.413142448 | 0.472220997 | 0.095230711 |
| 02286_07 | Benign | 0.579783093 | 0.592646656 | 0.511556626 |
| 02280_06 | Cancer | 0.63217575 | 0.691868201 | 0.099662074 |
| 01123_06 | Benign | 0.970401502 | 0.960509966 | 0.135058473 |
| 00156_07 | Cancer | 0.410508013 | 0.420941591 | 0.169227194 |
| 00781_09 | Benign | 0.66239628 | 0.750630821 | 0.354326419 |
| 00539_08 | Cancer | 0.669120134 | 0.709650629 | 0.10284732 |
| 02241_07 | Cancer | 0.750758064 | 0.746256999 | 0.038909707 |
| 02226_05 | Benign | 0.458412581 | 0.481592392 | 0.018558615 |
| PC_03 | | 0.911123338 | 0.905521528 | 0.030055933 |
| 00542_08 | NA | 0.681520531 | 0.7155287 | 0.086441503 |
| 02497_10 | NA | 0.674987734 | 0.756495063 | 0.171716375 |
| 02224_05 | Benign | 0.766516315 | 0.801369643 | 0.210932665 |
| 00748_09 | Cancer | 0.558857667 | 0.595268614 | 0.330658658 |
| 03630_09 | Benign | 0.738165641 | 0.732702422 | 0.122462084 |
| 02279_07 | Cancer | 0.454388 | 0.531584781 | 0.138464592 |
| PC_04 | | 0.711879952 | 0.795783423 | 0.031180525 |
| PC_01 | | 0.850456831 | 0.982088585 | 0.039234794 |
| NYU806 | Benign | 0.485057637 | 0.511564771 | 0.102371296 |
| NYU777 | Cancer | 0.685079511 | 0.757825393 | 0.059968758 |
| NYU176 | Benign | 0.618779114 | 0.706281524 | 0.005952263 |
| NYU888 | Cancer | 0.577206255 | 0.605568104 | 0.04588913 |
| NYU1117 | Benign | 0.625725216 | 0.657511405 | 0.535542606 |
| NYU1201 | Cancer | 0.661002543 | 0.714987993 | 0.214463452 |
| PC_02 | | 0.816338043 | 0.903258518 | 0.0334592 |
| NYU887 | Cancer | 0.833248479 | 0.8694931 | 0.102404415 |
| NYU815 | Benign | 0.62106511 | 0.669899242 | 0.074008212 |
| NYU927 | Cancer | 0.50842217 | 0.581836011 | 0.226102623 |
| NYU1030 | Benign | 0.761401341 | 0.789355237 | 0.190954 |
| NYU1151 | Cancer | 0.433321676 | 0.48737091 | 0.242885687 |
| NYU1005 | Benign | 1.12491111 | 1.303717218 | 0.208826976 |
| NYU522 | Benign | 0.854008143 | 0.955343969 | 0.09104529 |
| NYU389 | Cancer | 0.488288074 | 0.521285938 | 0.15396803 |
| PC_03 | | 0.763271293 | 0.903130514 | 0.029783506 |
| NYU729 | Cancer | 0.487103964 | 0.496035461 | 0.314049247 |
| NYU430 | Benign | 0.844321625 | 0.991735387 | 0.070609482 |
| NYU144 | Benign | 0.760069455 | 0.795435225 | 0.008629685 |
| NYU256 | Cancer | 0.748785824 | 0.73462539 | 0.065551163 |
| NYU1000 | Benign | 0.947473752 | 0.903534842 | 0.050514738 |
| NYU575 | Cancer | 0.726430056 | 0.6712768 | 0.012836029 |
| PC_04 | | 0.953206261 | 0.949350421 | 0.034914953 |

TABLE 11E
PV2 fidelity small nodule batch all transitions (normalized)

| | | LGG- | LGG- | LQSLFD- |
|---|---|---|---|---|
| | | PEAGLGEYLFER_804.40_525.30 | PEAGLGEYLFER_804.40_913.40 | SPDFSK_692.34_1142.60 |
| msfile- | | (SEQ ID | (SEQ ID | (SEQ ID No.: |
| name | status | No.: 50) | No.: 50) | 68) |
| PC_01 | | 0.038554459 | 0.036120215 | 1.765432159 |
| ZCO489_02 | Benign | 0.073592529 | 0.054497729 | 1.586378777 |
| ZCO436_02 | Cancer | 0.077673137 | 0.066303335 | 1.708293197 |
| ZCO512_02 | Cancer | 0.209194542 | 0.21494463 | 1.73445266 |
| ZCO475_02 | Benign | 0.17621848 | 0.153949618 | 1.80536783 |
| ZCO485_02 | Benign | 0.089087893 | 0.086073903 | 1.62410579 |
| ZCO536_02 | Cancer | 0.217692961 | 0.172418364 | 1.448827094 |
| PC_02 | | 0.035995794 | 0.023927689 | 1.803523286 |
| ZCO496_02 | Benign | 0.03228154 | 0.020569016 | 2.103903547 |
| ZCO502_02 | Cancer | 0.148571609 | 0.133049649 | 2.345228584 |
| ZCO382_02 | Benign | 0.070969497 | 0.069210735 | 1.873274606 |
| ZCO431_02 | Cancer | 0.08992654 | 0.067820845 | 1.942972731 |
| ZCO449_02 | Cancer | 0.676686766 | 0.660013278 | 1.487341937 |
| ZCO537_02 | Benign | 0.117971248 | 0.117940655 | 1.359478175 |
| ZCO362_02 | Benign | 0.017621106 | 0.010116651 | 1.772408083 |
| ZCO488_02 | Benign | 0.036074192 | 0.01539941 | 2.449135421 |
| PC_03 | | 0.036791598 | 0.028328086 | 1.871078192 |
| ZCO535_02 | Benign | 0.21899049 | 0.203313091 | 2.539222994 |
| ZCO443_02 | Cancer | 0.171215985 | 0.154638862 | 1.656571376 |
| ZCO393_02 | Benign | 0.150305206 | 0.143821845 | 1.88859011 |
| ZCO503_02 | Cancer | 0.0942704 | 0.09453189 | 1.807574691 |
| ZCO438_02 | Cancer | 0.281585838 | 0.28705589 | 1.906446749 |
| ZCO406_02 | Benign | 0.666742621 | 0.776810853 | 3.25360525 |
| PC_04 | | 0.042862707 | 0.030260939 | 1.829695167 |
| PC_01 | | 0.04399596 | 0.02945243 | 1.745588128 |
| 00082_07 | Cancer | 0.123771832 | 0.106138246 | 1.897990062 |
| 02286_07 | Benign | 0.565268693 | 0.621708987 | 1.97225443 |
| 02280_06 | Cancer | 0.112476391 | 0.136236143 | 1.043908722 |
| 01123_06 | Benign | 0.134426478 | 0.140390427 | 1.506291416 |
| 00156_07 | Cancer | 0.206263665 | 0.167480709 | 1.758389827 |
| 00781_09 | Benign | 0.354512834 | 0.394216635 | 1.428208631 |
| 00539_08 | Cancer | 0.097862022 | 0.098665623 | 1.499616799 |
| 02241_07 | Cancer | 0.058683769 | 0.046905377 | 1.932192223 |
| 02226_05 | Benign | 0.042185379 | 0.022621871 | 2.072024638 |
| PC_03 | | 0.045598196 | 0.031588294 | 1.771807265 |
| 00542_08 | NA | 0.106733461 | 0.091640906 | 1.654718087 |
| 02497_10 | NA | 0.206194505 | 0.184667736 | 1.642933804 |
| 02224_05 | Benign | 0.244839005 | 0.228451904 | 1.776757807 |
| 00748_09 | Cancer | 0.359267967 | 0.325786817 | 1.534812384 |
| 03630_09 | Benign | 0.143967889 | 0.13158887 | 1.622180504 |
| 02279_07 | Cancer | 0.139552422 | 0.127062426 | 1.897637765 |
| PC_04 | | 0.05275638 | 0.036725111 | 1.670412757 |
| PC_01 | | 0.05642519 | 0.032903157 | 1.70674995 |
| NYU806 | Benign | 0.129683582 | 0.108297185 | 1.708421236 |
| NYU777 | Cancer | 0.072971393 | 0.068910326 | 1.618593364 |
| NYU176 | Benign | 0.01232397 | 0.014506745 | 1.474086651 |
| NYU888 | Cancer | 0.050280342 | 0.042596819 | 1.58901714 |
| NYU1117 | Benign | 0.662356982 | 0.640776334 | 1.959149358 |
| NYU1201 | Cancer | 0.21567413 | 0.206220977 | 2.009830085 |
| PC_02 | | 0.048239109 | 0.031945287 | 1.640095795 |
| NYU887 | Cancer | 0.123818818 | 0.114835526 | 1.675784212 |
| NYU815 | Benign | 0.088244391 | 0.068502312 | 2.144946292 |
| NYU927 | Cancer | 0.245612411 | 0.234527082 | 1.753922586 |
| NYU1030 | Benign | 0.190220539 | 0.166076825 | 1.520620993 |
| NYU1151 | Cancer | 0.276467194 | 0.3116029 | 2.113195051 |
| NYU1005 | Benign | 0.220242061 | 0.197526081 | 1.759318564 |
| NYU522 | Benign | 0.128209198 | 0.09278456 | 1.784348332 |
| NYU389 | Cancer | 0.181349925 | 0.16982168 | 2.15593723 |
| PC_03 | | 0.048207527 | 0.032807525 | 1.607683274 |
| NYU729 | Cancer | 0.351811018 | 0.364531234 | 1.913858062 |
| NYU430 | Benign | 0.078953416 | 0.071638172 | 1.673681959 |
| NYU144 | Benign | 0.017742479 | 0.010255227 | 1.607590107 |
| NYU256 | Cancer | 0.098516241 | 0.062505905 | 1.384851528 |
| NYU1000 | Benign | 0.070598556 | 0.04888533 | 1.589628456 |
| NYU575 | Cancer | 0.018636081 | 0.008901971 | 1.776131185 |
| PC_04 | | 0.050728447 | 0.03334697 | 1.78266488 |

| | | LQSLFD- | LQSLFD- | LQSLFD- | LQSLFD- |
|---|---|---|---|---|---|
| | | SPDFSK_692.34_242.20 | SPDFSK_692.34_329.20 | SPDFSK_692.34_593.30 | SPDFSK_692.34_942.50 |
| msfile- | | (SEQ ID No.: | (SEQ ID No.: | (SEQ ID No.: | (SEQ ID |
| name | status | 68) | 68) | 68) | No.: 68) |
| PC_01 | | 1.942539552 | 1.875976304 | 1.781592163 | 1.945789175 |
| ZCO489_02 | Benign | 1.675357988 | 1.796593459 | 1.772831175 | 1.666702749 |
| ZCO436_02 | Cancer | 1.747014735 | 2.136049744 | 1.840188133 | 1.868023509 |
| ZCO512_02 | Cancer | 1.883944812 | 2.146035402 | 1.822385871 | 1.692625784 |
| ZCO475_02 | Benign | 1.838920317 | 2.121514223 | 1.935825824 | 1.976907933 |
| ZCO485_02 | Benign | 1.764400938 | 1.989263405 | 1.910694695 | 1.763688075 |
| ZCO536_02 | Cancer | 1.89343805 | 1.974481876 | 1.660410804 | 1.611623549 |
| PC_02 | | 1.841784775 | 1.964936806 | 1.619676773 | 1.730343878 |
| ZCO496_02 | Benign | 2.162580446 | 2.382536448 | 2.116479724 | 2.002833962 |
| ZCO502_02 | Cancer | 2.675049984 | 3.045742786 | 2.994221399 | 2.808858956 |
| ZCO382_02 | Benign | 1.93408144 | 2.114663445 | 1.956247752 | 1.949192253 |
| ZCO431_02 | Cancer | 1.823644188 | 2.278026757 | 1.905202857 | 1.946585992 |
| ZCO449_02 | Cancer | 1.761996669 | 1.864626273 | 1.786241463 | 1.586025906 |
| ZCO537_02 | Benign | 1.356810249 | 1.795758377 | 1.362399366 | 1.529045069 |
| ZCO362_02 | Benign | 1.789835687 | 1.919945474 | 1.91319845 | 1.774678189 |
| ZCO488_02 | Benign | 2.428362325 | 2.575464476 | 2.253448087 | 2.35782166 |
| PC_03 | | 1.89777425 | 2.071724037 | 2.130525853 | 1.941448183 |
| ZCO535_02 | Benign | 2.592553526 | 3.192030619 | 2.668041215 | 2.729709431 |
| ZCO443_02 | Cancer | 1.615357925 | 1.874085757 | 1.722557905 | 1.69069925 |
| ZCO393_02 | Benign | 2.000046304 | 2.107092079 | 2.100755772 | 1.877089741 |
| ZCO503_02 | Cancer | 1.843334364 | 2.192553218 | 1.941397683 | 1.839698334 |
| ZCO438_02 | Cancer | 1.975738799 | 2.386677456 | 1.871985759 | 2.148271758 |
| ZCO406_02 | Benign | 3.397698008 | 3.566698882 | 3.370894185 | 3.156358887 |
| PC_04 | | 2.049743352 | 2.316435558 | 2.147432745 | 1.85191677 |
| PC_01 | | 1.746530612 | 2.215179262 | 2.101250934 | 1.70827035 |
| 00082_07 | Cancer | 1.877242238 | 2.053960426 | 2.039041585 | 2.139116158 |
| 02286_07 | Benign | 2.339896047 | 2.67116626 | 2.558048409 | 2.299672897 |
| 02280_06 | Cancer | 1.117539433 | 1.22844092 | 1.176357642 | 1.207608647 |
| 01123_06 | Benign | 1.600628284 | 1.838966433 | 1.661819283 | 1.495294217 |
| 00156_07 | Cancer | 1.925615687 | 2.138098596 | 1.950090356 | 1.690880976 |
| 00781_09 | Benign | 1.51490762 | 1.996394431 | 1.648711633 | 1.700812076 |
| 00539_08 | Cancer | 1.621306499 | 1.772847533 | 1.458940041 | 1.399888744 |
| 02241_07 | Cancer | 1.758821889 | 2.066603923 | 1.848200962 | 1.733284084 |
| 02226_05 | Benign | 1.998694171 | 2.150460166 | 2.275281153 | 2.054403987 |
| PC_03 | | 1.88733707 | 2.030833647 | 2.069746007 | 1.85314561 |
| 00542_08 | NA | 2.061825359 | 2.028977874 | 1.872038882 | 1.815479364 |
| 02497_10 | NA | 1.752416968 | 2.141067373 | 1.902116117 | 1.702863214 |
| 02224_05 | Benign | 1.766160681 | 2.246102057 | 1.854973013 | 1.87956186 |
| 00748_09 | Cancer | 1.907330662 | 2.092637995 | 1.926180188 | 1.861472582 |
| 03630_09 | Benign | 1.700708069 | 2.119208691 | 1.926579817 | 1.754529332 |
| 02279_07 | Cancer | 2.053336143 | 2.204989884 | 2.080720087 | 1.976818148 |
| PC_04 | | 1.814527174 | 1.977781563 | 1.706044242 | 1.78016696 |
| PC_01 | | 1.874485231 | 2.155724619 | 2.051182892 | 1.876416057 |
| NYU806 | Benign | 1.800749486 | 2.335912401 | 1.943705311 | 1.992752165 |
| NYU777 | Cancer | 1.59665169 | 1.890508221 | 1.61285573 | 1.554575466 |
| NYU176 | Benign | 1.679743302 | 1.811087568 | 1.7068991 | 1.518926225 |
| NYU888 | Cancer | 1.790883043 | 2.051058147 | 1.87714179 | 1.630289604 |
| NYU1117 | Benign | 1.804011915 | 2.269808799 | 1.935836838 | 1.97780537 |
| NYU1201 | Cancer | 2.091470394 | 2.513619865 | 2.263274313 | 2.074504082 |
| PC_02 | | 1.717308831 | 2.055048192 | 1.79352316 | 1.835441594 |
| NYU887 | Cancer | 1.870834368 | 2.135242049 | 1.814586691 | 1.910868978 |
| NYU815 | Benign | 2.64634629 | 2.652790985 | 2.318704233 | 2.166724345 |
| NYU927 | Cancer | 1.66714939 | 2.114793161 | 1.674869166 | 1.709789864 |
| NYU1030 | Benign | 1.691220349 | 1.971848674 | 1.602915403 | 1.679993305 |
| NYU1151 | Cancer | 2.047166746 | 2.434464449 | 2.095245668 | 2.265576852 |
| NYU1005 | Benign | 1.872098827 | 2.317668284 | 1.883241798 | 1.972931179 |
| NYU522 | Benign | 2.009565689 | 2.06792207 | 1.898737159 | 1.762096773 |
| NYU389 | Cancer | 2.110052923 | 2.427932717 | 2.332551334 | 2.171708867 |
| PC_03 | | 1.846866493 | 2.180579969 | 1.753178219 | 1.911855984 |
| NYU729 | Cancer | 1.737136644 | 1.872042469 | 1.946104733 | 1.973800638 |
| NYU430 | Benign | 1.743195701 | 1.855279061 | 2.16768636 | 1.70979712 |
| NYU144 | Benign | 1.744356949 | 1.93280403 | 1.765743144 | 1.671589307 |
| NYU256 | Cancer | 1.475105658 | 1.517975011 | 1.312048938 | 1.24753975 |
| NYU1000 | Benign | 1.546766039 | 2.042826784 | 1.651387308 | 1.839538435 |
| NYU575 | Cancer | 2.07240169 | 2.191794118 | 1.974811979 | 1.873233062 |
| PC_04 | | 1.787809938 | 2.302328159 | 1.969334484 | 1.809324799 |

TABLE 11F
PV2 fidelity small nodule batch all transitions (normalized)

| | | LTLLAPLNSVFK_658.40_512.30 | LTLLAPLNSVFK_658.40_804.50 |
|---|---|---|---|
| msfile- | | (SEQ ID | (SEQ ID |
| name | status | No.: 46) | No.: 46) |
| PC_01 | | 1.397019775 | 1.440438817 |
| ZCO489_02 | Benign | 1.248372238 | 1.257550712 |
| ZCO436_02 | Cancer | 1.14998825 | 1.198781653 |
| ZCO512_02 | Cancer | 1.298691948 | 1.287300649 |
| ZCO475_02 | Benign | 1.394008635 | 1.375906455 |
| ZCO485_02 | Benign | 1.564462757 | 1.543963292 |
| ZCO536_02 | Cancer | 2.016527204 | 2.023578087 |
| PC_02 | | 1.326360733 | 1.264182106 |
| ZCO496_02 | Benign | 1.301369896 | 1.310644033 |
| ZCO502_02 | Cancer | 1.090994052 | 1.0300183 |
| ZCO382_02 | Benign | 0.833444785 | 0.832621479 |
| ZCO431_02 | Cancer | 0.886868669 | 0.990611631 |
| ZCO449_02 | Cancer | 1.547547047 | 1.580291665 |
| ZCO537_02 | Benign | 1.572411812 | 1.519120984 |
| ZCO362_02 | Benign | 0.767169538 | 0.777174131 |
| ZCO488_02 | Benign | 1.454825525 | 1.413965873 |
| PC_03 | | 1.36708042 | 1.369045929 |
| ZCO535_02 | Benign | 0.714796903 | 0.760840551 |
| ZCO443_02 | Cancer | 1.326278954 | 1.39914195 |
| ZCO393_02 | Benign | 1.202176119 | 1.26986427 |
| ZCO503_02 | Cancer | 1.183898333 | 1.22215624 |
| ZCO438_02 | Cancer | 1.503069176 | 1.515731362 |
| ZCO406_02 | Benign | 1.905394777 | 1.854087722 |
| PC_04 | | 1.480682041 | 1.421632852 |
| PC_01 | | 1.41960685 | 1.372496446 |
| 00082_07 | Cancer | 1.535229885 | 1.657175755 |
| 02286_07 | Benign | 1.551089982 | 1.55609209 |
| 02280_06 | Cancer | 1.34525595 | 1.439836948 |
| 01123_06 | Benign | 1.55800292 | 1.492237393 |
| 00156_07 | Cancer | 1.687960144 | 1.632424321 |
| 00781_09 | Benign | 2.235668602 | 2.17674569 |
| 00539_08 | Cancer | 1.285722204 | 1.30334384 |
| 02241_07 | Cancer | 1.082222201 | 1.120984794 |
| 02226_05 | Benign | 1.616736686 | 1.629091702 |
| PC_03 | | 1.414076108 | 1.530005699 |
| 00542_08 | NA | 1.458646284 | 1.39966386 |
| 02497_10 | NA | 1.83390026 | 1.783296155 |
| 02224_05 | Benign | 1.8091712 | 1.748036919 |
| 00748_09 | Cancer | 1.287263073 | 1.322675499 |
| 03630_09 | Benign | 1.503087374 | 1.44608336 |
| 02279_07 | Cancer | 1.306177062 | 1.277258106 |
| PC_04 | | 1.356357136 | 1.407416626 |
| PC_01 | | 1.391528036 | 1.480970747 |
| NYU806 | Benign | 1.331117277 | 1.359452087 |
| NYU777 | Cancer | 1.07779325 | 1.014586332 |
| NYU176 | Benign | 1.498223403 | 1.537471813 |
| NYU888 | Cancer | 1.307841105 | 1.378455859 |
| NYU1117 | Benign | 1.168152742 | 1.171928217 |
| NYU1201 | Cancer | 1.054141873 | 1.102004179 |
| PC_02 | | 1.311253724 | 1.400528282 |
| NYU887 | Cancer | 1.431161601 | 1.539649799 |
| NYU815 | Benign | 1.449295278 | 1.417166496 |
| NYU927 | Cancer | 1.323825757 | 1.328964099 |
| NYU1030 | Benign | 1.380621371 | 1.484141052 |
| NYU1151 | Cancer | 1.558434039 | 1.576736275 |
| NYU1005 | Benign | 2.34001241 | 2.387945416 |
| NYU522 | Benign | 1.40442773 | 1.480809064 |
| NYU389 | Cancer | 1.061187422 | 1.023308665 |
| PC_03 | | 1.307831291 | 1.422596669 |
| NYU729 | Cancer | 1.571044996 | 1.6020581 |
| NYU430 | Benign | 1.114704773 | 1.191817122 |
| NYU144 | Benign | 1.711263664 | 1.756990303 |
| NYU256 | Cancer | 1.062643845 | 1.144548794 |
| NYU1000 | Benign | 1.215751159 | 1.424990734 |
| NYU575 | Cancer | 1.062224757 | 1.093109211 |
| PC_04 | | 1.438307541 | 1.382155039 |
| | | LTLLAPLNSVFK_658.40_875.50 | QITVNDLPVGR_606.30_428.30 |
| msfile- | | (SEQ ID | (SEQ ID |
| name | status | No.: 46) | No.: 58) |
| PC_01 | | 1.408320389 | 0.140036856 |
| ZCO489_02 | Benign | 1.265195424 | 0.368097138 |
| ZCO436_02 | Cancer | 1.156780759 | 0.342026932 |
| ZCO512_02 | Cancer | 1.301703575 | 0.41026912 |
| ZCO475_02 | Benign | 1.360896226 | 0.740034792 |
| ZCO485_02 | Benign | 1.444034077 | 0.714120326 |
| ZCO536_02 | Cancer | 2.052326172 | 1.489438136 |
| PC_02 | | 1.3099451 | 0.094821076 |
| ZCO496_02 | Benign | 1.298069763 | 0.658680927 |
| ZCO502_02 | Cancer | 0.991102367 | 0.441575472 |
| ZCO382_02 | Benign | 0.808928742 | 0.148374361 |
| ZCO431_02 | Cancer | 0.907993266 | 0.544123251 |
| ZCO449_02 | Cancer | 1.529918218 | 0.462641275 |
| ZCO537_02 | Benign | 1.624342357 | 0.392673881 |
| ZCO362_02 | Benign | 0.77091823 | 0.08193127 |
| ZCO488_02 | Benign | 1.432081227 | 0.639309416 |
| PC_03 | | 1.368135651 | 0.082179578 |
| ZCO535_02 | Benign | 0.685501208 | 1.460044819 |
| ZCO443_02 | Cancer | 1.384651088 | 0.906263536 |
| ZCO393_02 | Benign | 1.164154495 | 0.150077788 |
| ZCO503_02 | Cancer | 1.142216538 | 0.367134903 |
| ZCO438_02 | Cancer | 1.52737559 | 0.312207395 |
| ZCO406_02 | Benign | 1.883230938 | 0.689984066 |
| PC_04 | | 1.370810583 | 0.091022526 |
| PC_01 | | 1.383506317 | 0.09218022 |
| 00082_07 | Cancer | 1.446449816 | 0.442238684 |
| 02286_07 | Benign | 1.508277494 | 0.391968732 |
| 02280_06 | Cancer | 1.430086213 | 0.278475318 |
| 01123_06 | Benign | 1.50977305 | 0.317843837 |
| 00156_07 | Cancer | 1.56912655 | 0.428683661 |
| 00781_09 | Benign | 2.067038413 | 0.467632972 |
| 00539_08 | Cancer | 1.299652439 | 0.391847577 |
| 02241_07 | Cancer | 1.132727899 | 0.205185454 |
| 02226_05 | Benign | 1.731411833 | 0.24677982 |
| PC_03 | | 1.555737889 | 0.101331218 |
| 00542_08 | NA | 1.41315531 | 0.205362102 |
| 02497_10 | NA | 1.639862023 | 0.157254386 |
| 02224_05 | Benign | 1.665068787 | 0.216326452 |
| 00748_09 | Cancer | 1.226273772 | 0.626520726 |
| 03630_09 | Benign | 1.562117498 | 0.633935473 |
| 02279_07 | Cancer | 1.276392361 | 0.663737282 |
| PC_04 | | 1.353649935 | 0.094348058 |
| PC_01 | | 1.420145306 | 0.091638163 |
| NYU806 | Benign | 1.309450367 | 3.790621014 |
| NYU777 | Cancer | 1.048223272 | 0.729776699 |
| NYU176 | Benign | 1.469807867 | 0.394314508 |
| NYU888 | Cancer | 1.375802411 | 0.437481689 |
| NYU1117 | Benign | 1.107437116 | 0.379747836 |
| NYU1201 | Cancer | 1.053419806 | 0.522505753 |
| PC_02 | | 1.286772984 | 0.084077035 |
| NYU887 | Cancer | 1.582864754 | 0.32970087 |
| NYU815 | Benign | 1.413101139 | 0.433810008 |
| NYU927 | Cancer | 1.402704425 | 0.38104063 |
| NYU1030 | Benign | 1.401211524 | 0.244739708 |
| NYU1151 | Cancer | 1.581026453 | 0.645301436 |
| NYU1005 | Benign | 2.357944664 | 0.653943035 |
| NYU522 | Benign | 1.422573078 | 0.599539459 |
| NYU389 | Cancer | 1.045240838 | 0.509607849 |
| PC_03 | | 1.425534637 | 0.090815167 |
| NYU729 | Cancer | 1.515391409 | 0.308012701 |
| NYU430 | Benign | 1.149396391 | 0.423282629 |
| NYU144 | Benign | 1.893851951 | 0.610951435 |
| NYU256 | Cancer | 1.036833381 | 0.260987318 |
| NYU1000 | Benign | 1.374138913 | 0.35271459 |
| NYU575 | Cancer | 1.072566385 | 0.441835699 |
| PC_04 | | 1.471701265 | 0.085559114 |
| | | QITVNDLPVGR_606.30_770.40 | QITVNDLPVGR_606.30_970.50 |
| msfile- | | (SEQ ID | (SEQ ID |
| name | status | No.: 58) | No.: 58) |
| PC_01 | | 0.133841723 | 0.134340656 |
| ZCO489_02 | Benign | 0.344569936 | 0.327282944 |
| ZCO436_02 | Cancer | 0.330249049 | 0.359799682 |
| ZCO512_02 | Cancer | 0.411436366 | 0.428489838 |
| ZCO475_02 | Benign | 0.725962804 | 0.698053406 |
| ZCO485_02 | Benign | 0.628583382 | 0.668137369 |
| ZCO536_02 | Cancer | 1.601101751 | 1.583268915 |
| PC_02 | | 0.101718509 | 0.093425751 |
| ZCO496_02 | Benign | 0.666485575 | 0.61449894 |
| ZCO502_02 | Cancer | 0.476940556 | 0.473511033 |
| ZCO382_02 | Benign | 0.150925084 | 0.133317652 |
| ZCO431_02 | Cancer | 0.465191577 | 0.503644005 |
| ZCO449_02 | Cancer | 0.458879365 | 0.474761462 |
| ZCO537_02 | Benign | 0.363404259 | 0.394794869 |
| ZCO362_02 | Benign | 0.08758701 | 0.080825527 |
| ZCO488_02 | Benign | 0.641375067 | 0.741769175 |
| PC_03 | | 0.084904301 | 0.093672737 |
| ZCO535_02 | Benign | 1.515887099 | 1.488774865 |
| ZCO443_02 | Cancer | 0.981605149 | 0.952251064 |
| ZCO393_02 | Benign | 0.134203155 | 0.139742993 |
| ZCO503_02 | Cancer | 0.373887621 | 0.390737216 |
| ZCO438_02 | Cancer | 0.300753938 | 0.330903534 |
| ZCO406_02 | Benign | 0.681955631 | 0.783801505 |
| PC_04 | | 0.081568779 | 0.08731202 |
| PC_01 | | 0.08590282 | 0.083739409 |
| 00082_07 | Cancer | 0.459305224 | 0.434193992 |
| 02286_07 | Benign | 0.381738552 | 0.406814381 |
| 02280_06 | Cancer | 0.28241687 | 0.282848162 |
| 01123_06 | Benign | 0.34766754 | 0.344895956 |
| 00156_07 | Cancer | 0.430863443 | 0.462490344 |
| 00781_09 | Benign | 0.484566226 | 0.4624234 |
| 00539_08 | Cancer | 0.367029944 | 0.41946979 |
| 02241_07 | Cancer | 0.209126528 | 0.207153955 |
| 02226_05 | Benign | 0.21707405 | 0.230335795 |
| PC_03 | | 0.094507381 | 0.096407947 |
| 00542_08 | NA | 0.212570861 | 0.22459793 |
| 02497_10 | NA | 0.160755983 | 0.148305 |
| 02224_05 | Benign | 0.229751467 | 0.217676529 |
| 00748_09 | Cancer | 0.634291294 | 0.683112641 |
| 03630_09 | Benign | 0.666180143 | 0.615976033 |
| 02279_07 | Cancer | 0.672731362 | 0.685137029 |
| PC_04 | | 0.10739817 | 0.111548467 |
| PC_01 | | 0.095408397 | 0.092906733 |
| NYU806 | Benign | 3.759575263 | 4.073354282 |
| NYU777 | Cancer | 0.704831811 | 0.718348154 |
| NYU176 | Benign | 0.415015184 | 0.404594201 |
| NYU888 | Cancer | 0.461984786 | 0.421479958 |
| NYU1117 | Benign | 0.357406388 | 0.345429654 |
| NYU1201 | Cancer | 0.628612248 | 0.531211309 |
| PC_02 | | 0.094042385 | 0.08667037 |
| NYU887 | Cancer | 0.352324669 | 0.349157484 |
| NYU815 | Benign | 0.432750854 | 0.410063603 |
| NYU927 | Cancer | 0.390174887 | 0.411977347 |
| NYU1030 | Benign | 0.239294233 | 0.245158545 |
| NYU1151 | Cancer | 0.610138376 | 0.690791214 |
| NYU1005 | Benign | 0.731222267 | 0.771790256 |
| NYU522 | Benign | 0.578544015 | 0.604597387 |
| NYU389 | Cancer | 0.578731929 | 0.599429304 |
| PC_03 | | 0.078349713 | 0.073937085 |
| NYU729 | Cancer | 0.313818668 | 0.356745449 |
| NYU430 | Benign | 0.426927488 | 0.458903895 |
| NYU144 | Benign | 0.692975397 | 0.691138704 |
| NYU256 | Cancer | 0.266877087 | 0.286864756 |
| NYU1000 | Benign | 0.348783259 | 0.3578193 |
| NYU575 | Cancer | 0.457111621 | 0.447763533 |
| PC_04 | | 0.085671779 | 0.089047258 |

| | | SGYLL- |
|---|---|---|
| msfile- | | PDTK_497.27_308.10 |
| name | status | (SEQ ID No.: 49) |
| PC_01 | | 0.25200544 |
| ZCO489_02 | Benign | 0.275702255 |
| ZCO436_02 | Cancer | 0.237543303 |
| ZCO512_02 | Cancer | 0.285664279 |
| ZCO475_02 | Benign | 0.275715977 |
| ZCO485_02 | Benign | 0.273465876 |
| ZCO536_02 | Cancer | 0.365913415 |
| PC_02 | | 0.20658164 |
| ZCO496_02 | Benign | 0.140198796 |
| ZCO502_02 | Cancer | 0.649969869 |
| ZCO382_02 | Benign | 0.129000788 |
| ZCO431_02 | Cancer | 0.34926771 |
| ZCO449_02 | Cancer | 0.431369923 |
| ZCO537_02 | Benign | 0.411144419 |
| ZCO362_02 | Benign | 0.172834493 |
| ZCO488_02 | Benign | 0.281204914 |
| PC_03 | | 0.2147304 |
| ZCO535_02 | Benign | 0.229092353 |
| ZCO443_02 | Cancer | 0.368838333 |
| ZCO393_02 | Benign | 0.140637809 |
| ZCO503_02 | Cancer | 0.246231267 |
| ZCO438_02 | Cancer | 0.386312282 |
| ZCO406_02 | Benign | 0.253501275 |
| PC_04 | | 0.205200937 |
| PC_01 | | 0.233904143 |
| 00082_07 | Cancer | 0.210837827 |
| 02286_07 | Benign | 0.230369362 |
| 02280_06 | Cancer | 0.150260267 |
| 01123_06 | Benign | 0.138757497 |
| 00156_07 | Cancer | 0.146687738 |
| 00781_09 | Benign | 0.253555335 |
| 00539_08 | Cancer | 0.142060017 |
| 02241_07 | Cancer | 0.114690297 |
| 02226_05 | Benign | 0.404136964 |
| PC_03 | | 0.277911928 |
| 00542_08 | NA | 0.200664214 |
| 02497_10 | NA | 0.16741174 |
| 02224_05 | Benign | 0.234358581 |
| 00748_09 | Cancer | 0.156667324 |
| 03630_09 | Benign | 0.249270454 |
| 02279_07 | Cancer | 0.166528815 |
| PC_04 | | 0.252708732 |
| PC_01 | | 0.263322053 |
| NYU806 | Benign | 0.203829927 |
| NYU777 | Cancer | 0.186476658 |
| NYU176 | Benign | 0.305316437 |
| NYU888 | Cancer | 0.205331169 |
| NYU1117 | Benign | 0.260245221 |
| NYU1201 | Cancer | 0.252420373 |
| PC_02 | | 0.216969241 |
| NYU887 | Cancer | 0.164017508 |
| NYU815 | Benign | 0.150519949 |
| NYU927 | Cancer | 0.208405145 |
| NYU1030 | Benign | 0.202679834 |
| NYU1151 | Cancer | 0.28324733 |
| NYU1005 | Benign | 0.269867542 |
| NYU522 | Benign | 0.206984185 |
| NYU389 | Cancer | 0.261759458 |
| PC_03 | | 0.209947368 |
| NYU729 | Cancer | 0.201124706 |
| NYU430 | Benign | 0.126281518 |
| NYU144 | Benign | 0.300081632 |
| NYU256 | Cancer | 0.142178097 |
| NYU1000 | Benign | 0.261181015 |
| NYU575 | Cancer | 0.648869277 |
| PC_04 | | 0.286895772 |

TABLE 11G
PV2 fidelity small nodule batch all transitions (normalized)

| | | SGYLL- | SGYLL- | | |
|---|---|---|---|---|---|
| msfile- | | PDTK_497.27_460.20 | PDTK_497.27_573.30 | SLEDLQLTHNK_433.23_201.10 | SLEDLQLTHNK_433.23_398.20 |
| name | status | (SEQ ID No.: 49) | (SEQ ID No.: 49) | (SEQ ID No.: 69) | (SEQ ID No.: 69) |
| PC_01 | | 0.259039262 | 0.219077441 | 11.57925495 | 10.39641991 |
| ZCO489_02 | Benign | 0.249417254 | 0.329040995 | 10.73518681 | 10.55524849 |
| ZCO436_02 | Cancer | 0.182775959 | 0.249187938 | 12.91610824 | 12.84337424 |
| ZCO512_02 | Cancer | 0.235629552 | 0.25546791 | 8.704645661 | 7.155892204 |
| ZCO475_02 | Benign | 0.248094646 | 0.282197704 | 10.23615869 | 7.694189657 |
| ZCO485_02 | Benign | 0.282432761 | 0.245450562 | 11.89260436 | 12.03732741 |
| ZCO536_02 | Cancer | 0.260545425 | 0.292649264 | 9.756107747 | 9.571351027 |
| PC_02 | | 0.195003637 | 0.222538734 | 9.887590589 | 10.30154087 |
| ZCO496_02 | Benign | 0.112294816 | 0.168430306 | 11.03086777 | 10.55433999 |
| ZCO502_02 | Cancer | 0.51908916 | 0.706454894 | 12.233955 | 12.20174079 |
| ZCO382_02 | Benign | 0.168493941 | 0.134887786 | 9.339815037 | 10.92709606 |
| ZCO431_02 | Cancer | 0.267889273 | 0.336145026 | 8.480073896 | 6.902496276 |
| ZCO449_02 | Cancer | 0.357813393 | 0.410711223 | 9.604240971 | 10.26634765 |
| ZCO537_02 | Benign | 0.365861619 | 0.341780607 | 11.86147691 | 10.94603564 |
| ZCO362_02 | Benign | 0.182205838 | 0.190755753 | 8.462651763 | 7.087385169 |
| ZCO488_02 | Benign | 0.221708484 | 0.2856137 | 9.322091671 | 10.79907558 |
| PC_03 | | 0.225363578 | 0.246174148 | 12.60518377 | 10.81960615 |
| ZCO535_02 | Benign | 0.216753595 | 0.193506617 | 7.393684534 | 8.546255579 |
| ZCO443_02 | Cancer | 0.285716716 | 0.336714246 | 10.28126101 | 9.845567391 |
| ZCO393_02 | Benign | 0.106774474 | 0.11700565 | 9.172544334 | 10.54345532 |
| ZCO503_02 | Cancer | 0.215161689 | 0.229405795 | 9.687927401 | 9.669586255 |
| ZCO438_02 | Cancer | 0.317377171 | 0.381061452 | 9.415485671 | 9.174224286 |
| ZCO406_02 | Benign | 0.27135467 | 0.359586071 | 8.562187393 | 7.553260723 |
| PC_04 | | 0.164071275 | 0.212036546 | 11.22538013 | 11.16794117 |
| PC_01 | | 0.188912869 | 0.206754472 | 11.69053575 | 9.763695813 |
| 00082_07 | Cancer | 0.165929767 | 0.235976801 | 8.542926752 | 8.922374916 |
| 02286_07 | Benign | 0.184126678 | 0.198521586 | 9.028030052 | 8.316545975 |
| 02280_06 | Cancer | 0.117195084 | 0.125489379 | 8.988549312 | 9.054020603 |
| 01123_06 | Benign | 0.120882359 | 0.122613127 | 10.84563062 | 10.83008678 |
| 00156_07 | Cancer | 0.100270442 | 0.145839292 | 7.403127299 | 7.485749029 |
| 00781_09 | Benign | 0.225070947 | 0.277238564 | 9.716518085 | 8.922351562 |
| 00539_08 | Cancer | 0.109651306 | 0.100593969 | 9.368864709 | 8.595833069 |
| 02241_07 | Cancer | 0.106389454 | 0.101013635 | 10.15359823 | 10.22009348 |
| 02226_05 | Benign | 0.343387872 | 0.308596368 | 10.43247628 | 9.347133462 |
| PC_03 | | 0.200908725 | 0.203932077 | 11.29560435 | 11.20804061 |
| 00542_08 | NA | 0.198919386 | 0.228148544 | 7.384308429 | 8.217479242 |
| 02497_10 | NA | 0.157511596 | 0.174724326 | 9.090094286 | 7.543671081 |
| 02224_05 | Benign | 0.179032099 | 0.19294407 | 8.44040586 | 7.431227513 |
| 00748_09 | Cancer | 0.086376585 | 0.142273161 | 6.562339663 | 5.812465188 |
| 03630_09 | Benign | 0.144193898 | 0.190540532 | 8.340320874 | 8.238816 |
| 02279_07 | Cancer | 0.118615413 | 0.178100914 | 9.15917887 | 6.642332314 |
| PC_04 | | 0.223877959 | 0.234280697 | 10.81992991 | 11.53034528 |
| PC_01 | | 0.231386956 | 0.225308176 | 11.14697453 | 10.92803043 |
| NYU806 | Benign | 0.184179426 | 0.214978401 | 6.91820576 | 7.378357334 |
| NYU777 | Cancer | 0.150378048 | 0.194502454 | 8.773566408 | 8.851017381 |
| NYU176 | Benign | 0.299365624 | 0.336849163 | 7.437428491 | 7.588040151 |
| NYU888 | Cancer | 0.129565896 | 0.147584823 | 12.25968742 | 11.90947396 |
| NYU1117 | Benign | 0.225774472 | 0.243344234 | 9.340885553 | 8.177442803 |
| NYU1201 | Cancer | 0.168870122 | 0.207045319 | 8.830845646 | 7.17797761 |
| PC_02 | | 0.174296532 | 0.19821593 | 9.814448217 | 10.05361694 |
| NYU887 | Cancer | 0.106823432 | 0.14065701 | 11.062029 | 11.6043805 |
| NYU815 | Benign | 0.140654335 | 0.128478286 | 6.631685857 | 6.859559359 |
| NYU927 | Cancer | 0.167794059 | 0.221649256 | 23.743224 | 18.09012219 |
| NYU1030 | Benign | 0.149672834 | 0.161176463 | 12.30938555 | 12.58922293 |
| NYU1151 | Cancer | 0.222644292 | 0.21184856 | 9.965813752 | 12.02665119 |
| NYU1005 | Benign | 0.269322136 | 0.218264919 | 7.240708496 | 6.104904518 |
| NYU522 | Benign | 0.179091953 | 0.161527401 | 8.193726412 | 8.134422763 |
| NYU389 | Cancer | 0.226600985 | 0.255218663 | 13.78680838 | 11.84165017 |
| PC_03 | | 0.193557542 | 0.200261169 | 10.58016012 | 11.00922827 |
| NYU729 | Cancer | 0.143378385 | 0.212629672 | 9.617827705 | 9.257839863 |
| NYU430 | Benign | 0.100540417 | 0.106299763 | 9.292095998 | 7.585321069 |
| NYU144 | Benign | 0.153602866 | 0.233960756 | 11.2417074 | 11.24212476 |
| NYU256 | Cancer | 0.102957489 | 0.1193556 | 10.25763503 | 9.175316754 |
| NYU1000 | Benign | 0.235933744 | 0.245722129 | 13.0055322 | 11.440134 |
| NYU575 | Cancer | 0.656053444 | 0.629702703 | 8.756843627 | 8.841111643 |
| PC_04 | | 0.186289483 | 0.221204317 | 11.46206466 | 10.34865302 |

| msfile- | | SLEDLQLTHNK_433.23_499.30 | SLEDLQLTHNK_433.23_549.30 | STGGAPTFNVTVTK_690.40_1006.60 |
|---|---|---|---|---|
| name | status | (SEQ ID No.: 69) | (SEQ ID No.: 69) | (SEQ ID No.: 59) |
| PC_01 | | 8.663397254 | 9.999242891 | 1.142968007 |
| ZCO489_02 | Benign | 10.59086608 | 10.1242332 | 5.380295112 |
| ZCO436_02 | Cancer | 12.2085805 | 12.42367468 | 1.326718344 |
| ZCO512_02 | Cancer | 6.970339585 | 7.524573956 | 3.472889972 |
| ZCO475_02 | Benign | 6.460621068 | 9.006398041 | 2.255173628 |
| ZCO485_02 | Benign | 9.615596081 | 9.615904997 | 1.787692571 |
| ZCO536_02 | Cancer | 7.12408201 | 9.997842178 | 1.863201978 |
| PC_02 | | 8.313424621 | 10.97273253 | 0.200718037 |
| ZCO496_02 | Benign | 10.23417503 | 11.12587706 | 1.601688592 |
| ZCO502_02 | Cancer | 11.63713908 | 12.33951 | 8.351675963 |
| ZCO382_02 | Benign | 8.477026939 | 8.972779477 | 0.615714724 |
| ZCO431_02 | Cancer | 6.147584103 | 7.484066038 | 7.032595597 |
| ZCO449_02 | Cancer | 9.522149718 | 9.908429897 | 3.657794104 |
| ZCO537_02 | Benign | 10.22278412 | 12.13474159 | 2.597102887 |
| ZCO362_02 | Benign | 6.93362411 | 7.859011582 | 0.29986413 |
| ZCO488_02 | Benign | 9.310245818 | 10.02037198 | 2.144289829 |
| PC_03 | | 8.155979498 | 10.03658055 | 0.171283411 |
| ZCO535_02 | Benign | 6.631012748 | 7.256760964 | 1.928595864 |
| ZCO443_02 | Cancer | 8.747238873 | 10.24520434 | 10.76552705 |
| ZCO393_02 | Benign | 9.674823538 | 9.31974161 | 0.622374681 |
| ZCO503_02 | Cancer | 9.847385384 | 9.564025811 | 3.494740954 |
| ZCO438_02 | Cancer | 8.15145506 | 7.635939814 | 4.228342912 |
| ZCO406_02 | Benign | 7.120049044 | 8.509069483 | 1.373313009 |
| PC_04 | | 8.695105229 | 10.43022381 | 0.178458126 |
| PC_01 | | 9.482514689 | 10.92692696 | 0.173126018 |
| 00082_07 | Cancer | 6.5399985 | 8.363316237 | 0.12047598 |
| 02286_07 | Benign | 8.557462421 | 8.959897054 | 0.170735668 |
| 02280_06 | Cancer | 7.857754161 | 10.69350292 | 0.081254189 |
| 01123_06 | Benign | 8.239742349 | 10.09731843 | 0.085846412 |
| 00156_07 | Cancer | 6.777689091 | 8.756654339 | 0.107937913 |
| 00781_09 | Benign | 8.075850907 | 9.09732579 | 0.093620966 |
| 00539_08 | Cancer | 8.168375073 | 8.44215454 | 0.100014553 |
| 02241_07 | Cancer | 10.05647486 | 11.5086463 | 0.131739911 |
| 02226_05 | Benign | 8.691123853 | 11.16539669 | 0.22969415 |
| PC_03 | | 10.31283316 | 9.629261683 | 0.187805798 |
| 00542_08 | NA | 6.99628777 | 9.025756929 | 0.089758113 |
| 02497_10 | NA | 6.899435369 | 8.715953965 | 0.092429943 |
| 02224_05 | Benign | 6.775835594 | 9.439486591 | 0.098560453 |
| 00748_09 | Cancer | 5.322002706 | 6.898127257 | 0.143447572 |
| 03630_09 | Benign | 7.670530004 | 8.856862045 | 0.228114787 |
| 02279_07 | Cancer | 6.595751937 | 6.271615403 | 0.166049272 |
| PC_04 | | 7.80380286 | 10.48520116 | 0.182471201 |
| PC_01 | | 7.877702583 | 10.48224891 | 0.170653681 |
| NYU806 | Benign | 5.333116062 | 6.562916105 | 1.311153821 |
| NYU777 | Cancer | 6.691213338 | 9.039073958 | 1.789595468 |
| NYU176 | Benign | 6.530690391 | 7.968856545 | 0.859754289 |
| NYU888 | Cancer | 9.682329133 | 10.63122893 | 0.712138635 |
| NYU1117 | Benign | 7.983833074 | 8.017893943 | 0.196702753 |
| NYU1201 | Cancer | 6.778633359 | 9.644425387 | 0.255842608 |
| PC_02 | | 7.82668019 | 11.08562831 | 0.148263017 |
| NYU887 | Cancer | 9.157503928 | 9.581615668 | 0.622925567 |
| NYU815 | Benign | 5.58683508 | 6.644158481 | 0.61874537 |
| NYU927 | Cancer | 20.48875754 | 20.91810768 | 0.734492975 |
| NYU1030 | Benign | 12.72107421 | 13.63422733 | 0.303388296 |
| NYU1151 | Cancer | 8.078260641 | 9.234120074 | 0.840325318 |
| NYU1005 | Benign | 5.039250067 | 5.902503336 | 3.712835952 |
| NYU522 | Benign | 7.080564738 | 7.595608466 | 1.215293234 |
| NYU389 | Cancer | 10.19739253 | 10.41992457 | 2.713271299 |
| PC_03 | | 8.404635139 | 9.886730891 | 0.173477967 |
| NYU729 | Cancer | 9.463517804 | 10.33609953 | 2.523751621 |
| NYU430 | Benign | 8.277757442 | 8.919339759 | 2.963548973 |
| NYU144 | Benign | 9.905118411 | 11.92773688 | 0.939075077 |
| NYU256 | Cancer | 9.314330924 | 10.33916006 | 0.21068248 |
| NYU1000 | Benign | 9.944989388 | 11.53391777 | 0.686895277 |
| NYU575 | Cancer | 8.802021235 | 9.682501805 | 10.64047698 |
| PC_04 | | 9.271280586 | 10.65020864 | 0.170857572 |

TABLE 11H

PV2 fidelity small nodule batch all transitions (normalized)

| msfile- | | STGGAPTFNVTVTK_690.40_189.10 | STGGAPTFNVTVTK_690.40_374.20 | STGGAPTFNVTVTK_690.40_503.80 |
|---|---|---|---|---|
| name | status | (SEQ ID No.: 59) | (SEQ ID No.: 59) | (SEQ ID No.: 59) |
| PC_01 | | 1.189949781 | 0.969013493 | 1.036176191 |
| ZCO489_02 | Benign | 4.620953931 | 4.919834447 | 5.830387389 |
| ZCO436_02 | Cancer | 1.351900373 | 1.162646171 | 1.253201412 |
| ZCO512_02 | Cancer | 3.629861444 | 3.234378614 | 3.402986127 |
| ZCO475_02 | Benign | 1.962964765 | 2.078819139 | 2.217894338 |
| ZCO485_02 | Benign | 1.698050857 | 1.799860613 | 1.681015115 |
| ZCO536_02 | Cancer | 2.328040798 | 1.949159306 | 1.843767986 |
| PC_02 | | 0.208874889 | 0.176355654 | 0.194706306 |
| ZCO496_02 | Benign | 1.60659048 | 1.860426657 | 1.821429035 |
| ZCO502_02 | Cancer | 7.291452309 | 8.426445852 | 9.346632406 |
| ZCO382_02 | Benign | 0.75164589 | 0.679464608 | 0.723910905 |
| ZCO431_02 | Cancer | 7.413183207 | 5.681562681 | 6.270280261 |
| ZCO449_02 | Cancer | 4.409776148 | 4.048685652 | 4.259454168 |
| ZCO537_02 | Benign | 3.099846203 | 2.18696353 | 2.652757211 |
| ZCO362_02 | Benign | 0.418502061 | 0.312557535 | 0.257297597 |
| ZCO488_02 | Benign | 2.543454877 | 2.190791613 | 2.272822258 |
| PC_03 | | 0.163185958 | 0.18255317 | 0.173612589 |
| ZCO535_02 | Benign | 1.781267077 | 2.085113981 | 1.758116489 |
| ZCO443_02 | Cancer | 9.754515701 | 8.409271104 | 9.768793419 |
| ZCO393_02 | Benign | 0.783929907 | 0.767060372 | 0.703601727 |
| ZCO503_02 | Cancer | 3.71970436 | 4.01773478 | 3.296708197 |
| ZCO438_02 | Cancer | 4.8618824 | 4.041951952 | 5.182986286 |
| ZCO406_02 | Benign | 1.446128543 | 1.356393288 | 1.679824566 |
| PC_04 | | 0.179595149 | 0.162767556 | 0.189729703 |
| PC_01 | | 0.103249258 | 0.162207759 | 0.192544011 |
| 00082_07 | Cancer | 0.073232673 | 0.12966599 | 0.117183262 |
| 02286_07 | Benign | 0.1170579 | 0.154073924 | 0.173939045 |
| 02280_06 | Cancer | 0.076787303 | 0.127107121 | 0.076743593 |
| 01123_06 | Benign | 0.077899179 | 0.093530474 | 0.068734046 |
| 00156_07 | Cancer | 0.080962846 | 0.131813207 | 0.09562331 |
| 00781_09 | Benign | 0.095837639 | 0.101941366 | 0.102503388 |
| 00539_08 | Cancer | 0.133887564 | 0.122529152 | 0.096869824 |
| 02241_07 | Cancer | 0.149739748 | 0.170197688 | 0.146477626 |
| 02226_05 | Benign | 0.201908415 | 0.292195976 | 0.216936257 |
| PC_03 | | 0.167612859 | 0.204200336 | 0.137909747 |
| 00542_08 | NA | 0.069816506 | 0.134316206 | 0.110516916 |
| 02497_10 | NA | 0.094835625 | 0.102094401 | 0.066275407 |
| 02224_05 | Benign | 0.067665967 | 0.141559076 | 0.069374682 |
| 00748_09 | Cancer | 0.15155278 | 0.165273083 | 0.146103828 |
| 03630_09 | Benign | 0.154496771 | 0.23746089 | 0.239488331 |
| 02279_07 | Cancer | 0.16734067 | 0.189633152 | 0.146880961 |
| PC_04 | | 0.148976959 | 0.172638409 | 0.160256636 |
| PC_01 | | 0.171072995 | 0.184315169 | 0.19766307 |
| NYU806 | Benign | 1.36468122 | 1.24735286 | 1.568464998 |
| NYU777 | Cancer | 1.669445384 | 1.661986165 | 1.942547972 |
| NYU176 | Benign | 0.835169126 | 0.722325779 | 0.808706151 |
| NYU888 | Cancer | 0.779955019 | 0.644984296 | 0.875168505 |
| NYU1117 | Benign | 0.20649441 | 0.194336128 | 0.201848218 |
| NYU1201 | Cancer | 0.167783276 | 0.227639308 | 0.21613797 |
| PC_02 | | 0.216201866 | 0.176864356 | 0.169253229 |
| NYU887 | Cancer | 0.551312556 | 0.635613618 | 0.513406235 |
| NYU815 | Benign | 0.812444178 | 0.655842644 | 0.735383189 |
| NYU927 | Cancer | 0.717993912 | 0.766999812 | 0.659775142 |
| NYU1030 | Benign | 0.222384201 | 0.294842923 | 0.240606864 |
| NYU1151 | Cancer | 0.76886674 | 0.724251662 | 0.77576766 |
| NYU1005 | Benign | 4.943001883 | 3.952654021 | 4.25529731 |
| NYU522 | Benign | 1.334830284 | 1.321292647 | 1.310400265 |
| NYU389 | Cancer | 3.153398187 | 2.960427455 | 2.895069524 |
| PC_03 | | 0.202400832 | 0.173716853 | 0.177407046 |
| NYU729 | Cancer | 2.799490114 | 2.591317472 | 3.65849017 |
| NYU430 | Benign | 3.393586195 | 3.106498294 | 3.213322218 |
| NYU144 | Benign | 1.020351786 | 0.8455283 | 0.923926514 |
| NYU256 | Cancer | 0.136102572 | 0.170306505 | 0.210346966 |
| NYU1000 | Benign | 0.755545204 | 0.608507889 | 0.758393217 |
| NYU575 | Cancer | 9.370854581 | 8.379748974 | 10.65591399 |
| PC_04 | | 0.256751531 | 0.175977771 | 0.182963976 |

| msfile- | | TASDFITK_441.73_173.10 | TASDFITK_441.73_508.30 | TASDFITK_441.73_710.40 | TASDFITK_441.73_781.40 |
|---|---|---|---|---|---|
| name | status | (SEQ ID No.: 64) | (SEQ ID No.: 64) | (SEQ No.: 64) | (SEQ ID No.: 64) |
| PC_01 | | 0.49459641 | 0.486394681 | 0.507071405 | 0.509703713 |
| ZCO489_02 | Benign | 0.458478046 | 0.533938526 | 0.60390872 | 0.509533114 |
| ZCO436_02 | Cancer | 0.296002356 | 0.32329638 | 0.314401607 | 0.30528425 |
| ZCO512_02 | Cancer | 0.255278048 | 0.246625416 | 0.255024711 | 0.264197356 |
| ZCO475_02 | Benign | 0.330346276 | 0.358364281 | 0.382697435 | 0.32997927 |
| ZCO485_02 | Benign | 0.484038468 | 0.460932834 | 0.499774861 | 0.479965645 |
| ZCO536_02 | Cancer | 0.366089666 | 0.426889248 | 0.445901022 | 0.407319812 |
| PC_02 | | 0.41791411 | 0.408874975 | 0.427102477 | 0.453630992 |
| ZCO496_02 | Benign | 0.503999744 | 0.452130759 | 0.489181184 | 0.505450838 |
| ZCO502_02 | Cancer | 0.355552536 | 0.364941238 | 0.384201125 | 0.412888951 |
| ZCO382_02 | Benign | 0.33125267 | 0.375606259 | 0.378901681 | 0.358819812 |
| ZCO431_02 | Cancer | 0.296399036 | 0.301015116 | 0.309282461 | 0.316636966 |
| ZCO449_02 | Cancer | 0.488275503 | 0.537707344 | 0.594498454 | 0.546875537 |
| ZCO537_02 | Benign | 0.479134102 | 0.488148643 | 0.544376163 | 0.535850651 |
| ZCO362_02 | Benign | 0.444009721 | 0.505752707 | 0.492502088 | 0.477235573 |
| ZCO488_02 | Benign | 0.444544763 | 0.519100176 | 0.540363647 | 0.476375639 |
| PC_03 | | 0.4601642 | 0.50258403 | 0.535348062 | 0.477717507 |
| ZCO535_02 | Benign | 0.437123899 | 0.45220741 | 0.450781955 | 0.47921145 |
| ZCO443_02 | Cancer | 0.340905903 | 0.3964135 | 0.408159712 | 0.375341658 |
| ZCO393_02 | Benign | 0.392115192 | 0.42285587 | 0.433317077 | 0.478271697 |
| ZCO503_02 | Cancer | 0.414083604 | 0.459524618 | 0.512173633 | 0.477236992 |
| ZCO438_02 | Cancer | 0.194579805 | 0.212248453 | 0.204323394 | 0.186087391 |
| ZCO406_02 | Benign | 0.368553069 | 0.388582605 | 0.428996038 | 0.405106449 |
| PC_04 | | 0.452066692 | 0.548488675 | 0.487692163 | 0.506700956 |
| PC_01 | | 0.473200498 | 0.56809841 | 0.55406269 | 0.564363566 |
| 00082_07 | Cancer | 0.386187751 | 0.420653163 | 0.445243176 | 0.42607336 |
| 02286_07 | Benign | 0.414915303 | 0.50287086 | 0.518923987 | 0.503674295 |
| 02280_06 | Cancer | 0.424805063 | 0.450352865 | 0.463086207 | 0.460293614 |
| 01123_06 | Benign | 0.559501125 | 0.633757057 | 0.616080873 | 0.661784062 |
| 00156_07 | Cancer | 0.222469259 | 0.28503586 | 0.27574027 | 0.260910541 |
| 00781_09 | Benign | 0.448771145 | 0.5304434 | 0.534545544 | 0.501334687 |
| 00539_08 | Cancer | 0.638668681 | 0.672223157 | 0.701812384 | 0.718042326 |
| 02241_07 | Cancer | 0.619561872 | 0.640561366 | 0.670091384 | 0.631696524 |
| 02226_05 | Benign | 0.377293235 | 0.413488006 | 0.370716448 | 0.40331382 |
| PC_03 | | 0.516530587 | 0.569289744 | 0.614636777 | 0.633929133 |
| 00542_08 | NA | 0.361556963 | 0.402800607 | 0.444191661 | 0.376767946 |
| 02497_10 | NA | 0.443549893 | 0.497099087 | 0.53199765 | 0.480236775 |
| 02224_05 | Benign | 0.41844047 | 0.53371495 | 0.499271682 | 0.494468044 |
| 00748_09 | Cancer | 0.357350016 | 0.420271276 | 0.41150019 | 0.42306665 |
| 03630_09 | Benign | 0.441634251 | 0.459741664 | 0.5179871 | 0.512272436 |
| 02279_07 | Cancer | 0.465548477 | 0.441129255 | 0.538369076 | 0.523602757 |
| PC_04 | | 0.519773303 | 0.479353267 | 0.524131518 | 0.538350952 |
| PC_01 | | 0.539686023 | 0.539112862 | 0.542974643 | 0.561181104 |
| NYU806 | Benign | 0.367140129 | 0.385414699 | 0.378598904 | 0.435744729 |
| NYU777 | Cancer | 0.432315925 | 0.515451875 | 0.494591864 | 0.541002277 |
| NYU176 | Benign | 0.427771172 | 0.456555363 | 0.475645565 | 0.46324018 |
| NYU888 | Cancer | 0.491868465 | 0.536135948 | 0.549561599 | 0.556075535 |
| NYU1117 | Benign | 0.469580468 | 0.460944911 | 0.505952082 | 0.537708242 |
| NYU1201 | Cancer | 0.397994925 | 0.476088676 | 0.490172618 | 0.451025721 |
| PC_02 | | 0.453300715 | 0.549556397 | 0.534580335 | 0.5254141 |
| NYU887 | Cancer | 0.379263411 | 0.39500895 | 0.412319446 | 0.402171783 |
| NYU815 | Benign | 0.422318543 | 0.472109772 | 0.501296351 | 0.491571943 |
| NYU927 | Cancer | 0.45918252 | 0.519782815 | 0.549628671 | 0.538270156 |
| NYU1030 | Benign | 0.471423543 | 0.499487118 | 0.520700004 | 0.507518824 |
| NYU1151 | Cancer | 0.309717053 | 0.395665111 | 0.316980095 | 0.338919958 |
| NYU1005 | Benign | 0.416175563 | 0.505086184 | 0.468979894 | 0.489515837 |
| NYU522 | Benign | 0.511811269 | 0.613797414 | 0.664364981 | 0.621353055 |
| NYU389 | Cancer | 0.414186206 | 0.445788863 | 0.415405634 | 0.460854079 |
| PC_03 | | 0.484115037 | 0.531826075 | 0.594038127 | 0.532518503 |
| NYU729 | Cancer | 0.250642721 | 0.249039614 | 0.271026177 | 0.291734624 |
| NYU430 | Benign | 0.456839862 | 0.586750677 | 0.553736087 | 0.55722498 |
| NYU144 | Benign | 0.391207165 | 0.407449865 | 0.424726188 | 0.43826024 |
| NYU256 | Cancer | 0.323214707 | 0.395300487 | 0.369736486 | 0.410786943 |
| NYU1000 | Benign | 0.447333034 | 0.683863969 | 0.568104523 | 0.590875857 |
| NYU575 | Cancer | 0.408082014 | 0.447958234 | 0.464701159 | 0.479207455 |
| PC_04 | | 0.539401312 | 0.566074489 | 0.635465994 | 0.597174964 |

TABLE 11I
PV2 fidelity small nodule batch all transitions (normalized)

| msfile | | TGVITSPDFPNPYPK_816.92_1074.50 | TGVITSPDFPNPYPK_816.92_1262.60 | TGVITSPDFPNPYPK_816.92_258.10 |
|---|---|---|---|---|
| name | status | (SEQ ID No.: 65) | (SEQ ID No.: 65) | (SEQ ID No.: 65) |
| PC_01 | | 0.274942325 | 0.294434025 | 0.387930241 |
| ZCO489_02 | Benign | 0.386416729 | 0.626207929 | 0.501054517 |
| ZCO436_02 | Cancer | 0.256214405 | 0.238533793 | 0.379176506 |
| ZCO512_02 | Cancer | 0.294530407 | 0.294426257 | 0.398279662 |
| ZCO475_02 | Benign | 0.398478031 | 0.358046576 | 0.508910412 |
| ZCO485_02 | Benign | 0.371589119 | 0.369424981 | 0.539966001 |
| ZCO536_02 | Cancer | 0.42064913 | 0.419273049 | 0.588831894 |
| PC_02 | | 0.250479047 | 0.271549936 | 0.35564938 |
| ZCO496_02 | Benign | 0.247057402 | 0.235194327 | 0.313896305 |
| ZCO502_02 | Cancer | 0.235372347 | 0.218117777 | 0.339417409 |
| ZCO382_02 | Benign | 0.288320382 | 0.274472937 | 0.383660241 |
| ZCO431_02 | Cancer | 0.338365328 | 0.352936816 | 0.461338239 |
| ZCO449_02 | Cancer | 0.394296564 | 0.371508169 | 0.506913954 |
| ZCO537_02 | Benign | 0.407926871 | 0.392877144 | 0.454410291 |
| ZCO362_02 | Benign | 0.224967335 | 0.236613958 | 0.326314227 |
| ZCO488_02 | Benign | 0.325465266 | 0.340313629 | 0.393393161 |
| PC_03 | | 0.281686659 | 0.300252735 | 0.368562549 |
| ZCO535_02 | Benign | 0.314821685 | 0.296415482 | 0.430263193 |
| ZCO443_02 | Cancer | 0.301254797 | 0.300093448 | 0.731197366 |
| ZCO393_02 | Benign | NA | NA | 0.434736779 |
| ZCO503_02 | Cancer | 0.373432468 | 0.648704079 | 0.414309406 |
| ZCO438_02 | Cancer | 0.299909745 | 0.271515844 | 0.37081918 |
| ZCO406_02 | Benign | 0.424586271 | 0.405393241 | 0.634224495 |
| PC_04 | | 0.260166337 | 0.262808361 | 0.370212505 |
| PC_01 | | 0.269237828 | 0.229901491 | 0.361821993 |
| 00082_07 | Cancer | 0.271889389 | 0.169400118 | 0.351018965 |
| 02286_07 | Benign | 0.342387798 | 0.339098552 | 0.372671351 |
| 02280_06 | Cancer | NA | 0.341880353 | 0.451177221 |
| 01123_06 | Benign | 0.110246757 | 0.317727626 | 0.384694739 |
| 00156_07 | Cancer | NA | 0.144682654 | 0.382674384 |
| 00781_09 | Benign | 0.435910306 | 0.457321138 | 0.484450881 |
| 00539_08 | Cancer | 0.159905152 | NA | 0.387482384 |
| 02241_07 | Cancer | 0.312441811 | 0.301791081 | 0.359303316 |
| 02226_05 | Benign | 0.441313783 | 0.868397059 | 0.511441537 |
| PC_03 | | NA | 0.403048829 | 0.352386088 |
| 00542_08 | NA | 0.211511543 | 0.33474463 | 0.40699555 |
| 02497_10 | NA | 0.324734355 | 0.287418813 | 0.360615786 |
| 02224_05 | Benign | 0.364170512 | 0.342104686 | 0.400828695 |
| 00748_09 | Cancer | 0.291765728 | 0.118473046 | 0.360062767 |
| 03630_09 | Benign | 0.30558686 | 0.377471463 | 0.430549832 |
| 02279_07 | Cancer | 0.275606233 | 0.268953939 | 0.385835855 |
| PC_04 | | 0.28451702 | 0.253391103 | 0.334325556 |
| PC_01 | | 0.179074421 | 0.255269705 | 0.348735991 |
| NYU806 | Benign | 0.354115392 | 0.311176075 | 0.383427748 |
| NYU777 | Cancer | 0.391369958 | 0.394751741 | 0.448114978 |
| NYU176 | Benign | 0.29733621 | 0.28945936 | 0.375507764 |
| NYU888 | Cancer | 0.152479442 | 0.105784247 | 0.272851073 |
| NYU1117 | Benign | 0.009857224 | NA | 0.535764706 |
| NYU1201 | Cancer | 0.345591222 | 0.297905848 | 0.364715477 |
| PC_02 | | 0.254475647 | 0.222636788 | 0.310394161 |
| NYU887 | Cancer | 0.331242414 | 0.312771673 | 0.444586416 |
| NYU815 | Benign | 0.380961767 | 0.36706044 | 0.472542798 |
| NYU927 | Cancer | 0.337624251 | 0.295033468 | 0.378088454 |
| NYU1030 | Benign | 0.141167687 | NA | 0.305936373 |
| NYU1151 | Cancer | 0.225543382 | 0.300765011 | 0.410540494 |
| NYU1005 | Benign | NA | 0.341386695 | 0.430532246 |
| NYU522 | Benign | 0.166721136 | 0.284336439 | 0.34459966 |
| NYU389 | Cancer | 0.286538993 | 0.5812878 | 0.373990992 |
| PC_03 | | NA | NA | 0.349242226 |
| NYU729 | Cancer | 188.9129305 | NA | 2.446036131 |
| NYU430 | Benign | 0.225122985 | 0.215164926 | 0.305350214 |
| NYU144 | Benign | 0.266119432 | 0.29426018 | 0.36226741 |
| NYU256 | Cancer | 0.401227067 | 0.35551106 | 0.472762458 |
| NYU1000 | Benign | 0.260179967 | 0.269792107 | 0.333538057 |
| NYU575 | Cancer | 0.287601789 | 0.297853282 | 0.368399783 |
| PC_04 | | 0.162856409 | 0.093679005 | 0.340183007 |

| msfile | | TGVITSPDFPNPYPK_816.92_715.40 | TVLWPNGLSLDIPAGR_855.00_1209.70 |
|---|---|---|---|
| name | status | (SEQ ID No.: 65) | (SEQ ID No.: 57) |
| PC_01 | | 0.313687198 | 0.024336736 |
| ZCO489_02 | Benign | 0.371098896 | 0.030724537 |
| ZCO436_02 | Cancer | 0.266504853 | 0.018384378 |
| ZCO512_02 | Cancer | 0.358204735 | 0.021708138 |
| ZCO475_02 | Benign | 0.26541615 | 0.025521114 |
| ZCO485_02 | Benign | 0.431162086 | 0.038315684 |
| ZCO536_02 | Cancer | 0.461656539 | 0.040891397 |
| PC_02 | | 0.235946775 | 0.028548975 |
| ZCO496_02 | Benign | 0.262914251 | 0.027488396 |
| ZCO502_02 | Cancer | 0.226621528 | 0.029143645 |
| ZCO382_02 | Benign | 0.265533031 | 0.016356725 |
| ZCO431_02 | Cancer | 0.265005494 | 0.02057335 |
| ZCO449_02 | Cancer | 0.321697994 | 0.024290384 |
| ZCO537_02 | Benign | 0.30543116 | 0.036165076 |
| ZCO362_02 | Benign | 0.282540989 | 0.013297179 |
| ZCO488_02 | Benign | 0.37464508 | 0.027232478 |
| PC_03 | | 0.299836932 | 0.020669493 |
| ZCO535_02 | Benign | 0.343588009 | 0.029806443 |
| ZCO443_02 | Cancer | 0.43423048 | 0.035262216 |
| ZCO393_02 | Benign | 0.683122563 | 0.017875412 |
| ZCO503_02 | Cancer | 0.395550935 | 0.029086331 |
| ZCO438_02 | Cancer | 0.311859041 | 0.025619734 |
| ZCO406_02 | Benign | 0.445189924 | 0.01565807 |
| PC_04 | | 0.295760605 | 0.024960581 |
| PC_01 | | 0.171396503 | 0.027587383 |
| 00082_07 | Cancer | 0.243442138 | 0.035291209 |
| 02286_07 | Benign | 0.384797518 | 0.035251538 |
| 02280_06 | Cancer | 0.562098083 | 0.042219407 |
| 01123_06 | Benign | 0.334317053 | 0.037976025 |
| 00156_07 | Cancer | 0.345232238 | 0.034744807 |
| 00781_09 | Benign | 0.56079471 | 0.038714715 |
| 00539_08 | Cancer | 0.313817246 | 0.041870064 |
| 02241_07 | Cancer | 0.35952093 | 0.034253706 |
| 02226_05 | Benign | NA | 0.041345393 |
| PC_03 | | NA | 0.02956282 |
| 00542_08 | NA | 0.210725786 | 0.022512195 |
| 02497_10 | NA | 0.299669722 | 0.030004135 |
| 02224_05 | Benign | 0.376310491 | 0.0375988 |
| 00748_09 | Cancer | 0.209003788 | 0.034204408 |
| 03630_09 | Benign | 0.345131469 | 0.039758117 |
| 02279_07 | Cancer | 0.295009079 | 0.035600185 |
| PC_04 | | 0.307951309 | 0.029784484 |
| PC_01 | | 0.334797481 | 0.024953814 |
| NYU806 | Benign | 0.379057127 | 0.03450794 |
| NYU777 | Cancer | 0.443179443 | 0.030415492 |
| NYU176 | Benign | 0.269008356 | 0.03482741 |
| NYU888 | Cancer | 0.118100384 | 0.038536869 |
| NYU1117 | Benign | 0.26814854 | 0.02996094 |
| NYU1201 | Cancer | 0.302932311 | 0.039543512 |
| PC_02 | | 0.310900525 | 0.020758159 |
| NYU887 | Cancer | 0.351647055 | 0.035737934 |
| NYU815 | Benign | 0.462586234 | 0.033047805 |
| NYU927 | Cancer | 0.178548639 | 0.033866408 |
| NYU1030 | Benign | 0.293286713 | 0.032621811 |
| NYU1151 | Cancer | 0.38613866 | 0.043754435 |
| NYU1005 | Benign | 0.243445821 | 0.025601405 |
| NYU522 | Benign | NA | 0.024872068 |
| NYU389 | Cancer | 0.134764361 | 0.040505087 |
| PC_03 | | 0.767152277 | 0.025799004 |
| NYU729 | Cancer | 31.91482133 | 0.042179563 |
| NYU430 | Benign | 0.254280558 | 0.02314015 |
| NYU144 | Benign | 0.32543046 | 0.048520132 |
| NYU256 | Cancer | 0.407163807 | 0.044367501 |
| NYU1000 | Benign | 0.29270535 | 0.053924113 |
| NYU575 | Cancer | 0.315319686 | 0.025332753 |
| PC_04 | | 0.282632139 | 0.026554915 |

| msfile | | TVLWPNGLSLDIPAGR_855.00_314.20 | TVLWPNGLSLDIPAGR_855.00_400.20 |
|---|---|---|---|
| name | status | (SEQ ID No.: 57) | (SEQ ID No.: 57) |
| PC_01 | | 0.004405061 | 0.018903818 |
| ZCO489_02 | Benign | 0.020188871 | 0.024343008 |
| ZCO436_02 | Cancer | 0.030142371 | NA |
| ZCO512_02 | Cancer | 0.022366049 | 0.026938002 |
| ZCO475_02 | Benign | 0.019521698 | 0.028238463 |
| ZCO485_02 | Benign | 0.030439696 | 0.050718775 |
| ZCO536_02 | Cancer | 0.0512681 | 0.056127472 |
| PC_02 | | 0.031093864 | 0.037142523 |
| ZCO496_02 | Benign | NA | 0.057391568 |
| ZCO502_02 | Cancer | 0.036157447 | 0.017131107 |
| ZCO382_02 | Benign | 0.022633925 | NA |
| ZCO431_02 | Cancer | 0.03103499 | 0.025604178 |
| ZCO449_02 | Cancer | 0.087903137 | 0.020199955 |
| ZCO537_02 | Benign | 0.046046417 | 0.02836914 |
| ZCO362_02 | Benign | 0.016169716 | 0.015008629 |
| ZCO488_02 | Benign | 0.0348481 | 0.025812051 |
| PC_03 | | 0.022183943 | 0.034050735 |
| ZCO535_02 | Benign | 0.044226956 | 0.029604696 |
| ZCO443_02 | Cancer | 0.051800587 | 0.054985515 |
| ZCO393_02 | Benign | 0.010117057 | NA |
| ZCO503_02 | Cancer | 0.039002351 | 0.034072094 |
| ZCO438_02 | Cancer | 0.039387595 | 0.040000096 |
| ZCO406_02 | Benign | NA | 0.029358732 |
| PC_04 | | 0.021816709 | 0.025974063 |
| PC_01 | | 0.032274353 | 0.036487102 |
| 00082_07 | Cancer | NA | 0.028929264 |
| 02286_07 | Benign | 0.050482999 | 0.059588946 |
| 02280_06 | Cancer | NA | 0.053574065 |
| 01123_06 | Benign | 0.04381684 | 0.037637823 |
| 00156_07 | Cancer | 0.033160086 | 0.045619499 |
| 00781_09 | Benign | 0.052359125 | 0.029004833 |
| 00539_08 | Cancer | 0.070653372 | 0.040619409 |
| 02241_07 | Cancer | 0.0679639 | 0.055322878 |
| 02226_05 | Benign | 0.039973049 | NA |
| PC_03 | | 0.023612405 | NA |
| 00542_08 | NA | 0.036117363 | 0.019938154 |
| 02497_10 | NA | 0.028728405 | 0.033636684 |
| 02224_05 | Benign | 0.029557414 | 0.038045333 |
| 00748_09 | Cancer | 0.006332442 | 0.038673519 |
| 03630_09 | Benign | 0.060559766 | 0.077657132 |
| 02279_07 | Cancer | NA | NA |
| PC_04 | | NA | NA |
| PC_01 | | 0.036430224 | 0.028147418 |
| NYU806 | Benign | 0.031911416 | 0.032128348 |
| NYU777 | Cancer | 0.043492829 | 0.033863252 |
| NYU176 | Benign | 0.047885278 | 0.038998429 |
| NYU888 | Cancer | 0.064154626 | 0.048527679 |
| NYU1117 | Benign | NA | 0.035450915 |
| NYU1201 | Cancer | 0.030107866 | 0.035881627 |
| PC_02 | | 0.029728346 | 0.035395008 |
| NYU887 | Cancer | 0.057892629 | 0.05433076 |
| NYU815 | Benign | 0.038626192 | 0.033774771 |
| NYU927 | Cancer | 0.067994965 | 0.048759907 |
| NYU1030 | Benign | 0.035739927 | 0.042833442 |
| NYU1151 | Cancer | 0.038630057 | 0.042289067 |
| NYU1005 | Benign | 0.03367156 | 0.052821592 |
| NYU522 | Benign | 0.039452562 | 0.053163757 |
| NYU389 | Cancer | 0.02963033 | 0.075064151 |
| PC_03 | | NA | 0.02272884 |
| NYU729 | Cancer | 0.086885145 | 0.076657619 |
| NYU430 | Benign | 0.032346816 | 0.038309358 |
| NYU144 | Benign | 0.051476553 | 0.04634643 |
| NYU256 | Cancer | 0.065822926 | 0.058352679 |
| NYU1000 | Benign | 0.031385597 | 0.08732303 |
| NYU575 | Cancer | 0.010537921 | NA |
| PC_04 | | 0.036324242 | 0.027321479 |

TABLE 11J
PV2 fidelity small nodule batch all transitions (normalized)

| msfile | | TVLWPNGLSLDIPAGR_855.00_500.30 | TVLWPNGLSLDIPAGR_855.00_605.30 | TWNDPSVQQDIK_715.85_260.20 |
|---|---|---|---|---|
| name | status | (SEQ ID No.: 57) | (SEQ ID No.: 57) | (SEQ ID No.: 52) |
| PC_01 | | NA | NA | 1.431903408 |
| ZCO489_02 | Benign | 0.032768233 | 0.017381381 | 1.58801347 |
| ZCO436_02 | Cancer | 0.033327029 | 0.006057702 | 1.324048724 |
| ZCO512_02 | Cancer | NA | NA | 1.152959285 |
| ZCO475_02 | Benign | 0.032461592 | 0.033063459 | 1.610438625 |
| ZCO485_02 | Benign | NA | 0.02460675 | 1.124556038 |
| ZCO536_02 | Cancer | NA | 0.034277568 | 1.411509416 |
| PC_02 | | 0.055681256 | 0.00619548 | 0.898966232 |
| ZCO496_02 | Benign | 0.02368928 | 0.022827869 | 0.816839613 |
| ZCO502_02 | Cancer | 0.024526155 | 0.035814327 | 3.180027781 |
| ZCO382_02 | Benign | 0.023522618 | NA | 0.879197674 |
| ZCO431_02 | Cancer | 0.040257438 | 0.022398652 | 1.335724674 |
| ZCO449_02 | Cancer | NA | 0.027360641 | 1.553362142 |
| ZCO537_02 | Benign | 0.034240123 | 0.026326642 | 1.098547556 |
| ZCO362_02 | Benign | 0.029014186 | 0.008489188 | 0.960763956 |
| ZCO488_02 | Benign | 0.050166347 | 0.024930029 | 1.544485913 |
| PC_03 | | NA | 0.026464348 | 1.267072453 |
| ZCO535_02 | Benign | 0.043347525 | 0.016932441 | 1.367276955 |
| ZCO443_02 | Cancer | 0.064243681 | 0.038708433 | 2.382940846 |
| ZCO393_02 | Benign | NA | 0.032521166 | 0.773444302 |
| ZCO503_02 | Cancer | 0.064533269 | 0.03277381 | 1.461371297 |
| ZCO438_02 | Cancer | NA | 0.028588252 | 1.257666275 |
| ZCO406_02 | Benign | NA | NA | 0.747632906 |
| PC_04 | | NA | 0.016602949 | 0.977901906 |
| PC_01 | | 0.022354436 | 0.031801844 | 1.296744613 |
| 00082_07 | Cancer | 0.005115966 | 0.04115921 | 0.556674419 |
| 02286_07 | Benign | 0.031180377 | 0.032771211 | 0.887260669 |
| 02280_06 | Cancer | 0.060077968 | 0.022812592 | 1.047316412 |
| 01123_06 | Benign | 0.043141283 | 0.04993089 | 0.884118243 |
| 00156_07 | Cancer | 0.034406653 | 0.035235544 | 0.596498487 |
| 00781_09 | Benign | 0.054855309 | 0.042196629 | 0.774301555 |
| 00539_08 | Cancer | 0.073685292 | 0.039008317 | 0.687864216 |
| 02241_07 | Cancer | 0.036098514 | 0.049638813 | 0.909111326 |
| 02226_05 | Benign | 0.029001066 | 0.053516623 | 0.890796972 |
| PC_03 | | NA | 0.026852498 | 1.073338427 |
| 00542_08 | NA | 0.035322097 | 0.026561735 | 0.780540076 |
| 02497_10 | NA | 0.044647722 | 0.018162496 | 0.75814843 |
| 02224_05 | Benign | 0.043768793 | 0.036842522 | 0.752606752 |
| 00748_09 | Cancer | NA | 0.03033514 | 0.843318354 |
| 03630_09 | Benign | 0.032350385 | 0.068506881 | 1.344495278 |
| 02279_07 | Cancer | NA | 0.016664633 | 0.61981917 |
| PC_04 | | 0.030441887 | 0.013355459 | 1.386708523 |
| PC_01 | | NA | 0.026246666 | 0.824261833 |
| NYU806 | Benign | 0.046587191 | 0.030862468 | 1.006653335 |
| NYU777 | Cancer | 0.037240957 | 0.029535584 | 1.153690221 |
| NYU176 | Benign | 0.057959556 | 0.026336581 | 1.061589892 |
| NYU888 | Cancer | 0.045696689 | 0.04217951 | 0.826180628 |
| NYU1117 | Benign | 0.03475556 | 0.022284065 | 1.583108294 |
| NYU1201 | Cancer | 0.050755841 | 0.039254029 | 1.148191141 |
| PC_02 | | 0.047725115 | 0.038872326 | 1.141574092 |
| NYU887 | Cancer | 0.073531978 | 0.029004875 | 0.9833617 |
| NYU815 | Benign | 0.014877039 | 0.03952594 | 0.96206858 |
| NYU927 | Cancer | 0.03417933 | 0.037821103 | 1.195016343 |
| NYU1030 | Benign | 0.050782936 | 0.049033676 | 0.717955583 |
| NYU1151 | Cancer | 0.033858435 | 0.032220451 | 1.952928065 |
| NYU1005 | Benign | NA | 0.038472686 | 0.789668266 |
| NYU522 | Benign | 0.044262094 | 0.023393883 | 0.588226663 |
| NYU389 | Cancer | 0.062971013 | 0.028160916 | 1.108065605 |
| PC_03 | | NA | 0.017757676 | 0.95582342 |
| NYU729 | Cancer | 0.041936541 | 0.032908147 | 1.016450994 |
| NYU430 | Benign | 0.043800851 | 0.034487131 | 0.823089982 |
| NYU144 | Benign | 0.060358985 | 0.060337695 | 0.972611329 |
| NYU256 | Cancer | 0.047050695 | 0.046100103 | 0.808311067 |
| NYU1000 | Benign | 0.019003724 | 0.037718253 | 1.112966041 |
| NYU575 | Cancer | NA | NA | 2.468006181 |
| PC_04 | | 0.057143891 | 0.035579405 | 1.029190185 |

| msfile | | TWNDPSVQQDIK_715.85_288.10 | TWNDPSVQQDIK_715.85_517.20 |
|---|---|---|---|
| name | status | (SEQ ID No.: 52) | (SEQ ID No.: 52) |
| PC_01 | | 0.159508385 | 0.136449648 |
| ZCO489_02 | Benign | 0.203082548 | 0.171495068 |
| ZCO436_02 | Cancer | 0.146439347 | 0.128478471 |
| ZCO512_02 | Cancer | 0.154932207 | 0.153812406 |
| ZCO475_02 | Benign | 0.137142298 | 0.127853924 |
| ZCO485_02 | Benign | 0.113837413 | 0.119515441 |
| ZCO536_02 | Cancer | 0.137588909 | 0.135466039 |
| PC_02 | | 0.157143658 | 0.123823278 |
| ZCO496_02 | Benign | 0.114910288 | 0.085520429 |
| ZCO502_02 | Cancer | 0.306742678 | 0.288319622 |
| ZCO382_02 | Benign | 0.087568762 | 0.058279827 |
| ZCO431_02 | Cancer | 0.195121128 | 0.145245409 |
| ZCO449_02 | Cancer | 0.171061408 | 0.183535337 |
| ZCO537_02 | Benign | 0.171410034 | 0.122329689 |
| ZCO362_02 | Benign | 0.083159378 | 0.056761611 |
| ZCO488_02 | Benign | 0.1644119 | 0.130309094 |
| PC_03 | | 0.138832096 | 0.147086169 |
| ZCO535_02 | Benign | 0.122018368 | 0.111726563 |
| ZCO443_02 | Cancer | 0.250523788 | 0.285774724 |
| ZCO393_02 | Benign | 0.084591376 | 0.080611799 |
| ZCO503_02 | Cancer | 0.203288154 | 0.158134472 |
| ZCO438_02 | Cancer | 0.21461978 | 0.150603439 |
| ZCO406_02 | Benign | 0.117915629 | 0.080983514 |
| PC_04 | | 0.157668644 | 0.15171547 |
| PC_01 | | 0.139901055 | 0.128664698 |
| 00082_07 | Cancer | 0.069552065 | 0.09987679 |
| 02286_07 | Benign | 0.119751979 | 0.094897211 |
| 02280_06 | Cancer | 0.093923656 | 0.093174733 |
| 01123_06 | Benign | 0.105327229 | 0.107991239 |
| 00156_07 | Cancer | 0.106914499 | 0.110971164 |
| 00781_09 | Benign | 0.113291451 | 0.127026439 |
| 00539_08 | Cancer | 0.084377848 | 0.079281685 |
| 02241_07 | Cancer | 0.095826464 | 0.096461076 |
| 02226_05 | Benign | 0.100096976 | 0.116388954 |
| PC_03 | | 0.142428812 | 0.167108259 |
| 00542_08 | NA | 0.108560935 | 0.112037073 |
| 02497_10 | NA | 0.121168161 | 0.100227082 |
| 02224_05 | Benign | 0.098550753 | 0.0850397 |
| 00748_09 | Cancer | 0.080283103 | 0.088930171 |
| 03630_09 | Benign | 0.132056136 | 0.131044715 |
| 02279_07 | Cancer | 0.107048786 | 0.133370037 |
| PC_04 | | 0.166816338 | 0.179129202 |
| PC_01 | | 0.140624602 | 0.127492748 |
| NYU806 | Benign | 0.096422046 | 0.12257464 |
| NYU777 | Cancer | 0.138871673 | 0.163273881 |
| NYU176 | Benign | 0.144667548 | 0.09856075 |
| NYU888 | Cancer | 0.106164465 | 0.1087592 |
| NYU1117 | Benign | 0.127488087 | 0.126473558 |
| NYU1201 | Cancer | 0.088929521 | 0.084445045 |
| PC_02 | | 0.14044024 | 0.135675817 |
| NYU887 | Cancer | 0.140107376 | 0.149468224 |
| NYU815 | Benign | 0.158798007 | 0.141830461 |
| NYU927 | Cancer | 0.151589691 | 0.135165428 |
| NYU1030 | Benign | 0.109893573 | 0.129170623 |
| NYU1151 | Cancer | 0.130419567 | 0.125009038 |
| NYU1005 | Benign | 0.097062021 | 0.086112499 |
| NYU522 | Benign | 0.100761719 | 0.103285489 |
| NYU389 | Cancer | 0.139484872 | 0.134244456 |
| PC_03 | | 0.14337368 | 0.174242889 |
| NYU729 | Cancer | 0.156901766 | 0.179138126 |
| NYU430 | Benign | 0.107745392 | 0.107921576 |
| NYU144 | Benign | 0.161754986 | 0.174846247 |
| NYU256 | Cancer | 0.11187711 | 0.103320604 |
| NYU1000 | Benign | 0.120476712 | 0.136708805 |
| NYU575 | Cancer | 0.234080377 | 0.296528899 |
| PC_04 | | 0.157074531 | 0.142064389 |

| | | | VE- |
|---|---|---|---|
| msfile | | TWNDPSVQQDIK_715.85_914.50 | IFYR_413.73_229.10 |
| name | status | (SEQ ID No.: 52) | (SEQ ID No.: 56) |
| PC_01 | | 0.1626744 | 1.14431003 |
| ZCO489_02 | Benign | 0.187624893 | 0.680456408 |
| ZCO436_02 | Cancer | 0.135521211 | 1.636530042 |
| ZCO512_02 | Cancer | 0.172954348 | 0.92035874 |
| ZCO475_02 | Benign | 0.147028685 | 0.851729773 |
| ZCO485_02 | Benign | 0.122123477 | 2.038086987 |
| ZCO536_02 | Cancer | 0.217203897 | 1.129859348 |
| PC_02 | | 0.156149336 | 1.040080248 |
| ZCO496_02 | Benign | 0.118551925 | 3.751246344 |
| ZCO502_02 | Cancer | 0.362396231 | 1.492958157 |
| ZCO382_02 | Benign | 0.104640216 | 0.594565781 |
| ZCO431_02 | Cancer | 0.181215759 | 1.517575792 |
| ZCO449_02 | Cancer | 0.159130008 | 1.345287181 |
| ZCO537_02 | Benign | 0.198279164 | 1.565575261 |
| ZCO362_02 | Benign | 0.09264149 | 1.014367724 |
| ZCO488_02 | Benign | 0.164970453 | 2.095690582 |
| PC_03 | | 0.157764883 | 1.174970344 |
| ZCO535_02 | Benign | 0.15866715 | 1.402927976 |
| ZCO443_02 | Cancer | 0.294188957 | 1.215937498 |
| ZCO393_02 | Benign | 0.100847311 | 1.62026608 |
| ZCO503_02 | Cancer | 0.20485771 | 0.520895347 |
| ZCO438_02 | Cancer | 0.187281018 | 1.652941497 |
| ZCO406_02 | Benign | 0.109616928 | 1.688045592 |
| PC_04 | | 0.180948798 | 1.272460333 |
| PC_01 | | 0.163998599 | 1.128288775 |
| 00082_07 | Cancer | 0.084713163 | 1.512335242 |
| 02286_07 | Benign | 0.104814804 | 1.23740708 |
| 02280_06 | Cancer | 0.118689466 | 0.866126573 |
| 01123_06 | Benign | 0.150686334 | 0.522060265 |
| 00156_07 | Cancer | 0.117791267 | 1.45768743 |
| 00781_09 | Benign | 0.114892589 | 2.033642232 |
| 00539_08 | Cancer | 0.109567893 | 0.419795436 |
| 02241_07 | Cancer | 0.095350954 | 0.772844815 |
| 02226_05 | Benign | 0.126430566 | 3.113030846 |
| PC_03 | | 0.180313014 | 1.352227667 |
| 00542_08 | NA | 0.136881101 | 1.327838444 |
| 02497_10 | NA | 0.126117962 | 0.840551825 |
| 02224_05 | Benign | 0.110736604 | 0.981917018 |
| 00748_09 | Cancer | 0.116312645 | 0.798931973 |
| 03630_09 | Benign | 0.135264576 | 1.131381488 |
| 02279_07 | Cancer | 0.130522794 | 0.883709782 |
| PC_04 | | 0.171645746 | 1.17425384 |
| PC_01 | | 0.151945903 | 1.417275665 |
| NYU806 | Benign | 0.128097546 | 1.065481691 |
| NYU777 | Cancer | 0.172098834 | 1.518115332 |
| NYU176 | Benign | 0.155474411 | 1.83548066 |
| NYU888 | Cancer | 0.109537749 | 0.451284206 |
| NYU1117 | Benign | 0.165043904 | 1.107641756 |
| NYU1201 | Cancer | 0.11557104 | 0.768532339 |
| PC_02 | | 0.166048121 | 1.306269488 |
| NYU887 | Cancer | 0.161387654 | 0.926291687 |
| NYU815 | Benign | 0.177546434 | 1.170400778 |
| NYU927 | Cancer | 0.155104027 | 1.206735576 |
| NYU1030 | Benign | 0.130198065 | 1.697910607 |
| NYU1151 | Cancer | 0.140722493 | 0.735688854 |
| NYU1005 | Benign | 0.102829843 | 1.484373449 |
| NYU522 | Benign | 0.106899011 | 1.014289009 |
| NYU389 | Cancer | 0.119884021 | 1.596331539 |
| PC_03 | | 0.183477297 | 1.24701396 |
| NYU729 | Cancer | 0.195560472 | 2.262741438 |
| NYU430 | Benign | 0.099577064 | 2.071945023 |
| NYU144 | Benign | 0.215428096 | 1.179988966 |
| NYU256 | Cancer | 0.136216123 | 4.825963672 |
| NYU1000 | Benign | 0.153050141 | 1.165044485 |
| NYU575 | Cancer | 0.306730177 | 1.003603908 |
| PC_04 | | 0.187014302 | 1.181503823 |

TABLE 11K
PV2 fidelity small nodule batch all transitions (normalized)

| | | VE- | VE- | VI- | VI- |
|---|---|---|---|---|---|
| msfile- | | IFYR_413.73_485.30 | IFYR_413.73_598.30 | TEPIPVSDLR_669.89_213.20 | TEPIPVSDLR_669.89_288.20 |
| name | status | (SEQ ID No.: 56) | (SEQ ID No.: 56) | (SEQ ID No.: 70) | (SEQ ID No.: 70) |
| PC_01 | | 1.185377324 | 0.981858931 | 0.190003007 | 0.25966457 |
| ZCO489_02 | Benign | 0.712626071 | 0.746480771 | 0.232402915 | 0.27204687 |
| ZCO436_02 | Cancer | 1.850286215 | 1.868160266 | 0.149900903 | 0.166522209 |
| ZCO512_02 | Cancer | 0.856488182 | 0.923872611 | 0.16644378 | 0.25845205 |
| ZCO475_02 | Benign | 0.898358414 | 0.761748845 | 0.168763285 | 0.290211213 |
| ZCO485_02 | Benign | 2.036007549 | 1.814700073 | 0.180990816 | 0.265238163 |
| ZCO536_02 | Cancer | 1.060640647 | 1.175600546 | 0.197634205 | 0.21798459 |
| PC_02 | | 1.124269825 | 1.112617961 | 0.226906043 | 0.242720238 |
| ZCO496_02 | Benign | 4.129676436 | 3.438994921 | 0.148362949 | 0.24041611 |
| ZCO502_02 | Cancer | 1.535259366 | 1.637869805 | 0.157797213 | 0.17752036 |
| ZCO382_02 | Benign | 0.597750095 | 0.561589112 | 0.245177878 | 0.260529191 |
| ZCO431_02 | Cancer | 1.700541394 | 1.439681904 | 0.215478475 | 0.30576355 |
| ZCO449_02 | Cancer | 1.522681093 | 1.337431203 | 0.182373129 | 0.268803293 |
| ZCO537_02 | Benign | 1.792135731 | 1.608554132 | 0.142502046 | 0.221916014 |
| ZCO362_02 | Benign | 1.101519075 | 1.13292218 | 0.235528851 | 0.313307943 |
| ZCO488_02 | Benign | 1.89436522 | 2.40769232 | 0.122809478 | 0.15700534 |
| PC_03 | | 1.32889926 | 1.241445296 | 0.224678286 | 0.324826177 |
| ZCO535_02 | Benign | 1.316682724 | 1.310266004 | 0.149422558 | 0.30535255 |
| ZCO443_02 | Cancer | 1.153436573 | 1.290910198 | 0.222961232 | 0.289913932 |
| ZCO393_02 | Benign | 1.905653312 | 1.669484623 | 0.196784562 | 0.22791578 |
| ZCO503_02 | Cancer | 0.589153419 | 0.697379349 | 0.200047494 | 0.242758097 |
| ZCO438_02 | Cancer | 2.065952169 | 1.973116233 | 0.139543137 | 0.182652086 |
| ZCO406_02 | Benign | 1.439305785 | 1.742586332 | 0.257647144 | 0.284332889 |
| PC_04 | | 1.360541053 | 1.168570432 | 0.251080937 | 0.263424697 |
| PC_01 | | 1.398987533 | 1.171265273 | 0.200016325 | 0.250442769 |
| 00082_07 | Cancer | 1.520781746 | 1.511805657 | 0.139981585 | 0.279124017 |
| 02286_07 | Benign | 1.14062114 | 1.130661549 | 0.144797272 | 0.233511592 |
| 02280_06 | Cancer | 0.907427212 | 0.967913982 | 0.168470642 | 0.221981398 |
| 01123_06 | Benign | 0.570016674 | 0.494505513 | 0.171706664 | 0.307454091 |
| 00156_07 | Cancer | 1.33968236 | 1.286636993 | 0.169913506 | 0.282137577 |
| 00781_09 | Benign | 1.950828074 | 1.804822859 | 0.213843438 | 0.287410603 |
| 00539_08 | Cancer | 0.504935567 | 0.427462056 | 0.143404061 | 0.212571932 |
| 02241_07 | Cancer | 0.735941086 | 0.887224928 | 0.143514642 | 0.182102531 |
| 02226_05 | Benign | 3.011680747 | 2.804493538 | 0.146853502 | 0.276757307 |
| PC_03 | | 1.374248304 | 1.342472871 | 0.190115554 | 0.299582872 |
| 00542_08 | NA | 1.567787376 | 1.165946835 | 0.130374377 | 0.249864043 |
| 02497_10 | NA | 0.96680498 | 0.824059576 | 0.183416628 | 0.285206309 |
| 02224_05 | Benign | 0.866238582 | 0.863121283 | 0.168091107 | 0.287435518 |
| 00748_09 | Cancer | 0.841028099 | 0.751929378 | 0.159579459 | 0.266164736 |
| 03630_09 | Benign | 1.096720873 | 1.142307729 | 0.186807642 | 0.254577079 |
| 02279_07 | Cancer | 0.990877363 | 1.030837596 | 0.150683937 | 0.166260562 |
| PC_04 | | 1.188880323 | 1.224428739 | 0.181077757 | 0.264466199 |
| PC_01 | | 1.201859363 | 1.204984716 | 0.183542749 | 0.27236094 |
| NYU806 | Benign | 1.319297217 | 1.126548468 | 0.154485606 | 0.196308372 |
| NYU777 | Cancer | 1.665413448 | 1.753075069 | 0.209778657 | 0.256587977 |
| NYU176 | Benign | 1.316721682 | 1.792309875 | 0.275362472 | 0.275986698 |
| NYU888 | Cancer | 0.520972757 | 0.466187434 | 0.17846081 | 0.181054252 |
| NYU1117 | Benign | 1.241527809 | 1.21103684 | 0.200531043 | 0.229404993 |
| NYU1201 | Cancer | 0.890630081 | 0.817265963 | 0.218825396 | 0.227318479 |
| PC_02 | | 1.08641051 | 1.066328425 | 0.185177428 | 0.32105068 |
| NYU887 | Cancer | 0.850988458 | 0.884561315 | 0.216721613 | 0.227745888 |
| NYU815 | Benign | 1.213231897 | 1.174591635 | 0.125908217 | 0.243008423 |
| NYU927 | Cancer | 1.305365531 | 1.214397986 | 0.197166351 | 0.197962027 |
| NYU1030 | Benign | 1.443085687 | 1.569928664 | 0.176064987 | 0.222752548 |
| NYU1151 | Cancer | 0.715569388 | 0.794433787 | 0.189354478 | 0.23121749 |
| NYU1005 | Benign | 1.549026026 | 1.313697733 | 0.168565879 | 0.291870758 |
| NYU522 | Benign | 1.113891827 | 1.118159874 | 0.206030443 | 0.279239333 |
| NYU389 | Cancer | 1.416389345 | 1.435622801 | 0.195633901 | 0.244875229 |
| PC_03 | | 1.192978593 | 1.250350366 | 0.210385442 | 0.30064569 |
| NYU729 | Cancer | 2.07378768 | 2.561895738 | 0.287604891 | 0.45415791 |
| NYU430 | Benign | 2.086481745 | 2.153346597 | 0.162537582 | 0.207581674 |
| NYU144 | Benign | 1.181593763 | 1.229662151 | 0.16717989 | 0.218159468 |
| NYU256 | Cancer | 4.22052202 | 4.623789602 | 0.219295124 | 0.289208632 |
| NYU1000 | Benign | 1.18148021 | 1.234725445 | 0.229656982 | 0.311369265 |
| NYU575 | Cancer | 1.083591249 | 1.092996549 | 0.204843217 | 0.275727736 |
| PC_04 | | 1.592847754 | 1.439432813 | 0.183753985 | 0.277644701 |

| | | VI- | VI- | VI- |
|---|---|---|---|---|
| msfile- | | TEPIPVSDLR_669.89_314.20 | TEPIPVSDLR_669.89_686.40 | TEPIPVSDLR_669.89_896.50 |
| name | status | (SEQ ID No.: 70) | (SEQ ID No.: 70) | (SEQ ID No.: 70) |
| PC_01 | | 0.357499248 | 0.267622659 | 0.272531408 |
| ZCO489_02 | Benign | 0.327782779 | 0.280660242 | 0.287890838 |
| ZCO436_02 | Cancer | 0.304207435 | 0.18247518 | 0.196154152 |
| ZCO512_02 | Cancer | 0.233810319 | 0.23039763 | 0.262613742 |
| ZCO475_02 | Benign | 0.328435196 | 0.310518428 | 0.294686929 |
| ZCO485_02 | Benign | 0.287665838 | 0.236209948 | 0.262468507 |
| ZCO536_02 | Cancer | 0.234134025 | 0.284448536 | 0.298101666 |
| PC_02 | | 0.352556629 | 0.245247532 | 0.282027647 |
| ZCO496_02 | Benign | 0.308126734 | 0.210612757 | 0.218125265 |
| ZCO502_02 | Cancer | 0.188717818 | 0.188431671 | 0.190637387 |
| ZCO382_02 | Benign | 0.192007059 | 0.237048314 | 0.305550968 |
| ZCO431_02 | Cancer | 0.372212504 | 0.303521987 | 0.28260926 |
| ZCO449_02 | Cancer | 0.359070904 | 0.255616456 | 0.274312144 |
| ZCO537_02 | Benign | 0.608758879 | 0.226235211 | 0.227670693 |
| ZCO362_02 | Benign | 0.247694323 | 0.290058625 | 0.32422178 |
| ZCO488_02 | Benign | 0.279327832 | 0.171211985 | 0.208423766 |
| PC_03 | | 0.343759023 | 0.270104581 | 0.268574543 |
| ZCO535_02 | Benign | 0.327639267 | 0.291454069 | 0.265510923 |
| ZCO443_02 | Cancer | 2.167526234 | 0.28484199 | 0.290477261 |
| ZCO393_02 | Benign | 0.248079948 | 0.222898026 | 0.261243132 |
| ZCO503_02 | Cancer | NA | 0.265700862 | 0.269431067 |
| ZCO438_02 | Cancer | 1.813051178 | 0.202208587 | 0.266848581 |
| ZCO406_02 | Benign | 0.333586168 | 0.233671278 | 0.298116427 |
| PC_04 | | 0.415046518 | 0.259607146 | 0.319749963 |
| PC_01 | | 0.383095398 | 0.253474122 | 0.27182786 |
| 00082_07 | Cancer | 2.69561362 | 0.23640644 | 0.281992587 |
| 02286_07 | Benign | 0.30606456 | 0.276234524 | 0.266034801 |
| 02280_06 | Cancer | 1.531808341 | 0.213330652 | 0.279100951 |
| 01123_06 | Benign | 0.468084905 | 0.278990125 | 0.307317197 |
| 00156_07 | Cancer | 1.495227978 | 0.223153476 | 0.25778124 |
| 00781_09 | Benign | 0.389860766 | 0.341771295 | 0.389240391 |
| 00539_08 | Cancer | 0.389647635 | 0.183607938 | 0.209358452 |
| 02241_07 | Cancer | 0.225750969 | 0.211038504 | 0.23297441 |
| 02226_05 | Benign | 1.225197522 | 0.173471722 | 0.237278541 |
| PC_03 | | 0.395224701 | 0.287198429 | 0.280946955 |
| 00542_08 | NA | 0.255178773 | 0.238476107 | 0.254855829 |
| 02497_10 | NA | 0.30310879 | 0.282016628 | 0.307746081 |
| 02224_05 | Benign | 0.504077494 | 0.299180971 | 0.319832891 |
| 00748_09 | Cancer | 0.29579502 | 0.266890564 | 0.320389228 |
| 03630_09 | Benign | 0.264855376 | 0.257279928 | 0.288096594 |
| 02279_07 | Cancer | 1.363162218 | 0.198176808 | 0.235840813 |
| PC_04 | | 0.458162777 | 0.267238337 | 0.29000516 |
| PC_01 | | 0.370439694 | 0.271508882 | 0.283738697 |
| NYU806 | Benign | 2.513216972 | 0.20597495 | 0.268238498 |
| NYU777 | Cancer | 0.692855826 | 0.250191118 | 0.279371662 |
| NYU176 | Benign | 0.204952779 | 0.260494527 | 0.305522547 |
| NYU888 | Cancer | 1.611353708 | 0.173025027 | 0.215307899 |
| NYU1117 | Benign | 0.339497837 | 0.23551398 | 0.273144929 |
| NYU1201 | Cancer | 0.774172652 | 0.266499176 | 0.271516939 |
| PC_02 | | 0.310972032 | 0.280393848 | 0.294079142 |
| NYU887 | Cancer | 0.250058138 | 0.252608269 | 0.257254245 |
| NYU815 | Benign | 0.284488928 | 0.255620978 | 0.275696816 |
| NYU927 | Cancer | 0.250422369 | 0.179487785 | 0.225557821 |
| NYU1030 | Benign | 0.21457607 | 0.21739441 | 0.225327932 |
| NYU1151 | Cancer | 1.218708603 | 0.17640146 | 0.236468112 |
| NYU1005 | Benign | NA | 0.318252411 | 0.347466911 |
| NYU522 | Benign | 0.302012878 | 0.277747101 | 0.286902331 |
| NYU389 | Cancer | 1.093807352 | 0.216832283 | 0.222127714 |
| PC_03 | | 0.443201986 | 0.279881332 | 0.283351509 |
| NYU729 | Cancer | NA | 0.219825506 | 0.278208402 |
| NYU430 | Benign | 0.347853903 | 0.238975243 | 0.277258779 |
| NYU144 | Benign | NA | 0.210196631 | 0.208673978 |
| NYU256 | Cancer | 0.330192527 | 0.270793127 | 0.288092981 |
| NYU1000 | Benign | 0.608991461 | 0.304861894 | 0.34054149 |
| NYU575 | Cancer | 0.274039152 | 0.25478525 | 0.277641016 |
| PC_04 | | 0.501433707 | 0.279370749 | 0.307443449 |

TABLE 11L
PV2 fidelity small nodule batch all transitions (normalized)

| | | YEV- | YEV- | | |
|---|---|---|---|---|---|
| msfile- | | TVVSVR_526.29_293.10 | TVVSVR_526.29_660.40 | YEV-TVVSVR_526.29_759.50 | YVSELHLTR_373.21_263.10 |
| name | status | (SEQ ID No.: 60) | (SEQ ID No.: 60) | (SEQ ID No.: 60) | (SEQ ID No.: 55) |
| PC_01 | | 0.715043069 | 0.77282955 | 0.643875456 | 0.506555218 |
| ZCO489_02 | Benign | 0.625029917 | 0.627170527 | 0.650817326 | 0.374904316 |
| ZCO436_02 | Cancer | 0.49116788 | 0.448328197 | 0.408567563 | 0.207142928 |
| ZCO512_02 | Cancer | 0.499213482 | 0.523484383 | 0.473903155 | 0.297205955 |
| ZCO475_02 | Benign | 0.601955185 | 0.628535711 | 0.549014407 | 0.316166053 |
| ZCO485_02 | Benign | 0.585695029 | 0.682970961 | 0.605347856 | 0.428266352 |
| ZCO536_02 | Cancer | 0.550757325 | 0.622087967 | 0.441650578 | 0.360970845 |
| PC_02 | | 0.689879381 | 0.649195525 | 0.63205638 | 0.446017566 |
| ZCO496_02 | Benign | 0.468331611 | 0.432415759 | 0.434869761 | 0.390882789 |
| ZCO502_02 | Cancer | 0.424577059 | 0.371605494 | 0.430028294 | 0.239048863 |
| ZCO382_02 | Benign | 0.585234517 | 0.61930386 | 0.66379927 | 0.414385294 |
| ZCO431_02 | Cancer | 0.452328912 | 0.415640557 | 0.398041019 | 0.298141172 |
| ZCO449_02 | Cancer | 0.803215412 | 0.765003073 | 0.891420258 | 0.313073796 |
| ZCO537_02 | Benign | 1.193518718 | 1.352934709 | 0.966312621 | 0.33758803 |
| ZCO362_02 | Benign | 0.467542739 | 0.640062814 | 0.511813147 | 0.453549018 |
| ZCO488_02 | Benign | 0.968481935 | 0.873641311 | 0.981672345 | 0.510857236 |
| PC_03 | | 0.72536496 | 0.769938529 | 0.941388746 | 0.475272248 |
| ZCO535_02 | Benign | 0.429867113 | 0.567154709 | 0.504132591 | 0.32951823 |
| ZCO443_02 | Cancer | 0.701856974 | 0.720022198 | 0.47868326 | 0.440234415 |
| ZCO393_02 | Benign | 0.501075534 | 0.545789452 | 0.467820883 | 0.38580852 |
| ZCO503_02 | Cancer | 0.565821184 | 0.586645168 | 0.718989975 | 0.326757997 |
| ZCO438_02 | Cancer | 0.465451696 | 0.356025326 | 0.365710523 | 0.165929325 |
| ZCO406_02 | Benign | 0.545631352 | 0.54293144 | 0.430368258 | 0.27851723 |
| PC_04 | | 0.707006234 | 0.909467584 | 0.803113276 | 0.485325416 |
| PC_01 | | 0.752743325 | 0.858483831 | 0.753013507 | 0.514928147 |
| 00082_07 | Cancer | 0.452447843 | 0.425805862 | 0.49759802 | 0.21100876 |
| 02286_07 | Benign | 0.542800282 | 0.572056873 | 0.508347433 | 0.258362566 |
| 02280_06 | Cancer | 0.51811225 | 0.526441109 | 0.583441479 | 0.433770685 |
| 01123_06 | Benign | 0.863124557 | 0.889062093 | 0.893478731 | 0.412709845 |
| 00156_07 | Cancer | 0.398413782 | 0.414555967 | 0.415628493 | 0.257845019 |
| 00781_09 | Benign | 0.486133795 | 0.524971457 | 0.562031012 | 0.362969883 |
| 00539_08 | Cancer | 0.606209877 | 0.607691068 | 0.538114255 | 0.282717077 |
| 02241_07 | Cancer | 0.446268901 | 0.401554145 | 0.440266476 | 0.453269604 |
| 02226_05 | Benign | 0.468274134 | 0.425067286 | 0.53307431 | 0.229061234 |
| PC_03 | | 0.954603534 | 0.795857814 | 0.870889698 | 0.4506214 |
| 00542_08 | NA | 0.958598473 | 0.801585241 | 0.898569664 | 0.204356381 |
| 02497_10 | NA | 0.555011435 | 0.581526716 | 0.563058571 | 0.263033194 |
| 02224_05 | Benign | 0.607911646 | 0.605187177 | 0.482684749 | 0.278914607 |
| 00748_09 | Cancer | 0.534663717 | 0.384265678 | 0.473118465 | 0.263705103 |
| 03630_09 | Benign | 0.525133696 | 0.491962837 | 0.555944288 | 0.361545001 |
| 02279_07 | Cancer | 0.508396893 | 0.501195431 | 0.423130329 | 0.244199856 |
| PC_04 | | 0.745756556 | 0.789882337 | 0.6634281 | 0.424989707 |
| PC_01 | | 0.715105882 | 0.803894516 | 0.705539433 | 0.416145616 |
| NYU806 | Benign | 0.406633817 | 0.513188857 | 0.428389998 | 0.135991544 |
| NYU777 | Cancer | 0.638982086 | 0.558030353 | 0.667354052 | 0.307311369 |
| NYU176 | Benign | 0.671289682 | 0.719325305 | 0.731835316 | 0.554839691 |
| NYU888 | Cancer | 0.697394859 | 0.681161461 | 0.635409235 | 0.249867718 |
| NYU1117 | Benign | 0.42099334 | 0.473389473 | 0.499157941 | 0.380875651 |
| NYU1201 | Cancer | 0.510962366 | 0.54158388 | 0.448587965 | 0.279667097 |
| PC_02 | | 0.676021274 | 0.768105794 | 0.722825167 | 0.389087664 |
| NYU887 | Cancer | 0.571945086 | 0.601656256 | 0.65639156 | 0.341688978 |
| NYU815 | Benign | 0.638614092 | 0.572159768 | 0.6510733 | 0.385729146 |
| NYU927 | Cancer | 0.59757421 | 0.580878491 | 0.575455912 | 0.305616909 |
| NYU1030 | Benign | 0.428916327 | 0.552394307 | 0.466160374 | 0.21683767 |
| NYU1151 | Cancer | 0.584186331 | 0.550659993 | 0.555687378 | 0.401430737 |
| NYU1005 | Benign | 0.64086204 | 0.626318045 | 0.582804662 | 0.412087596 |
| NYU522 | Benign | 1.070133718 | 1.087120571 | 1.093669401 | 0.325663099 |
| NYU389 | Cancer | 0.631536333 | 0.670268064 | 0.689968234 | 0.233423041 |
| PC_03 | | 0.79870931 | 0.653692201 | 0.681319599 | 0.407110378 |
| NYU729 | Cancer | 0.69516025 | 0.551130386 | 0.61918102 | 0.150997328 |
| NYU430 | Benign | 0.525108882 | 0.607477171 | 0.596875752 | 0.305367067 |
| NYU144 | Benign | 1.232862263 | 1.177435297 | 1.290275649 | 0.407143128 |
| NYU256 | Cancer | 0.620483355 | 0.640358673 | 0.594397346 | 0.368101892 |
| NYU1000 | Benign | 0.902243335 | 0.921117039 | 0.737710918 | 0.30180146 |
| NYU575 | Cancer | 0.487846798 | 0.477801464 | 0.512720254 | 0.249804456 |
| PC_04 | | 0.839577029 | 0.806193827 | 0.701607538 | 0.428217291 |

| msfile- | | YVSELHLTR_373.21_428.30 | YVSELHLTR_373.21_526.30 | YVSELHLTR_559.30_855.50 |
|---|---|---|---|---|
| name | status | (SEQ ID No.: 55) | (SEQ ID No.: 55) | (SEQ ID No.: 55) |
| PC_01 | | 0.52600757 | 0.544348366 | 0.490205799 |
| ZCO489_02 | Benign | 0.418856583 | 0.513178508 | 0.417881095 |
| ZCO436_02 | Cancer | 0.282920347 | 0.290856366 | 0.266128773 |
| ZCO512_02 | Cancer | 0.334774545 | 0.37397234 | 0.347079417 |
| ZCO475_02 | Benign | 0.351142711 | 0.392649532 | 0.317095721 |
| ZCO485_02 | Benign | 0.42973392 | 0.470509831 | 0.396083376 |
| ZCO536_02 | Cancer | 0.416953865 | 0.409299842 | 0.350956549 |
| PC_02 | | 0.483683874 | 0.595668035 | 0.571270925 |
| ZCO496_02 | Benign | 0.419136681 | 0.440558925 | 0.39359143 |
| ZCO502_02 | Cancer | 0.245510127 | 0.26778992 | 0.202083213 |
| ZCO382_02 | Benign | 0.454290423 | 0.492223039 | 0.497652247 |
| ZCO431_02 | Cancer | 0.314414924 | 0.351938241 | 0.305640502 |
| ZCO449_02 | Cancer | 0.327492923 | 0.352361358 | 0.316372718 |
| ZCO537_02 | Benign | 0.366156695 | 0.424783089 | 0.339086481 |
| ZCO362_02 | Benign | 0.505177456 | 0.518428483 | 0.436149511 |
| ZCO488_02 | Benign | 0.611578187 | 0.610228269 | 0.488007709 |
| PC_03 | | 0.564305328 | 0.630778062 | 0.506931336 |
| ZCO535_02 | Benign | 0.356303061 | 0.359217737 | 0.299614436 |
| ZCO443_02 | Cancer | 0.473099402 | 0.493811246 | 0.399742475 |
| ZCO393_02 | Benign | 0.411800156 | 0.42919049 | 0.364664078 |
| ZCO503_02 | Cancer | 0.346343776 | 0.398536174 | 0.317762487 |
| ZCO438_02 | Cancer | 0.147404214 | 0.20480617 | 0.123337078 |
| ZCO406_02 | Benign | 0.377407 | 0.450255558 | 0.375181921 |
| PC_04 | | 0.571395341 | 0.622958058 | 0.575941596 |
| PC_01 | | 0.556861468 | 0.536765352 | 0.488120094 |
| 00082_07 | Cancer | 0.236532409 | 0.224358624 | 0.241549614 |
| 02286_07 | Benign | 0.325855205 | 0.312250736 | 0.298466978 |
| 02280_06 | Cancer | 0.507902067 | 0.506247702 | 0.455969947 |
| 01123_06 | Benign | 0.502904193 | 0.539821839 | 0.515626738 |
| 00156_07 | Cancer | 0.282904675 | 0.273571892 | 0.2828297 |
| 00781_09 | Benign | 0.39926759 | 0.468051896 | 0.36071456 |
| 00539_08 | Cancer | 0.326126027 | 0.378118027 | 0.299442432 |
| 02241_07 | Cancer | 0.533661101 | 0.492229735 | 0.506932972 |
| 02226_05 | Benign | 0.293646302 | 0.32299766 | 0.267461736 |
| PC_03 | | 0.584232304 | 0.62254197 | 0.515078241 |
| 00542_08 | NA | 0.221331588 | 0.262208041 | 0.207208555 |
| 02497_10 | NA | 0.285273196 | 0.29983914 | 0.268121708 |
| 02224_05 | Benign | 0.318541493 | 0.33573911 | 0.293257348 |
| 00748_09 | Cancer | 0.32171685 | 0.332099153 | 0.333929767 |
| 03630_09 | Benign | 0.407981097 | 0.457248698 | 0.383996891 |
| 02279_07 | Cancer | 0.286681753 | 0.28452828 | 0.242156498 |
| PC_04 | | 0.525161575 | 0.568895093 | 0.469736845 |
| PC_01 | | 0.522433074 | 0.546468924 | 0.467568329 |
| NYU806 | Benign | 0.176138804 | 0.183137317 | 0.16608957 |
| NYU777 | Cancer | 0.384682052 | 0.41242755 | 0.389082517 |
| NYU176 | Benign | 0.641081063 | 0.715026769 | 0.568743823 |
| NYU888 | Cancer | 0.377873601 | 0.369212104 | 0.337058297 |
| NYU1117 | Benign | 0.502771887 | 0.561062766 | 0.515739008 |
| NYU1201 | Cancer | 0.360351445 | 0.434901711 | 0.3504042 |
| PC_02 | | 0.461398046 | 0.541328871 | 0.504602481 |
| NYU887 | Cancer | 0.417587443 | 0.445035912 | 0.441980699 |
| NYU815 | Benign | 0.489782839 | 0.568034906 | 0.453188864 |
| NYU927 | Cancer | 0.382408797 | 0.443790054 | 0.35997525 |
| NYU1030 | Benign | 0.319259068 | 0.324628276 | 0.294226621 |
| NYU1151 | Cancer | 0.538213965 | 0.54798511 | 0.556499897 |
| NYU1005 | Benign | 0.450576466 | 0.484060642 | 0.506456106 |
| NYU522 | Benign | 0.418064577 | 0.444094216 | 0.415060204 |
| NYU389 | Cancer | 0.255723118 | 0.240399969 | 0.20913483 |
| PC_03 | | 0.465914659 | 0.541837768 | 0.527512555 |
| NYU729 | Cancer | 0.221683545 | 0.205922161 | 0.188504231 |
| NYU430 | Benign | 0.359859903 | 0.390569226 | 0.344372041 |
| NYU144 | Benign | 0.608001062 | 0.594274141 | 0.509692938 |
| NYU256 | Cancer | 0.561999174 | 0.564840089 | 0.545318003 |
| NYU1000 | Benign | 0.379369581 | 0.403854734 | 0.397683581 |
| NYU575 | Cancer | 0.335263602 | 0.364197685 | 0.301811073 |
| PC_04 | | 0.57512524 | 0.597968594 | 0.591453859 |

TABLE 11M
PV2 fidelity small nodule batch all transitions (normalized)

| | | YYIAASYVK_539.28_327.10 | YYIAASYVK_539.28_567.30 |
|---|---|---|---|
| msfilename | status | (SEQ ID No.: 51) | (SEQ ID No.: 51) |
| PC_01 | | 0.214882781 | 0.262382136 |
| ZCO489_02 | Benign | 0.189725597 | 0.302324442 |
| ZCO436_02 | Cancer | 0.338460701 | 0.369972325 |
| ZCO512_02 | Cancer | 0.139638041 | 0.183183202 |
| ZCO475_02 | Benign | 0.158977544 | 0.213554386 |
| ZCO485_02 | Benign | 0.158915047 | 0.198415248 |
| ZCO536_02 | Cancer | 0.23524574 | 0.316112824 |
| PC_02 | | 0.254786228 | 0.263628021 |
| ZCO496_02 | Benign | 0.20000143 | 0.228744466 |
| ZCO502_02 | Cancer | 0.296573255 | 0.232179936 |
| ZCO382_02 | Benign | 0.29869956 | 0.298071888 |
| ZCO431_02 | Cancer | 0.210938861 | 0.241308436 |
| ZCO449_02 | Cancer | 0.147154321 | 0.295480744 |
| ZCO537_02 | Benign | 0.240816236 | 0.326321668 |
| ZCO362_02 | Benign | 0.216149273 | 0.192744458 |
| ZCO488_02 | Benign | 0.241509973 | 0.33467281 |
| PC_03 | | 0.332010719 | 0.245582048 |
| ZCO535_02 | Benign | 0.162271094 | 0.311125392 |
| ZCO443_02 | Cancer | 0.35112887 | 0.406307263 |
| ZCO393_02 | Benign | 0.145139001 | 0.18520178 |
| ZCO503_02 | Cancer | 0.48685129 | 0.538295082 |
| ZCO438_02 | Cancer | 0.224105327 | 0.342169057 |
| ZCO406_02 | Benign | 0.332851621 | 0.327904959 |
| PC_04 | | 0.32831609 | 0.32516808 |
| PC_01 | | 0.333553782 | 0.300129901 |
| 00082_07 | Cancer | 0.216655016 | 0.204005317 |
| 02286_07 | Benign | 0.146741869 | 0.175223928 |
| 02280_06 | Cancer | 0.30011835 | 0.363836459 |
| 01123_06 | Benign | 0.155625871 | 0.183496256 |
| 00156_07 | Cancer | 0.511030094 | 0.410603693 |
| 00781_09 | Benign | 0.281452331 | 0.38713335 |
| 00539_08 | Cancer | 0.199709057 | 0.207150477 |
| 02241_07 | Cancer | 0.093773866 | 0.104254108 |
| 02226_05 | Benign | 0.242872972 | 0.259913094 |
| PC_03 | | 0.299855333 | 0.34284319 |
| 00542_08 | NA | 0.329885555 | 0.245581916 |
| 02497_10 | NA | 0.182082247 | 0.229355394 |
| 02224_05 | Benign | 0.170206939 | 0.143938669 |
| 00748_09 | Cancer | 0.189400194 | 0.168373189 |
| 03630_09 | Benign | 0.297427502 | 0.354569011 |
| 02279_07 | Cancer | 0.322841031 | 0.257140348 |
| PC_04 | | 0.317970017 | 0.285108325 |
| PC_01 | | 0.244987828 | 0.302518103 |
| NYU806 | Benign | 0.209341159 | 0.457058613 |
| NYU777 | Cancer | 0.224047613 | 0.29126364 |
| NYU176 | Benign | 0.215591092 | 0.164108433 |
| NYU888 | Cancer | 0.429225254 | 0.43452679 |
| NYU1117 | Benign | 0.141787389 | 0.183689784 |
| NYU1201 | Cancer | 0.289551981 | 0.185304854 |
| PC_02 | | 0.203598263 | 0.229141121 |
| NYU887 | Cancer | 0.23240879 | 0.28533565 |
| NYU815 | Benign | 0.122605415 | 0.12774684 |
| NYU927 | Cancer | 0.13062957 | 0.163939166 |
| NYU1030 | Benign | 0.193876884 | 0.21774014 |
| NYU1151 | Cancer | 0.187023228 | 0.19602555 |
| NYU1005 | Benign | 0.175331475 | 0.261157331 |
| NYU522 | Benign | 0.125996325 | 0.171423928 |
| NYU389 | Cancer | 0.282144088 | 0.311490631 |
| PC_03 | | 0.2736282 | 0.354405931 |
| NYU729 | Cancer | 0.163808358 | 0.306489063 |
| NYU430 | Benign | 0.193856904 | 0.265625089 |
| NYU144 | Benign | 0.370103603 | 0.506132547 |
| NYU256 | Cancer | 0.225980753 | 0.17884423 |
| NYU1000 | Benign | 0.155917153 | 0.18381643 |
| NYU575 | Cancer | 0.234951179 | 0.261100911 |
| PC_04 | | 0.306215539 | 0.261721536 |
| | | YYIAASYVK_539.28_638.40 | YYIAASYVK_539.28_751.40 |
| msfilename | status | (SEQ ID No.: 51) | (SEQ ID No.: 51) |
| PC_01 | | 0.322342571 | 0.235896902 |
| ZCO489_02 | Benign | 0.250362289 | 0.174638378 |
| ZCO436_02 | Cancer | 0.305363024 | 0.21532763 |
| ZCO512_02 | Cancer | 0.194266457 | 0.187343705 |
| ZCO475_02 | Benign | 0.219717125 | 0.148248509 |
| ZCO485_02 | Benign | 0.204408449 | 0.157893291 |
| ZCO536_02 | Cancer | 0.285633047 | 0.258031573 |
| PC_02 | | 0.283236205 | 0.279571289 |
| ZCO496_02 | Benign | 0.237676305 | 0.249833642 |
| ZCO502_02 | Cancer | 0.221305802 | 0.265631518 |
| ZCO382_02 | Benign | 0.283330494 | 0.275818296 |
| ZCO431_02 | Cancer | 0.257479852 | 0.147067961 |
| ZCO449_02 | Cancer | 0.221346932 | 0.168575851 |
| ZCO537_02 | Benign | 0.273931193 | 0.255940247 |
| ZCO362_02 | Benign | 0.172044378 | 0.189600303 |
| ZCO488_02 | Benign | 0.32586649 | 0.258264891 |
| PC_03 | | 0.303976613 | 0.29665481 |
| ZCO535_02 | Benign | 0.258239217 | 0.153498811 |
| ZCO443_02 | Cancer | 0.394714161 | 0.408145743 |
| ZCO393_02 | Benign | 0.214738332 | 0.145226342 |
| ZCO503_02 | Cancer | 0.508816323 | 0.498118315 |
| ZCO438_02 | Cancer | 0.283637288 | 0.200027261 |
| ZCO406_02 | Benign | 0.373342717 | 0.280954827 |
| PC_04 | | 0.314959896 | 0.276302248 |
| PC_01 | | 0.294108799 | 0.298045133 |
| 00082_07 | Cancer | 0.227617268 | 0.188589106 |
| 02286_07 | Benign | 0.164824992 | 0.130477815 |
| 02280_06 | Cancer | 0.258099164 | 0.31469993 |
| 01123_06 | Benign | 0.150843864 | 0.140566429 |
| 00156_07 | Cancer | 0.507647165 | 0.442888081 |
| 00781_09 | Benign | 0.369365507 | 0.295699273 |
| 00539_08 | Cancer | 0.223817813 | 0.204987217 |
| 02241_07 | Cancer | 0.115972399 | 0.103778429 |
| 02226_05 | Benign | 0.259778873 | 0.246685789 |
| PC_03 | | 0.338040968 | 0.297816537 |
| 00542_08 | NA | 0.292444128 | 0.285931107 |
| 02497_10 | NA | 0.261519847 | 0.187466915 |
| 02224_05 | Benign | 0.235324944 | 0.215546853 |
| 00748_09 | Cancer | 0.204942963 | 0.142499979 |
| 03630_09 | Benign | 0.264578832 | 0.238974558 |
| 02279_07 | Cancer | 0.339809114 | 0.253320835 |
| PC_04 | | 0.291762119 | 0.264789581 |
| PC_01 | | 0.26737881 | 0.313039422 |
| NYU806 | Benign | 0.28525922 | 0.222592844 |
| NYU777 | Cancer | 0.321111153 | 0.191679099 |
| NYU176 | Benign | 0.215634494 | 0.16241181 |
| NYU888 | Cancer | 0.398216446 | 0.397448587 |
| NYU1117 | Benign | 0.138842438 | 0.117987802 |
| NYU1201 | Cancer | 0.210584021 | 0.19467434 |
| PC_02 | | 0.275793893 | 0.322607937 |
| NYU887 | Cancer | 0.236851961 | 0.228345185 |
| NYU815 | Benign | 0.177400236 | 0.116546756 |
| NYU927 | Cancer | 0.143086835 | 0.113838031 |
| NYU1030 | Benign | 0.223566301 | 0.226383594 |
| NYU1151 | Cancer | 0.241928632 | 0.177788155 |
| NYU1005 | Benign | 0.241643811 | 0.126822387 |
| NYU522 | Benign | 0.166936773 | 0.112105938 |
| NYU389 | Cancer | 0.256655884 | 0.182464411 |
| PC_03 | | 0.299238857 | 0.27992114 |
| NYU729 | Cancer | 0.205665436 | 0.200859709 |
| NYU430 | Benign | 0.27867877 | 0.219251629 |
| NYU144 | Benign | 0.491042254 | 0.344768742 |
| NYU256 | Cancer | 0.27965313 | 0.188431293 |
| NYU1000 | Benign | 0.149526371 | 0.124230064 |
| NYU575 | Cancer | 0.251753723 | 0.226431877 |
| PC_04 | | 0.283387092 | 0.325952884 |

TABLE 12
Nucleotide and Amino Acid Sequences for Genes of Interest

| | | Seq. |
|---|---|---|
| Gene Name | Nucleotide and Amino Acid Sequences | ID. |
| BGH3_HUMAN | ATGGCGCTGTTTGTGCGCCTGCTGGCGCTGGCGCTGGCGCTGGCGCTGGGCCCGGCGGCGACCCTGGCGGGCCCGGCG | 1 |
| | AAAAGCCCGTATCAGCTGGTGCTGCAGCATAGCCGCCTGCGCGGCCGCCAGCATGGCCCGAACGTGTGCGCGGTGCAG | |
| | AAAGTGATTGGCACCAACCGCAAATATTTTACCAACTGCAAACAGTGGTATCAGCGCAAAATTTGCGGCAAAAGCACC | |
| | GTGATTAGCTATGAATGCTGCCCGGGCTATGAAAAAGTGCCGGGCGAAAAAGGCTGCCCGGCGGCGCTGCCGCTGAGC | |
| | AACCTGTATGAAACCCTGGGCGTGGTGGGCAGCACCACCACCCAGCTGTATACCGATCGCACCGAAAAACTGCGCCCG | |
| | GAAATGGAAGGCCCGGGCAGCTTTACCATTTTTGCGCCGAGCAACGAAGCGTGGGCGAGCCTGCCGGCGGAAGTGCTG | |
| | GATAGCCTGGTGAGCAACGTGAACATTGAACTGCTGAACGCGCTGCGCTATCATATGGTGGGCCGCCGCGTGCTGACC | |
| | GATGAACTGAAACATGGCATGACCCTGACCAGCATGTATCAGAACAGCAACATTCAGATTCATCATTATCCGAACGGC | |
| | ATTGTGACCGTGAACTGCGCGCGCCTGCTGAAAGCGGATCATCATGCGACCAACGGCGTGGTGCATCTGATTGATAAA | |
| | GTGATTAGCACCATTACCAACAACATTCAGCAGATTATTGAAATTGAAGATACCTTTGAAACCCTGCGCGCGGCGGTG | |
| | GCGGCGAGCGGCCTGAACACCATGCTGGAAGGCAACGGCCAGTATACCCTGCTGGCGCCGACCAACGAAGCGTTTGAA | |
| | AAAATTCCGAGCGAAACCCTGAACCGCATTCTGGGCGATCCGGAAGCGCTGCGCGATCTGCTGAACAACCATATTCTG | |
| | AAAAGCGCGATGTGCGCGGAAGCGATTGTGGCGGGCCTGAGCGTGGAAACCCTGGAAGGCACCACCCTGGAAGTGGGC | |
| | TGCAGCGGCGATATGCTGACCATTAACGGCAAAGCGATTATTAGCAACAAAGATATTCTGGCGACCAACGGCGTGATT | |
| | CATTATATTGATGAACTGCTGATTCCGGATAGCGCGAAAACCCTGTTTGAACTGGCGGCGGAAAGCGATGTGAGCACC | |
| | GCGATTGATCTGTTTCGCCAGGCGGGCCTGGGCAACCATCTGAGCGGCAGCGAACGCCTGACCCTGCTGGCGCCGCTG | |
| | AACAGCGTGTTTAAAGATGGCACCCCGCCGATTGATGCGCATACCCGCAACCTGCTGCGCAACCATATTATTAAAGAT | |
| | CAGCTGGCGAGCAAATATCTGTATCATGGCCAGACCCTGGAAACCCTGGGCGGCAAAAAACTGCGCGTGTTTGTGTAT | |
| | CGCAACAGCCTGTGCATTGAAAACAGCTGCATTGCGGCGCATGATAAACGCGGCCGCTATGGCACCCTGTTTACCATG | |
| | GATCGCGTGCTGACCCCGCCGATGGGCACCGTGATGGATGTGCTGAAAGGCGATAACCGCTTTAGCATGCTGGTGGCG | |
| | GCGATTCAGAGCGCGGGCCTGACCGAAACCCTGAACCGCGAAGGCGTGTATACCGTGTTTGCGCCGACCAACGAAGCG | |
| | TTTCGCGCGCTGCCGCCGCGCGAACGCAGCCGCCTGCTGGGCGATGCGAAAGAACTGGCGAACATTCTGAAATATCAT | |
| | ATTGGCGATGAAATTCTGGTGAGCGGCGGCATTGGCGCGCTGGTGCGCCTGAAAAGCCTGCAGGGCGATAAACTGGAA | |
| | GTGAGCCTGAAAAACAACGTGGTGAGCGTGAACAAAGAACCGGTGGCGGAACCGGATATTATGGCGACCAACGGCGTG | |
| | GTGCATGTGATTACCAACGTGCTGCAGCCGCCGGCGAACCGCCCGCAGGAACGCGGCGATGAACTGGCGGATAGCGCG | |
| | CTGGAAATTTTTAAACAGGCGAGCGCGTTTAGCCGCGCGAGCCAGCGCAGCGTGCGCCTGGCGCCGGTGTATCAGAAA | |
| | CTGCTGGAACGCATGAAACAT | |
| BGH3_HUMAN | MALFVRLLALALALALGPAATLAGPAKSPYQLVLQHSRLRGRQHGPNVCAVQKVIGTNRKYFTNCKQWYQRKICGKST | 2 |
| | VISYECCPGYEKVPGEKGCPAALPLSNLYETLGVVGSTTTQLYTDRTEKLRPEMEGPGSFTIFAPSNEAWASLPAEVL | |
| | DSLVSNVNIELLNALRYHMVGRRVLTDELKHGMTLTSMYQNSNIQIHHYPNGIVTVNCARLLKADHHATNGVVHLIDK | |
| | VISTITNNIQQIIEIEDTFETLRAAVAASGLNTMLEGNGQYTLLAPTNEAFEKIPSETLNRILGDPEALRDLLNNHIL | |
| | KSAMCAEAIVAGLSVETLEGTTLEVGCSGDMLTINGKAIISNKDILATNGVIHYIDELLIPDSAKTLFELAAESDVST | |
| | AIDLFRQAGLGNHLSGSERLTLLAPLNSVFKDGTPPIDAHTRNLLRNHIIKDQLASKYLYHGQTLETLGGKKLRVFVY | |
| | RNSLCTENSCIAAHDKRGRYGTLFTMDRVLTPPMGTVMDVLKGDNRFSMLVAAIQSAGLTETLNREGVYTVFAPTNEA | |
| | FRALPPRERSRLLGDAKELANILKYHIGDEILVSGGIGALVRLKSLQGDKLEVSLKNNVVSVNKEPVAEPDIMATNGV | |
| | VHVITNVLQPPANRPQERGDELADSALEIFKQASAFSRASQRSVRLAPVYQKLLERMKH | |
| GGH_HUMAN | ATGGCGAGCCCGGGCTGCCTGCTGTGCGTGCTGGGCCTGCTGCTGTGCGGCGCGGCGAGCCTGGAACTGAGCCGCCCG | 3 |
| | CATGGCGATACCGCGAAAAAACCGATTATTGGCATTCTGATGCAGAAATGCCGCAACAAAGTGATGAAAAACTATGGC | |
| | CGCTATTATATTGCGGCGAGCTATGTGAAATATCTGGAAAGCGCGGGCGCGCGCGTGGTGCCGGTGCGCCTGGATCTG | |
| | ACCGAAAAAGATTATGAAATTCTGTTTAAAAGCATTAACGGCATTCTGTTTCCGGGCGGCAGCGTGGATCTGCGCCGC | |
| | AGCGATTATGCGAAAGTGGCGAAAATTTTTTATAACCTGAGCATTCAGAGCTTTGATGATGGCGATTATTTTCCGGTG | |
| | TGGGGCACCTGCCTGGGCTTTGAAGAACTGAGCCTGCTGATTAGCGGCGAATGCCTGCTGACCGCGACCGATACCGTG | |
| | GATGTGGCGATGCCGCTGAACTTTACCGGCGGCCAGCTGCATAGCCGCATGTTTCAGAACTTTCCGACCGAACTGCTG | |
| | CTGAGCCTGGCGGTGGAACCGCTGACCGCGAACTTTCATAAATGGAGCCTGAGCGTGAAAAACTTTACCATGAACGAA | |
| | AAACTGAAAAAATTTTTTAACGTGCTGACCACCAACACCGATGGCAAAATTGAATTTATTAGCACCATGGAAGGCTAT | |
| | AAATATCCGGTGTATGGCGTGCAGTGGCATCCGGAAAAAGCGCCGTATGAATGGAAAAACCTGGATGGCATTAGCCAT | |
| | GCGCCGAACGCGGTGAAAACCGCGTTTTATCTGGCGGAATTTTTTGTGAACGAAGCGCGCAAAAACAACCATCATTTT | |
| | AAAAGCGAAAGCGAAGAAGAAAAAGCGCTGATTTATCAGTTTAGCCCGATTTATACCGGCAACATTAGCAGCTTTCAG | |
| | CAGTGCTATATTTTTGAT | |
| GGH_HUMAN | MASPGCLLCVLGLLLCGAASLELSRPHGDTAKKPIIGILMQKCRNKVMKNYGRYYIAASYVKYLESAGARVVPVRLDL | 4 |
| | TEKDYEILFKSINGILFPGGSVDLRRSDYAKVAKIFYNLSIQSFDDGDYFPVWGTCLGFEELSLLISGECLLTATDTV | |
| | DVAMPLNFTGGQLHSRMFQNFPTELLLSLAVEPLTANFHKWSLSVKNFTMNEKLKKFFNVLTTNTDGKIEFISTMEGY | |
| | KYPVYGVQWHPEKAPYEWKNLDGISHAPNAVKTAFYLAEFFVNEARKNNHHFKSESEEEKALIYQFSPIYTGNISSFQ | |
| | QCYIFD | |
| LG3BP_HUMAN | ATGACCCCTCCGAGGCTCTTCTGGGTGTGGCTGCTGGTTGCAGGAACCCAAGGCGTGAACGATGGTGACATGCGGCTG | 5 |
| | GCCGATGGGGGCGCCACCAACCAGGGCCGCGTGGAGATCTTCTACAGAGGCCAGTGGGGCACTGTGTGTGACAACCTG | |
| | TGGGACCTGACTGATGCCAGCGTCGTCTGCCGGGCCCTGGGCTTCGAGAACGCCACCCAGGCTCTGGGCAGAGCTGCC | |
| | TTCGGGCAAGGATCAGGCCCCATCATGCTGGATGAGGTCCAGTGCACGGGAACCGAGGCCTCACTGGCCGACTGCAAG | |
| | TCCCTGGGCTGGCTGAAGAGCAACTGCAGGCACGAGAGAGACGCTGGTGTGGTCTGCACCAATGAAACCAGGAGCACC | |
| | CACACCCTGGACCTCTCCAGGGAGCTCTCGGAGGCCCTTGGCCAGATCTTTGACAGCCAGCGGGGCTGCGACCTGTCC | |
| | ATCAGCGTGAATGTGCAGGGCGAGGACGCCCTGGGCTTCTGTGGCCACACGGTCATCCTGACTGCCAACCTGGAGGCC | |
| | CAGGCCCTGTGGAAGGAGCCGGGCAGCAATGTCACCATGAGTGTGGATGCTGAGTGTGTGCCCATGGTCAGGGACCTT | |
| | CTCAGGTACTTCTACTCCCGAAGGATTGACATCACCCTGTCGTCAGTCAAGTGCTTCCACAAGCTGGCCTCTGCCTAT | |
| | GGGGCCAGGCAGCTGCAGGGCTACTGCGCAAGCCTCTTTGCCATCCTCCTCCCCCAGGACCCCTCGTTCCAGATGCCC | |
| | CTGGACCTGTATGCCTATGCAGTGGCCACAGGGGACGCCCTGCTGGAGAAGCTCTGCCTACAGTTCCTGGCCTGGAAC | |
| | TTCGAGGCCTTGACGCAGGCCGAGGCCTGGCCCAGTGTCCCCACAGACCTGCTCCAACTGCTGCTGCCCAGGAGCGAC | |
| | CTGGCGGTGCCCAGCGAGCTGGCCCTACTGAAGGCCGTGGACACCTGGAGCTGGGGGGAGCGTGCCTCCCATGAGGAG | |
| | GTGGAGGGCTTGGTGGAGAAGATCCGCTTCCCCATGATGCTCCCTGAGGAGCTCTTTGAGCTGCAGTTCAACCTGTCC | |
| | CTGTACTGGAGCCACGAGGCCCTGTTCCAGAAGAAGACTCTGCAGGCCCTGGAATTCCACACTGTGCCCTTCCAGTTG | |
| | CTGGCCCGGTACAAAGGCCTGAACCTCACCGAGGATACCTACAAGCCCCGGATTTACACCTCGCCCACCTGGAGTGCC | |
| | TTTGTGACAGACAGTTCCTGGAGTGCACGGAAGTCACAACTGGTCTATCAGTCCAGACGGGGGCCTTTGGTCAAATAT | |
| | TCTTCTGATTACTTCCAAGCCCCCTCTGACTACAGATACTACCCCTACCAGTCCTTCCAGACTCCACAACACCCCAGC | |
| | TTCCTCTTCCAGGACAAGAGGGTGTCCTGGTCCCTGGTCTACCTCCCCACCATCCAGAGCTGCTGGAACTACGGCTTC | |
| | TCCTGCTCCTCGGACGAGCTCCCTGTCCTGGGCCTCACCAAGTCTGGCGGCTCAGATCGCACCATTGCCTACGAAAAC | |
| | AAAGCCCTGATGCTCTGCGAAGGGCTCTTCGTGGCAGACGTCACCGATTTCGAGGGCTGGAAGGCTGCGATTCCCAGT | |
| | GCCCTGGACACCAACAGCTCGAAGAGCACCTCCTCCTTCCCCTGCCCGGCAGGGCACTTCAACGGCTTCCGCACGGTC | |
| | ATCCGCCCCTTCTACCTGACCAACTCCTCAGGTGTGGACTAG | |
| LG3BP_HUMAN | MTPPRLFWVWLLVAGTQGVNDGDMRLADGGATNQGRVEIFYRGQWGTVCDNLWDLTDASVVCRALGFENATQALGRAA | 6 |
| | FGQGSGPIMLDEVQCTGTEASLADCKSLGWLKSNCRHERDAGVVCTNETRSTHTLDLSRELSEALGQIFDSQRGCDLS | |
| | ISVNVQGEDALGFCGHTVILTANLEAQALWKEPGSNVTMSVDAECVPMVRDLLRYFYSRRIDITLSSVKCFHKLASAY | |
| | GARQLQGYCASLFAILLPQDPSFQMPLDLYAYAVATGDALLEKLCLQFLAWNFEALTQAEAWPSVPTDLLQLLLPRSD | |
| | LAVPSELALLKAVDTWSWGERASHEEVEGLVEKIRFPMMLPEELFELQFNLSLYWSHEALFQKKTLQALEFHTVPFQL | |
| | LARYKGLNLTEDTYKPRIYTSPTWSAFVTDSSWSARKSQLVYQSRRGPLVKYSSDYFQAPSDYRYYPYQSFQTPQHPS | |
| | FLFQDKRVSWSLVYLPTIQSCWNYGFSCSSDELPVLGLTKSGGSDRTIAYENKALMLCEGLFVADVTDFEGWKAAIPS | |
| | ALDTNSSKSTSSFPCPAGHFNGFRTVIRPFYLTNSSGVD | |
| PRDX1_HUMA | ATGAGCAGCGGCAACGCGAAAATTGGCCATCCGGCGCCGAACTTTAAAGCGACCGCGGTGATGCCGGATGGCCAGTTT | 7 |
| | AAAGATATTAGCCTGAGCGATTATAAAGGCAAATATGTGGTGTTTTTTTTTTATCCGCTGGATTTTACCTTTGTGTGC | |
| | CCGACCGAAATTATTGCGTTTAGCGATCGCGCGGAAGAATTTAAAAAACTGAACTGCCAGGTGATTGGCGCGAGCGTG | |
| | GATAGCCATTTTTGCCATCTGGCGTGGGTGAACACCCCGAAAAAACAGGGCGGCCTGGGCCCGATGAACATTCCGCTG | |
| | GTGAGCGATCCGAAACGCACCATTGCGCAGGATTATGGCGTGCTGAAAGCGGATGAAGGCATTAGCTTTCGCGGCCTG | |
| | TTTATTATTGATGATAAAGGCATTCTGCGCCAGATTACCGTGAACGATCTGCCGGTGGGCCGCAGCGTGGATGAAACC | |
| | CTGCGCCTGGTGCAGGCGTTTCAGTTTACCGATAAACATGGCGAAGTGTGCCCGGCGGGCTGGAAACCGGGCAGCGAT | |
| | ACCATTAAACCGGATGTGCAGAAAAGCAAAGAATATTTTAGCAAACAGAAA | |
| PRDX1_HUMAN | MSSGNAKIGHPAPNFKATAVMPDGQFKDISLSDYKGKYVVFFFYPLDFTFVCPTEIIAFSDRAEEFKKLNCQVIGASV | 8 |
| | DSHFCHLAWVNTPKKQGGLGPMNIPLVSDPKRTIAQDYGVLKADEGISFRGLFIIDDKGILRQITVNDLPVGRSVDET | |
| | LRLVQAFQFTDKHGEVCPAGWKPGSDTIKPDVQKSKEYFSKQK | |
| TSP1_HUMAN | ATGGGGCTGGCCTGGGGACTAGGCGTCCTGTTCCTGATGCATGTGTGTGGCACCAACCGCATTCCAGAGTCTGGCGGA | 9 |
| | GACAACAGCGTGTTTGACATCTTTGAACTCACCGGGGCCGCCCGCAAGGGGTCTGGGCGCCGACTGGTGAAGGGCCCC | |
| | GACCCTTCCAGCCCAGCTTTCCGCATCGAGGATGCCAACCTGATCCCCCCTGTGCCTGATGACAAGTTCCAAGACCTG | |
| | GTGGATGCTGTGCGGGCAGAAAAGGGTTTCCTCCTTCTGGCATCCCTGAGGCAGATGAAGAAGACCCGGGGCACGCTG | |
| | CTGGCCCTGGAGCGGAAAGACCACTCTGGCCAGGTCTTCAGCGTGGTGTCCAATGGCAAGGCGGGCACCCTGGACCTC | |
| | AGCCTGACCGTCCAAGGAAAGCAGCACGTGGTGTCTGTGGAAGAAGCTCTCCTGGCAACCGGCCAGTGGAAGAGCATC | |
| | ACCCTGTTTGTGCAGGAAGACAGGGCCCAGCTGTACATCGACTGTGAAAAGATGGAGAATGCTGAGTTGGACGTCCCC | |
| | ATCCAAAGCGTCTTCACCAGAGACCTGGCCAGCATCGCCAGACTCCGCATCGCAAAGGGGGGCGTCAATGACAATTTC | |
| | CAGGGGGTGCTGCAGAATGTGAGGTTTGTCTTTGGAACCACACCAGAAGACATCCTCAGGAACAAAGGCTGCTCCAGC | |
| | TCTACCAGTGTCCTCCTCACCCTTGACAACAACGTGGTGAATGGTTCCAGCCCTGCCATCCGCACTAACTACATTGGC | |
| | CACAAGACAAAGGACTTGCAAGCCATCTGCGGCATCTCCTGTGATGAGCTGTCCAGCATGGTCCTGGAACTCAGGGGC | |
| | CTGCGCACCATTGTGACCACGCTGCAGGACAGCATCCGCAAAGTGACTGAAGAGAACAAAGAGTTGGCCAATGAGCTG | |
| | AGGCGGCCTCCCCTATGCTATCACAACGGAGTTCAGTACAGAAATAACGAGGAATGGACTGTTGATAGCTGCACTGAG | |
| | TGTCACTGTCAGAACTCAGTTACCATCTGCAAAAAGGTGTCCTGCCCCATCATGCCCTGCTCCAATGCCACAGTTCCT | |
| | GATGGAGAATGCTGTCCTCGCTGTTGGCCCAGCGACTCTGCGGACGATGGCTGGTCTCCATGGTCCGAGTGGACCTCC | |
| | TGTTCTACGAGCTGTGGCAATGGAATTCAGCAGCGCGGCCGCTCCTGCGATAGCCTCAACAACCGATGTGAGGGCTCC | |
| | TCGGTCCAGACACGGACCTGCCACATTCAGGAGTGTGACAAGAGATTTAAACAGGATGGTGGCTGGAGCCACTGGTCC | |
| | CCGTGGTCATCTTGTTCTGTGACATGTGGTGATGGTGTGATCACAAGGATCCGGCTCTGCAACTCTCCCAGCCCCCAG | |
| | ATGAACGGGAAACCCTGTGAAGGCGAAGCGCGGGAGACCAAAGCCTGCAAGAAAGACGCCTGCCCCATCAATGGAGGC | |
| | TGGGGTCCTTGGTCACCATGGGACATCTGTTCTGTCACCTGTGGAGGAGGGGTACAGAAACGTAGTCGTCTCTGCAAC | |
| | AACCCCACACCCCAGTTTGGAGGCAAGGACTGCGTTGGTGATGTAACAGAAAACCAGATCTGCAACAAGCAGGACTGT | |
| | CCAATTGATGGATGCCTGTCCAATCCCTGCTTTGCCGGCGTGAAGTGTACTAGCTACCCTGATGGCAGCTGGAAATGT | |
| | GGTGCTTGTCCCCCTGGTTACAGTGGAAATGGCATCCAGTGCACAGATGTTGATGAGTGCAAAGAAGTGCCTGATGCC | |
| | TGCTTCAACCACAATGGAGAGCACCGGTGTGAGAACACGGACCCCGGCTACAACTGCCTGCCCTGCCCCCCACGCTTC | |
| | ACCGGCTCACAGCCCTTCGGCCAGGGTGTCGAACATGCCACGGCCAACAAACAGGTGTGCAAGCCCCGTAACCCCTGC | |
| | ACGGATGGGACCCACGACTGCAACAAGAACGCCAAGTGCAACTACCTGGGCCACTATAGCGACCCCATGTACCGCTGC | |
| | GAGTGCAAGCCTGGCTACGCTGGCAATGGCATCATCTGCGGGGAGGACACAGACCTGGATGGCTGGCCCAATGAGAAC | |
| | CTGGTGTGCGTGGCCAATGCGACTTACCACTGCAAAAAGGATAATTGCCCCAACCTTCCCAACTCAGGGCAGGAAGAC | |
| | TATGACAAGGATGGAATTGGTGATGCCTGTGATGATGACGATGACAATGATAAAATTCCAGATGACAGGGACAACTGT | |
| | CCATTCCATTACAACCCAGCTCAGTATGACTATGACAGAGATGATGTGGGAGACCGCTGTGACAACTGTCCCTACAAC | |
| | CACAACCCAGATCAGGCAGACACAGACAACAATGGGGAAGGAGACGCCTGTGCTGCAGACATTGATGGAGACGGTATC | |
| | CTCAATGAACGGGACAACTGCCAGTACGTCTACAATGTGGACCAGAGAGACACTGATATGGATGGGGTTGGAGATCAG | |
| | TGTGACAATTGCCCCTTGGAACACAATCCGGATCAGCTGGACTCTGACTCAGACCGCATTGGAGATACCTGTGACAAC | |
| | AATCAGGATATTGATGAAGATGGCCACCAGAACAATCTGGACAACTGTCCCTATGTGCCCAATGCCAACCAGGCTGAC | |
| | CATGACAAAGATGGCAAGGGAGATGCCTGTGACCACGATGATGACAACGATGGCATTCCTGATGACAAGGACAACTGC | |
| | AGACTCGTGCCCAATCCCGACCAGAAGGACTCTGACGGCGATGGTCGAGGTGATGCCTGCAAAGATGATTTTGACCAT | |
| | GACAGTGTGCCAGACATCGATGACATCTGTCCTGAGAATGTTGACATCAGTGAGACCGATTTCCGCCGATTCCAGATG | |
| | ATTCCTCTGGACCCCAAAGGGACATCCCAAAATGACCCTAACTGGGTTGTACGCCATCAGGGTAAAGAACTCGTCCAG | |
| | ACTGTCAACTGTGATCCTGGACTCGCTGTAGGTTATGATGAGTTTAATGCTGTGGACTTCAGTGGCACCTTCTTCATC | |
| | AACACCGAAAGGGACGATGACTATGCTGGATTTGTCTTTGGCTACCAGTCCAGCAGCCGCTTTTATGTTGTGATGTGG | |
| | AAGCAAGTCACCCAGTCCTACTGGGACACCAACCCCACGAGGGCTCAGGGATACTCGGGCCTTTCTGTGAAAGTTGTA | |
| | AACTCCACCACAGGGCCTGGCGAGCACCTGCGGAACGCCCTGTGGCACACAGGAAACACCCCTGGCCAGGTGCGCACC | |
| | CTGTGGCATGACCCTCGTCACATAGGCTGGAAAGATTTCACCGCCTACAGATGGCGTCTCAGCCACAGGCCAAAGACG | |
| | GGTTTCATTAGAGTGGTGATGTATGAAGGGAAGAAAATCATGGCTGACTCAGGACCCATCTATGATAAAACCTATGCT | |
| | GGTGGTAGACTAGGGTTGTTTGTCTTCTCTCAAGAAATGGTGTTCTTCTCTGACCTGAAATACGAATGTAGAGATCCC | |
| | TAA | |
| TSP1_HUMAN | MGLAWGLGVLFLMHVCGTNRIPESGGDNSVFDIFELTGAARKGSGRRLVKGPDPSSPAFRIEDANLIPPVPDDKFQDL | 10 |
| | VDAVRAEKGFLLLASLRQMKKTRGTLLALERKDHSGQVFSVVSNGKAGTLDLSLTVQGKQHVVSVEEALLATGQWKSI | |
| | TLFVQEDRAQLYIDCEKMENAELDVPIQSVFTRDLASIARLRIAKGGVNDNFQGVLQNVREVEGTTPEDILRNKGCSS | |
| | STSVLLTLDNNVVNGSSPAIRTNYIGHKTKDLQAICGISCDELSSMVLELRGLRTIVTTLQDSIRKVTEENKELANEL | |
| | RRPPLCYHNGVQYRNNEEWTVDSCTECHCQNSVTICKKVSCPIMPCSNATVPDGECCPRCWPSDSADDGWSPWSEWTS | |
| | CSTSCGNGIQQRGRSCDSLNNRCEGSSVQTRTCHIQECDKRFKQDGGWSHWSPWSSCSVTCGDGVITRIRLCNSPSPQ | |
| | MNGKPCEGEARETKACKKDACPINGGWGPWSPWDICSVTCGGGVQKRSRLCNNPTPQFGGKDCVGDVTENQICNKQDC | |
| | PIDGCLSNPCFAGVKCTSYPDGSWKCGACPPGYSGNGIQCTDVDECKEVPDACFNHNGEHRCENTDPGYNCLPCPPRF | |
| | TGSQPFGQGVEHATANKQVCKPRNPCTDGTHDCNKNAKCNYLGHYSDPMYRCECKPGYAGNGIICGEDTDLDGWPNEN | |
| | LVCVANATYHCKKDNCPNLPNSGQEDYDKDGIGDACDDDDDNDKIPDDRDNCPFHYNPAQYDYDRDDVGDRCDNCPYN | |
| | HNPDQADTDNNGEGDACAADIDGDGILNERDNCQYVYNVDQRDTDMDGVGDQCDNCPLEHNPDQLDSDSDRIGDTCDN | |
| | NQDIDEDGHQNNLDNCPYVPNANQADHDKDGKGDACDHDDDNDGIPDDKDNCRLVPNPDQKDSDGDGRGDACKDDFDH | |
| | DSVPDIDDICPENVDISETDFRRFQMIPLDPKGTSQNDPNWVVRHQGKELVQTVNCDPGLAVGYDEFNAVDFSGTFFI | |
| | NTERDDDYAGFVEGYQSSSRFYVVMWKQVTQSYWDTNPTRAQGYSGLSVKVVNSTTGPGEHLRNALWHTGNTPGQVRT | |
| | LWHDPRHIGWKDFTAYRWRLSHRPKTGFIRVVMYEGKKIMADSGPIYDKTYAGGRLGLFVFSQEMVFFSDLKYECRDP | |
| CD44_HUMAN | ATGGATAAATTTTGGTGGCATGCGGCGTGGGGCCTGTGCCTGGTGCCGCTGAGCCTGGCGCAGATTGATCTGAACATT | 11 |
| | ACCTGCCGCTTTGCGGGCGTGTTTCATGTGGAAAAAAACGGCCGCTATAGCATTAGCCGCACCGAAGCGGCGGATCTG | |
| | TGCAAAGCGTTTAACAGCACCCTGCCGACCATGGCGCAGATGGAAAAAGCGCTGAGCATTGGCTTTGAAACCTGCCGC | |
| | TATGGCTTTATTGAAGGCCATGTGGTGATTCCGCGCATTCATCCGAACAGCATTTGCGCGGCGAACAACACCGGCGTG | |
| | TATATTCTGACCAGCAACACCAGCCAGTATGATACCTATTGCTTTAACGCGAGCGCGCCGCCGGAAGAAGATTGCACC | |
| | AGCGTGACCGATCTGCCGAACGCGTTTGATGGCCCGATTACCATTACCATTGTGAACCGCGATGGCACCCGCTATGTG | |
| | CAGAAAGGCGAATATCGCACCAACCCGGAAGATATTTATCCGAGCAACCCGACCGATGATGATGTGAGCAGCGGCAGC | |
| | AGCAGCGAACGCAGCAGCACCAGCGGCGGCTATATTTTTTATACCTTTAGCACCGTGCATCCGATTCCGGATGAAGAT | |
| | AGCCCGTGGATTACCGATAGCACCGATCGCATTCCGGCGACCACCCTGATGAGCACCAGCGCGACCGCGACCGAAACC | |
| | GCGACCAAACGCCAGGAAACCTGGGATTGGTTTAGCTGGCTGTTTCTGCCGAGCGAAAGCAAAAACCATCTGCATACC | |
| | ACCACCCAGATGGCGGGCACCAGCAGCAACACCATTAGCGCGGGCTGGGAACCGAACGAAGAAAACGAAGATGAACGC | |
| | GATCGCCATCTGAGCTTTAGCGGCAGCGGCATTGATGATGATGAAGATTTTATTAGCAGCACCATTAGCACCACCCCG | |
| | CGCGCGTTTGATCATACCAAACAGAACCAGGATTGGACCCAGTGGAACCCGAGCCATAGCAACCCGGAAGTGCTGCTG | |
| | CAGACCACCACCCGCATGACCGATGTGGATCGCAACGGCACCACCGCGTATGAAGGCAACTGGAACCCGGAAGCGCAT | |
| | CCGCCGCTGATTCATCATGAACATCATGAAGAAGAAGAAACCCCGCATAGCACCAGCACCATTCAGGCGACCCCGAGC | |
| | AGCACCACCGAAGAAACCGCGACCCAGAAAGAACAGTGGTTTGGCAACCGCTGGCATGAAGGCTATCGCCAGACCCCG | |
| | AAAGAAGATAGCCATAGCACCACCGGCACCGCGGCGGCGAGCGCGCATACCAGCCATCCGATGCAGGGCCGCACCACC | |
| | CCGAGCCCGGAAGATAGCAGCTGGACCGATTTTTTTAACCCGATTAGCCATCCGATGGGCCGCGGCCATCAGGCGGGC | |
| | CGCCGCATGGATATGGATAGCAGCCATAGCATTACCCTGCAGCCGACCGCGAACCCGAACACCGGCCTGGTGGAAGAT | |
| | CTGGATCGCACCGGCCCGCTGAGCATGACCACCCAGCAGAGCAACAGCCAGAGCTTTAGCACCAGCCATGAAGGCCTG | |
| | GAAGAAGATAAAGATCATCCGACCACCAGCACCCTGACCAGCAGCAACCGCAACGATGTGACCGGCGGCCGCCGCGAT | |
| | CCGAACCATAGCGAAGGCAGCACCACCCTGCTGGAAGGCTATACCAGCCATTATCCGCATACCAAAGAAAGCCGCACC | |
| | TTTATTCCGGTGACCAGCGCGAAAACCGGCAGCTTTGGCGTGACCGCGGTGACCGTGGGCGATAGCAACAGCAACGTG | |
| | AACCGCAGCCTGAGCGGCGATCAGGATACCTTTCATCCGAGCGGCGGCAGCCATACCACCCATGGCAGCGAAAGCGAT | |
| | GGCCATAGCCATGGCAGCCAGGAAGGCGGCGCGAACACCACCAGCGGCCCGATTCGCACCCCGCAGATTCCGGAATGG | |
| | CTGATTATTCTGGCGAGCCTGCTGGCGCTGGCGCTGATTCTGGCGGTGTGCATTGCGGTGAACAGCCGCCGCCGCTGC | |
| | GGCCAGAAAAAAAAACTGGTGATTAACAGCGGCAACGGCGCGGTGGAAGATCGCAAACCGAGCGGCCTGAACGGCGAA | |
| | GCGAGCAAAAGCCAGGAAATGGTGCATCTGGTGAACAAAGAAAGCAGCGAAACCCCGGATCAGTTTATGACCGCGGAT | |
| | GAAACCCGCAACCTGCAGAACGTGGATATGAAAATTGGCGTG | |
| CD44_HUMAN | MDKFWWHAAWGLCLVPLSLAQIDLNITCRFAGVFHVEKNGRYSISRTEAADLCKAFNSTLPTMAQMEKALSIGFETCR | 12 |
| | YGFIEGHVVIPRIHPNSICAANNTGVYILTSNTSQYDTYCFNASAPPEEDCTSVTDLPNAFDGPITITIVNRDGTRYV | |
| | QKGEYRTNPEDIYPSNPTDDDVSSGSSSERSSTSGGYIFYTFSTVHPIPDEDSPWITDSTDRIPATTLMSTSATATET | |
| | ATKRQETWDWFSWLFLPSESKNHLHTTTQMAGTSSNTISAGWEPNEENEDERDRHLSFSGSGIDDDEDFISSTISTTP | |
| | RAFDHTKQNQDWTQWNPSHSNPEVLLQTTTRMTDVDRNGTTAYEGNWNPEAHPPLIHHEHHEEEETPHSTSTIQATPS | |
| | STTEETATQKEQWFGNRWHEGYRQTPKEDSHSTTGTAAASAHTSHPMQGRTTPSPEDSSWTDFFNPISHPMGRGHQAG | |
| | RRMDMDSSHSITLQPTANPNTGLVEDLDRTGPLSMTTQQSNSQSFSTSHEGLEEDKDHPTTSTLTSSNRNDVTGGRRD | |
| | PNHSEGSTTLLEGYTSHYPHTKESRTFIPVTSAKTGSFGVTAVTVGDSNSNVNRSLSGDQDTFHPSGGSHTTHGSESD | |
| | GHSHGSQEGGANTTSGPIRTPQIPEWLIILASLLALALILAVCIAVNSRRRCGQKKKLVINSGNGAVEDRKPSGLNGE | |
| | ASKSQEMVHLVNKESSETPDQFMTADETRNLQNVDMKIGV | |
| ENPL_HUMAN | ATGCGCGCGCTGTGGGTGCTGGGCCTGTGCTGCGTGCTGCTGACCTTTGGCAGCGTGCGCGCGGATGATGAAGTGGAT | 13 |
| | GTGGATGGCACCGTGGAAGAAGATCTGGGCAAAAGCCGCGAAGGCAGCCGCACCGATGATGAAGTGGTGCAGCGCGAA | |
| | GAAGAAGCGATTCAGCTGGATGGCCTGAACGCGAGCCAGATTCGCGAACTGCGCGAAAAAAGCGAAAAATTTGCGTTT | |
| | CAGGCGGAAGTGAACCGCATGATGAAACTGATTATTAACAGCCTGTATAAAAACAAAGAAATTTTTCTGCGCGAACTG | |
| | ATTAGCAACGCGAGCGATGCGCTGGATAAAATTCGCCTGATTAGCCTGACCGATGAAAACGCGCTGAGCGGCAACGAA | |
| | GAACTGACCGTGAAAATTAAATGCGATAAAGAAAAAAACCTGCTGCATGTGACCGATACCGGCGTGGGCATGACCCGC | |
| | GAAGAACTGGTGAAAAACCTGGGCACCATTGCGAAAAGCGGCACCAGCGAATTTCTGAACAAAATGACCGAAGCGCAG | |
| | GAAGATGGCCAGAGCACCAGCGAACTGATTGGCCAGTTTGGCGTGGGCTTTTATAGCGCGTTTCTGGTGGCGGATAAA | |
| | GTGATTGTGACCAGCAAACATAACAACGATACCCAGCATATTTGGGAAAGCGATAGCAACGAATTTAGCGTGATTGCG | |
| | GATCCGCGCGGCAACACCCTGGGCCGCGGCACCACCATTACCCTGGTGCTGAAAGAAGAAGCGAGCGATTATCTGGAA | |
| | CTGGATACCATTAAAAACCTGGTGAAAAAATATAGCCAGTTTATTAACTTTCCGATTTATGTGTGGAGCAGCAAAACC | |
| | GAAACCGTGGAAGAACCGATGGAAGAAGAAGAAGCGGCGAAAGAAGAAAAAGAAGAAAGCGATGATGAAGCGGCGGTG | |
| | GAAGAAGAAGAAGAAGAAAAAAAACCGAAAACCAAAAAAGTGGAAAAAACCGTGTGGGATTGGGAACTGATGAACGAT | |
| | ATTAAACCGATTTGGCAGCGCCCGAGCAAAGAAGTGGAAGAAGATGAATATAAAGCGTTTTATAAAAGCTTTAGCAAA | |
| | GAAAGCGATGATCCGATGGCGTATATTCATTTTACCGCGGAAGGCGAAGTGACCTTTAAAAGCATTCTGTTTGTGCCG | |
| | ACCAGCGCGCCGCGCGGCCTGTTTGATGAATATGGCAGCAAAAAAAGCGATTATATTAAACTGTATGTGCGCCGCGTG | |
| | TTTATTACCGATGATTTTCATGATATGATGCCGAAATATCTGAACTTTGTGAAAGGCGTGGTGGATAGCGATGATCTG | |
| | CCGCTGAACGTGAGCCGCGAAACCCTGCAGCAGCATAAACTGCTGAAAGTGATTCGCAAAAAACTGGTGCGCAAAACC | |
| | CTGGATATGATTAAAAAAATTGCGGATGATAAATATAACGATACCTTTTGGAAAGAATTTGGCACCAACATTAAACTG | |
| | GGCGTGATTGAAGATCATAGCAACCGCACCCGCCTGGCGAAACTGCTGCGCTTTCAGAGCAGCCATCATCCGACCGAT | |
| | ATTACCAGCCTGGATCAGTATGTGGAACGCATGAAAGAAAAACAGGATAAAATTTATTTTATGGCGGGCAGCAGCCGC | |
| | AAAGAAGCGGAAAGCAGCCCGTTTGTGGAACGCCTGCTGAAAAAAGGCTATGAAGTGATTTATCTGACCGAACCGGTG | |
| | GATGAATATTGCATTCAGGCGCTGCCGGAATTTGATGGCAAACGCTTTCAGAACGTGGCGAAAGAAGGCGTGAAATTT | |
| | GATGAAAGCGAAAAAACCAAAGAAAGCCGCGAAGCGGTGGAAAAAGAATTTGAACCGCTGCTGAACTGGATGAAAGAT | |
| | AAAGCGCTGAAAGATAAAATTGAAAAAGCGGTGGTGAGCCAGCGCCTGACCGAAAGCCCGTGCGCGCTGGTGGCGAGC | |
| | CAGTATGGCTGGAGCGGCAACATGGAACGCATTATGAAAGCGCAGGCGTATCAGACCGGCAAAGATATTAGCACCAAC | |
| | TATTATGCGAGCCAGAAAAAAACCTTTGAAATTAACCCGCGCCATCCGCTGATTCGCGATATGCTGCGCCGCATTAAA | |
| | GAAGATGAAGATGATAAAACCGTGCTGGATCTGGCGGTGGTGCTGTTTGAAACCGCGACCCTGCGCAGCGGCTATCTG | |
| | CTGCCGGATACCAAAGCGTATGGCGATCGCATTGAACGCATGCTGCGCCTGAGCCTGAACATTGATCCGGATGCGAAA | |
| | GTGGAAGAAGAACCGGAAGAAGAACCGGAAGAAACCGCGGAAGATACCACCGAAGATACCGAACAGGATGAAGATGAA | |
| | GAAATGGATGTGGGCACCGATGAAGAAGAAGAAACCGCGAAAGAAAGCACCGCGGAAAAAGATGAACTG | |
| ENPL_HUMAN | MRALWVLGLCCVLLTFGSVRADDEVDVDGTVEEDLGKSREGSRTDDEVVQREEEAIQLDGLNASQIRELREKSEKFAF | 14 |
| | QAEVNRMMKLIINSLYKNKEIFLRELISNASDALDKIRLISLTDENALSGNEELTVKIKCDKEKNLLHVTDTGVGMTR | |
| | EELVKNLGTIAKSGTSEFLNKMTEAQEDGQSTSELIGQFGVGFYSAFLVADKVIVTSKHNNDTQHIWESDSNEFSVIA | |
| | DPRGNTLGRGTTITLVLKEEASDYLELDTIKNLVKKYSQFINEPIYVWSSKTETVEEPMEEEEAAKEEKEESDDEAAV | |
| | EEEEEEKKPKTKKVEKTVWDWELMNDIKPIWQRPSKEVEEDEYKAFYKSFSKESDDPMAYIHFTAEGEVTFKSILFVP | |
| | TSAPRGLFDEYGSKKSDYIKLYVRRVFITDDFHDMMPKYLNFVKGVVDSDDLPLNVSRETLQQHKLLKVIRKKLVRKT | |
| | LDMIKKIADDKYNDTFWKEFGTNIKLGVIEDHSNRTRLAKLLRFQSSHHPTDITSLDQYVERMKEKQDKIYFMAGSSR | |
| | KEAESSPFVERLLKKGYEVIYLTEPVDEYCIQALPEFDGKREQNVAKEGVKFDESEKTKESREAVEKEFEPLLNWMKD | |
| | KALKDKIEKAVVSQRLTESPCALVASQYGWSGNMERIMKAQAYQTGKDISTNYYASQKKTFEINPRHPLIRDMLRRIK | |
| | EDEDDKTVLDLAVVLFETATLRSGYLLPDTKAYGDRIERMLRLSLNIDPDAKVEEEPEEEPEETAEDTTEDTEQDEDE | |
| | EMDVGTDEEEETAKESTAEKDEL | |
| TENX_HUMAN | ATGATGCCGGCGCAGTATGCGCTGACCAGCAGCCTGGTGCTGCTGGTGCTGCTGAGCACCGCGCGCGCGGGCCCGTTT | 15 |
| | AGCAGCCGCAGCAACGTGACCCTGCCGGCGCCGCGCCCGCCGCCGCAGCCGGGCGGCCATACCGTGGGCGCGGGCGTG | |
| | GGCAGCCCGAGCAGCCAGCTGTATGAACATACCGTGGAAGGCGGCGAAAAACAGGTGGTGTTTACCCATCGCATTAAC | |
| | CTGCCGCCGAGCACCGGCTGCGGCTGCCCGCCGGGCACCGAACCGCCGGTGCTGGCGAGCGAAGTGCAGGCGCTGCGC | |
| | GTGCGCCTGGAAATTCTGGAAGAACTGGTGAAAGGCCTGAAAGAACAGTGCACCGGCGGCTGCTGCCCGGCGAGCGCG | |
| | CAGGCGGGCACCGGCCAGACCGATGTGCGCACCCTGTGCAGCCTGCATGGCGTGTTTGATCTGAGCCGCTGCACCTGC | |
| | AGCTGCGAACCGGGCTGGGGCGGCCCGACCTGCAGCGATCCGACCGATGCGGAAATTCCGCCGAGCAGCCCGCCGAGC | |
| | GCGAGCGGCAGCTGCCCGGATGATTGCAACGATCAGGGCCGCTGCGTGCGCGGCCGCTGCGTGTGCTTTCCGGGCTAT | |
| | ACCGGCCCGAGCTGCGGCTGGCCGAGCTGCCCGGGCGATTGCCAGGGCCGCGGCCGCTGCGTGCAGGGCGTGTGCGTG | |
| | TGCCGCGCGGGCTTTAGCGGCCCGGATTGCAGCCAGCGCAGCTGCCCGCGCGGCTGCAGCCAGCGCGGCCGCTGCGAA | |
| | GGCGGCCGCTGCGTGTGCGATCCGGGCTATACCGGCGATGATTGCGGCATGCGCAGCTGCCCGCGCGGCTGCAGCCAG | |
| | CGCGGCCGCTGCGAAAACGGCCGCTGCGTGTGCAACCCGGGCTATACCGGCGAAGATTGCGGCGTGCGCAGCTGCCCG | |
| | CGCGGCTGCAGCCAGCGCGGCCGCTGCAAAGATGGCCGCTGCGTGTGCGATCCGGGCTATACCGGCGAAGATTGCGGC | |
| | ACCCGCAGCTGCCCGTGGGATTGCGGCGAAGGCGGCCGCTGCGTGGATGGCCGCTGCGTGTGCTGGCCGGGCTATACC | |
| | GGCGAAGATTGCAGCACCCGCACCTGCCCGCGCGATTGCCGCGGCCGCGGCCGCTGCGAAGATGGCGAATGCATTTGC | |
| | GATACCGGCTATAGCGGCGATGATTGCGGCGTGCGCAGCTGCCCGGGCGATTGCAACCAGCGCGGCCGCTGCGAAGAT | |
| | GGCCGCTGCGTGTGCTGGCCGGGCTATACCGGCACCGATTGCGGCAGCCGCGCGTGCCCGCGCGATTGCCGCGGCCGC | |
| | GGCCGCTGCGAAAACGGCGTGTGCGTGTGCAACGCGGGCTATAGCGGCGAAGATTGCGGCGTGCGCAGCTGCCCGGGC | |
| | GATTGCCGCGGCCGCGGCCGCTGCGAAAGCGGCCGCTGCATGTGCTGGCCGGGCTATACCGGCCGCGATTGCGGCACC | |
| | CGCGCGTGCCCGGGCGATTGCCGCGGCCGCGGCCGCTGCGTGGATGGCCGCTGCGTGTGCAACCCGGGCTTTACCGGC | |
| | GAAGATTGCGGCAGCCGCCGCTGCCCGGGCGATTGCCGCGGCCATGGCCTGTGCGAAGATGGCGTGTGCGTGTGCGAT | |
| | GCGGGCTATAGCGGCGAAGATTGCAGCACCCGCAGCTGCCCGGGCGGCTGCCGCGGCCGCGGCCAGTGCCTGGATGGC | |
| | CGCTGCGTGTGCGAAGATGGCTATAGCGGCGAAGATTGCGGCGTGCGCCAGTGCCCGAACGATTGCAGCCAGCATGGC | |
| | GTGTGCCAGGATGGCGTGTGCATTTGCTGGGAAGGCTATGTGAGCGAAGATTGCAGCATTCGCACCTGCCCGAGCAAC | |
| | TGCCATGGCCGCGGCCGCTGCGAAGAAGGCCGCTGCCTGTGCGATCCGGGCTATACCGGCCCGACCTGCGCGACCCGC | |
| | ATGTGCCCGGCGGATTGCCGCGGCCGCGGCCGCTGCGTGCAGGGCGTGTGCCTGTGCCATGTGGGCTATGGCGGCGAA | |
| | GATTGCGGCCAGGAAGAACCGCCGGCGAGCGCGTGCCCGGGCGGCTGCGGCCCGCGCGAACTGTGCCGCGCGGGCCAG | |
| | TGCGTGTGCGTGGAAGGCTTTCGCGGCCCGGATTGCGCGATTCAGACCTGCCCGGGCGATTGCCGCGGCCGCGGCGAA | |
| | TGCCATGATGGCAGCTGCGTGTGCAAAGATGGCTATGCGGGCGAAGATTGCGGCGAAGCGCGCGTGCCGAGCAGCGCG | |
| | AGCGCGTATGATCAGCGCGGCCTGGCGCCGGGCCAGGAATATCAGGTGACCGTGCGCGCGCTGCGCGGCACCAGCTGG | |
| | GGCCTGCCGGCGAGCAAAACCATTACCACCATGATTGATGGCCCGCAGGATCTGCGCGTGGTGGCGGTGACCCCGACC | |
| | ACCCTGGAACTGGGCTGGCTGCGCCCGCAGGCGGAAGTGGATCGCTTTGTGGTGAGCTATGTGAGCGCGGGCAACCAG | |
| | CGCGTGCGCCTGGAAGTGCCGCCGGAAGCGGATGGCACCCTGCTGACCGATCTGATGCCGGGCGTGGAATATGTGGTG | |
| | ACCGTGACCGCGGAACGCGGCCGCGCGGTGAGCTATCCGGCGAGCGTGCGCGCGAACACCGAAGAACGCGAAGAAGAA | |
| | AGCCCGCCGCGCCCGAGCCTGAGCCAGCCGCCGCGCCGCCCGTGGGGCAACCTGACCGCGGAACTGAGCCGCTTTCGC | |
| | GGCACCGTGCAGGATCTGGAACGCCATCTGCGCGCGCATGGCTATCCGCTGCGCGCGAACCAGACCTATACCAGCGTG | |
| | GCGCGCCATATTCATGAATATCTGCAGCGCCAGGTGCTGGGCAGCAGCGCGGATGGCGCGCTGCTGGTGAGCCTGGAT | |
| | GGCCTGCGCGGCCAGTTTGAACGCGTGGTGCTGCGCTGGCGCCCGCAGCCGCCGGCGGAAGGCCCGGGCGGCGAACTG | |
| | ACCGTGCCGGGCACCACCCGCACCGTGAGCCTGCCGGATCTGCGCCCGGGCACCACCTATCATGTGGAAGTGCATGGC | |
| | GTGCGCGCGGGCCAGACCAGCAAAAGCTATGCGTTTATTACCACCACCGGCCCGAGCACCACCCAGGGCGCGCAGGCG | |
| | CCGCTGCTGCAGCAGCGCCCGCAGGAACTGGGCGAACTGCGCGTGCTGGGCCGCGATGAAACCGGCCGCCTGCGCGTG | |
| | GTGTGGACCGCGCAGCCGGATACCTTTGCGTATTTTCAGCTGCGCATGCGCGTGCCGGAAGGCCCGGGCGCGCATGAA | |
| | GAAGTGCTGCCGGGCGATGTGCGCCAGGCGCTGGTGCCGCCGCCGCCGCCGGGCACCCCGTATGAACTGAGCCTGCAT | |
| | GGCGTGCCGCCGGGCGGCAAACCGAGCGATCCGATTATTTATCAGGGCATTATGGATAAAGATGAAGAAAAACCGGGC | |
| | AAAAGCAGCGGCCCGCCGCGCCTGGGCGAACTGACCGTGACCGATCGCACCAGCGATAGCCTGCTGCTGCGCTGGACC | |
| | GTGCCGGAAGGCGAATTTGATAGCTTTGTGATTCAGTATAAAGATCGCGATGGCCAGCCGCAGGTGGTGCCGGTGGAA | |
| | GGCCCGCAGCGCAGCGCGGTGATTACCAGCCTGGATCCGGGCCGCAAATATAAATTTGTGCTGTATGGCTTTGTGGGC | |
| | AAAAAACGCCATGGCCCGCTGGTGGCGGAAGCGAAAATTCTGCCGCAGAGCGATCCGAGCCCGGGCACCCCGCCGCAT | |
| | CTGGGCAACCTGTGGGTGACCGATCCGACCCCGGATAGCCTGCATCTGAGCTGGACCGTGCCGGAAGGCCAGTTTGAT | |
| | ACCTTTATGGTGCAGTATCGCGATCGCGATGGCCGCCCGCAGGTGGTGCCGGTGGAAGGCCCGGAACGCAGCTTTGTG | |
| | GTGAGCAGCCTGGATCCGGATCATAAATATCGCTTTACCCTGTTTGGCATTGCGAACAAAAAACGCTATGGCCCGCTG | |
| | ACCGCGGATGGCACCACCGCGCCGGAACGCAAAGAAGAACCGCCGCGCCCGGAATTTCTGGAACAGCCGCTGCTGGGC | |
| | GAACTGACCGTGACCGGCGTGACCCCGGATAGCCTGCGCCTGAGCTGGACCGTGGCGCAGGGCCCGTTTGATAGCTTT | |
| | ATGGTGCAGTATAAAGATGCGCAGGGCCAGCCGCAGGCGGTGCCGGTGGCGGGCGATGAAAACGAAGTGACCGTGCCG | |
| | GGCCTGGATCCGGATCGCAAATATAAAATGAACCTGTATGGCCTGCGCGGCCGCCAGCGCGTGGGCCCGGAAAGCGTG | |
| | GTGGCGAAAACCGCGCCGCAGGAAGATGTGGATGAAACCCCGAGCCCGACCGAACTGGGCACCGAAGCGCCGGAAAGC | |
| | CCGGAAGAACCGCTGCTGGGCGAACTGACCGTGACCGGCAGCAGCCCGGATAGCCTGAGCCTGTTTTGGACCGTGCCG | |
| | CAGGGCAGCTTTGATAGCTTTACCGTGCAGTATAAAGATCGCGATGGCCGCCCGCGCGCGGTGCGCGTGGGCGGCAAA | |
| | GAAAGCGAAGTGACCGTGGGCGGCCTGGAACCGGGCCATAAATATAAAATGCATCTGTATGGCCTGCATGAAGGCCAG | |
| | CGCGTGGGCCCGGTGAGCGCGGTGGGCGTGACCGCGCCGCAGCAGGAAGAAACCCCGCCGGCGACCGAAAGCCCGCTG | |
| | GAACCGCGCCTGGGCGAACTGACCGTGACCGATGTGACCCCGAACAGCGTGGGCCTGAGCTGGACCGTGCCGGAAGGC | |
| | CAGTTTGATAGCTTTATTGTGCAGTATAAAGATAAAGATGGCCAGCCGCAGGTGGTGCCGGTGGCGGCGGATCAGCGC | |
| | GAAGTGACCGTGTATAACCTGGAACCGGAACGCAAATATAAAATGAACATGTATGGCCTGCATGATGGCCAGCGCATG | |
| | GGCCCGCTGAGCGTGGTGATTGTGACCGCGCCGGCGACCGAAGCGAGCAAACCGCCGCTGGAACCGCGCCTGGGCGAA | |
| | CTGACCGTGACCGATATTACCCCGGATAGCGTGGGCCTGAGCTGGACCGTGCCGGAAGGCGAATTTGATAGCTTTGTG | |
| | GTGCAGTATAAAGATCGCGATGGCCAGCCGCAGGTGGTGCCGGTGGCGGCGGATCAGCGCGAAGTGACCATTCCGGAT | |
| | CTGGAACCGAGCCGCAAATATAAATTTCTGCTGTTTGGCATTCAGGATGGCAAACGCCGCAGCCCGGTGAGCGTGGAA | |
| | GCGAAAACCGTGGCGCGCGGCGATGCGAGCCCGGGCGCGCCGCCGCGCCTGGGCGAACTGTGGGTGACCGATCCGACC | |
| | CCGGATAGCCTGCGCCTGAGCTGGACCGTGCCGGAAGGCCAGTTTGATAGCTTTGTGGTGCAGTTTAAAGATAAAGAT | |
| | GGCCCGCAGGTGGTGCCGGTGGAAGGCCATGAACGCAGCGTGACCGTGACCCCGCTGGATGCGGGCCGCAAATATCGC | |
| | TTTCTGCTGTATGGCCTGCTGGGCAAAAAACGCCATGGCCCGCTGACCGCGGATGGCACCACCGAAGCGCGCAGCGCG | |
| | ATGGATGATACCGGCACCAAACGCCCGCCGAAACCGCGCCTGGGCGAAGAACTGCAGGTGACCACCGTGACCCAGAAC | |
| | AGCGTGGGCCTGAGCTGGACCGTGCCGGAAGGCCAGTTTGATAGCTTTGTGGTGCAGTATAAAGATCGCGATGGCCAG | |
| | CCGCAGGTGGTGCCGGTGGAAGGCAGCCTGCGCGAAGTGAGCGTGCCGGGCCTGGATCCGGCGCATCGCTATAAACTG | |
| | CTGCTGTATGGCCTGCATCATGGCAAACGCGTGGGCCCGATTAGCGCGGTGGCGATTACCGCGGGCCGCGAAGAAACC | |
| | GAAACCGAAACCACCGCGCCGACCCCGCCGGCGCCGGAACCGCATCTGGGCGAACTGACCGTGGAAGAAGCGACCAGC | |
| | CATACCCTGCATCTGAGCTGGATGGTGACCGAAGGCGAATTTGATAGCTTTGAAATTCAGTATACCGATCGCGATGGC | |
| | CAGCTGCAGATGGTGCGCATTGGCGGCGATCGCAACGATATTACCCTGAGCGGCCTGGAAAGCGATCATCGCTATCTG | |
| | GTGACCCTGTATGGCTTTAGCGATGGCAAACATGTGGGCCCGGTGCATGTGGAAGCGCTGACCGTGCCGGAAGAAGAA | |
| | AAACCGAGCGAACCGCCGACCGCGACCCCGGAACCGCCGATTAAACCGCGCCTGGGCGAACTGACCGTGACCGATGCG | |
| | ACCCCGGATAGCCTGAGCCTGAGCTGGACCGTGCCGGAAGGCCAGTTTGATCATTTTCTGGTGCAGTATCGCAACGGC | |
| | GATGGCCAGCCGAAAGCGGTGCGCGTGCCGGGCCATGAAGAAGGCGTGACCATTAGCGGCCTGGAACCGGATCATAAA | |
| | TATAAAATGAACCTGTATGGCTTTCATGGCGGCCAGCGCATGGGCCCGGTGAGCGTGGTGGGCGTGACCGAACCGAGC | |
| | ATGGAAGCGCCGGAACCGGCGGAAGAACCGCTGCTGGGCGAACTGACCGTGACCGGCAGCAGCCCGGATAGCCTGAGC | |
| | CTGAGCTGGACCGTGCCGCAGGGCCGCTTTGATAGCTTTACCGTGCAGTATAAAGATCGCGATGGCCGCCCGCAGGTG | |
| | GTGCGCGTGGGCGGCGAAGAAAGCGAAGTGACCGTGGGCGGCCTGGAACCGGGCCGCAAATATAAAATGCATCTGTAT | |
| | GGCCTGCATGAAGGCCGCCGCGTGGGCCCGGTGAGCGCGGTGGGCGTGACCGCGCCGGAAGAAGAAAGCCCGGATGCG | |
| | CCGCTGGCGAAACTGCGCCTGGGCCAGATGACCGTGCGCGATATTACCAGCGATAGCCTGAGCCTGAGCTGGACCGTG | |
| | CCGGAAGGCCAGTTTGATCATTTTCTGGTGCAGTTTAAAAACGGCGATGGCCAGCCGAAAGCGGTGCGCGTGCCGGGC | |
| | CATGAAGATGGCGTGACCATTAGCGGCCTGGAACCGGATCATAAATATAAAATGAACCTGTATGGCTTTCATGGCGGC | |
| | CAGCGCGTGGGCCCGGTGAGCGCGGTGGGCCTGACCGCGAGCACCGAACCGCCGACCCCGGAACCGCCGATTAAACCG | |
| | CGCCTGGAAGAACTGACCGTGACCGATGCGACCCCGGATAGCCTGAGCCTGAGCTGGACCGTGCCGGAAGGCCAGTTT | |
| | GATCATTTTCTGGTGCAGTATAAAAACGGCGATGGCCAGCCGAAAGCGACCCGCGTGCCGGGCCATGAAGATCGCGTG | |
| | ACCATTAGCGGCCTGGAACCGGATAACAAATATAAAATGAACCTGTATGGCTTTCATGGCGGCCAGCGCGTGGGCCCG | |
| | GTGAGCGCGATTGGCGTGACCGAAGAAGAAACCCCGAGCCCGACCGAACCGAGCATGGAAGCGCCGGAACCGCCGGAA | |
| | GAACCGCTGCTGGGCGAACTGACCGTGACCGGCAGCAGCCCGGATAGCCTGAGCCTGAGCTGGACCGTGCCGCAGGGC | |
| | CGCTTTGATAGCTTTACCGTGCAGTATAAAGATCGCGATGGCCGCCCGCAGGTGGTGCGCGTGGGCGGCGAAGAAAGC | |
| | GAAGTGACCGTGGGCGGCCTGGAACCGGGCCGCAAATATAAAATGCATCTGTATGGCCTGCATGAAGGCCGCCGCGTG | |
| | GGCCCGGTGAGCACCGTGGGCGTGACCGCGCCGCAGGAAGATGTGGATGAAACCCCGAGCCCGACCGAACCGGGCACC | |
| | GAAGCGCCGGGCCCGCCGGAAGAACCGCTGCTGGGCGAACTGACCGTGACCGGCAGCAGCCCGGATAGCCTGAGCCTG | |
| | AGCTGGACCGTGCCGCAGGGCCGCTTTGATAGCTTTACCGTGCAGTATAAAGATCGCGATGGCCGCCCGCAGGCGGTG | |
| | CGCGTGGGCGGCCAGGAAAGCAAAGTGACCGTGCGCGGCCTGGAACCGGGCCGCAAATATAAAATGCATCTGTATGGC | |
| | CTGCATGAAGGCCGCCGCCTGGGCCCGGTGAGCGCGGTGGGCGTGACCGAAGATGAAGCGGAAACCACCCAGGCGGTG | |
| | CCGACCATGACCCCGGAACCGCCGATTAAACCGCGCCTGGGCGAACTGACCATGACCGATGCGACCCCGGATAGCCTG | |
| | AGCCTGAGCTGGACCGTGCCGGAAGGCCAGTTTGATCATTTTCTGGTGCAGTATCGCAACGGCGATGGCCAGCCGAAA | |
| | GCGGTGCGCGTGCCGGGCCATGAAGATGGCGTGACCATTAGCGGCCTGGAACCGGATCATAAATATAAAATGAACCTG | |
| | TATGGCTTTCATGGCGGCCAGCGCGTGGGCCCGATTAGCGTGATTGGCGTGACCGAAGAAGAAACCCCGAGCCCGACC | |
| | GAACTGAGCACCGAAGCGCCGGAACCGCCGGAAGAACCGCTGCTGGGCGAACTGACCGTGACCGGCAGCAGCCCGGAT | |
| | AGCCTGAGCCTGAGCTGGACCATTCCGCAGGGCCATTTTGATAGCTTTACCGTGCAGTATAAAGATCGCGATGGCCGC | |
| | CCGCAGGTGATGCGCGTGCGCGGCGAAGAAAGCGAAGTGACCGTGGGCGGCCTGGAACCGGGCCGCAAATATAAAATG | |
| | CATCTGTATGGCCTGCATGAAGGCCGCCGCGTGGGCCCGGTGAGCACCGTGGGCGTGACCGTGCCGACCACCACCCCG | |
| | GAACCGCCGAACAAACCGCGCCTGGGCGAACTGACCGTGACCGATGCGACCCCGGATAGCCTGAGCCTGAGCTGGATG | |
| | GTGCCGGAAGGCCAGTTTGATCATTTTCTGGTGCAGTATCGCAACGGCGATGGCCAGCCGAAAGTGGTGCGCGTGCCG | |
| | GGCCATGAAGATGGCGTGACCATTAGCGGCCTGGAACCGGATCATAAATATAAAATGAACCTGTATGGCTTTCATGGC | |
| | GGCCAGCGCGTGGGCCCGATTAGCGTGATTGGCGTGACCGAAGAAGAAACCCCGGCGCCGACCGAACCGAGCACCGAA | |
| | GCGCCGGAACCGCCGGAAGAACCGCTGCTGGGCGAACTGACCGTGACCGGCAGCAGCCCGGATAGCCTGAGCCTGAGC | |
| | TGGACCATTCCGCAGGGCCGCTTTGATAGCTTTACCGTGCAGTATAAAGATCGCGATGGCCGCCCGCAGGTGGTGCGC | |
| | GTGCGCGGCGAAGAAAGCGAAGTGACCGTGGGCGGCCTGGAACCGGGCTGCAAATATAAAATGCATCTGTATGGCCTG | |
| | CATGAAGGCCAGCGCGTGGGCCCGGTGAGCGCGGTGGGCGTGACCGCGCCGAAAGATGAAGCGGAAACCACCCAGGCG | |
| | GTGCCGACCATGACCCCGGAACCGCCGATTAAACCGCGCCTGGGCGAACTGACCGTGACCGATGCGACCCCGGATAGC | |
| | CTGAGCCTGAGCTGGATGGTGCCGGAAGGCCAGTTTGATCATTTTCTGGTGCAGTATCGCAACGGCGATGGCCAGCCG | |
| | AAAGCGGTGCGCGTGCCGGGCCATGAAGATGGCGTGACCATTAGCGGCCTGGAACCGGATCATAAATATAAAATGAAC | |
| | CTGTATGGCTTTCATGGCGGCCAGCGCGTGGGCCCGGTGAGCGCGATTGGCGTGACCGAAGAAGAAACCCCGAGCCCG | |
| | ACCGAACCGAGCACCGAAGCGCCGGAAGCGCCGGAAGAACCGCTGCTGGGCGAACTGACCGTGACCGGCAGCAGCCCG | |
| | GATAGCCTGAGCCTGAGCTGGACCGTGCCGCAGGGCCGCTTTGATAGCTTTACCGTGCAGTATAAAGATCGCGATGGC | |
| | CAGCCGCAGGTGGTGCGCGTGCGCGGCGAAGAAAGCGAAGTGACCGTGGGCGGCCTGGAACCGGGCCGCAAATATAAA | |
| | ATGCATCTGTATGGCCTGCATGAAGGCCAGCGCGTGGGCCCGGTGAGCACCGTGGGCATTACCGCGCCGCTGCCGACC | |
| | CCGCTGCCGGTGGAACCGCGCCTGGGCGAACTGGCGGTGGCGGCGGTGACCAGCGATAGCGTGGGCCTGAGCTGGACC | |
| | GTGGCGCAGGGCCCGTTTGATAGCTTTCTGGTGCAGTATCGCGATGCGCAGGGCCAGCCGCAGGCGGTGCCGGTGAGC | |
| | GGCGATCTGCGCGCGGTGGCGGTGAGCGGCCTGGATCCGGCGCGCAAATATAAATTTCTGCTGTTTGGCCTGCAGAAC | |
| | GGCAAACGCCATGGCCCGGTGCCGGTGGAAGCGCGCACCGCGCCGGATACCAAACCGAGCCCGCGCCTGGGCGAACTG | |
| | ACCGTGACCGATGCGACCCCGGATAGCGTGGGCCTGAGCTGGACCGTGCCGGAAGGCGAATTTGATAGCTTTGTGGTG | |
| | CAGTATAAAGATAAAGATGGCCGCCTGCAGGTGGTGCCGGTGGCGGCGAACCAGCGCGAAGTGACCGTGCAGGGCCTG | |
| | GAACCGAGCCGCAAATATCGCTTTCTGCTGTATGGCCTGAGCGGCCGCAAACGCCTGGGCCCGATTAGCGCGGATAGC | |
| | ACCACCGCGCCGCTGGAAAAAGAACTGCCGCCGCATCTGGGCGAACTGACCGTGGCGGAAGAAACCAGCAGCAGCCTG | |
| | CGCCTGAGCTGGACCGTGGCGCAGGGCCCGTTTGATAGCTTTGTGGTGCAGTATCGCGATACCGATGGCCAGCCGCGC | |
| | GCGGTGCCGGTGGCGGCGGATCAGCGCACCGTGACCGTGGAAGATCTGGAACCGGGCAAAAAATATAAATTTCTGCTG | |
| | TATGGCCTGCTGGGCGGCAAACGCCTGGGCCCGGTGAGCGCGCTGGGCATGACCGCGCCGGAAGAAGATACCCCGGCG | |
| | CCGGAACTGGCGCCGGAAGCGCCGGAACCGCCGGAAGAACCGCGCCTGGGCGTGCTGACCGTGACCGATACCACCCCG | |
| | GATAGCATGCGCCTGAGCTGGAGCGTGGCGCAGGGCCCGTTTGATAGCTTTGTGGTGCAGTATGAAGATACCAACGGC | |
| | CAGCCGCAGGCGCTGCTGGTGGATGGCGATCAGAGCAAAATTCTGATTAGCGGCCTGGAACCGAGCACCCCGTATCGC | |
| | TTTCTGCTGTATGGCCTGCATGAAGGCAAACGCCTGGGCCCGCTGAGCGCGGAAGGCACCACCGGCCTGGCGCCGGCG | |
| | GGCCAGACCAGCGAAGAAAGCCGCCCGCGCCTGAGCCAGCTGAGCGTGACCGATGTGACCACCAGCAGCCTGCGCCTG | |
| | AACTGGGAAGCGCCGCCGGGCGCGTTTGATAGCTTTCTGCTGCGCTTTGGCGTGCCGAGCCCGAGCACCCTGGAACCG | |
| | CATCCGCGCCCGCTGCTGCAGCGCGAACTGATGGTGCCGGGCACCCGCCATAGCGCGGTGCTGCGCGATCTGCGCAGC | |
| | GGCACCCTGTATAGCCTGACCCTGTATGGCCTGCGCGGCCCGCATAAAGCGGATAGCATTCAGGGCACCGCGCGCACC | |
| | CTGAGCCCGGTGCTGGAAAGCCCGCGCGATCTGCAGTTTAGCGAAATTCGCGAAACCAGCGCGAAAGTGAACTGGATG | |
| | CCGCCGCCGAGCCGCGCGGATAGCTTTAAAGTGAGCTATCAGCTGGCGGATGGCGGCGAACCGCAGAGCGTGCAGGTG | |
| | GATGGCCAGGCGCGCACCCAGAAACTGCAGGGCCTGATTCCGGGCGCGCGCTATGAAGTGACCGTGGTGAGCGTGCGC | |
| | GGCTTTGAAGAAAGCGAACCGCTGACCGGCTTTCTGACCACCGTGCCGGATGGCCCGACCCAGCTGCGCGCGCTGAAC | |
| | CTGACCGAAGGCTTTGCGGTGCTGCATTGGAAACCGCCGCAGAACCCGGTGGATACCTATGATGTGCAGGTGACCGCG | |
| | CCGGGCGCGCCGCCGCTGCAGGCGGAAACCCCGGGCAGCGCGGTGGATTATCCGCTGCATGATCTGGTGCTGCATACC | |
| | AACTATACCGCGACCGTGCGCGGCCTGCGCGGCCCGAACCTGACCAGCCCGGCGAGCATTACCTTTACCACCGGCCTG | |
| | GAAGCGCCGCGCGATCTGGAAGCGAAAGAAGTGACCCCGCGCACCGCGCTGCTGACCTGGACCGAACCGCCGGTGCGC | |
| | CCGGCGGGCTATCTGCTGAGCTTTCATACCCCGGGCGGCCAGAACCAGGAAATTCTGCTGCCGGGCGGCATTACCAGC | |
| | CATCAGCTGCTGGGCCTGTTTCCGAGCACCAGCTATAACGCGCGCCTGCAGGCGATGTGGGGCCAGAGCCTGCTGCCG | |
| | CCGGTGAGCACCAGCTTTACCACCGGCGGCCTGCGCATTCCGTTTCCGCGCGATTGCGGCGAAGAAATGCAGAACGGC | |
| | GCGGGCGCGAGCCGCACCAGCACCATTTTTCTGAACGGCAACCGCGAACGCCCGCTGAACGTGTTTTGCGATATGGAA | |
| | ACCGATGGCGGCGGCTGGCTGGTGTTTCAGCGCCGCATGGATGGCCAGACCGATTTTTGGCGCGATTGGGAAGATTAT | |
| | GCGCATGGCTTTGGCAACATTAGCGGCGAATTTTGGCTGGGCAACGAAGCGCTGCATAGCCTGACCCAGGCGGGCGAT | |
| | TATAGCATGCGCGTGGATCTGCGCGCGGGCGATGAAGCGGTGTTTGCGCAGTATGATAGCTTTCATGTGGATAGCGCG | |
| | GCGGAATATTATCGCCTGCATCTGGAAGGCTATCATGGCACCGCGGGCGATAGCATGAGCTATCATAGCGGCAGCGTG | |
| | TTTAGCGCGCGCGATCGCGATCCGAACAGCCTGCTGATTAGCTGCGCGGTGAGCTATCGCGGCGCGTGGTGGTATCGC | |
| | AACTGCCATTATGCGAACCTGAACGGCCTGTATGGCAGCACCGTGGATCATCAGGGCGTGAGCTGGTATCATTGGAAA | |
| | GGCTTTGAATTTAGCGTGCCGTTTACCGAAATGAAACTGCGCCCGCGCAACTTTCGCAGCCCGGCGGGCGGCGGC | |
| TENX_HUMAN | MMPAQYALTSSLVLLVLLSTARAGPFSSRSNVTLPAPRPPPQPGGHTVGAGVGSPSSQLYEHTVEGGEKQVVFTHRIN | 16 |
| | LPPSTGCGCPPGTEPPVLASEVQALRVRLEILEELVKGLKEQCTGGCCPASAQAGTGQTDVRTLCSLHGVFDLSRCTC | |
| | SCEPGWGGPTCSDPTDAEIPPSSPPSASGSCPDDCNDQGRCVRGRCVCFPGYTGPSCGWPSCPGDCQGRGRCVQGVCV | |
| | CRAGFSGPDCSQRSCPRGCSQRGRCEGGRCVCDPGYTGDDCGMRSCPRGCSQRGRCENGRCVCNPGYTGEDCGVRSCP | |
| | RGCSQRGRCKDGRCVCDPGYTGEDCGTRSCPWDCGEGGRCVDGRCVCWPGYTGEDCSTRTCPRDCRGRGRCEDGECIC | |
| | DTGYSGDDCGVRSCPGDCNQRGRCEDGRCVCWPGYTGTDCGSRACPRDCRGRGRCENGVCVCNAGYSGEDCGVRSCPG | |
| | DCRGRGRCESGRCMCWPGYTGRDCGTRACPGDCRGRGRCVDGRCVCNPGFTGEDCGSRRCPGDCRGHGLCEDGVCVCD | |
| | AGYSGEDCSTRSCPGGCRGRGQCLDGRCVCEDGYSGEDCGVRQCPNDCSQHGVCQDGVCICWEGYVSEDCSIRTCPSN | |
| | CHGRGRCEEGRCLCDPGYTGPTCATRMCPADCRGRGRCVQGVCLCHVGYGGEDCGQEEPPASACPGGCGPRELCRAGQ | |
| | CVCVEGFRGPDCAIQTCPGDCRGRGECHDGSCVCKDGYAGEDCGEARVPSSASAYDQRGLAPGQEYQVTVRALRGTSW | |
| | GLPASKTITTMIDGPQDLRVVAVTPTTLELGWLRPQAEVDREVVSYVSAGNQRVRLEVPPEADGTLLTDLMPGVEYVV | |
| | TVTAERGRAVSYPASVRANTEEREEESPPRPSLSQPPRRPWGNLTAELSRFRGTVQDLERHLRAHGYPLRANQTYTSV | |
| | ARHIHEYLQRQVLGSSADGALLVSLDGLRGQFERVVLRWRPQPPAEGPGGELTVPGTTRTVSLPDLRPGTTYHVEVHG | |
| | VRAGQTSKSYAFITTTGPSTTQGAQAPLLQQRPQELGELRVLGRDETGRLRVVWTAQPDTFAYFQLRMRVPEGPGAHE | |
| | EVLPGDVRQALVPPPPPGTPYELSLHGVPPGGKPSDPIIYQGIMDKDEEKPGKSSGPPRLGELTVTDRTSDSLLLRWT | |
| | VPEGEFDSEVIQYKDRDGQPQVVPVEGPQRSAVITSLDPGRKYKFVLYGFVGKKRHGPLVAEAKILPQSDPSPGTPPH | |
| | LGNLWVTDPTPDSLHLSWTVPEGQFDTFMVQYRDRDGRPQVVPVEGPERSFVVSSLDPDHKYRFTLFGIANKKRYGPL | |
| | TADGTTAPERKEEPPRPEFLEQPLLGELTVTGVTPDSLRLSWTVAQGPFDSFMVQYKDAQGQPQAVPVAGDENEVTVP | |
| | GLDPDRKYKMNLYGLRGRQRVGPESVVAKTAPQEDVDETPSPTELGTEAPESPEEPLLGELTVTGSSPDSLSLFWTVP | |
| | QGSFDSFTVQYKDRDGRPRAVRVGGKESEVTVGGLEPGHKYKMHLYGLHEGQRVGPVSAVGVTAPQQEETPPATESPL | |
| | EPRLGELTVTDVTPNSVGLSWTVPEGQFDSFIVQYKDKDGQPQVVPVAADQREVTVYNLEPERKYKMNMYGLHDGQRM | |
| | GPLSVVIVTAPATEASKPPLEPRLGELTVTDITPDSVGLSWTVPEGEFDSFVVQYKDRDGQPQVVPVAADQREVTIPD | |
| | LEPSRKYKFLLFGIQDGKRRSPVSVEAKTVARGDASPGAPPRLGELWVTDPTPDSLRLSWTVPEGQFDSFVVQFKDKD | |
| | GPQVVPVEGHERSVTVTPLDAGRKYRFLLYGLLGKKRHGPLTADGTTEARSAMDDTGTKRPPKPRLGEELQVTTVTQN | |
| | SVGLSWTVPEGQFDSFVVQYKDRDGQPQVVPVEGSLREVSVPGLDPAHRYKLLLYGLHHGKRVGPISAVAITAGREET | |
| | ETETTAPTPPAPEPHLGELTVEEATSHTLHLSWMVTEGEFDSFEIQYTDRDGQLQMVRIGGDRNDITLSGLESDHRYL | |
| | VTLYGESDGKHVGPVHVEALTVPEEEKPSEPPTATPEPPIKPRLGELTVTDATPDSLSLSWTVPEGQFDHELVQYRNG | |
| | DGQPKAVRVPGHEEGVTISGLEPDHKYKMNLYGFHGGQRMGPVSVVGVTEPSMEAPEPAEEPLLGELTVTGSSPDSLS | |
| | LSWTVPQGRFDSFTVQYKDRDGRPQVVRVGGEESEVTVGGLEPGRKYKMHLYGLHEGRRVGPVSAVGVTAPEEESPDA | |
| | PLAKLRLGQMTVRDITSDSLSLSWTVPEGQFDHELVQFKNGDGQPKAVRVPGHEDGVTISGLEPDHKYKMNLYGFHGG | |
| | QRVGPVSAVGLTASTEPPTPEPPIKPRLEELTVTDATPDSLSLSWTVPEGQFDHELVQYKNGDGQPKATRVPGHEDRV | |
| | TISGLEPDNKYKMNLYGFHGGQRVGPVSAIGVTEEETPSPTEPSMEAPEPPEEPLLGELTVTGSSPDSLSLSWTVPQG | |
| | RFDSFTVQYKDRDGRPQVVRVGGEESEVTVGGLEPGRKYKMHLYGLHEGRRVGPVSTVGVTAPQEDVDETPSPTEPGT | |
| | EAPGPPEEPLLGELTVTGSSPDSLSLSWTVPQGRFDSFTVQYKDRDGRPQAVRVGGQESKVTVRGLEPGRKYKMHLYG | |
| | LHEGRRLGPVSAVGVTEDEAETTQAVPTMTPEPPIKPRLGELTMTDATPDSLSLSWTVPEGQFDHELVQYRNGDGQPK | |
| | AVRVPGHEDGVTISGLEPDHKYKMNLYGFHGGQRVGPISVIGVTEEETPSPTELSTEAPEPPEEPLLGELTVTGSSPD | |
| | SLSLSWTIPQGHFDSFTVQYKDRDGRPQVMRVRGEESEVTVGGLEPGRKYKMHLYGLHEGRRVGPVSTVGVTVPTTTP | |
| | EPPNKPRLGELTVTDATPDSLSLSWMVPEGQFDHELVQYRNGDGQPKVVRVPGHEDGVTISGLEPDHKYKMNLYGFHG | |
| | GQRVGPISVIGVTEEETPAPTEPSTEAPEPPEEPLLGELTVTGSSPDSLSLSWTIPQGRFDSFTVQYKDRDGRPQVVR | |
| | VRGEESEVTVGGLEPGCKYKMHLYGLHEGQRVGPVSAVGVTAPKDEAETTQAVPTMTPEPPIKPRLGELTVTDATPDS | |
| | LSLSWMVPEGQFDHFLVQYRNGDGQPKAVRVPGHEDGVTISGLEPDHKYKMNLYGFHGGQRVGPVSAIGVTEEETPSP | |
| | TEPSTEAPEAPEEPLLGELTVTGSSPDSLSLSWTVPQGRFDSFTVQYKDRDGQPQVVRVRGEESEVTVGGLEPGRKYK | |
| | MHLYGLHEGQRVGPVSTVGITAPLPTPLPVEPRLGELAVAAVTSDSVGLSWTVAQGPFDSFLVQYRDAQGQPQAVPVS | |
| | GDLRAVAVSGLDPARKYKFLLFGLQNGKRHGPVPVEARTAPDTKPSPRLGELTVTDATPDSVGLSWTVPEGEFDSFVV | |
| | QYKDKDGRLQVVPVAANQREVTVQGLEPSRKYRELLYGLSGRKRLGPISADSTTAPLEKELPPHLGELTVAEETSSSL | |
| | RLSWTVAQGPFDSFVVQYRDTDGQPRAVPVAADQRTVTVEDLEPGKKYKFLLYGLLGGKRLGPVSALGMTAPEEDTPA | |
| | PELAPEAPEPPEEPRLGVLTVTDTTPDSMRLSWSVAQGPFDSFVVQYEDTNGQPQALLVDGDQSKILISGLEPSTPYR | |
| | FLLYGLHEGKRLGPLSAEGTTGLAPAGQTSEESRPRLSQLSVTDVTTSSLRLNWEAPPGAFDSFLLREGVPSPSTLEP | |
| | HPRPLLQRELMVPGTRHSAVLRDLRSGTLYSLTLYGLRGPHKADSIQGTARTLSPVLESPRDLQFSEIRETSAKVNWM | |
| | PPPSRADSFKVSYQLADGGEPQSVQVDGQARTQKLQGLIPGARYEVTVVSVRGFEESEPLTGFLTTVPDGPTQLRALN | |
| | LTEGFAVLHWKPPQNPVDTYDVQVTAPGAPPLQAETPGSAVDYPLHDLVLHTNYTATVRGLRGPNLTSPASITFTTGL | |
| | EAPRDLEAKEVTPRTALLTWTEPPVRPAGYLLSFHTPGGQNQEILLPGGITSHQLLGLFPSTSYNARLQAMWGQSLLP | |
| | PVSTSFTTGGLRIPFPRDCGEEMQNGAGASRTSTIFLNGNRERPLNVECDMETDGGGWLVFQRRMDGQTDEWRDWEDY | |
| | AHGEGNISGEFWLGNEALHSLTQAGDYSMRVDLRAGDEAVFAQYDSFHVDSAAEYYRLHLEGYHGTAGDSMSYHSGSV | |
| | FSARDRDPNSLLISCAVSYRGAWWYRNCHYANLNGLYGSTVDHQGVSWYHWKGFEFSVPFTEMKLRPRNFRSPAGGG | |
| CLUS_HUMAN | ATGATGAAAACCCTGCTGCTGTTTGTGGGCCTGCTGCTGACCTGGGAAAGCGGCCAGGTGCTGGGCGATCAGACCGTG | 17 |
| | AGCGATAACGAACTGCAGGAAATGAGCAACCAGGGCAGCAAATATGTGAACAAAGAAATTCAGAACGCGGTGAACGGC | |
| | GTGAAACAGATTAAAACCCTGATTGAAAAAACCAACGAAGAACGCAAAACCCTGCTGAGCAACCTGGAAGAAGCGAAA | |
| | AAAAAAAAAGAAGATGCGCTGAACGAAACCCGCGAAAGCGAAACCAAACTGAAAGAACTGCCGGGCGTGTGCAACGAA | |
| | ACCATGATGGCGCTGTGGGAAGAATGCAAACCGTGCCTGAAACAGACCTGCATGAAATTTTATGCGCGCGTGTGCCGC | |
| | AGCGGCAGCGGCCTGGTGGGCCGCCAGCTGGAAGAATTTCTGAACCAGAGCAGCCCGTTTTATTTTTGGATGAACGGC | |
| | GATCGCATTGATAGCCTGCTGGAAAACGATCGCCAGCAGACCCATATGCTGGATGTGATGCAGGATCATTTTAGCCGC | |
| | GCGAGCAGCATTATTGATGAACTGTTTCAGGATCGCTTTTTTACCCGCGAACCGCAGGATACCTATCATTATCTGCCG | |
| | TTTAGCCTGCCGCATCGCCGCCCGCATTTTTTTTTTCCGAAAAGCCGCATTGTGCGCAGCCTGATGCCGTTTAGCCCG | |
| | TATGAACCGCTGAACTTTCATGCGATGTTTCAGCCGTTTCTGGAAATGATTCATGAAGCGCAGCAGGCGATGGATATT | |
| | CATTTTCATAGCCCGGCGTTTCAGCATCCGCCGACCGAATTTATTCGCGAAGGCGATGATGATCGCACCGTGTGCCGC | |
| | GAAATTCGCCATAACAGCACCGGCTGCCTGCGCATGAAAGATCAGTGCGATAAATGCCGCGAAATTCTGAGCGTGGAT | |
| | TGCAGCACCAACAACCCGAGCCAGGCGAAACTGCGCCGCGAACTGGATGAAAGCCTGCAGGTGGCGGAACGCCTGACC | |
| | CGCAAATATAACGAACTGCTGAAAAGCTATCAGTGGAAAATGCTGAACACCAGCAGCCTGCTGGAACAGCTGAACGAA | |
| | CAGTTTAACTGGGTGAGCCGCCTGGCGAACCTGACCCAGGGCGAAGATCAGTATTATCTGCGCGTGACCACCGTGGCG | |
| | AGCCATACCAGCGATAGCGATGTGCCGAGCGGCGTGACCGAAGTGGTGGTGAAACTGTTTGATAGCGATCCGATTACC | |
| | GTGACCGTGCCGGTGGAAGTGAGCCGCAAAAACCCGAAATTTATGGAAACCGTGGCGGAAAAAGCGCTGCAGGAATAT | |
| | CGCAAAAAACATCGCGAAGAA | |
| CLUS_HUMAN | MMKTLLLFVGLLLTWESGQVLGDQTVSDNELQEMSNQGSKYVNKEIQNAVNGVKQIKTLIEKTNEERKTLLSNLEEAK | |
| | KKKEDALNETRESETKLKELPGVCNETMMALWEECKPCLKQTCMKFYARVCRSGSGLVGRQLEEFLNQSSPFYFWMNG | |
| | DRIDSLLENDRQQTHMLDVMQDHFSRASSIIDELFQDRFFTREPQDTYHYLPFSLPHRRPHFFFPKSRIVRSLMPFSP | 18 |
| | YEPLNFHAMFQPFLEMIHEAQQAMDIHFHSPAFQHPPTEFIREGDDDRTVCREIRHNSTGCLRMKDQCDKCREILSVD | |
| | CSTNNPSQAKLRRELDESLQVAERLTRKYNELLKSYQWKMLNTSSLLEQLNEQFNWVSRLANLTQGEDQYYLRVTTVA | |
| | SHTSDSDVPSGVTEVVVKLFDSDPITVTVPVEVSRKNPKFMETVAEKALQEYRKKHREE | |
| IBP3_HUMAN | ATGCAGCGCGCGCGCCCGACCCTGTGGGCGGCGGCGCTGACCCTGCTGGTGCTGCTGCGCGGCCCGCCGGTGGCGCGC | 19 |
| | GCGGGCGCGAGCAGCGCGGGCCTGGGCCCGGTGGTGCGCTGCGAACCGTGCGATGCGCGCGCGCTGGCGCAGTGCGCG | |
| | CCGCCGCCGGCGGTGTGCGCGGAACTGGTGCGCGAACCGGGCTGCGGCTGCTGCCTGACCTGCGCGCTGAGCGAAGGC | |
| | CAGCCGTGCGGCATTTATACCGAACGCTGCGGCAGCGGCCTGCGCTGCCAGCCGAGCCCGGATGAAGCGCGCCCGCTG | |
| | CAGGCGCTGCTGGATGGCCGCGGCCTGTGCGTGAACGCGAGCGCGGTGAGCCGCCTGCGCGCGTATCTGCTGCCGGCG | |
| | CCGCCGGCGCCGGGCAACGCGAGCGAAAGCGAAGAAGATCGCAGCGCGGGCAGCGTGGAAAGCCCGAGCGTGAGCAGC | |
| | ACCCATCGCGTGAGCGATCCGAAATTTCATCCGCTGCATAGCAAAATTATTATTATTAAAAAAGGCCATGCGAAAGAT | |
| | AGCCAGCGCTATAAAGTGGATTATGAAAGCCAGAGCACCGATACCCAGAACTTTAGCAGCGAAAGCAAACGCGAAACC | |
| | GAATATGGCCCGTGCCGCCGCGAAATGGAAGATACCCTGAACCATCTGAAATTTCTGAACGTGCTGAGCCCGCGCGGC | |
| | GTGCATATTCCGAACTGCGATAAAAAAGGCTTTTATAAAAAAAAACAGTGCCGCCCGAGCAAAGGCCGCAAACGCGGC | |
| | TTTTGCTGGTGCGTGGATAAATATGGCCAGCCGCTGCCGGGCTATACCACCAAAGGCAAAGAAGATGTGCATTGCTAT | |
| | AGCATGCAGAGCAAA | |
| IBP3_HUMAN | MQRARPTLWAAALTLLVLLRGPPVARAGASSAGLGPVVRCEPCDARALAQCAPPPAVCAELVREPGCGCCLTCALSEG | 20 |
| | QPCGIYTERCGSGLRCQPSPDEARPLQALLDGRGLCVNASAVSRLRAYLLPAPPAPGNASESEEDRSAGSVESPSVSS | |
| | THRVSDPKFHPLHSKIIIIKKGHAKDSQRYKVDYESQSTDTQNFSSESKRETEYGPCRREMEDTLNHLKFLNVLSPRG | |
| | VHIPNCDKKGFYKKKQCRPSKGRKRGFCWCVDKYGQPLPGYTTKGKEDVHCYSMQSK | |
| GELS_HUMAN | ATGGCGCCGCATCGCCCGGCGCCGGCGCTGCTGTGCGCGCTGAGCCTGGCGCTGTGCGCGCTGAGCCTGCCGGTGCGC | 21 |
| | GCGGCGACCGCGAGCCGCGGCGCGAGCCAGGCGGGCGCGCCGCAGGGCCGCGTGCCGGAAGCGCGCCCGAACAGCATG | |
| | GTGGTGGAACATCCGGAATTTCTGAAAGCGGGCAAAGAACCGGGCCTGCAGATTTGGCGCGTGGAAAAATTTGATCTG | |
| | GTGCCGGTGCCGACCAACCTGTATGGCGATTTTTTTACCGGCGATGCGTATGTGATTCTGAAAACCGTGCAGCTGCGC | |
| | AACGGCAACCTGCAGTATGATCTGCATTATTGGCTGGGCAACGAATGCAGCCAGGATGAAAGCGGCGCGGCGGCGATT | |
| | TTTACCGTGCAGCTGGATGATTATCTGAACGGCCGCGCGGTGCAGCATCGCGAAGTGCAGGGCTTTGAAAGCGCGACC | |
| | TTTCTGGGCTATTTTAAAAGCGGCCTGAAATATAAAAAAGGCGGCGTGGCGAGCGGCTTTAAACATGTGGTGCCGAAC | |
| | GAAGTGGTGGTGCAGCGCCTGTTTCAGGTGAAAGGCCGCCGCGTGGTGCGCGCGACCGAAGTGCCGGTGAGCTGGGAA | |
| | AGCTTTAACAACGGCGATTGCTTTATTCTGGATCTGGGCAACAACATTCATCAGTGGTGCGGCAGCAACAGCAACCGC | |
| | TATGAACGCCTGAAAGCGACCCAGGTGAGCAAAGGCATTCGCGATAACGAACGCAGCGGCCGCGCGCGCGTGCATGTG | |
| | AGCGAAGAAGGCACCGAACCGGAAGCGATGCTGCAGGTGCTGGGCCCGAAACCGGCGCTGCCGGCGGGCACCGAAGAT | |
| | ACCGCGAAAGAAGATGCGGCGAACCGCAAACTGGCGAAACTGTATAAAGTGAGCAACGGCGCGGGCACCATGAGCGTG | |
| | AGCCTGGTGGCGGATGAAAACCCGTTTGCGCAGGGCGCGCTGAAAAGCGAAGATTGCTTTATTCTGGATCATGGCAAA | |
| | GATGGCAAAATTTTTGTGTGGAAAGGCAAACAGGCGAACACCGAAGAACGCAAAGCGGCGCTGAAAACCGCGAGCGAT | |
| | TTTATTACCAAAATGGATTATCCGAAACAGACCCAGGTGAGCGTGCTGCCGGAAGGCGGCGAAACCCCGCTGTTTAAA | |
| | CAGTTTTTTAAAAACTGGCGCGATCCGGATCAGACCGATGGCCTGGGCCTGAGCTATCTGAGCAGCCATATTGCGAAC | |
| | GTGGAACGCGTGCCGTTTGATGCGGCGACCCTGCATACCAGCACCGCGATGGCGGCGCAGCATGGCATGGATGATGAT | |
| | GGCACCGGCCAGAAACAGATTTGGCGCATTGAAGGCAGCAACAAAGTGCCGGTGGATCCGGCGACCTATGGCCAGTTT | |
| | TATGGCGGCGATAGCTATATTATTCTGTATAACTATCGCCATGGCGGCCGCCAGGGCCAGATTATTTATAACTGGCAG | |
| | GGCGCGCAGAGCACCCAGGATGAAGTGGCGGCGAGCGCGATTCTGACCGCGCAGCTGGATGAAGAACTGGGCGGCACC | |
| | CCGGTGCAGAGCCGCGTGGTGCAGGGCAAAGAACCGGCGCATCTGATGAGCCTGTTTGGCGGCAAACCGATGATTATT | |
| | TATAAAGGCGGCACCAGCCGCGAAGGCGGCCAGACCGCGCCGGCGAGCACCCGCCTGTTTCAGGTGCGCGCGAACAGC | |
| | GCGGGCGCGACCCGCGCGGTGGAAGTGCTGCCGAAAGCGGGCGCGCTGAACAGCAACGATGCGTTTGTGCTGAAAACC | |
| | CCGAGCGCGGCGTATCTGTGGGTGGGCACCGGCGCGAGCGAAGCGGAAAAAACCGGCGCGCAGGAACTGCTGCGCGTG | |
| | CTGCGCGCGCAGCCGGTGCAGGTGGCGGAAGGCAGCGAACCGGATGGCTTTTGGGAAGCGCTGGGCGGCAAAGCGGCG | |
| | TATCGCACCAGCCCGCGCCTGAAAGATAAAAAAATGGATGCGCATCCGCCGCGCCTGTTTGCGTGCAGCAACAAAATT | |
| | GGCCGCTTTGTGATTGAAGAAGTGCCGGGCGAACTGATGCAGGAAGATCTGGCGACCGATGATGTGATGCTGCTGGAT | |
| | ACCTGGGATCAGGTGTTTGTGTGGGTGGGCAAAGATAGCCAGGAAGAAGAAAAAACCGAAGCGCTGACCAGCGCGAAA | |
| | CGCTATATTGAAACCGATCCGGCGAACCGCGATCGCCGCACCCCGATTACCGTGGTGAAACAGGGCTTTGAACCGCCG | |
| | AGCTTTGTGGGCTGGTTTCTGGGCTGGGATGATGATTATTGGAGCGTGGATCCGCTGGATCGCGCGATGGCGGAACTG | |
| | GCGGCGGGCTGCGGCTGCGGCTGCTGCTGCGGCTGCACCGGCTGCGCGGGCGGCTGCGGCTGCACCGGCTGCACCGGC | |
| | GGCGCGACCGGCGGCTGCTGCGGCTGCGGCGGCTGCTGCACCGGCACCGGCTGCGGCACCGGCGCGGCGTGCGGCTGC | |
| | GGCGCGGGCTGCGGCTGCGGCGGCACCGGCGCGGGCTGCTGCGGCTGCTGCACCGGCTGCGGCTGCGGCTGCGGCACC | |
| | GCGACCTGCACCGGCTGCACCGGCTGCTGCGGCGGCTGCGGCTGCTGCGGCTGCTGCGGCGGCTGCGGCTGCTGCGGC | |
| | GGCGGCTGCGGCGCGGCGTGCTGCGGCTGCTGCGGCGGCTGCGGCTGCTGCGGCGGCGGCTGCGCGGCGTGCGGCTGC | |
| | GGCGCGGGCTGCGGCGCGGCGGCGGGCTGCGGCGCGGCGGGCGCGGCGGGCGCGACCTGCGGCTGCGCGGGCTGCGGC | |
| | TGCGGCGGCGGCTGCGCGGGCTGCGGCACCGGCGGCGCGGCGGCGGGCTGCTGCTGCGGCGCGGGCTGCGGCACCGGC | |
| | GCGGGCTGCGCGGGCTGCGCGTGCTGCTGCGCGACCTGCGGCTGCGGCACCGGCGCGGGCTGCGGCGCGACCTGCTGC | |
| | GGCGCGGCGGCGACCACCACCTGCGCGACCTGCTGCGGCTGCACCGGCTGCGCGACCGCGGGCTGCGCGGCGGCGGCG | |
| | ACCACCGCGACCACCGCGACCACCGCGACCACCGCGGCGGCGGCGGCGGCGGGCGGCTGCTGCGCGACCGGCTGCGGC | |
| | GCGGCGGCGGGCGCGACCGCGGGCTGCTGCGCGGGCTGCGGCTGCACCGCGACCGCGGCGGCGGGCACCGGCGGCGCG | |
| | ACCACCGCGACCGGCGCGGCGGCGGGCTGCTGCGCGGGCGCGGGCTGCGCGTGCTGCGGCGCGACCGCGTGCTGCTGC | |
| | GCGGGCGCGGCGTGCACCACCACCGCGGGCTGCGCGGGCTGCGGCGCGGCGGCGGGCTGCGCGGCGGCGTGCGGCTGC | |
| | GGCGCGGCGGCGTGCTGCGGCGCGGCGACCGCGACCGGCGGCTGCTGCTGCGGCACCGGCTGCTGCGGCTGCTGCGGC | |
| | TGCGGCGCGGCGGCGACCGGCGGCGCGGCGGGCGCGACCGCGTGCTGCTGCACCGGCGCGGCGTGCTGCGCGACCTGC | |
| | ACCGGCGCGGCGGCGACCACCACCTGCACCGGCGCGGCGTGCGGCACCGGCTGCACCGGCGCGGGCTGCTGCTGCGGC | |
| | TGCGGCTGCGGCGGCTGCGGCACCGGCTGCGCGACCGCGACCACCTGCTGCGGCGCGGCGTGCACCGGCTGCGGCGCG | |
| | ACCGCGGCGGCGGCGGCGGCGGGCGGCTGCACCACCACCACCGCGACCGCGGCGGCGGCGGCGGCGGCGGCGGCGTGC | |
| | GCGGGCACCGGCTGCTGCGGCTGCTGCTGCGGCGCGGGCTGCGCGGCGGCGGGCGGCTGCTGCGGCTGCGCGGCGGCG | |
| | TGCGGCTGCGGCGGCTGCACCACCACCACCGGCTGCACCGGCGGCACCGGCTGCGGCACCGGCGGCGCGACCGCGGCG | |
| | GCGACCGCGACCGGCGGCTGCTGCGCGGGCTGCTGCGGCTGCACCGGCTGCTGCGGCGGCGGCTGCACCGCGACCGCG | |
| | TGCTGCGCGTGCTGCGCGGCGGCGGGCGGCTGCGCGGCGGCGGGCGCGGCGGGCGCGACCGGCACCGGCTGCGCGACC | |
| | ACCGGCTGCACCGCGACCGCGGGCTGCGCGACCGGCTGCGCGGGCGCGGGCTGCGCGGCGGCGGCGCGCCCGCTGCAG | |
| | GCGCTGCTGGATGGCCGCGGCCTGTGCGTGAACGCGAGCGCGGTGAGCCGCCTGCGCGCGTATCTGCTGCCGGCGCCG | |
| | CCGGCGCCGGGCGAACCGCCGGCGCCGGGCAACGCGAGCGAAAGCGAAGAAGATCGCAGCGCGGGCAGCGTGGAAAGC | |
| | CCGAGCGTGAGCAGCACCCATCGCGTGAGCGATCCGAAATTTCATCCGCTGCATAGCAAAATTATTATTATTAAAAAA | |
| | GGCCATGCGAAAGATAGCCAGCGCTATAAAGTGGATTATGAAAGCCAGAGCACCGATACCCAGAACTTTAGCAGCGAA | |
| | AGCAAACGCGAAACCGAATATGGCCCGTGCCGCCGCGAAATGGAAGATACCCTGAACCATCTGAAATTTCTGAACGTG | |
| | CTGAGCCCGCGCGGCGTGCATATTCCGAACTGCGATAAAAAAGGCTTTTATAAAAAAAAACAGTGCCGCCCGAGCAAA | |
| | GGCCGCAAACGCGGCTTTTGCTGGTGCGTGGATAAATATGGCCAGCCGCTGCCGGGCTATACCACCAAAGGCAAAGAA | |
| | GATGTGCATTGCTATAGCATGCAGAGCAAA | |
| GELS_HUMAN | MAPHRPAPALLCALSLALCALSLPVRAATASRGASQAGAPQGRVPEARPNSMVVEHPEFLKAGKEPGLQIWRVEKFDL | 22 |
| | VPVPTNLYGDFFTGDAYVILKTVQLRNGNLQYDLHYWLGNECSQDESGAAAIFTVQLDDYLNGRAVQHREVQGFESAT | |
| | FLGYFKSGLKYKKGGVASGFKHVVPNEVVVQRLFQVKGRRVVRATEVPVSWESFNNGDCFILDLGNNIHQWCGSNSNR | |
| | YERLKATQVSKGIRDNERSGRARVHVSEEGTEPEAMLQVLGPKPALPAGTEDTAKEDAANRKLAKLYKVSNGAGTMSV | |
| | SLVADENPFAQGALKSEDCFILDHGKDGKIFVWKGKQANTEERKAALKTASDFITKMDYPKQTQVSVLPEGGETPLFK | |
| | QFFKNWRDPDQTDGLGLSYLSSHIANVERVPFDAATLHTSTAMAAQHGMDDDGTGQKQIWRIEGSNKVPVDPATYGQF | |
| | YGGDSYIILYNYRHGGRQGQIIYNWQGAQSTQDEVAASAILTAQLDEELGGTPVQSRVVQGKEPAHLMSLFGGKPMII | |
| | YKGGTSREGGQTAPASTRLFQVRANSAGATRAVEVLPKAGALNSNDAFVLKTPSAAYLWVGTGASEAEKTGAQELLRV | |
| | LRAQPVQVAEGSEPDGFWEALGGKAAYRTSPRLKDKKMDAHPPRLFACSNKIGRFVIEEVPGELMQEDLATDDVMLLD | |
| | TWDQVFVWVGKDSQEEEKTEALTSAKRYIETDPANRDRRTPITVVKQGFEPPSFVGWFLGWDDDYWSVDPLDRAMAEL | |
| | AAGCGCGCCCGCTGCAGGCGCTGCTGGATGGCCGCGGCCTGTGCGTGAACGCGAGCGCGGTGAGCCGCCTGCGCGCGT | |
| | ATCTGCTGCCGGCGCCGCCGGCGCCGGGCGAACCGCCGGCGCCGGGCAACGCGAGCGAAAGCGAAGAAGATCGCAGCG | |
| | CGGGCAGCGTGGAAAGCCCGAGCGTGAGCAGCACCCATCGCGTGAGCGATCCGAAATTTCATCCGCTGCATAGCAAAA | |
| | TTATTATTATTAAAAAAGGCCATGCGAAAGATAGCCAGCGCTATAAAGTGGATTATGAAAGCCAGAGCACCGATACCC | |
| | AGAACTTTAGCAGCGAAAGCAAACGCGAAACCGAATATGGCCCGTGCCGCCGCGAAATGGAAGATACCCTGAACCATC | |
| | TGAAATTTCTGAACGTGCTGAGCCCGCGCGGCGTGCATATTCCGAACTGCGATAAAAAAGGCTTTTATAAAAAAAAAC | |
| | AGTGCCGCCCGAGCAAAGGCCGCAAACGCGGCTTTTGCTGGTGCGTGGATAAATATGGCCAGCCGCTGCCGGGCTATA | |
| | CCACCAAAGGCAAAGAAGATGTGCATTGCTATAGCATGCAGAGCAAA | |
| MASP1_HUMAN | ATGCGCTGGCTGCTGCTGTATTATGCGCTGTGCTTTAGCCTGAGCAAAGCGAGCGCGCATACCGTGGAACTGAACAAC | 23 |
| | ATGTTTGGCCAGATTCAGAGCCCGGGCTATCCGGATAGCTATCCGAGCGATAGCGAAGTGACCTGGAACATTACCGTG | |
| | CCGGATGGCTTTCGCATTAAACTGTATTTTATGCATTTTAACCTGGAAAGCAGCTATCTGTGCGAATATGATTATGTG | |
| | AAAGTGGAAACCGAAGATCAGGTGCTGGCGACCTTTTGCGGCCGCGAAACCACCGATACCGAACAGACCCCGGGCCAG | |
| | GAAGTGGTGCTGAGCCCGGGCAGCTTTATGAGCATTACCTTTCGCAGCGATTTTAGCAACGAAGAACGCTTTACCGGC | |
| | TTTGATGCGCATTATATGGCGGTGGATGTGGATGAATGCAAAGAACGCGAAGATGAAGAACTGAGCTGCGATCATTAT | |
| | TGCCATAACTATATTGGCGGCTATTATTGCAGCTGCCGCTTTGGCTATATTCTGCATACCGATAACCGCACCTGCCGC | |
| | GTGGAATGCAGCGATAACCTGTTTACCCAGCGCACCGGCGTGATTACCAGCCCGGATTTTCCGAACCCGTATCCGAAA | |
| | AGCAGCGAATGCCTGTATACCATTGAACTGGAAGAAGGCTTTATGGTGAACCTGCAGTTTGAAGATATTTTTGATATT | |
| | GAAGATCATCCGGAAGTGCCGTGCCCGTATGATTATATTAAAATTAAAGTGGGCCCGAAAGTGCTGGGCCCGTTTTGC | |
| | GGCGAAAAAGCGCCGGAACCGATTAGCACCCAGAGCCATAGCGTGCTGATTCTGTTTCATAGCGATAACAGCGGCGAA | |
| | AACCGCGGCTGGCGCCTGAGCTATCGCGCGGCGGGCAACGAATGCCCGGAACTGCAGCCGCCGGTGCATGGCAAAATT | |
| | GAACCGAGCCAGGCGAAATATTTTTTTAAAGATCAGGTGCTGGTGAGCTGCGATACCGGCTATAAAGTGCTGAAAGAT | |
| | AACGTGGAAATGGATACCTTTCAGATTGAATGCCTGAAAGATGGCACCTGGAGCAACAAAATTCCGACCTGCAAAATT | |
| | GTGGATTGCCGCGCGCCGGGCGAACTGGAACATGGCCTGATTACCTTTAGCACCCGCAACAACCTGACCACCTATAAA | |
| | AGCGAAATTAAATATAGCTGCCAGGAACCGTATTATAAAATGCTGAACAACAACACCGGCATTTATACCTGCAGCGCG | |
| | CAGGGCGTGTGGATGAACAAAGTGCTGGGCCGCAGCCTGCCGACCTGCCTGCCGGTGTGCGGCCTGCCGAAATTTAGC | |
| | CGCAAACTGATGGCGCGCATTTTTAACGGCCGCCCGGCGCAGAAAGGCACCACCCCGTGGATTGCGATGCTGAGCCAT | |
| | CTGAACGGCCAGCCGTTTTGCGGCGGCAGCCTGCTGGGCAGCAGCTGGATTGTGACCGCGGCGCATTGCCTGCATCAG | |
| | AGCCTGGATCCGGAAGATCCGACCCTGCGCGATAGCGATCTGCTGAGCCCGAGCGATTTTAAAATTATTCTGGGCAAA | |
| | CATTGGCGCCTGCGCAGCGATGAAAACGAACAGCATCTGGGCGTGAAACATACCACCCTGCATCCGCAGTATGATCCG | |
| | AACACCTTTGAAAACGATGTGGCGCTGGTGGAACTGCTGGAAAGCCCGGTGCTGAACGCGTTTGTGATGCCGATTTGC | |
| | CTGCCGGAAGGCCCGCAGCAGGAAGGCGCGATGGTGATTGTGAGCGGCTGGGGCAAACAGTTTCTGCAGCGCTTTCCG | |
| | GAAACCCTGATGGAAATTGAAATTCCGATTGTGGATCATAGCACCTGCCAGAAAGCGTATGCGCCGCTGAAAAAAAAA | |
| | GTGACCCGCGATATGATTTGCGCGGGCGAAAAAGAAGGCGGCAAAGATGCGTGCGCGGGCGATAGCGGCGGCCCGATG | |
| | GTGACCCTGAACCGCGAACGCGGCCAGTGGTATCTGGTGGGCACCGTGAGCTGGGGCGATGATTGCGGCAAAAAAGAT | |
| | CGCTATGGCGTGTATAGCTATATTCATCATAACAAAGATTGGATTCAGCGCGTGACCGGCGTGCGCAAC | |
| MASP1_HUMAN | MRWLLLYYALCFSLSKASAHTVELNNMFGQIQSPGYPDSYPSDSEVTWNITVPDGFRIKLYFMHFNLESSYLCEYDYV | 24 |
| | KVETEDQVLATFCGRETTDTEQTPGQEVVLSPGSFMSITFRSDFSNEERFTGFDAHYMAVDVDECKEREDEELSCDHY | |
| | CHNYIGGYYCSCRFGYILHTDNRTCRVECSDNLFTQRTGVITSPDFPNPYPKSSECLYTIELEEGFMVNLQFEDIFDI | |
| | EDHPEVPCPYDYIKIKVGPKVLGPFCGEKAPEPISTQSHSVLILFHSDNSGENRGWRLSYRAAGNECPELQPPVHGKI | |
| | EPSQAKYFFKDQVLVSCDTGYKVLKDNVEMDTFQIECLKDGTWSNKIPTCKIVDCRAPGELEHGLITFSTRNNLTTYK | |
| | SEIKYSCQEPYYKMLNNNTGIYTCSAQGVWMNKVLGRSLPTCLPVCGLPKFSRKLMARIFNGRPAQKGTTPWIAMLSH | |
| | LNGQPFCGGSLLGSSWIVTAAHCLHQSLDPEDPTLRDSDLLSPSDFKIILGKHWRLRSDENEQHLGVKHTTLHPQYDP | |
| | NTFENDVALVELLESPVLNAFVMPICLPEGPQQEGAMVIVSGWGKQFLQRFPETLMEIEIPIVDHSTCQKAYAPLKKK | |
| | VTRDMICAGEKEGGKDACAGDSGGPMVTLNRERGQWYLVGTVSWGDDCGKKDRYGVYSYIHHNKDWIQRVTGVRN | |
| COIA1_HUMAN | ATGGCGCCGTATCCGTGCGGCTGCCATATTCTGCTGCTGCTGTTTTGCTGCCTGGCGGCGGCGCGCGCGAACCTGCTG | 25 |
| | AACCTGAACTGGCTGTGGTTTAACAACGAAGATACCAGCCATGCGGCGACCACCATTCCGGAACCGCAGGGCCCGCTG | |
| | CCGGTGCAGCCGACCGCGGATACCACCACCCATGTGACCCCGCGCAACGGCAGCACCGAACCGGCGACCGCGCCGGGC | |
| | AGCCCGGAACCGCCGAGCGAACTGCTGGAAGATGGCCAGGATACCCCGACCAGCGCGGAAAGCCCGGATGCGCCGGAA | |
| | GAAAACATTGCGGGCGTGGGCGCGGAAATTCTGAACGTGGCGAAAGGCATTCGCAGCTTTGTGCAGCTGTGGAACGAT | |
| | ACCGTGCCGACCGAAAGCCTGGCGCGCGCGGAAACCCTGGTGCTGGAAACCCCGGTGGGCCCGCTGGCGCTGGCGGGC | |
| | CCGAGCAGCACCCCGCAGGAAAACGGCACCACCCTGTGGCCGAGCCGCGGCATTCCGAGCAGCCCGGGCGCGCATACC | |
| | ACCGAAGCGGGCACCCTGCCGGCGCCGACCCCGAGCCCGCCGAGCCTGGGCCGCCCGTGGGCGCCGCTGACCGGCCCG | |
| | AGCGTGCCGCCGCCGAGCAGCGGCCGCGCGAGCCTGAGCAGCCTGCTGGGCGGCGCGCCGCCGTGGGGCAGCCTGCAG | |
| | GATCCGGATAGCCAGGGCCTGAGCCCGGCGGCGGCGGCGCCGAGCCAGCAGCTGCAGCGCCCGGATGTGCGCCTGCGC | |
| | ACCCCGCTGCTGCATCCGCTGGTGATGGGCAGCCTGGGCAAACATGCGGCGCCGAGCGCGTTTAGCAGCGGCCTGCCG | |
| | GGCGCGCTGAGCCAGGTGGCGGTGACCACCCTGACCCGCGATAGCGGCGCGTGGGTGAGCCATGTGGCGAACAGCGTG | |
| | GGCCCGGGCCTGGCGAACAACAGCGCGCTGCTGGGCGCGGATCCGGAAGCGCCGGCGGGCCGCTGCCTGCCGCTGCCG | |
| | CCGAGCCTGCCGGTGTGCGGCCATCTGGGCATTAGCCGCTTTTGGCTGCCGAACCATCTGCATCATGAAAGCGGCGAA | |
| | CAGGTGCGCGCGGGCGCGCGCGCGTGGGGCGGCCTGCTGCAGACCCATTGCCATCCGTTTCTGGCGTGGTTTTTTTGC | |
| | CTGCTGCTGGTGCCGCCGTGCGGCAGCGTGCCGCCGCCGGCGCCGCCGCCGTGCTGCCAGTTTTGCGAAGCGCTGCAG | |
| | GATGCGTGCTGGAGCCGCCTGGGCGGCGGCCGCCTGCCGGTGGCGTGCGCGAGCCTGCCGACCCAGGAAGATGGCTAT | |
| | TGCGTGCTGATTGGCCCGGCGGCGGAACGCATTAGCGAAGAAGTGGGCCTGCTGCAGCTGCTGGGCGATCCGCCGCCG | |
| | CAGCAGGTGACCCAGACCGATGATCCGGATGTGGGCCTGGCGTATGTGTTTGGCCCGGATGCGAACAGCGGCCAGGTG | |
| | GCGCGCTATCATTTTCCGAGCCTGTTTTTTCGCGATTTTAGCCTGCTGTTTCATATTCGCCCGGCGACCGAAGGCCCG | |
| | GGCGTGCTGTTTGCGATTACCGATAGCGCGCAGGCGATGGTGCTGCTGGGCGTGAAACTGAGCGGCGTGCAGGATGGC | |
| | CATCAGGATATTAGCCTGCTGTATACCGAACCGGGCGCGGGCCAGACCCATACCGCGGCGAGCTTTCGCCTGCCGGCG | |
| | TTTGTGGGCCAGTGGACCCATCTGGCGCTGAGCGTGGCGGGCGGCTTTGTGGCGCTGTATGTGGATTGCGAAGAATTT | |
| | CAGCGCATGCCGCTGGCGCGCAGCAGCCGCGGCCTGGAACTGGAACCGGGCGCGGGCCTGTTTGTGGCGCAGGCGGGC | |
| | GGCGCGGATCCGGATAAATTTCAGGGCGTGATTGCGGAACTGAAAGTGCGCCGCGATCCGCAGGTGAGCCCGATGCAT | |
| | TGCCTGGATGAAGAAGGCGATGATAGCGATGGCGCGAGCGGCGATAGCGGCAGCGGCCTGGGCGATGCGCGCGAACTG | |
| | CTGCGCGAAGAAACCGGCGCGGCGCTGAAACCGCGCCTGCCGGCGCCGCCGCCGGTGACCACCCCGCCGCTGGCGGGC | |
| | GGCAGCAGCACCGAAGATAGCCGCAGCGAAGAAGTGGAAGAACAGACCACCGTGGCGAGCCTGGGCGCGCAGACCCTG | |
| | CCGGGCAGCGATAGCGTGAGCACCTGGGATGGCAGCGTGCGCACCCCGGGCGGCCGCGTGAAAGAAGGCGGCCTGAAA | |
| | GGCCAGAAAGGCGAACCGGGCGTGCCGGGCCCGCCGGGCCGCGCGGGCCCGCCGGGCAGCCCGTGCCTGCCGGGCCCG | |
| | CCGGGCCTGCCGTGCCCGGTGAGCCCGCTGGGCCCGGCGGGCCCGGCGCTGCAGACCGTGCCGGGCCCGCAGGGCCCG | |
| | CCGGGCCCGCCGGGCCGCGATGGCACCCCGGGCCGCGATGGCGAACCGGGCGATCCGGGCGAAGATGGCAAACCGGGC | |
| | GATACCGGCCCGCAGGGCTTTCCGGGCACCCCGGGCGATGTGGGCCCGAAAGGCGATAAAGGCGATCCGGGCGTGGGC | |
| | GAACGCGGCCCGCCGGGCCCGCAGGGCCCGCCGGGCCCGCCGGGCCCGAGCTTTCGCCATGATAAACTGACCTTTATT | |
| | GATATGGAAGGCAGCGGCTTTGGCGGCGATCTGGAAGCGCTGCGCGGCCCGCGCGGCTTTCCGGGCCCGCCGGGCCCG | |
| | CCGGGCGTGCCGGGCCTGCCGGGCGAACCGGGCCGCTTTGGCGTGAACAGCAGCGATGTGCCGGGCCCGGCGGGCCTG | |
| | CCGGGCGTGCCGGGCCGCGAAGGCCCGCCGGGCTTTCCGGGCCTGCCGGGCCCGCCGGGCCCGCCGGGCCGCGAAGGC | |
| | CCGCCGGGCCGCACCGGCCAGAAAGGCAGCCTGGGCGAAGCGGGCGCGCCGGGCCATAAAGGCAGCAAAGGCGCGCCG | |
| | GGCCCGGCGGGCGCGCGCGGCGAAAGCGGCCTGGCGGGCGCGCCGGGCCCGGCGGGCCCGCCGGGCCCGCCGGGCCCG | |
| | CCGGGCCCGCCGGGCCCGGGCCTGCCGGCGGGCTTTGATGATATGGAAGGCAGCGGCGGCCCGTTTTGGAGCACCGCG | |
| | CGCAGCGCGGATGGCCCGCAGGGCCCGCCGGGCCTGCCGGGCCTGAAAGGCGATCCGGGCGTGCCGGGCCTGCCGGGC | |
| | GCGAAAGGCGAAGTGGGCGCGGATGGCGTGCCGGGCTTTCCGGGCCTGCCGGGCCGCGAAGGCATTGCGGGCCCGCAG | |
| | GGCCCGAAAGGCGATCGCGGCAGCCGCGGCGAAAAAGGCGATCCGGGCAAAGATGGCGTGGGCCAGCCGGGCCTGCCG | |
| | GGCCCGCCGGGCCCGCCGGGCCCGGTGGTGTATGTGAGCGAACAGGATGGCAGCGTGCTGAGCGTGCCGGGCCCGGAA | |
| | GGCCGCCCGGGCTTTGCGGGCTTTCCGGGCCCGGCGGGCCCGAAAGGCAACCTGGGCAGCAAAGGCGAACGCGGCAGC | |
| | CCGGGCCCGAAAGGCGAAAAAGGCGAACCGGGCAGCATTTTTAGCCCGGATGGCGGCGCGCTGGGCCCGGCGCAGAAA | |
| | GGCGCGAAAGGCGAACCGGGCTTTCGCGGCCCGCCGGGCCCGTATGGCCGCCCGGGCTATAAAGGCGAAATTGGCTTT | |
| | CCGGGCCGCCCGGGCCGCCCGGGCATGAACGGCCTGAAAGGCGAAAAAGGCGAACCGGGCGATGCGAGCCTGGGCTTT | |
| | GGCATGCGCGGCATGCCGGGCCCGCCGGGCCCGCCGGGCCCGCCGGGCCCGCCGGGCACCCCGGTGTATGATAGCAAC | |
| | GTGTTTGCGGAAAGCAGCCGCCCGGGCCCGCCGGGCCTGCCGGGCAACCAGGGCCCGCCGGGCCCGAAAGGCGCGAAA | |
| | GGCGAAGTGGGCCCGCCGGGCCCGCCGGGCCAGTTTCCGTTTGATTTTCTGCAGCTGGAAGCGGAAATGAAAGGCGAA | |
| | AAAGGCGATCGCGGCGATGCGGGCCAGAAAGGCGAACGCGGCGAACCGGGCGGCGGCGGCTTTTTTGGCAGCAGCCTG | |
| | CCGGGCCCGCCGGGCCCGCCGGGCCCGCCGGGCCCGCGCGGCTATCCGGGCATTCCGGGCCCGAAAGGCGAAAGCATT | |
| | CGCGGCCAGCCGGGCCCGCCGGGCCCGCAGGGCCCGCCGGGCATTGGCTATGAAGGCCGCCAGGGCCCGCCGGGCCCG | |
| | CCGGGCCCGCCGGGCCCGCCGAGCTTTCCGGGCCCGCATCGCCAGACCATTAGCGTGCCGGGCCCGCCGGGCCCGCCG | |
| | GGCCCGCCGGGCCCGCCGGGCACCATGGGCGCGAGCAGCGGCGTGCGCCTGTGGGCGACCCGCCAGGCGATGCTGGGC | |
| | CAGGTGCATGAAGTGCCGGAAGGCTGGCTGATTTTTGTGGCGGAACAGGAAGAACTGTATGTGCGCGTGCAGAACGGC | |
| | TTTCGCAAAGTGCAGCTGGAAGCGCGCACCCCGCTGCCGCGCGGCACCGATAACGAAGTGGCGGCGCTGCAGCCGCCG | |
| | GTGGTGCAGCTGCATGATAGCAACCCGTATCCGCGCCGCGAACATCCGCATCCGACCGCGCGCCCGTGGCGCGCGGAT | |
| | GATATTCTGGCGAGCCCGCCGCGCCTGCCGGAACCGCAGCCGTATCCGGGCGCGCCGCATCATAGCAGCTATGTGCAT | |
| | CTGCGCCCGGCGCGCCCGACCAGCCCGCCGGCGCATAGCCATCGCGATTTTCAGCCGGTGCTGCATCTGGTGGCGCTG | |
| | AACAGCCCGCTGAGCGGCGGCATGCGCGGCATTCGCGGCGCGGATTTTCAGTGCTTTCAGCAGGCGCGCGCGGTGGGC | |
| | CTGGCGGGCACCTTTCGCGCGTTTCTGAGCAGCCGCCTGCAGGATCTGTATAGCATTGTGCGCCGCGCGGATCGCGCG | |
| | GCGGTGCCGATTGTGAACCTGAAAGATGAACTGCTGTTTCCGAGCTGGGAAGCGCTGTTTAGCGGCAGCGAAGGCCCG | |
| | CTGAAACCGGGCGCGCGCATTTTTAGCTTTGATGGCAAAGATGTGCTGCGCCATCCGACCTGGCCGCAGAAAAGCGTG | |
| | TGGCATGGCAGCGATCCGAACGGCCGCCGCCTGACCGAAAGCTATTGCGAAACCTGGCGCACCGAAGCGCCGAGCGCG | |
| | ACCGGCCAGGCGAGCAGCCTGCTGGGCGGCCGCCTGCTGGGCCAGAGCGCGGCGAGCTGCCATCATGCGTATATTGTG | |
| | CTGTGCATTGAAAACAGCTTTATGACCGCGAGCAAA | |
| COIA1_HUMAN | MAPYPCGCHILLLLFCCLAAARANLLNLNWLWFNNEDTSHAATTIPEPQGPLPVQPTADTTTHVTPRNGSTEPATAPG | 26 |
| | SPEPPSELLEDGQDTPTSAESPDAPEENIAGVGAEILNVAKGIRSFVQLWNDTVPTESLARAETLVLETPVGPLALAG | |
| | PSSTPQENGTTLWPSRGIPSSPGAHTTEAGTLPAPTPSPPSLGRPWAPLTGPSVPPPSSGRASLSSLLGGAPPWGSLQ | |
| | DPDSQGLSPAAAAPSQQLQRPDVRLRTPLLHPLVMGSLGKHAAPSAFSSGLPGALSQVAVTTLTRDSGAWVSHVANSV | |
| | GPGLANNSALLGADPEAPAGRCLPLPPSLPVCGHLGISRFWLPNHLHHESGEQVRAGARAWGGLLQTHCHPFLAWFFC | |
| | LLLVPPCGSVPPPAPPPCCQFCEALQDACWSRLGGGRLPVACASLPTQEDGYCVLIGPAAERISEEVGLLQLLGDPPP | |
| | QQVTQTDDPDVGLAYVEGPDANSGQVARYHFPSLFERDFSLLFHIRPATEGPGVLFATTDSAQAMVLLGVKLSGVQDG | |
| | HQDISLLYTEPGAGQTHTAASFRLPAFVGQWTHLALSVAGGFVALYVDCEEFQRMPLARSSRGLELEPGAGLFVAQAG | |
| | GADPDKFQGVIAELKVRRDPQVSPMHCLDEEGDDSDGASGDSGSGLGDARELLREETGAALKPRLPAPPPVTTPPLAG | |
| | GSSTEDSRSEEVEEQTTVASLGAQTLPGSDSVSTWDGSVRTPGGRVKEGGLKGQKGEPGVPGPPGRAGPPGSPCLPGP | |
| | PGLPCPVSPLGPAGPALQTVPGPQGPPGPPGRDGTPGRDGEPGDPGEDGKPGDTGPQGFPGTPGDVGPKGDKGDPGVG | |
| | ERGPPGPQGPPGPPGPSFRHDKLTFIDMEGSGFGGDLEALRGPRGFPGPPGPPGVPGLPGEPGRFGVNSSDVPGPAGL | |
| | PGVPGREGPPGFPGLPGPPGPPGREGPPGRTGQKGSLGEAGAPGHKGSKGAPGPAGARGESGLAGAPGPAGPPGPPGP | |
| | PGPPGPGLPAGFDDMEGSGGPFWSTARSADGPQGPPGLPGLKGDPGVPGLPGAKGEVGADGVPGFPGLPGREGIAGPQ | |
| | GPKGDRGSRGEKGDPGKDGVGQPGLPGPPGPPGPVVYVSEQDGSVLSVPGPEGRPGFAGFPGPAGPKGNLGSKGERGS | |
| | PGPKGEKGEPGSIFSPDGGALGPAQKGAKGEPGFRGPPGPYGRPGYKGEIGFPGRPGRPGMNGLKGEKGEPGDASLGF | |
| | GMRGMPGPPGPPGPPGPPGTPVYDSNVFAESSRPGPPGLPGNQGPPGPKGAKGEVGPPGPPGQFPFDFLQLEAEMKGE | |
| | KGDRGDAGQKGERGEPGGGGFEGSSLPGPPGPPGPPGPRGYPGIPGPKGESIRGQPGPPGPQGPPGIGYEGRQGPPGP | |
| | PGPPGPPSFPGPHRQTISVPGPPGPPGPPGPPGTMGASSGVRLWATRQAMLGQVHEVPEGWLIFVAEQEELYVRVQNG | |
| | FRKVQLEARTPLPRGTDNEVAALQPPVVQLHDSNPYPRREHPHPTARPWRADDILASPPRLPEPQPYPGAPHHSSYVH | |
| | LRPARPTSPPAHSHRDFQPVLHLVALNSPLSGGMRGIRGADFQCFQQARAVGLAGTFRAFLSSRLQDLYSIVRRADRA | |
| | AVPIVNLKDELLFPSWEALFSGSEGPLKPGARIFSFDGKDVLRHPTWPQKSVWHGSDPNGRRLTESYCETWRTEAPSA | |
| | TGQASSLLGGRLLGQSAASCHHAYIVLCIENSFMTASK | |
| GRP78_HUMAN | ATGAAACTGAGCCTGGTGGCGGCGATGCTGCTGCTGCTGAGCGCGGCGCGCGCGGAAGAAGAAGATAAAAAAGAAGAT | 27 |
| | GTGGGCACCGTGGTGGGCATTGATCTGGGCACCACCTATAGCTGCGTGGGCGTGTTTAAAAACGGCCGCGTGGAAATT | |
| | ATTGCGAACGATCAGGGCAACCGCATTACCCCGAGCTATGTGGCGTTTACCCCGGAAGGCGAACGCCTGATTGGCGAT | |
| | GCGGCGAAAAACCAGCTGACCAGCAACCCGGAAAACACCGTGTTTGATGCGAAACGCCTGATTGGCCGCACCTGGAAC | |
| | GATCCGAGCGTGCAGCAGGATATTAAATTTCTGCCGTTTAAAGTGGTGGAAAAAAAAACCAAACCGTATATTCAGGTG | |
| | GATATTGGCGGCGGCCAGACCAAAACCTTTGCGCCGGAAGAAATTAGCGCGATGGTGCTGACCAAAATGAAAGAAACC | |
| | GCGGAAGCGTATCTGGGCAAAAAAGTGACCCATGCGGTGGTGACCGTGCCGGCGTATTTTAACGATGCGCAGCGCCAG | |
| | GCGACCAAAGATGCGGGCACCATTGCGGGCCTGAACGTGATGCGCATTATTAACGAACCGACCGCGGCGGCGATTGCG | |
| | TATGGCCTGGATAAACGCGAAGGCGAAAAAAACATTCTGGTGTTTGATCTGGGCGGCGGCACCTTTGATGTGAGCCTG | |
| | CTGACCATTGATAACGGCGTGTTTGAAGTGGTGGCGACCAACGGCGATACCCATCTGGGCGGCGAAGATTTTGATCAG | |
| | CGCGTGATGGAACATTTTATTAAACTGTATAAAAAAAAAACCGGCAAAGATGTGCGCAAAGATAACCGCGCGGTGCAG | |
| | AAACTGCGCCGCGAAGTGGAAAAAGCGAAACGCGCGCTGAGCAGCCAGCATCAGGCGCGCATTGAAATTGAAAGCTTT | |
| | TATGAAGGCGAAGATTTTAGCGAAACCCTGACCCGCGCGAAATTTGAAGAACTGAACATGGATCTGTTTCGCAGCACC | |
| | ATGAAACCGGTGCAGAAAGTGCTGGAAGATAGCGATCTGAAAAAAAGCGATATTGATGAAATTGTGCTGGTGGGCGGC | |
| | AGCACCCGCATTCCGAAAATTCAGCAGCTGGTGAAAGAATTTTTTAACGGCAAAGAACCGAGCCGCGGCATTAACCCG | |
| | GATGAAGCGGTGGCGTATGGCGCGGCGGTGCAGGCGGGCGTGCTGAGCGGCGATCAGGATACCGGCGATCTGGTGCTG | |
| | CTGGATGTGTGCCCGCTGACCCTGGGCATTGAAACCGTGGGCGGCGTGATGACCAAACTGATTCCGCGCAACACCGTG | |
| | GTGCCGACCAAAAAAAGCCAGATTTTTAGCACCGCGAGCGATAACCAGCCGACCGTGACCATTAAAGTGTATGAAGGC | |
| | GAACGCCCGCTGACCAAAGATAACCATCTGCTGGGCACCTTTGATCTGACCGGCATTCCGCCGGCGCCGCGCGGCGTG | |
| | CCGCAGATTGAAGTGACCTTTGAAATTGATGTGAACGGCATTCTGCGCGTGACCGCGGAAGATAAAGGCACCGGCAAC | |
| | AAAAACAAAATTACCATTACCAACGATCAGAACCGCCTGACCCCGGAAGAAATTGAACGCATGGTGAACGATGCGGAA | |
| | AAATTTGCGGAAGAAGATAAAAAACTGAAAGAACGCATTGATACCCGCAACGAACTGGAAAGCTATGCGTATAGCCTG | |
| | AAAAACCAGATTGGCGATAAAGAAAAACTGGGCGGCAAACTGAGCAGCGAAGATAAAGAAACCATGGAAAAAGCGGTG | |
| | GAAGAAAAAATTGAATGGCTGGAAAGCCATCAGGATGCGGATATTGAAGATTTTAAAGCGAAAAAAAAAGAACTGGAA | |
| | GAAATTGTGCAGCCGATTATTAGCAAACTGTATGGCAGCGCGGGCCCGCCGCCGACCGGCGAAGAAGATACCGCGGAA | |
| | AAAGATGAACTG | |
| GRP78_HUMAN | MKLSLVAAMLLLLSAARAEEEDKKEDVGTVVGIDLGTTYSCVGVFKNGRVEIIANDQGNRITPSYVAFTPEGERLIGD | 28 |
| | AAKNQLTSNPENTVFDAKRLIGRTWNDPSVQQDIKFLPFKVVEKKTKPYIQVDIGGGQTKTFAPEEISAMVLTKMKET | |
| | AEAYLGKKVTHAVVTVPAYFNDAQRQATKDAGTIAGLNVMRIINEPTAAAIAYGLDKREGEKNILVFDLGGGTFDVSL | |
| | LTIDNGVFEVVATNGDTHLGGEDFDQRVMEHFIKLYKKKTGKDVRKDNRAVQKLRREVEKAKRALSSQHQARIEIESF | |
| | YEGEDFSETLTRAKFEELNMDLFRSTMKPVQKVLEDSDLKKSDIDEIVLVGGSTRIPKIQQLVKEFFNGKEPSRGINP | |
| | DEAVAYGAAVQAGVLSGDQDTGDLVLLDVCPLTLGIETVGGVMTKLIPRNTVVPTKKSQIFSTASDNQPTVTIKVYEG | |
| | ERPLTKDNHLLGTFDLTGIPPAPRGVPQIEVTFEIDVNGILRVTAEDKGTGNKNKITITNDQNRLTPEEIERMVNDAE | |
| | KFAEEDKKLKERIDTRNELESYAYSLKNQIGDKEKLGGKLSSEDKETMEKAVEEKIEWLESHQDADIEDFKAKKKELE | |
| | EIVQPIISKLYGSAGPPPTGEEDTAEKDEL | |
| KIT_HUMAN | ATGCGCGGCGCGCGCGGCGCGTGGGATTTTCTGTGCGTGCTGCTGCTGCTGCTGCGCGTGCAGACCGGCAGCAGCCAG | 29 |
| | CCGAGCGTGAGCCCGGGCGAACCGAGCCCGCCGAGCATTCATCCGGGCAAAAGCGATCTGATTGTGCGCGTGGGCGAT | |
| | GAAATTCGCCTGCTGTGCACCGATCCGGGCTTTGTGAAATGGACCTTTGAAATTCTGGATGAAACCAACGAAAACAAA | |
| | CAGAACGAATGGATTACCGAAAAAGCGGAAGCGACCAACACCGGCAAATATACCTGCACCAACAAACATGGCCTGAGC | |
| | AACAGCATTTATGTGTTTGTGCGCGATCCGGCGAAACTGTTTCTGGTGGATCGCAGCCTGTATGGCAAAGAAGATAAC | |
| | GATACCCTGGTGCGCTGCCCGCTGACCGATCCGGAAGTGACCAACTATAGCCTGAAAGGCTGCCAGGGCAAACCGCTG | |
| | CCGAAAGATCTGCGCTTTATTCCGGATCCGAAAGCGGGCATTATGATTAAAAGCGTGAAACGCGCGTATCATCGCCTG | |
| | TGCCTGCATTGCAGCGTGGATCAGGAAGGCAAAAGCGTGCTGAGCGAAAAATTTATTCTGAAAGTGCGCCCGGCGTTT | |
| | AAAGCGGTGCCGGTGGTGAGCGTGAGCAAAGCGAGCTATCTGCTGCGCGAAGGCGAAGAATTTACCGTGACCTGCACC | |
| | ATTAAAGATGTGAGCAGCAGCGTGTATAGCACCTGGAAACGCGAAAACAGCCAGACCAAACTGCAGGAAAAATATAAC | |
| | AGCTGGCATCATGGCGATTTTAACTATGAACGCCAGGCGACCCTGACCATTAGCAGCGCGCGCGTGAACGATAGCGGC | |
| | GTGTTTATGTGCTATGCGAACAACACCTTTGGCAGCGCGAACGTGACCACCACCCTGGAAGTGGTGGATAAAGGCTTT | |
| | ATTAACATTTTTCCGATGATTAACACCACCGTGTTTGTGAACGATGGCGAAAACGTGGATCTGATTGTGGAATATGAA | |
| | GCGTTTCCGAAACCGGAACATCAGCAGTGGATTTATATGAACCGCACCTTTACCGATAAATGGGAAGATTATCCGAAA | |
| | AGCGAAAACGAAAGCAACATTCGCTATGTGAGCGAACTGCATCTGACCCGCCTGAAAGGCACCGAAGGCGGCACCTAT | |
| | ACCTTTCTGGTGAGCAACAGCGATGTGAACGCGGCGATTGCGTTTAACGTGTATGTGAACACCAAACCGGAAATTCTG | |
| | ACCTATGATCGCCTGGTGAACGGCATGCTGCAGTGCGTGGCGGCGGGCTTTCCGGAACCGACCATTGATTGGTATTTT | |
| | TGCCCGGGCACCGAACAGCGCTGCAGCGCGAGCGTGCTGCCGGTGGATGTGCAGACCCTGAACAGCAGCGGCCCGCCG | |
| | TTTGGCAAACTGGTGGTGCAGAGCAGCATTGATAGCAGCGCGTTTAAACATAACGGCACCGTGGAATGCAAAGCGTAT | |
| | AACGATGTGGGCAAAACCAGCGCGTATTTTAACTTTGCGTTTAAAGGCAACAACAAAGAACAGATTCATCCGCATACC | |
| | CTGTTTACCCCGCTGCTGATTGGCTTTGTGATTGTGGCGGGCATGATGTGCATTATTGTGATGATTCTGACCTATAAA | |
| | TATCTGCAGAAACCGATGTATGAAGTGCAGTGGAAAGTGGTGGAAGAAATTAACGGCAACAACTATGTGTATATTGAT | |
| | CCGACCCAGCTGCCGTATGATCATAAATGGGAATTTCCGCGCAACCGCCTGAGCTTTGGCAAAACCCTGGGCGCGGGC | |
| | GCGTTTGGCAAAGTGGTGGAAGCGACCGCGTATGGCCTGATTAAAAGCGATGCGGCGATGACCGTGGCGGTGAAAATG | |
| | CTGAAACCGAGCGCGCATCTGACCGAACGCGAAGCGCTGATGAGCGAACTGAAAGTGCTGAGCTATCTGGGCAACCAT | |
| | ATGAACATTGTGAACCTGCTGGGCGCGTGCACCATTGGCGGCCCGACCCTGGTGATTACCGAATATTGCTGCTATGGC | |
| | GATCTGCTGAACTTTCTGCGCCGCAAACGCGATAGCTTTATTTGCAGCAAACAGGAAGATCATGCGGAAGCGGCGCTG | |
| | TATAAAAACCTGCTGCATAGCAAAGAAAGCAGCTGCAGCGATAGCACCAACGAATATATGGATATGAAACCGGGCGTG | |
| | AGCTATGTGGTGCCGACCAAAGCGGATAAACGCCGCAGCGTGCGCATTGGCAGCTATATTGAACGCGATGTGACCCCG | |
| | GCGATTATGGAAGATGATGAACTGGCGCTGGATCTGGAAGATCTGCTGAGCTTTAGCTATCAGGTGGCGAAAGGCATG | |
| | GCGTTTCTGGCGAGCAAAAACTGCATTCATCGCGATCTGGCGGCGCGCAACATTCTGCTGACCCATGGCCGCATTACC | |
| | AAAATTTGCGATTTTGGCCTGGCGCGCGATATTAAAAACGATAGCAACTATGTGGTGAAAGGCAACGCGCGCCTGCCG | |
| | GTGAAATGGATGGCGCCGGAAAGCATTTTTAACTGCGTGTATACCTTTGAAAGCGATGTGTGGAGCTATGGCATTTTT | |
| | CTGTGGGAACTGTTTAGCCTGGGCAGCAGCCCGTATCCGGGCATGCCGGTGGATAGCAAATTTTATAAAATGATTAAA | |
| | GAAGGCTTTCGCATGCTGAGCCCGGAACATGCGCCGGCGGAAATGTATGATATTATGAAAACCTGCTGGGATGCGGAT | |
| | CCGCTGAAACGCCCGACCTTTAAACAGATTGTGCAGCTGATTGAAAAACAGATTAGCGAAAGCACCAACCATATTTAT | |
| | AGCAACCTGGCGAACTGCAGCCCGAACCGCCAGAAACCGGTGGTGGATCATAGCGTGCGCATTAACAGCGTGGGCAGC | |
| | ACCGCGAGCAGCAGCCAGCCGCTGCTGGTGCATGATGATGTG | |
| KIT_HUMAN | MRGARGAWDFLCVLLLLLRVQTGSSQPSVSPGEPSPPSIHPGKSDLIVRVGDEIRLLCTDPGFVKWTFEILDETNENK | 30 |
| | QNEWITEKAEATNTGKYTCTNKHGLSNSIYVFVRDPAKLFLVDRSLYGKEDNDTLVRCPLTDPEVTNYSLKGCQGKPL | |
| | PKDLRFIPDPKAGIMIKSVKRAYHRLCLHCSVDQEGKSVLSEKFILKVRPAFKAVPVVSVSKASYLLREGEEFTVTCT | |
| | IKDVSSSVYSTWKRENSQTKLQEKYNSWHHGDFNYERQATLTISSARVNDSGVFMCYANNTFGSANVTTTLEVVDKGF | |
| | INTFPMINTTVFVNDGENVDLIVEYEAFPKPEHQQWIYMNRTFTDKWEDYPKSENESNIRYVSELHLTRLKGTEGGTY | |
| | TFLVSNSDVNAAIAFNVYVNTKPEILTYDRLVNGMLQCVAAGFPEPTIDWYFCPGTEQRCSASVLPVDVQTLNSSGPP | |
| | FGKLVVQSSIDSSAFKHNGTVECKAYNDVGKTSAYFNFAFKGNNKEQIHPHTLFTPLLIGFVIVAGMMCIIVMILTYK | |
| | YLQKPMYEVQWKVVEEINGNNYVYIDPTQLPYDHKWEFPRNRLSFGKTLGAGAFGKVVEATAYGLIKSDAAMTVAVKM | |
| | LKPSAHLTEREALMSELKVLSYLGNHMNIVNLLGACTIGGPTLVITEYCCYGDLLNFLRRKRDSFICSKQEDHAEAAL | |
| | YKNLLHSKESSCSDSTNEYMDMKPGVSYVVPTKADKRRSVRIGSYIERDVTPAIMEDDELALDLEDLLSFSYQVAKGM | |
| | AFLASKNCIHRDLAARNILLTHGRITKICDFGLARDIKNDSNYVVKGNARLPVKWMAPESIFNCVYTFESDVWSYGIF | |
| | LWELFSLGSSPYPGMPVDSKEYKMIKEGFRMLSPEHAPAEMYDIMKTCWDADPLKRPTFKQIVQLIEKQISESTNHIY | |
| | SNLANCSPNRQKPVVDHSVRINSVGSTASSSQPLLVHDDV | |
| PROF1_HUMAN | ATGGCGGGCTGGAACGCGTATATTGATAACCTGATGGCGGATGGCACCTGCCAGGATGCGGCGATTGTGGGCTATAAA | 31 |
| | GATAGCCCGAGCGTGTGGGCGGCGGTGCCGGGCAAAACCTTTGTGAACATTACCCCGGCGGAAGTGGGCGTGCTGGTG | |
| | GGCAAAGATCGCAGCAGCTTTTATGTGAACGGCCTGACCCTGGGCGGCCAGAAATGCAGCGTGATTCGCGATAGCCTG | |
| | CTGCAGGATGGCGAATTTAGCATGGATCTGCGCACCAAAAGCACCGGCGGCGCGCCGACCTTTAACGTGACCGTGACC | |
| | AAAACCGATAAAACCCTGGTGCTGCTGATGGGCAAAGAAGGCGTGCATGGCGGCCTGATTAACAAAAAATGCTATGAA | |
| | ATGGCGAGCCATCTGCGCCGCAGCCAGTAT | |
| PROF1_HUMAN | MAGWNAYIDNLMADGTCQDAAIVGYKDSPSVWAAVPGKTFVNITPAEVGVLVGKDRSSFYVNGLTLGGQKCSVIRDSL | 32 |
| | LQDGEFSMDLRTKSTGGAPTFNVTVTKTDKTLVLLMGKEGVHGGLINKKCYEMASHLRRSQY | |
| PEDF_HUMAN | ATGCAGGCGCTGGTGCTGCTGCTGTGCATTGGCGCGCTGCTGGGCCATAGCAGCTGCCAGAACCCGGCGAGCCCGCCG | 33 |
| | GAAGAAGGCAGCCCGGATCCGGATAGCACCGGCGCGCTGGTGGAAGAAGAAGATCCGTTTTTTAAAGTGCCGGTGAAC | |
| | AAACTGGCGGCGGCGGTGAGCAACTTTGGCTATGATCTGTATCGCGTGCGCAGCAGCACCAGCCCGACCACCAACGTG | |
| | CTGCTGAGCCCGCTGAGCGTGGCGACCGCGCTGAGCGCGCTGAGCCTGGGCGCGGAACAGCGCACCGAAAGCATTATT | |
| | CATCGCGCGCTGTATTATGATCTGATTAGCAGCCCGGATATTCATGGCACCTATAAAGAACTGCTGGATACCGTGACC | |
| | GCGCCGCAGAAAAACCTGAAAAGCGCGAGCCGCATTGTGTTTGAAAAAAAACTGCGCATTAAAAGCAGCTTTGTGGCG | |
| | CCGCTGGAAAAAAGCTATGGCACCCGCCCGCGCGTGCTGACCGGCAACCCGCGCCTGGATCTGCAGGAAATTAACAAC | |
| | TGGGTGCAGGCGCAGATGAAAGGCAAACTGGCGCGCAGCACCAAAGAAATTCCGGATGAAATTAGCATTCTGCTGCTG | |
| | GGCGTGGCGCATTTTAAAGGCCAGTGGGTGACCAAATTTGATAGCCGCAAAACCAGCCTGGAAGATTTTTATCTGGAT | |
| | GAAGAACGCACCGTGCGCGTGCCGATGATGAGCGATCCGAAAGCGGTGCTGCGCTATGGCCTGGATAGCGATCTGAGC | |
| | TGCAAAATTGCGCAGCTGCCGCTGACCGGCAGCATGAGCATTATTTTTTTTCTGCCGCTGAAAGTGACCCAGAACCTG | |
| | ACCCTGATTGAAGAAAGCCTGACCAGCGAATTTATTCATGATATTGATCGCGAACTGAAAACCGTGCAGGCGGTGCTG | |
| | ACCGTGCCGAAACTGAAACTGAGCTATGAAGGCGAAGTGACCAAAAGCCTGCAGGAAATGAAACTGCAGAGCCTGTTT | |
| | GATAGCCCGGATTTTAGCAAAATTACCGGCAAACCGATTAAACTGACCCAGGTGGAACATCGCGCGGGCTTTGAATGG | |
| | AACGAAGATGGCGCGGGCACCACCCCGAGCCCGGGCCTGCAGCCGGCGCATCTGACCTTTCCGCTGGATTATCATCTG | |
| | AACCAGCCGTTTATTTTTGTGCTGCGCGATACCGATACCGGCGCGCTGCTGTTTATTGGCAAAATTCTGGATCCGCGC | |
| | GGCCCG | |
| PEDF_HUMAN | MQALVLLLCIGALLGHSSCQNPASPPEEGSPDPDSTGALVEEEDPFEKVPVNKLAAAVSNEGYDLYRVRSSTSPTTNV | 34 |
| | LLSPLSVATALSALSLGAEQRTESIIHRALYYDLISSPDIHGTYKELLDTVTAPQKNLKSASRIVFEKKLRIKSSFVA | |
| | PLEKSYGTRPRVLTGNPRLDLQEINNWVQAQMKGKLARSTKEIPDEISILLLGVAHFKGQWVTKFDSRKTSLEDFYLD | |
| | EERTVRVPMMSDPKAVLRYGLDSDLSCKIAQLPLTGSMSIIFELPLKVTQNLTLIEESLTSEFIHDIDRELKTVQAVL | |
| | TVPKLKLSYEGEVTKSLQEMKLQSLFDSPDFSKITGKPIKLTQVEHRAGFEWNEDGAGTTPSPGLQPAHLTFPLDYHL | |
| | NQPFIFVLRDTDTGALLFIGKILDPRGP | |
| LUM_HUMAN | ATGAGCCTGAGCGCGTTTACCCTGTTTCTGGCGCTGATTGGCGGCACCAGCGGCCAGTATTATGATTATGATTTTCCG | 35 |
| | CTGAGCATTTATGGCCAGAGCAGCCCGAACTGCGCGCCGGAATGCAACTGCCCGGAAAGCTATCCGAGCGCGATGTAT | |
| | TGCGATGAACTGAAACTGAAAAGCGTGCCGATGGTGCCGCCGGGCATTAAATATCTGTATCTGCGCAACAACCAGATT | |
| | GATCATATTGATGAAAAAGCGTTTGAAAACGTGACCGATCTGCAGTGGCTGATTCTGGATCATAACCTGCTGGAAAAC | |
| | AGCAAAATTAAAGGCCGCGTGTTTAGCAAACTGAAACAGCTGAAAAAACTGCATATTAACCATAACAACCTGACCGAA | |
| | AGCGTGGGCCCGCTGCCGAAAAGCCTGGAAGATCTGCAGCTGACCCATAACAAAATTACCAAACTGGGCAGCTTTGAA | |
| | GGCCTGGTGAACCTGACCTTTATTCATCTGCAGCATAACCGCCTGAAAGAAGATGCGGTGAGCGCGGCGTTTAAAGGC | |
| | CTGAAAAGCCTGGAATATCTGGATCTGAGCTTTAACCAGATTGCGCGCCTGCCGAGCGGCCTGCCGGTGAGCCTGCTG | |
| | ACCCTGTATCTGGATAACAACAAAATTAGCAACATTCCGGATGAATATTTTAAACGCTTTAACGCGCTGCAGTATCTG | |
| | CGCCTGAGCCATAACGAACTGGCGGATAGCGGCATTCCGGGCAACAGCTTTAACGTGAGCAGCCTGGTGGAACTGGAT | |
| | CTGAGCTATAACAAACTGAAAAACATTCCGACCGTGAACGAAAACCTGGAAAACTATTATCTGGAAGTGAACCAGCTG | |
| | GAAAAATTTGATATTAAAAGCTTTTGCAAAATTCTGGGCCCGCTGAGCTATAGCAAAATTAAACATCTGCGCCTGGAT | |
| | GGCAACCGCATTAGCGAAACCAGCCTGCCGCCGGATATGTATGAATGCCTGCGCGTGGCGAACGAAGTGACCCTGAAC | |
| LUM_HUMAN | MSLSAFTLFLALIGGTSGQYYDYDFPLSIYGQSSPNCAPECNCPESYPSAMYCDELKLKSVPMVPPGIKYLYLRNNQI | 36 |
| | DHIDEKAFENVTDLQWLILDHNLLENSKIKGRVFSKLKQLKKLHINHNNLTESVGPLPKSLEDLQLTHNKITKLGSFE | |
| | GLVNLTFIHLQHNRLKEDAVSAAFKGLKSLEYLDLSFNQIARLPSGLPVSLLTLYLDNNKISNIPDEYFKRFNALQYL | |
| | RLSHNELADSGIPGNSFNVSSLVELDLSYNKLKNIPTVNENLENYYLEVNQLEKEDIKSFCKILGPLSYSKIKHLRLD | |
| | GNRISETSLPPDMYECLRVANEVTLN | |
| C163A_HUMAN | ATGAGCAAACTGCGCATGGTGCTGCTGGAAGATAGCGGCAGCGCGGATTTTCGCCGCCATTTTGTGAACCTGAGCCCG | 37 |
| | TTTACCATTACCGTGGTGCTGCTGCTGAGCGCGTGCTTTGTGACCAGCAGCCTGGGCGGCACCGATAAAGAACTGCGC | |
| | CTGGTGGATGGCGAAAACAAATGCAGCGGCCGCGTGGAAGTGAAAGTGCAGGAAGAATGGGGCACCGTGTGCAACAAC | |
| | GGCTGGAGCATGGAAGCGGTGAGCGTGATTTGCAACCAGCTGGGCTGCCCGACCGCGATTAAAGCGCCGGGCTGGGCG | |
| | AACAGCAGCGCGGGCAGCGGCCGCATTTGGATGGATCATGTGAGCTGCCGCGGCAACGAAAGCGCGCTGTGGGATTGC | |
| | AAACATGATGGCTGGGGCAAACATAGCAACTGCACCCATCAGCAGGATGCGGGCGTGACCTGCAGCGATGGCAGCAAC | |
| | CTGGAAATGCGCCTGACCCGCGGCGGCAACATGTGCAGCGGCCGCATTGAAATTAAATTTCAGGGCCGCTGGGGCACC | |
| | GTGTGCGATGATAACTTTAACATTGATCATGCGAGCGTGATTTGCCGCCAGCTGGAATGCGGCAGCGCGGTGAGCTTT | |
| | AGCGGCAGCAGCAACTTTGGCGAAGGCAGCGGCCCGATTTGGTTTGATGATCTGATTTGCAACGGCAACGAAAGCGCG | |
| | CTGTGGAACTGCAAACATCAGGGCTGGGGCAAACATAACTGCGATCATGCGGAAGATGCGGGCGTGATTTGCAGCAAA | |
| | GGCGCGGATCTGAGCCTGCGCCTGGTGGATGGCGTGACCGAATGCAGCGGCCGCCTGGAAGTGCGCTTTCAGGGCGAA | |
| | TGGGGCACCATTTGCGATGATGGCTGGGATAGCTATGATGCGGCGGTGGCGTGCAAACAGCTGGGCTGCCCGACCGCG | |
| | GTGACCGCGATTGGCCGCGTGAACGCGAGCAAAGGCTTTGGCCATATTTGGCTGGATAGCGTGAGCTGCCAGGGCCAT | |
| | GAACCGGCGATTTGGCAGTGCAAACATCATGAATGGGGCAAACATTATTGCAACCATAACGAAGATGCGGGCGTGACC | |
| | TGCAGCGATGGCAGCGATCTGGAACTGCGCCTGCGCGGCGGCGGCAGCCGCTGCGCGGGCACCGTGGAAGTGGAAATT | |
| | CAGCGCCTGCTGGGCAAAGTGTGCGATCGCGGCTGGGGCCTGAAAGAAGCGGATGTGGTGTGCCGCCAGCTGGGCTGC | |
| | GGCAGCGCGCTGAAAACCAGCTATCAGGTGTATAGCAAAATTCAGGCGACCAACACCTGGCTGTTTCTGAGCAGCTGC | |
| | AACGGCAACGAAACCAGCCTGTGGGATTGCAAAAACTGGCAGTGGGGCGGCCTGACCTGCGATCATTATGAAGAAGCG | |
| | AAAATTACCTGCAGCGCGCATCGCGAACCGCGCCTGGTGGGCGGCGATATTCCGTGCAGCGGCCGCGTGGAAGTGAAA | |
| | CATGGCGATACCTGGGGCAGCATTTGCGATAGCGATTTTAGCCTGGAAGCGGCGAGCGTGCTGTGCCGCGAACTGCAG | |
| | TGCGGCACCGTGGTGAGCATTCTGGGCGGCGCGCATTTTGGCGAAGGCAACGGCCAGATTTGGGCGGAAGAATTTCAG | |
| | TGCGAAGGCCATGAAAGCCATCTGAGCCTGTGCCCGGTGGCGCCGCGCCCGGAAGGCACCTGCAGCCATAGCCGCGAT | |
| | GTGGGCGTGGTGTGCAGCCGCTATACCGAAATTCGCCTGGTGAACGGCAAAACCCCGTGCGAAGGCCGCGTGGAACTG | |
| | AAAACCCTGGGCGCGTGGGGCAGCCTGTGCAACAGCCATTGGGATATTGAAGATGCGCATGTGCTGTGCCAGCAGCTG | |
| | AAATGCGGCGTGGCGCTGAGCACCCCGGGCGGCGCGCGCTTTGGCAAAGGCAACGGCCAGATTTGGCGCCATATGTTT | |
| | CATTGCACCGGCACCGAACAGCATATGGGCGATTGCCCGGTGACCGCGCTGGGCGCGAGCCTGTGCCCGAGCGAACAG | |
| | GTGGCGAGCGTGATTTGCAGCGGCAACCAGAGCCAGACCCTGAGCAGCTGCAACAGCAGCAGCCTGGGCCCGACCCGC | |
| | CCGACCATTCCGGAAGAAAGCGCGGTGGCGTGCATTGAAAGCGGCCAGCTGCGCCTGGTGAACGGCGGCGGCCGCTGC | |
| | GCGGGCCGCGTGGAAATTTATCATGAAGGCAGCTGGGGCACCATTTGCGATGATAGCTGGGATCTGAGCGATGCGCAT | |
| | GTGGTGTGCCGCCAGCTGGGCTGCGGCGAAGCGATTAACGCGACCGGCAGCGCGCATTTTGGCGAAGGCACCGGCCCG | |
| | ATTTGGCTGGATGAAATGAAATGCAACGGCAAAGAAAGCCGCATTTGGCAGTGCCATAGCCATGGCTGGGGCCAGCAG | |
| | AACTGCCGCCATAAAGAAGATGCGGGCGTGATTTGCAGCGAATTTATGAGCCTGCGCCTGACCAGCGAAGCGAGCCGC | |
| | GAAGCGTGCGCGGGCCGCCTGGAAGTGTTTTATAACGGCGCGTGGGGCACCGTGGGCAAAAGCAGCATGAGCGAAACC | |
| | ACCGTGGGCGTGGTGTGCCGCCAGCTGGGCTGCGCGGATAAAGGCAAAATTAACCCGGCGAGCCTGGATAAAGCGATG | |
| | AGCATTCCGATGTGGGTGGATAACGTGCAGTGCCCGAAAGGCCCGGATACCCTGTGGCAGTGCCCGAGCAGCCCGTGG | |
| | GAAAAACGCCTGGCGAGCCCGAGCGAAGAAACCTGGATTACCTGCGATAACAAAATTCGCCTGCAGGAAGGCCCGACC | |
| | AGCTGCAGCGGCCGCGTGGAAATTTGGCATGGCGGCAGCTGGGGCACCGTGTGCGATGATAGCTGGGATCTGGATGAT | |
| | GCGCAGGTGGTGTGCCAGCAGCTGGGCTGCGGCCCGGCGCTGAAAGCGTTTAAAGAAGCGGAATTTGGCCAGGGCACC | |
| | GGCCCGATTTGGCTGAACGAAGTGAAATGCAAAGGCAACGAAAGCAGCCTGTGGGATTGCCCGGCGCGCCGCTGGGGC | |
| | CATAGCGAATGCGGCCATAAAGAAGATGCGGCGGTGAACTGCACCGATATTAGCGTGCAGAAAACCCCGCAGAAAGCG | |
| | ACCACCGGCCGCAGCAGCCGCCAGAGCAGCTTTATTGCGGTGGGCATTCTGGGCGTGGTGCTGCTGGCGATTTTTGTG | |
| | GCGCTGTTTTTTCTGACCAAAAAACGCCGCCAGCGCCAGCGCCTGGCGGTGAGCAGCCGCGGCGAAAACCTGGTGCAT | |
| | CAGATTCAGTATCGCGAAATGAACAGCTGCCTGAACGCGGATGATCTGGATCTGATGAACAGCAGCGAAAACAGCCAT | |
| | GAAAGCGCGGATTTTAGCGCGGCGGAACTGATTAGCGTGAGCAAATTTCTGCCGATTAGCGGCATGGAAAAAGAAGCG | |
| | ATTCTGAGCCATACCGAAAAAGAAAACGGCAACCTG | |
| C163A_HUMAN | MSKLRMVLLEDSGSADERRHEVNLSPFTITVVLLLSACFVTSSLGGTDKELRLVDGENKCSGRVEVKVQEEWGTVCNN | 38 |
| | GWSMEAVSVICNQLGCPTAIKAPGWANSSAGSGRIWMDHVSCRGNESALWDCKHDGWGKHSNCTHQQDAGVTCSDGSN | |
| | LEMRLTRGGNMCSGRIEIKFQGRWGTVCDDNFNIDHASVICRQLECGSAVSFSGSSNFGEGSGPIWFDDLICNGNESA | |
| | LWNCKHQGWGKHNCDHAEDAGVICSKGADLSLRLVDGVTECSGRLEVRFQGEWGTICDDGWDSYDAAVACKQLGCPTA | |
| | VTAIGRVNASKGFGHIWLDSVSCQGHEPAIWQCKHHEWGKHYCNHNEDAGVTCSDGSDLELRLRGGGSRCAGTVEVEI | |
| | QRLLGKVCDRGWGLKEADVVCRQLGCGSALKTSYQVYSKIQATNTWLFLSSCNGNETSLWDCKNWQWGGLTCDHYEEA | |
| | KITCSAHREPRLVGGDIPCSGRVEVKHGDTWGSICDSDFSLEAASVLCRELQCGTVVSILGGAHFGEGNGQIWAEEFQ | |
| | CEGHESHLSLCPVAPRPEGTCSHSRDVGVVCSRYTEIRLVNGKTPCEGRVELKTLGAWGSLCNSHWDIEDAHVLCQQL | |
| | KCGVALSTPGGARFGKGNGQIWRHMFHCTGTEQHMGDCPVTALGASLCPSEQVASVICSGNQSQTLSSCNSSSLGPTR | |
| | PTIPEESAVACIESGQLRLVNGGGRCAGRVEIYHEGSWGTICDDSWDLSDAHVVCRQLGCGEAINATGSAHFGEGTGP | |
| | IWLDEMKCNGKESRIWQCHSHGWGQQNCRHKEDAGVICSEFMSLRLTSEASREACAGRLEVFYNGAWGTVGKSSMSET | |
| | TVGVVCRQLGCADKGKINPASLDKAMSIPMWVDNVQCPKGPDTLWQCPSSPWEKRLASPSEETWITCDNKIRLQEGPT | |
| | SCSGRVEIWHGGSWGTVCDDSWDLDDAQVVCQQLGCGPALKAFKEAEFGQGTGPIWLNEVKCKGNESSLWDCPARRWG | |
| | HSECGHKEDAAVNCTDISVQKTPQKATTGRSSRQSSFIAVGILGVVLLAIEVALFFLTKKRRQRQRLAVSSRGENLVH | |
| | QIQYREMNSCLNADDLDLMNSSENSHESADFSAAELISVSKFLPISGMEKEAILSHTEKENGNL | |
| PTPRJ_HUMAN | ATGAAACCGGCGGCGCGCGAAGCGCGCCTGCCGCCGCGCAGCCCGGGCCTGCGCTGGGCGCTGCCGCTGCTGCTGCTG | 39 |
| | CTGCTGCGCCTGGGCCAGATTCTGTGCGCGGGCGGCACCCCGAGCCCGATTCCGGATCCGAGCGTGGCGACCGTGGCG | |
| | ACCGGCGAAAACGGCATTACCCAGATTAGCAGCACCGCGGAAAGCTTTCATAAACAGAACGGCACCGGCACCCCGCAG | |
| | GTGGAAACCAACACCAGCGAAGATGGCGAAAGCAGCGGCGCGAACGATAGCCTGCGCACCCCGGAACAGGGCAGCAAC | |
| | GGCACCGATGGCGCGAGCCAGAAAACCCCGAGCAGCACCGGCCCGAGCCCGGTGTTTGATATTAAAGCGGTGAGCATT | |
| | AGCCCGACCAACGTGATTCTGACCTGGAAAAGCAACGATACCGCGGCGAGCGAATATAAATATGTGGTGAAACATAAA | |
| | ATGGAAAACGAAAAAACCATTACCGTGGTGCATCAGCCGTGGTGCAACATTACCGGCCTGCGCCCGGCGACCAGCTAT | |
| | GTGTTTAGCATTACCCCGGGCATTGGCAACGAAACCTGGGGCGATCCGCGCGTGATTAAAGTGATTACCGAACCGATT | |
| | CCGGTGAGCGATCTGCGCGTGGCGCTGACCGGCGTGCGCAAAGCGGCGCTGAGCTGGAGCAACGGCAACGGCACCGCG | |
| | AGCTGCCGCGTGCTGCTGGAAAGCATTGGCAGCCATGAAGAACTGACCCAGGATAGCCGCCTGCAGGTGAACATTAGC | |
| | GGCCTGAAACCGGGCGTGCAGTATAACATTAACCCGTATCTGCTGCAGAGCAACAAAACCAAAGGCGATCCGCTGGGC | |
| | ACCGAAGGCGGCCTGGATGCGAGCAACACCGAACGCAGCCGCGCGGGCAGCCCGACCGCGCCGGTGCATGATGAAAGC | |
| | CTGGTGGGCCCGGTGGATCCGAGCAGCGGCCAGCAGAGCCGCGATACCGAAGTGCTGCTGGTGGGCCTGGAACCGGGC | |
| | ACCCGCTATAACGCGACCGTGTATAGCCAGGCGGCGAACGGCACCGAAGGCCAGCCGCAGGCGATTGAATTTCGCACC | |
| | AACGCGATTCAGGTGTTTGATGTGACCGCGGTGAACATTAGCGCGACCAGCCTGACCCTGATTTGGAAAGTGAGCGAT | |
| | AACGAAAGCAGCAGCAACTATACCTATAAAATTCATGTGGCGGGCGAAACCGATAGCAGCAACCTGAACGTGAGCGAA | |
| | CCGCGCGCGGTGATTCCGGGCCTGCGCAGCAGCACCTTTTATAACATTACCGTGTGCCCGGTGCTGGGCGATATTGAA | |
| | GGCACCCCGGGCTTTCTGCAGGTGCATACCCCGCCGGTGCCGGTGAGCGATTTTCGCGTGACCGTGGTGAGCACCACC | |
| | GAAATTGGCCTGGCGTGGAGCAGCCATGATGCGGAAAGCTTTCAGATGCATATTACCCAGGAAGGCGCGGGCAACAGC | |
| | CGCGTGGAAATTACCACCAACCAGAGCATTATTATTGGCGGCCTGTTTCCGGGCACCAAATATTGCTTTGAAATTGTG | |
| | CCGAAAGGCCCGAACGGCACCGAAGGCGCGAGCCGCACCGTGTGCAACCGCACCGTGCCGAGCGCGGTGTTTGATATT | |
| | CATGTGGTGTATGTGACCACCACCGAAATGTGGCTGGATTGGAAAAGCCCGGATGGCGCGAGCGAATATGTGTATCAT | |
| | CTGGTGATTGAAAGCAAACATGGCAGCAACCATACCAGCACCTATGATAAAGCGATTACCCTGCAGGGCCTGATTCCG | |
| | GGCACCCTGTATAACATTACCATTAGCCCGGAAGTGGATCATGTGTGGGGCGATCCGAACAGCACCGCGCAGTATACC | |
| | CGCCCGAGCAACGTGAGCAACATTGATGTGAGCACCAACACCACCGCGGCGACCCTGAGCTGGCAGAACTTTGATGAT | |
| | GCGAGCCCGACCTATAGCTATTGCCTGCTGATTGAAAAAGCGGGCAACAGCAGCAACGCGACCCAGGTGGTGACCGAT | |
| | ATTGGCATTACCGATGCGACCGTGACCGAACTGATTCCGGGCAGCAGCTATACCGTGGAAATTTTTGCGCAGGTGGGC | |
| | GATGGCATTAAAAGCCTGGAACCGGGCCGCAAAAGCTTTTGCACCGATCCGGCGAGCATGGCGAGCTTTGATTGCGAA | |
| | GTGGTGCCGAAAGAACCGGCGCTGGTGCTGAAATGGACCTGCCCGCCGGGCGCGAACGCGGGCTTTGAACTGGAAGTG | |
| | AGCAGCGGCGCGTGGAACAACGCGACCCATCTGGAAAGCTGCAGCAGCGAAAACGGCACCGAATATCGCACCGAAGTG | |
| | ACCTATCTGAACTTTAGCACCAGCTATAACATTAGCATTACCACCGTGAGCTGCGGCAAAATGGCGGCGCCGACCCGC | |
| | AACACCTGCACCACCGGCATTACCGATCCGCCGCCGCCGGATGGCAGCCCGAACATTACCAGCGTGAGCCATAACAGC | |
| | GTGAAAGTGAAATTTAGCGGCTTTGAAGCGAGCCATGGCCCGATTAAAGCGTATGCGGTGATTCTGACCACCGGCGAA | |
| | GCGGGCCATCCGAGCGCGGATGTGCTGAAATATACCTATGAAGATTTTAAAAAAGGCGCGAGCGATACCTATGTGACC | |
| | TATCTGATTCGCACCGAAGAAAAAGGCCGCAGCCAGAGCCTGAGCGAAGTGCTGAAATATGAAATTGATGTGGGCAAC | |
| | GAAAGCACCACCCTGGGCTATTATAACGGCAAACTGGAACCGCTGGGCAGCTATCGCGCGTGCGTGGCGGGCTTTACC | |
| | AACATTACCTTTCATCCGCAGAACAAAGGCCTGATTGATGGCGCGGAAAGCTATGTGAGCTTTAGCCGCTATAGCGAT | |
| | GCGGTGAGCCTGCCGCAGGATCCGGGCGTGATTTGCGGCGCGGTGTTTGGCTGCATTTTTGGCGCGCTGGTGATTGTG | |
| | ACCGTGGGCGGCTTTATTTTTTGGCGCAAAAAACGCAAAGATGCGAAAAACAACGAAGTGAGCTTTAGCCAGATTAAA | |
| | CCGAAAAAAAGCAAACTGATTCGCGTGGAAAACTTTGAAGCGTATTTTAAAAAACAGCAGGCGGATAGCAACTGCGGC | |
| | TTTGCGGAAGAATATGAAGATCTGAAACTGGTGGGCATTAGCCAGCCGAAATATGCGGCGGAACTGGCGGAAAACCGC | |
| | GGCAAAAACCGCTATAACAACGTGCTGCCGTATGATATTAGCCGCGTGAAACTGAGCGTGCAGACCCATAGCACCGAT | |
| | GATTATATTAACGCGAACTATATGCCGGGCTATCATAGCAAAAAAGATTTTATTGCGACCCAGGGCCCGCTGCCGAAC | |
| | ACCCTGAAAGATTTTTGGCGCATGGTGTGGGAAAAAAACGTGTATGCGATTATTATGCTGACCAAATGCGTGGAACAG | |
| | GGCCGCACCAAATGCGAAGAATATTGGCCGAGCAAACAGGCGCAGGATTATGGCGATATTACCGTGGCGATGACCAGC | |
| | GAAATTGTGCTGCCGGAATGGACCATTCGCGATTTTACCGTGAAAAACATTCAGACCAGCGAAAGCCATCCGCTGCGC | |
| | CAGTTTCATTTTACCAGCTGGCCGGATCATGGCGTGCCGGATACCACCGATCTGCTGATTAACTTTCGCTATCTGGTG | |
| | CGCGATTATATGAAACAGAGCCCGCCGGAAAGCCCGATTCTGGTGCATTGCAGCGCGGGCGTGGGCCGCACCGGCACC | |
| | TTTATTGCGATTGATCGCCTGATTTATCAGATTGAAAACGAAAACACCGTGGATGTGTATGGCATTGTGTATGATCTG | |
| | CGCATGCATCGCCCGCTGATGGTGCAGACCGAAGATCAGTATGTGTTTCTGAACCAGTGCGTGCTGGATATTGTGCGC | |
| | AGCCAGAAAGATAGCAAAGTGGATCTGATTTATCAGAACACCACCGCGATGACCATTTATGAAAACCTGGCGCCGGTG | |
| | ACCACCTTTGGCAAAACCAACGGCTATATTGCG | |
| PTPRJ_HUMAN | MKPAAREARLPPRSPGLRWALPLLLLLLRLGQILCAGGTPSPIPDPSVATVATGENGITQISSTAESFHKQNGTGTPQ | 40 |
| | VETNTSEDGESSGANDSLRTPEQGSNGTDGASQKTPSSTGPSPVFDIKAVSISPTNVILTWKSNDTAASEYKYVVKHK | |
| | MENEKTITVVHQPWCNITGLRPATSYVFSITPGIGNETWGDPRVIKVITEPIPVSDLRVALTGVRKAALSWSNGNGTA | |
| | SCRVLLESIGSHEELTQDSRLQVNISGLKPGVQYNINPYLLQSNKTKGDPLGTEGGLDASNTERSRAGSPTAPVHDES | |
| | LVGPVDPSSGQQSRDTEVLLVGLEPGTRYNATVYSQAANGTEGQPQAIEFRTNAIQVFDVTAVNISATSLTLIWKVSD | |
| | NESSSNYTYKIHVAGETDSSNLNVSEPRAVIPGLRSSTFYNITVCPVLGDIEGTPGFLQVHTPPVPVSDERVTVVSTT | |
| | EIGLAWSSHDAESFQMHITQEGAGNSRVEITTNQSIIIGGLFPGTKYCFEIVPKGPNGTEGASRTVCNRTVPSAVEDI | |
| | HVVYVTTTEMWLDWKSPDGASEYVYHLVIESKHGSNHTSTYDKAITLQGLIPGTLYNITISPEVDHVWGDPNSTAQYT | |
| | RPSNVSNIDVSTNTTAATLSWQNFDDASPTYSYCLLIEKAGNSSNATQVVTDIGITDATVTELIPGSSYTVEIFAQVG | |
| | DGIKSLEPGRKSFCTDPASMASFDCEVVPKEPALVLKWTCPPGANAGFELEVSSGAWNNATHLESCSSENGTEYRTEV | |
| | TYLNESTSYNISITTVSCGKMAAPTRNTCTTGITDPPPPDGSPNITSVSHNSVKVKFSGFEASHGPIKAYAVILTTGE | |
| | AGHPSADVLKYTYEDFKKGASDTYVTYLIRTEEKGRSQSLSEVLKYEIDVGNESTTLGYYNGKLEPLGSYRACVAGFT | |
| | NITFHPQNKGLIDGAESYVSFSRYSDAVSLPQDPGVICGAVFGCIFGALVIVTVGGFIFWRKKRKDAKNNEVSFSQIK | |
| | PKKSKLIRVENFEAYFKKQQADSNCGFAEEYEDLKLVGISQPKYAAELAENRGKNRYNNVLPYDISRVKLSVQTHSTD | |
| | DYINANYMPGYHSKKDFIATQGPLPNTLKDFWRMVWEKNVYAIIMLTKCVEQGRTKCEEYWPSKQAQDYGDITVAMTS | |
| | EIVLPEWTIRDFTVKNIQTSESHPLRQFHFTSWPDHGVPDTTDLLINFRYLVRDYMKQSPPESPILVHCSAGVGRTGT | |
| | FIAIDRLIYQIENENTVDVYGIVYDLRMHRPLMVQTEDQYVFLNQCVLDIVRSQKDSKVDLIYQNTTAMTIYENLAPV | |
| | TTFGKTNGYIA | |
| ALDOA_HUMAN | ATGCCGTATCAGTATCCGGCGCTGACCCCGGAACAGAAAAAAGAACTGAGCGATATTGCGCATCGCATTGTGGCGCCG | 41 |
| | GGCAAAGGCATTCTGGCGGCGGATGAAAGCACCGGCAGCATTGCGAAACGCCTGCAGAGCATTGGCACCGAAAACACC | |
| | GAAGAAAACCGCCGCTTTTATCGCCAGCTGCTGCTGACCGCGGATGATCGCGTGAACCCGTGCATTGGCGGCGTGATT | |
| | CTGTTTCATGAAACCCTGTATCAGAAAGCGGATGATGGCCGCCCGTTTCCGCAGGTGATTAAAAGCAAAGGCGGCGTG | |
| | GTGGGCATTAAAGTGGATAAAGGCGTGGTGCCGCTGGCGGGCACCAACGGCGAAACCACCACCCAGGGCCTGGATGGC | |
| | CTGAGCGAACGCTGCGCGCAGTATAAAAAAGATGGCGCGGATTTTGCGAAATGGCGCTGCGTGCTGAAAATTGGCGAA | |
| | CATACCCCGAGCGCGCTGGCGATTATGGAAAACGCGAACGTGCTGGCGCGCTATGCGAGCATTTGCCAGCAGAACGGC | |
| | ATTGTGCCGATTGTGGAACCGGAAATTCTGCCGGATGGCGATCATGATCTGAAACGCTGCCAGTATGTGACCGAAAAA | |
| | GTGCTGGCGGCGGTGTATAAAGCGCTGAGCGATCATCATATTTATCTGGAAGGCACCCTGCTGAAACCGAACATGGTG | |
| | ACCCCGGGCCATGCGTGCACCCAGAAATTTAGCCATGAAGAAATTGCGATGGCGACCGTGACCGCGCTGCGCCGCACC | |
| | GTGCCGCCGGCGGTGACCGGCATTACCTTTCTGAGCGGCGGCCAGAGCGAAGAAGAAGCGAGCATTAACCTGAACGCG | |
| | ATTAACAAATGCCCGCTGCTGAAACCGTGGGCGCTGACCTTTAGCTATGGCCGCGCGCTGCAGGCGAGCGCGCTGAAA | |
| | GCGTGGGGCGGCAAAAAAGAAAACCTGAAAGCGGCGCAGGAAGAATATGTGAAACGCGCGCTGGCGAACAGCCTGGCG | |
| | TGCCAGGGCAAATATACCCCGAGCGGCCAGGCGGGCGCGGCGGCGAGCGAAAGCCTGTTTGTGAGCAACCATGCGTAT | |
| ALDOA_HUMAN | MPYQYPALTPEQKKELSDIAHRIVAPGKGILAADESTGSIAKRLQSIGTENTEENRRFYRQLLLTADDRVNPCIGGVI | 42 |
| | LFHETLYQKADDGRPFPQVIKSKGGVVGIKVDKGVVPLAGTNGETTTQGLDGLSERCAQYKKDGADFAKWRCVLKIGE | |
| | HTPSALAIMENANVLARYASICQQNGIVPIVEPEILPDGDHDLKRCQYVTEKVLAAVYKALSDHHIYLEGTLLKPNMV | |
| | TPGHACTQKFSHEEIAMATVTALRRTVPPAVTGITFLSGGQSEEEASINLNAINKCPLLKPWALTFSYGRALQASALK | |
| | AWGGKKENLKAAQEEYVKRALANSLACQGKYTPSGQAGAAASESLFVSNHAY | |
| FRIL_HUMAN | AGCAGCCAGATTCGCCAGAACTATAGCACCGATGTGGAAGCGGCGGTGAACAGCCTGGTGAACCTGTATCTGCAGGCG | 43 |
| | AGCTATACCTATCTGAGCCTGGGCTTTTATTTTGATCGCGATGATGTGGCGCTGGAAGGCGTGAGCCATTTTTTTCGC | |
| | GAACTGGCGGAAGAAAAACGCGAAGGCTATGAACGCCTGCTGAAAATGCAGAACCAGCGCGGCGGCCGCGCGCTGTTT | |
| | CAGGATATTAAAAAACCGGCGGAAGATGAATGGGGCAAAACCCCGGATGCGATGAAAGCGGCGATGGCGCTGGAAAAA | |
| | AAACTGAACCAGGCGCTGCTGGATCTGCATGCGCTGGGCAGCGCGCGCACCGATCCGCATCTGTGCGATTTTCTGGAA | |
| | ACCCATTTTCTGGATGAAGAAGTGAAACTGATTAAAAAAATGGGCGATCATCTGACCAACCTGCATCGCCTGGGCGGC | |
| | CCGGAAGCGGGCCTGGGCGAATATCTGTTTGAACGCCTGACCCTGAAACATGAT | |
| FRIL_HUMAN | MSSQIRQNYSTDVEAAVNSLVNLYLQASYTYLSLGFYFDRDDVALEGVSHFFRELAEEKREGYERLLKMQNQRGGRAL | 44 |
| | FQDIKKPAEDEWGKTPDAMKAAMALEKKLNQALLDLHALGSARTDPHLCDFLETHFLDEEVKLIKKMGDHLTNLHRLG | |
| | GPEAGLGEYLFERLTLKHD | |

1. A method of determining that a lung condition in a subject is cancer comprising:
(a) contacting a biological sample obtained from the subject with a proteolytic enzyme to produce peptide fragments from a panel of proteins present in the biological sample, wherein the panel comprises GGH_HUMAN (SEQ ID NO.: 4), ALDOA_HUMAN (SEQ ID NO.: 42), FRIL_HUMAN (SEQ ID NO.: 44), KIT_HUMAN (SEQ ID NO.: 30), and TSP1_HUMAN (SEQ ID NO.: 10);
(b) combining the produced peptide fragments from the panel from step (a) with labeled, synthetic peptide fragments which correspond to the produced peptide fragments from the panel;
(c) performing selected reaction monitoring mass spectrometry to measure the abundance of the peptide fragments from step (b), thereby determining the protein expression level of each of GGH_HUMAN (SEQ ID NO.: 4), ALDOA_HUMAN (SEQ ID NO.: 42), FRIL_HUMAN (SEQ ID NO.: 44), KIT_HUMAN (SEQ ID NO.: 30), and TSP1_HUMAN (SEQ ID NO.: 10);
(d) calculating a score based on the peptide fragment measurements of step (c); and
(e) determining that the lung condition is cancer if the score is equal or greater than a predetermined score.

2. The method of claim 1, wherein the subject has a pulmonary nodule.

3. The method of claim 2, wherein the pulmonary nodule is 30 mm or less.

4. The method of claim 3, wherein the pulmonary nodule is between 8-30 mm.

5. The method of claim 1, wherein said lung condition is cancer or a non-cancerous lung condition.

6. The method of claim 1, wherein said cancer is non-small cell lung cancer.

7. The method of claim 1, wherein said non-cancerous lung condition is chronic obstructive pulmonary disease, hamartoma, fibroma, neurofibroma, granuloma, sarcoidosis, bacterial infection or fungal infection.

8. The method of claim 1, wherein the subject is a human.

9. The method of claim 1, wherein said biological sample is tissue, blood, plasma, serum, whole blood, urine, saliva, genital secretions, cerebrospinal fluid, sweat, excreta, or bronchoalveolar lavage.

10. The method of claim 1, wherein the proteolytic enzyme is trypsin.

11. The method of claim 1, wherein at least one transition for each peptide is determined by liquid chromatography-selected reaction monitoring/mass spectrometry (LC-SRM-MS).

12. The method of claim 11, wherein the peptide transitions comprise at least YYIAASYVK (SEQ ID No.: 51) (539.28, 638.4), ALQASALK (SEQ ID No.: 45) (401.25, 617.4), LGG-PEAGLGEYLFER (SEQ ID No.: 50) (804.4, 1083.6), YVSELHLTR (SEQ ID No.: 55) (373.21, 428.3), and GFLLLASLR (SEQ ID No.: 61) (495.31, 559.4).

13. The method of claim 1, wherein said score is determined as score=1/[1+exp(−α−Σᵢ₌₁⁵βᵢ*\check{P}ᵢ)], wherein
$${\tilde{P}}_{i} = \frac{{P}_{i}^{{λ}_{i}} - 1.0}{{λ}_{i}} ,$$
and \check{P}ᵢ is the Box-Cox transformed and normalized intensity of peptide transition i in said sample, βᵢ is the corresponding logistic regression coefficient,) λᵢ is the corresponding Box-Cox transformation, α is a panel-specific constant, and N is the total number of transitions of the assessed proteins.

14. The method of claim 1, wherein the pre-determined score is calculated from a reference population comprising at least 100 subjects with a lung condition and wherein each subject in the reference population has been assigned a score based on the protein expression of at least each of GGH_HUMAN (SEQ ID NO.: 4), ALDOA_HUMAN (SEQ ID NO.: 42), FRIL_HUMAN (SEQ ID NO.: 44), KIT_HUMAN (SEQ ID NO.: 30), and TSP1_HUMAN (SEQ ID NO.: 10) obtained from a biological sample.

15. The method of claim 1, further comprising normalizing the protein expression level of at least each of GGH_HUMAN (SEQ ID NO.: 4), ALDOA_HUMAN (SEQ ID NO.: 42), FRIL_HUMAN (SEQ ID NO.: 44), KIT_HUMAN (SEQ ID NO.: 30), and TSP1_HUMAN (SEQ ID NO.: 10) against the protein expression level of at least one of PEDF_HUMAN (SEQ ID NO.: 34), MASP1_HUMAN (SEQ ID NO.: 24), GELS_HUMAN (SEQ ID NO.: 22), LUM_HUMAN (SEQ ID NO.: 36), C163A_HUMAN (SEQ ID NO.: 38), PTPRJ_HUMAN (SEQ ID NO.: 40), CD44 HUMAN (SEQ ID NO.: 12), TENX_HUMAN (SEQ ID NO.: 16), CLUS_HUMAN (SEQ ID NO.: 18), and IBP3_HUMAN (SEQ ID NO.: 20) in the sample.

16. The method of claim 1, wherein the score from the biological sample from the subject is calculated from a logistic regression model applied to the determined protein expression levels.

17. The method of claim 1, wherein the pre-determined score is determined from a plurality of scores obtained from a reference population.

18. The method of claim 1, wherein the score is within a range of possible values and the predetermined score is approximately 65% of the magnitude of the range.

19. The method of claim 1, wherein the score from the biological sample provides a positive predictive value (PPV) of at least 30%.

20. The method of claim 1, wherein the score from the biological sample provides a positive predictive value (PPV) of at least 50%.

21. The method of claim 1, further comprising treating the subject if the lung condition is cancer.

22. The method of claim 21, wherein said treatment is a pulmonary function test (PFT), pulmonary imaging, a biopsy, a surgery, a chemotherapy, a radiotherapy, or any combination thereof.

23. The method of claim 22, where said imaging is an x-ray, a chest computed tomography (CT) scan, or a positron emission tomography (PET) scan.

24. The method of claim 1, wherein at least one step is performed on a computer system.
